(19) United States
(12) Patent Application Publication
(10) Pub. No.: US 2026/0125389 A1
(43) Pub. Date: May 7, 2026

(54) MEMBRANE-ASSOCIATED TYROSINE AND THREONINE KINASE INHIBITOR AND USE THEREOF

(71) Applicant: HAINAN SIMCERE ZAIMING PHARMACEUTICAL CO., LTD., Haikou, Hainan (CN)

(72) Inventors: Dongxing ZHU, Shanghai (CN); Jianbin XUE, Shanghai (CN); Weikun WANG, Shanghai (CN); Lang LIU, Shanghai (CN); Xiang HUAN, Shanghai (CN); Yike ZHOU, Shanghai (CN); Tao WANG, Shanghai (CN); Wei ZHU, Shanghai (CN); Zhengtao LI, Shanghai (CN)

(21) Appl. No.: 19/114,329

(22) PCT Filed: Sep. 22, 2023
(86) PCT No.: PCT/CN2023/120664

§ 371 Date: Mar. 21, 2025

(30) Foreign Application Priority Data
Sep. 23, 2022 (CN) 202211163314.4
May 6, 2023 (CN) 202310505509.0
Jun. 30, 2023 (CN) 202310799876.6
Aug. 17, 2023 (CN) 202311040531.9

(51) Int. Cl. C07D 487/16; A61K 31/519; A61P 35/00; C07D 471/22; C07D 487/22; C07D 513/22

(52) U.S. Cl. CPC C07D 487/16; A61K 31/519; A61P 35/00; C07D 471/22; C07D 487/22; C07D 513/22

(57) ABSTRACT

The present disclosure relates to a PKMYT1 inhibitor compound represented by formula (I) or a pharmaceutically acceptable salt thereof, a preparation method therefor, a pharmaceutical composition containing the compound or the pharmaceutically acceptable salt thereof, and use thereof in preventing or treating a PKMYT1-related disease. X¹ to X⁵, R¹ to R⁴, R⁷, and R⁸ in formula (I) are as defined in the specification.

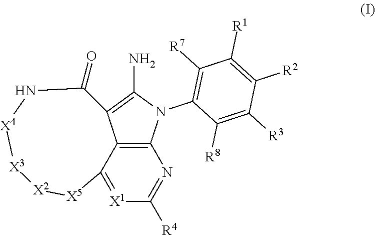

### CROSS-REFERENCE TO RELATED APPLICATION

The present application claims the benefit and priority to the following 4 Chinese Patent Applications, the contents of which are incorporated herein by reference in their entirety:
Chinese Patent Application No. 202211163314.4 filed with China National Intellectual Property Administration on Sep. 23, 2022;
Chinese Patent Application No. 202310505509.0 filed with China National Intellectual Property Administration on May 6, 2023;
Chinese Patent Application No. 202310799876.6 filed with China National Intellectual Property Administration on Jun. 30, 2023; and
Chinese Patent Application No. 202311040531.9 filed with China National Intellectual Property Administration on Aug. 17, 2023.

### TECHNICAL FIELD

The present application belongs to the field of medicines, and relates to a fused-ring compound or a pharmaceutically acceptable salt thereof serving as a PKMYT1 inhibitor, a preparation method therefor, a pharmaceutical composition containing the compound or the pharmaceutically acceptable salt thereof, and use thereof in preventing or treating diseases related to PKMYT1.

### BACKGROUND

The genomic DNA of cells is continuously exposed to various harmful factors originating from internal or external sources, which may cause DNA damage. Therefore, cells have evolved a series of complex DNA damage response mechanisms to counteract these harmful factors, thereby maintaining genomic integrity and preventing the occurrence of diseases, including tumors, caused by genomic instability. The activation of cell cycle checkpoint pathways is one of these important mechanisms. Cell cycle checkpoints include G1, S, G2, and M phase checkpoints. Unlike normal cells, the survival of tumor cells often relies on the dysregulation of DNA damage repair mechanisms. Many tumor cells, due to mutations in the p53 gene, lose their G1 checkpoint, and thus rely more on the G2 checkpoint for DNA damage repair to sustain their survival.

Membrane-associated tyrosine- and threonine-protein kinase (Myt1 kinase, also known as PKMYT1) is encoded by the PKMYT1 gene. PKMYT1 inhibits the activity of cdc2 by phosphorylating Thr-14 and Tyr-15 on cdc2, thereby regulating the cell cycle. This causes the cell cycle to pause at the G2-M phase, allowing for DNA damage repair. Research has shown that inhibiting PKMYT1 leads to the activation of cdc2, forcing cells to enter mitosis prematurely without repairing DNA damage, thus killing rapidly proliferating tumor cells.

Therefore, PKMYT1 inhibitors have the potential for inhibiting tumor proliferation, and the development of PKMYT1 inhibitors may provide a novel strategy for targeted tumor therapy.

### SUMMARY

In one aspect, the present application relates to a compound of formula (I) or a pharmaceutically acceptable salt thereof,

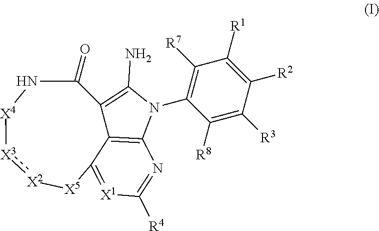

wherein
X¹ is selected from the group consisting of N and CR⁹;
[symbol] represents a single bond or a double bond;
X² is selected from the group consisting of CR⁵R⁶, NR⁵, CR⁵, and N;
X³ is selected from the group consisting of CR⁵′R⁶′, NR⁵′, CR⁵′, and N;
X⁴ and X⁵ are independently selected from the group consisting of (C(R¹⁰)₂)ₙ, NR¹⁰, and O;
R¹ and R³ are independently selected from the group consisting of hydrogen, hydroxy, amino, nitro, halogen, cyano, C₁-C₆ alkyl, C₁-C₆ alkoxy, C₃-C₉ cycloalkyl-O—, 4-9 membered heterocyclyl-O—, C₁-C₆ alkyl-C(O)O—, C₃-C₉ cycloalkyl-C(O)O—, 4-9 membered heterocyclyl-C(O)O—, P(O)(OH)₂O—, and NH₂C(O)O—, wherein the C₁-C₆ alkyl, C₁-C₆ alkoxy, C₃-C₉ cycloalkyl-O—, 4-9 membered heterocyclyl-O—, C₁-C₆ alkyl-C(O)O—, C₃-C₉ cycloalkyl-C(O)O—, 4-9 membered heterocyclyl-C(O)O—, P(O)(OH)₂O—, or NH₂C(O)O— is optionally substituted with one or more R¹¹;
R² is selected from the group consisting of hydrogen, cyano, halogen, C₁-C₆ alkyl, C₃-C₉ cycloalkyl, 4-9 membered heterocyclyl, C₆-C₁₀ aryl, 5-9 membered heteroaryl, C(═O)H, C₂-C₆ alkenyl, and C₂-C₆ alkynyl, wherein the C₁-C₆ alkyl, C₃-C₉ cycloalkyl, 4-9 membered heterocyclyl, C₆-C₁₀ aryl, 5-9 membered heteroaryl, C(═O)H, C₂-C₆ alkenyl, or C₂-C₆ alkynyl is optionally substituted with one or more R^{a};
or R¹ and R², together with the atoms linked thereto, form a 5-8 membered heterocyclic ring or 5-9 membered heteroaromatic ring;
R⁴, R⁶, R⁶′, and R¹⁰ are independently selected from the group consisting of hydrogen, deuterium, amino, hydroxy, sulfydryl, halogen, cyano, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, and —C(═O)NH₂, wherein the amino, hydroxy, sulfydryl, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, or —C(═O)NH₂ is optionally substituted with one or more R^{a};
R⁵ and R⁵′, together with the atoms linked thereto, form a C₃-C₉ saturated or partially saturated carbon ring, a C₆-C₁₀ aromatic ring, a 5-8 membered heterocyclic ring, and a 5-9 membered heteroaromatic ring, wherein the C₃-C₉ saturated or partially saturated carbon ring, C₆-C₁₀ aromatic ring, 5-8 membered heterocyclic ring, or 5-9 membered heteroaromatic ring is optionally substituted with one or more R^{a};
R⁷ and R⁸ are independently selected from the group consisting of hydrogen, halogen, and C₁-C₆ alkyl, wherein the C₁-C₆ alkyl is optionally substituted with deuterium;
R⁹ is selected from the group consisting of hydrogen, hydroxy, amino, cyano, halogen, C₁-C₆ alkyl, C₃-C₆ cycloalkyl, C₂-C₆ alkenyl, C₂-C₆ alkynyl, and C(═O)H, wherein the hydroxy, amino, C₁-C₆ alkyl, C₃-C₆ cycloalkyl, C₂-C₆ alkenyl, C₂-C₆ alkynyl, or C(═O)H is optionally substituted with one or more R^{a};
R¹¹ is selected from the group consisting of C₁-C₁₀ alkyl, C₃-C₉ cycloalkyl, 4-9 membered heterocyclyl, C₁-C₆ alkoxy, C₃-C₉ cycloalkyl-O—, 4-9 membered heterocyclyl-O—, C₁-C₆ alkyl-C(O)O—, C₃-C₉ cycloalkyl-C(O)O—, 4-9 membered heterocyclyl-C(O)O—, P(O)(OH)₂O, NH₂C(O)O—, and C₁-C₆ alkyl-OC(O)O—;
n is selected from the group consisting of 0, 1, and 2;
R^{a} is independently selected from the group consisting of deuterium, halogen, oxo, hydroxy, amino, cyano, C₁-C₆ alkyl, C₃-C₆ cycloalkyl, 4-7 membered heterocyclyl, C₆-C₁₀ aryl, and 5-9 membered heteroaryl, wherein the hydroxy, amino, C₁-C₆ alkyl, C₃-C₆ cycloalkyl, 4-7 membered heterocyclyl, C₆-C₁₀ aryl, or 5-9 membered heteroaryl is optionally substituted with one or more R^{b};
R^{b} is independently selected from the group consisting of halogen, hydroxy, amino, cyano, C₁-C₃ alkyl, NH(C₁-C₃ alkyl), N(C₁-C₃ alkyl)₂, C₃-C₆ cycloalkyl, 4-7 membered heterocyclyl, C₁-C₆ alkoxy, C₆-C₁₀ aryl, and 5-9 membered heteroaryl, wherein the C₁-C₃ alkyl, C₃-C₆ cycloalkyl, 4-7 membered heterocyclyl, C₁-C₆ alkoxy, C₆-C₁₀ aryl, or 5-9 membered heteroaryl is optionally substituted with one or more R^{c};
R^{c} is independently selected from the group consisting of halogen, hydroxy, amino, cyano, and C₁-C₃ alkyl.

In some embodiments, R⁴, R⁶, R⁶′, and R¹⁰ are independently selected from the group consisting of hydrogen, amino, hydroxy, sulfydryl, halogen, cyano, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, and —C(═O)NH₂, wherein the amino, hydroxy, sulfydryl, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, or —C(═O)NH₂ is optionally substituted with one or more R^{a}; R⁷ and R⁸ are independently selected from the group consisting of halogen and C₁-C₆ alkyl; R^{a} is independently selected from the group consisting of halogen, oxo, hydroxy, amino, cyano, C₁-C₆ alkyl, C₃-C₆ cycloalkyl, 4-7 membered heterocyclyl, C₆-C₁₀ aryl, and 5-9 membered heteroaryl, wherein the hydroxy, amino, C₁-C₆ alkyl, C₃-C₆ cycloalkyl, 4-7 membered heterocyclyl, C₆-C₁₀ aryl, or 5-9 membered heteroaryl is optionally substituted with one or more R^{b};
R^{b} is independently selected from the group consisting of halogen, hydroxy, amino, cyano, NH(C₁-C₃ alkyl), N(C₁-C₃ alkyl)₂, C₃-C₆ cycloalkyl, 4-7 membered heterocyclyl, C₁-C₆ alkoxy, C₆-C₁₀ aryl, and 5-9 membered heteroaryl, wherein the C₃-C₆ cycloalkyl, 4-7 membered heterocyclyl, C₁-C₆ alkoxy, C₆-C₁₀ aryl, or 5-9 membered heteroaryl is optionally substituted with one or more R^{c}.

In some embodiments, R^{b} is independently selected from the group consisting of halogen, hydroxy, amino, cyano, C₁-C₃ alkyl, NH(C₁-C₃ alkyl), N(C₁-C₃ alkyl)₂, C₃-C₆ cycloalkyl, 4-7 membered heterocyclyl, C₁-C₆ alkoxy, C₆-C₁₀ aryl, and 5-9 membered heteroaryl, wherein the C₃-C₆ cycloalkyl, 4-7 membered heterocyclyl, C₁-C₆ alkoxy, C₆-C₁₀ aryl, or 5-9 membered heteroaryl is optionally substituted with one or more R^{c}; R^{c} is independently selected from the group consisting of halogen, hydroxy, amino, cyano, and C₁-C₃ alkyl.

In some embodiments, X² is selected from the group consisting of CR⁵R⁶, NR⁵, and CR⁵.

In some embodiments, X² is selected from the group consisting of CR⁵R⁶ and CR⁵.

In some embodiments, X² is CR⁵R⁶. In some embodiments, X² is CR⁵.

In some embodiments, X³ is selected from the group consisting of CR⁵′R⁶′, NR⁵′, and CR⁵′.

In some embodiments, X³ is selected from the group consisting of CR⁵′R⁶′ and CR⁵′.

In some embodiments, X³ is CR⁵′R⁶′. In some embodiments, X³ is CR⁵′.

In some embodiments, X⁴ and X⁵ are independently selected from the group consisting of a bond, C(R¹⁰)₂, and O.

In some embodiments, X⁴ is selected from the group consisting of a bond and C(R¹⁰)₂.

In some embodiments, X⁴ and X⁵ are independently selected from the group consisting of (C(R¹⁰)₂)ₙ, and n is 0, which indicates that X⁴ and X⁵ are independently selected from the group consisting of a bond.

In some embodiments, X⁴ is a bond, —CH₂—, —CH(CH₃)—, or

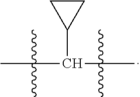 [SMILES: *C(*)C1CC1]

In some embodiments, X⁴ is a bond. In some embodiments, X⁴ is —CH₂—.

In some embodiments, X⁵ is selected from the group consisting of a bond and C(R¹⁰)₂.

In some embodiments, X⁵ is a bond.

In some embodiments, X⁵ is a bond, —O—, —CH(CH₃)—, —C(CH₃)₂—, or

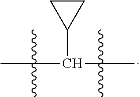 [SMILES: *C(*)C1CC1]

In some embodiments, X⁴ and X⁵ are both bonds.

In some embodiments, X⁴ is —CH₂—, and X⁵ is a bond.

In some embodiments, R¹ and R³ are independently selected from the group consisting of hydrogen and hydroxy. In some embodiments, R¹ is hydroxy, and R³ is hydrogen.

In some embodiments, R² is hydrogen.

In some embodiments, R¹ is hydroxy. In some embodiments, R² and R³ are both hydrogen. In some embodiments, R¹ is hydroxy, and R² and R³ are both hydrogen.

In some embodiments, R¹ is selected from the group consisting of hydroxy, C₁-C₆ alkoxy, C₁-C₆ alkyl-C(O)O—, 4-9 membered heterocyclyl-C(O)O—, P(O)(OH)₂O—, and NH₂C(O)O—, wherein the C₁-C₆ alkoxy, C₁-C₆ alkyl-C(O)O—, 4-9 membered heterocyclyl-C(O)O—, P(O)(OH)₂O—, and NH₂C(O)O— are each optionally substituted with one or more R¹¹. In some embodiments, R¹¹ is selected from the group consisting of C₁-C₁₀ alkyl, 4-9 membered heterocyclyl, C₁-C₆ alkyl-C(O)O—, P(O)(OH)₂O, and C₁-C₆ alkyl-OC(O)O—.

In some embodiments, R¹ is selected from the group consisting of hydroxy, C₁-C₆ alkoxy, C₁-C₆ alkyl-C(O)O—, 5- or 6-membered heterocyclyl-C(O)O—, P(O)(OH)₂O—, and NH₂C(O)O—, wherein the C₁-C₆ alkoxy, C₁-C₆ alkyl-C(O)O—, 5- or 6-membered heterocyclyl-C(O)O—, P(O)(OH)₂O—, and NH₂C(O)O— are each optionally substituted with one or more R¹¹. In some embodiments, R¹¹ is selected from the group consisting of C₁-C₁₀ alkyl, 5- or 6-membered heterocyclyl, C₁-C₆ alkyl-C(O)O—, P(O)(OH)₂O—, and C₁-C₆ alkyl-OC(O)O—.

In some embodiments, R¹ is —OH

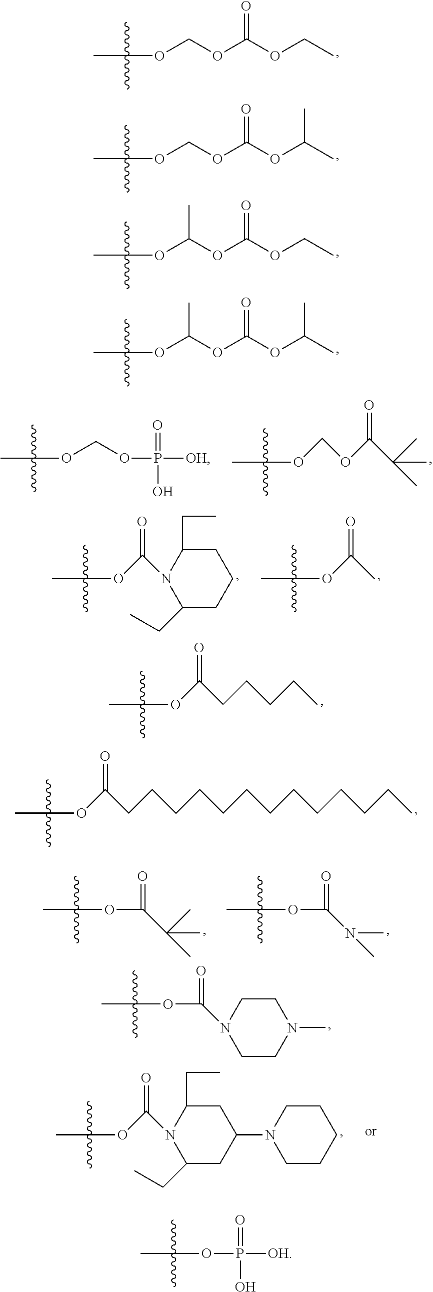

In some embodiments, R⁴, R⁶, R⁶′, and R¹⁰ are independently selected from the group consisting of hydrogen, amino, hydroxy, sulfydryl, halogen, cyano, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, and —C(═O)NH₂, wherein the amino, hydroxy, sulfydryl, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, or —C(═O)NH₂ is optionally substituted with one or more R^{a}.

In some embodiments, R⁴, R⁶, R⁶′, and R¹⁰ are independently selected from the group consisting of hydrogen, amino, hydroxy, C₁-C₆ alkyl, C₃-C₉ cycloalkyl, 4-9 membered heterocyclyl, C₆-C₁₀ aryl, and 5-9 membered heteroaryl, wherein the amino, hydroxy, C₁-C₆ alkyl, C₃-C₉ cycloalkyl, 4-9 membered heterocyclyl, C₆-C₁₀ aryl, or 5-9 membered heteroaryl is optionally substituted with one or more R^{a}.

In some embodiments, R⁴, R⁶, R⁶′, and R¹⁰ are independently selected from the group consisting of hydrogen, amino, hydroxy, C₁-C₃ alkyl, C₃-C₆ cycloalkyl, 4-6 membered heterocyclyl, and 5-6 membered heteroaryl, wherein the amino, hydroxy, C₁-C₃ alkyl, C₃-C₆ cycloalkyl, 4-6 membered heterocyclyl, or 5-6 membered heteroaryl is optionally substituted with one or more R^{a}.

In some embodiments, R⁴ is selected from the group consisting of hydrogen, amino, hydroxy, C₁-C₆ alkyl, C₃-C₉ cycloalkyl, 4-9 membered heterocyclyl, C₆-C₁₀ aryl, and 5-9 membered heteroaryl, wherein the amino, hydroxy, C₁-C₆ alkyl, C₃-C₉ cycloalkyl, 4-9 membered heterocyclyl, C₆-C₁₀ aryl, or 5-9 membered heteroaryl is optionally substituted with one or more R^{a}.

In some embodiments, R⁴ is selected from the group consisting of hydrogen, amino, hydroxy, C₁-C₃ alkyl, C₃-C₆ cycloalkyl, 4-6 membered heterocyclyl, and 5-6 membered heteroaryl, wherein the amino, hydroxy, C₁-C₃ alkyl, C₃-C₆ cycloalkyl, 4-6 membered heterocyclyl, or 5-6 membered heteroaryl is optionally substituted with one or more R^{a}.

In some embodiments, R⁴ is selected from the group consisting of —H, —CH₃, —CH(CH₃)₂, —OCH₃, —N(CH₃)₂, —CF₃, —CHF₂, —C(CH₃)₂(OH),

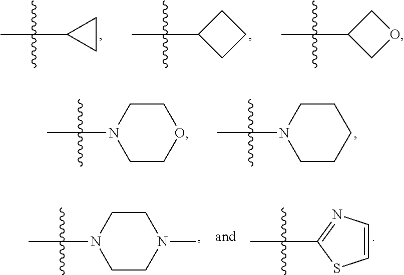

In some embodiments, R⁴ is selected from the group consisting of H and C₁-C₃ alkyl.

In some embodiments, R⁴ is selected from the group consisting of H and methyl.

In some embodiments, R⁴ is H. In some embodiments, R⁴ is methyl.

In some embodiments, each R¹⁰ is independently selected from the group consisting of hydrogen, C₁-C₆ alkyl, and C₃-C₆ cycloalkyl.

In some embodiments, R⁵ and R⁵′, together with the atoms linked thereto, form a C₄-C₇ saturated or partially saturated carbon ring, a benzene ring, a 5-6 membered heterocyclic ring, or a 5-6 membered heteroaromatic ring, wherein the C₄-C₇ saturated or partially saturated carbon ring, benzene ring, 5-6 heterocyclic ring, or 5-6 membered heteroaromatic ring is optionally substituted with one or more R^{a}.

In some embodiments, R⁵ and R⁵′, together with the atoms linked thereto, form a benzene ring or a 5-6 membered heteroaromatic ring, wherein the benzene ring or 5-6 membered heteroaromatic ring is optionally substituted with one or more R^{a}.

In some embodiments, X² is CR⁵; X³ is CR⁵′; X⁴ and X⁵ are both bonds.

In some embodiments, X² is CR⁵; X³ is CR⁵′; X⁴ is —CH₂—; X⁵ is a bond.

In some embodiments, X² is CR⁵, X³ is CR⁵′, and R⁵ and R⁵′, together with the atoms linked thereto, form a benzene ring or a 5-6 membered heteroaromatic ring, wherein the benzene ring or 5-6 membered heteroaromatic ring is optionally substituted with one or more R^{a}.

In some embodiments, X² is CR⁵, X³ is CR⁵′, and R⁵ and R⁵′, together with the atoms linked thereto, form a benzene ring, a pyridine ring, a pyrazole ring, a thiazole ring, an isothiazole ring, a pyrrole ring, or a furan ring, wherein the benzene ring, pyridine ring, pyrazole ring, thiazole ring, isothiazole ring, pyrrole ring, or furan ring is optionally substituted with one or more R^{a}.

In some embodiments, X² is CR⁵, X³ is CR⁵′, and R⁵ and R⁵′, together with the atoms linked thereto, form a benzene ring or a pyridine ring, wherein the benzene ring or pyridine ring is optionally substituted with one or more R^{a}.

In some embodiments, R⁷ and R⁸ are independently selected from the group consisting of halogen and C₁-C₆ alkyl.

In some embodiments, R⁷ and R⁸ are independently selected from the group consisting of C₁-C₃ alkyl.

In some embodiments, R⁷ and R⁸ are both methyl.

In some embodiments, X¹ is N.

In some embodiments, X¹ is CR⁹.

In some embodiments, X¹ is CCH₃.

In some embodiments, X¹ is N, and R⁴ is methyl.

In some embodiments, R⁹ is selected from the group consisting of hydrogen, hydroxy, amino, halogen, and C₁-C₃ alkyl, wherein the hydroxy, amino, and C₁-C₃ alkyl are each optionally substituted with one or more R^{a}.

In some embodiments, R⁹ is selected from the group consisting of hydrogen and methyl.

In some embodiments, R¹⁰ is selected from the group consisting of hydrogen, amino, hydroxy, C₁-C₃ alkyl, and C₃-C₆ cycloalkyl, wherein the hydroxy, amino, C₁-C₃ alkyl, or C₃-C₆ cycloalkyl is optionally substituted with one or more R^{a}.

In some embodiments, R¹⁰ is H.

In some embodiments, n is selected from the group consisting of 0 and 1.

In some embodiments, R^{a} is independently selected from the group consisting of halogen, oxo, hydroxy, amino, cyano, C₁-C₆ alkyl, C₃-C₆ cycloalkyl, 4-7 membered heterocyclyl, C₆-C₁₀ aryl, and 5-9 membered heteroaryl, wherein the hydroxy, amino, C₁-C₆ alkyl, C₃-C₆ cycloalkyl, 4-7 membered heterocyclyl, C₆-C₁₀ aryl, or 5-9 membered heteroaryl is optionally substituted with one or more R^{b}.

In some embodiments, R^{a} is independently selected from the group consisting of cyano, halogen, hydroxy, amino, C₁-C₆ alkyl, and C₃-C₆ cycloalkyl, wherein the hydroxy, amino, C₁-C₆ alkyl, and C₃-C₆ cycloalkyl are each optionally substituted with one or more R^{b}.

In some embodiments, R^{a} is independently selected from the group consisting of halogen, hydroxy, amino, C₁-C₆ alkyl, and C₃-C₆ cycloalkyl, wherein the hydroxy, amino, C₁-C₆ alkyl, and C₃-C₆ cycloalkyl are each optionally substituted with one or more R^{b}.

In some embodiments, R^{a} is independently selected from the group consisting of cyano, halogen, hydroxy, C₁-C₆ alkyl, and C₃-C₆ cycloalkyl, wherein the hydroxy, C₁-C₆ alkyl, and C₃-C₆ cycloalkyl are each optionally substituted with one or more R^{b}.

In some embodiments, R^{a} is independently selected from the group consisting of halogen, hydroxy, C₁-C₆ alkyl, and C₃-C₆ cycloalkyl, wherein the hydroxy, C₁-C₆ alkyl, and C₃-C₆ cycloalkyl are each optionally substituted with one or more R^{b}.

In some embodiments, R^{b} is independently selected from the group consisting of halogen, hydroxy, amino, cyano, NH(C₁-C₃ alkyl), N(C₁-C₃ alkyl)₂, C₃-C₆ cycloalkyl, 4-7 membered heterocyclyl, C₁-C₆ alkoxy, C₆-C₁₀ aryl, and 5-9 membered heteroaryl, wherein the C₃-C₆ cycloalkyl, 4-7 membered heterocyclyl, C₁-C₆ alkoxy, C₆-C₁₀ aryl, or 5-9 membered heteroaryl is optionally substituted with one or more R^{c}.

In some embodiments, R^{b} is independently selected from the group consisting of amino, NH(C₁-C₃ alkyl), N(C₁-C₃ alkyl)₂, C₃-C₆ cycloalkyl, 4-7 membered heterocyclyl, and C₁-C₆ alkoxy, wherein the C₃-C₆ cycloalkyl, 4-7 membered heterocyclyl, and C₁-C₆ alkoxy are each optionally substituted with one or more R^{c}.

In some embodiments, R^{b} is independently selected from the group consisting of halogen, hydroxy, C₁-C₃ alkyl, N(C₁-C₃ alkyl)₂, C₃-C₆ cycloalkyl, 4-7 membered heterocyclyl, and C₁-C₆ alkoxy, wherein the C₁-C₃ alkyl, C₃-C₆ cycloalkyl, 4-7 membered heterocyclyl, and C₁-C₆ alkoxy are each optionally substituted with one or more R^{c}.

In some embodiments, R^{b} is independently selected from the group consisting of halogen, hydroxy, C₁-C₃ alkyl, and C₃-C₆ cycloalkyl, wherein the hydroxy, C₁-C₃ alkyl, and C₃-C₆ cycloalkyl are each optionally substituted with one or more R^{c}.

In some embodiments, R^{c} is independently selected from the group consisting of halogen and C₁-C₃ alkyl.

In some embodiments, R^{c} is independently selected from the group consisting of C₁-C₃ alkyl. In some embodiments, R^{c} is independently selected from the group consisting of halogen (e.g., F).

In some embodiments, R^{a} is independently selected from the group consisting of cyano, halogen, C₁-C₃ alkyl, C₁-C₃ alkoxy, C₁-C₃ haloalkyl, C₁-C₃ haloalkoxy, C₁-C₃ alkoxy-C₁-C₃ alkylene, C₃-C₆ cycloalkyl, C₃-C₆ cycloalkyloxy, and hydroxymethyl.

In some embodiments, R^{a} is independently selected from the group consisting of cyano, halogen, C₁-C₃ alkyl, C₁-C₃ alkoxy, C₁-C₃ haloalkyl, C₁-C₃ haloalkoxy, C₃-C₆ cycloalkyl, C₃-C₆ cycloalkyloxy, and hydroxymethyl.

In some embodiments, R^{a} is independently selected from the group consisting of halogen, C₁-C₃ alkyl, C₁-C₃ alkoxy, C₁-C₃ haloalkyl, C₁-C₃ haloalkoxy, C₃-C₆ cycloalkyl, C₃-C₆ cycloalkyloxy, and hydroxymethyl.

In some embodiments, R^{a} is independently selected from the group consisting of halogen, C₁-C₃ alkyl, C₁-C₃ alkoxy, and C₁-C₃ haloalkyl.

In some embodiments, R^{a} is independently selected from the group consisting of —OH, —CH₃, —C₂H₅, —OCH₃, —OCH₂CH₃, —CH₂CF₃, —F, —OCF₂H, —OCF₃, —CN, —CF₃, —CH₂OH, —OCH₂F, —CHF₂, —CH₂CH₂OCH₃, —CH(CH₃)₂, —N(CH₃)₂, —CH₂N(CH₃)₂, —CH₂OCH₃,

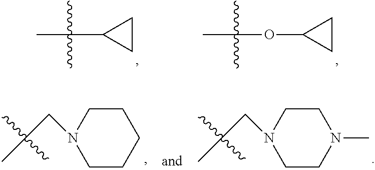

In some embodiments, R⁴ is selected from the group consisting of hydrogen, amino, hydroxy, C₁-C₃ alkyl, C₃-C₆ cycloalkyl, 4-6 membered heterocyclyl, and 5-6 membered heteroaryl, wherein the amino, hydroxy, C₁-C₃ alkyl, C₃-C₆ cycloalkyl, 4-6 membered heterocyclyl, or 5-6 membered heteroaryl is optionally substituted with one or more R^{a}; and R^{a} is independently selected from the group consisting of halogen (e.g., F), hydroxy, and C₁-C₆ alkyl (e.g., methyl).

In some embodiments, X² is CR⁵, X³ is CR⁵′, and R⁵ and R⁵′, together with the atoms linked thereto, form a benzene ring or a 5-6 membered heteroaromatic ring, wherein the benzene ring or 5-6 membered heteroaromatic ring is optionally substituted with one or more R^{a}; R^{a} is independently selected from the group consisting of halogen, hydroxy, amino, cyano, C₁-C₆ alkyl, and C₃-C₆ cycloalkyl, wherein the hydroxy, amino, C₁-C₆ alkyl, and C₃-C₆ cycloalkyl are each optionally substituted with one or more R^{b}; R^{b} is independently selected from the group consisting of halogen, hydroxy, C₁-C₃ alkyl, N(C₁-C₃ alkyl)₂, C₃-C₆ cycloalkyl, 4-7 membered heterocyclyl, and C₁-C₆ alkoxy, wherein the C₁-C₃ alkyl, N(C₁-C₃ alkyl)₂, C₃-C₆ cycloalkyl, 4-7 membered heterocyclyl, and C₁-C₆ alkoxy are each optionally substituted with one or more R^{c}; R^{c} is independently selected from the group consisting of halogen.

In some embodiments, X² is CR⁵, X³ is CR⁵′, and R⁵ and R⁵′, together with the atoms linked thereto, form a benzene ring, a pyridine ring, a pyrazole ring, a thiazole ring, an isothiazole ring, a pyrrole ring, or a furan ring, wherein the benzene ring, pyridine ring, pyrazole ring, thiazole ring, isothiazole ring, pyrrole ring, or furan ring is optionally substituted with one or more (e.g., 1 or 2) R^{a}; R^{a} is independently selected from the group consisting of —OH, —CH₃, —C₂H₅, —OCH₃, —OCH₂CH₃, —CH₂CF₃, —F, —OCF₂H, —OCF₃, —CN, —CF₃, —CH₂OH, —OCH₂F, —CHF₂, —CH₂CH₂OCH₃, —CH(CH₃)₂, —N(CH₃)₂, —CH₂N(CH₃)₂, —CH₂OCH₃,

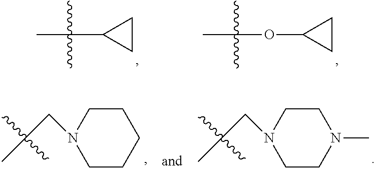

In some embodiments, the 5-6 membered heterocyclyl, 4-7 membered heterocyclyl, 4-9 membered heterocyclyl, 4-6 membered heterocyclic ring, 5-6 membered heterocyclic ring, or 5-8 membered heterocyclic ring independently contains 1, 2, or 3 heteroatoms independently selected from the group consisting of N, O, and S. In some embodiments, the 4-membered heterocyclyl, 5-membered heterocyclyl, 6-membered heterocyclyl, 4-membered heterocyclic ring, 5-membered heterocyclic ring, or 6-membered heterocyclic ring independently contains 1 or 2 heteroatoms independently selected from the group consisting of N and O.

In some embodiments, the 5-6 membered heteroaryl, 5-9 membered heteroaryl, 5-6 membered heteroaromatic ring, or 5-9 membered heteroaromatic ring independently contains 1, 2, or 3 heteroatoms independently selected from the group consisting of N, O, and S. In some embodiments, the 5-membered heteroaryl, 6-membered heteroaryl, 5-membered heteroaromatic ring, or 6-membered heteroaromatic ring independently contains 1 or 2 heteroatoms independently selected from the group consisting of N, O, and S.

In some embodiments, the compound of formula (I) or the pharmaceutically acceptable salt thereof of the present application is selected from the group consisting of a compound of formula (II) or a pharmaceutically acceptable salt thereof,

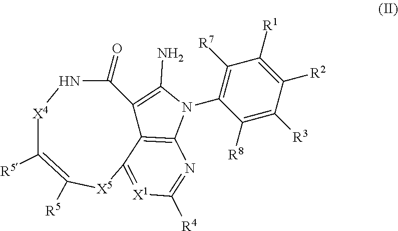

wherein R⁵ and R⁵′, together with the atoms linked thereto, form a C₆-C₁₀ aromatic ring or a 5-9 membered heteroaromatic ring, wherein the C₆-C₁₀ aromatic ring or 5-9 membered heteroaromatic ring is optionally substituted with one or more R^{a}; R¹, R², R³, R⁴, R⁷, R⁸, X¹, X⁴, X⁵, and R^{a} are as defined above.

In some embodiments, R⁵ and R⁵′, together with the atoms linked thereto, form a benzene ring, a pyridine ring, a pyrazole ring, a thiazole ring, or an isothiazole ring, wherein the benzene ring, pyridine ring, pyrazole ring, thiazole ring, or isothiazole ring is optionally substituted with one or more R^{a}.

In some embodiments, R⁵ and R⁵′, together with the atoms linked thereto, form a benzene ring, a pyridine ring, or a pyrazole ring, wherein the benzene ring, pyridine ring, or pyrazole ring is optionally substituted with one or more R^{a}.

In some embodiments, the compound of formula (I) or the pharmaceutically acceptable salt thereof of the present application is selected from the group consisting of a compound of formula (III) or a pharmaceutically acceptable salt thereof,

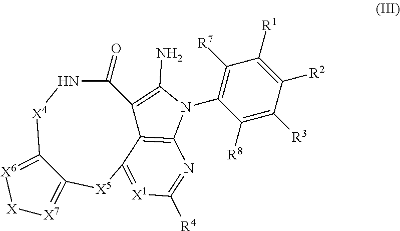

wherein X⁶ and X⁷ are independently selected from the group consisting of CH and N, wherein the CH is optionally substituted with halogen, C₁-C₃ alkyl, C₁-C₃ alkoxy, C₁-C₃ haloalkyl, or C₁-C₃ haloalkoxy; X is selected from the group consisting of NR^{a} and O; R¹, R², R³, R⁴, R⁷, R⁸, X¹, X⁴, X⁵, and R^{a} are as defined above.

In some embodiments, X⁶ and X⁷ in the compound of formula (III) are independently selected from the group consisting of CH, C—(C₁-C₃ alkyl), C-halogen, and N.

In some embodiments, X⁶ and X⁷ in the compound of formula (III) are independently selected from the group consisting of CH, C-halogen, and N.

In some embodiments, the compound of formula (I) or the pharmaceutically acceptable salt thereof of the present application is selected from the group consisting of the following compounds or the pharmaceutically acceptable salts thereof,

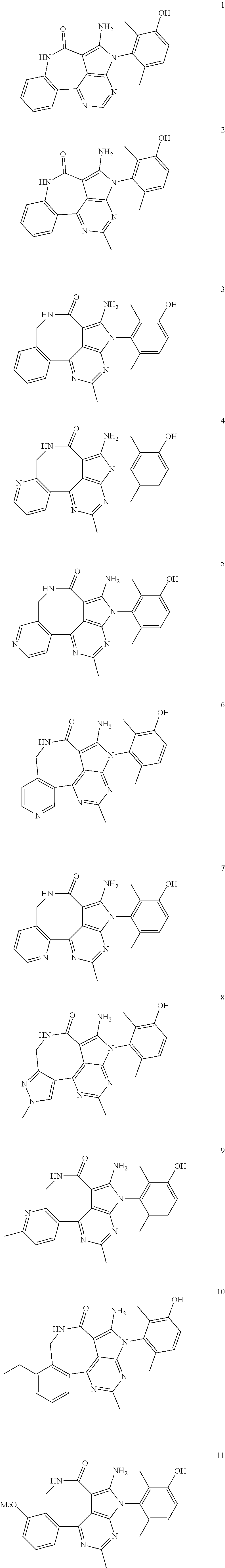

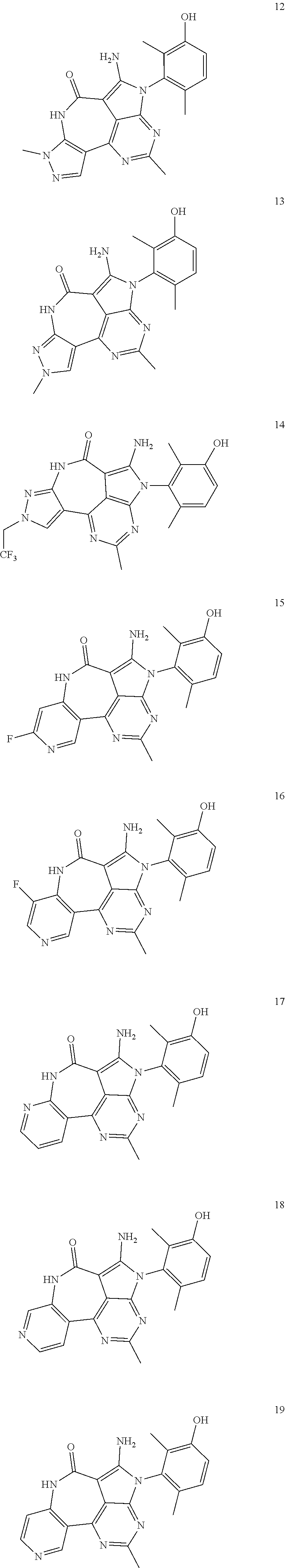

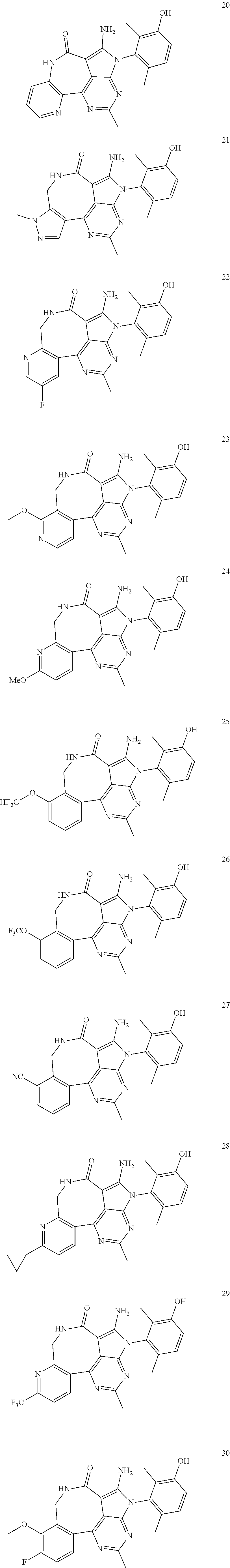

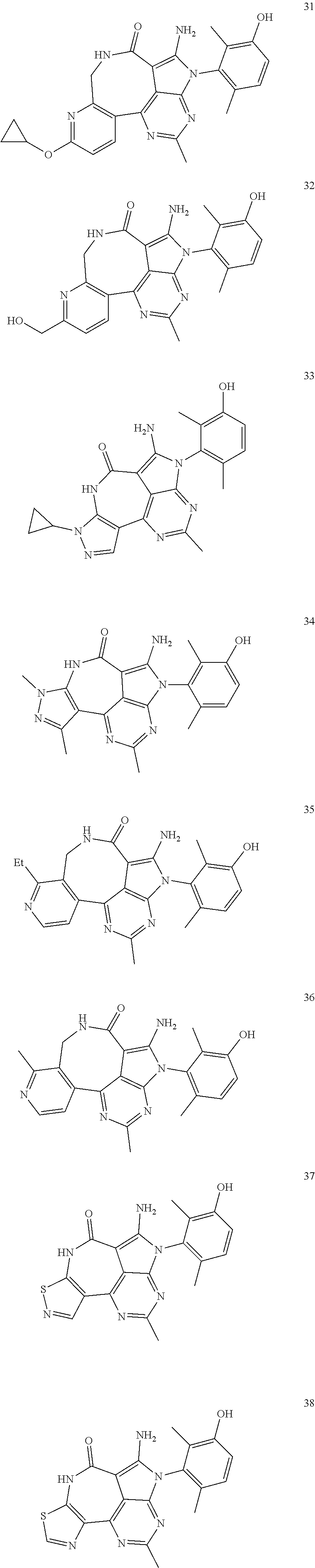

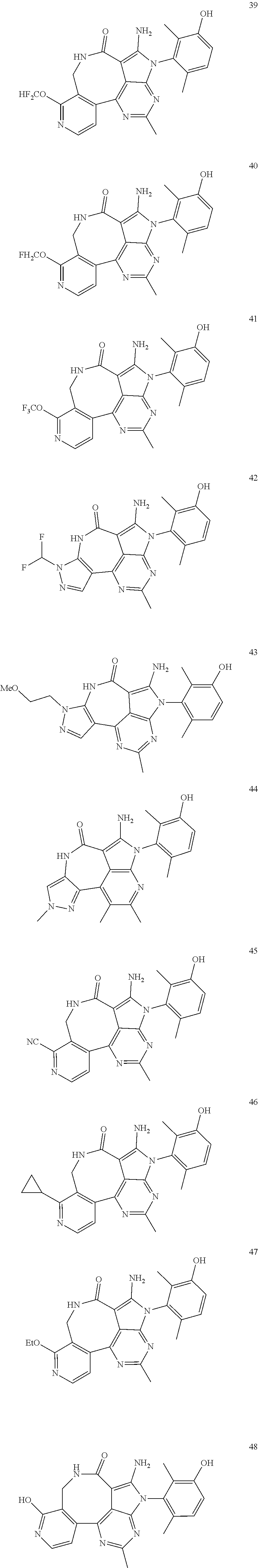

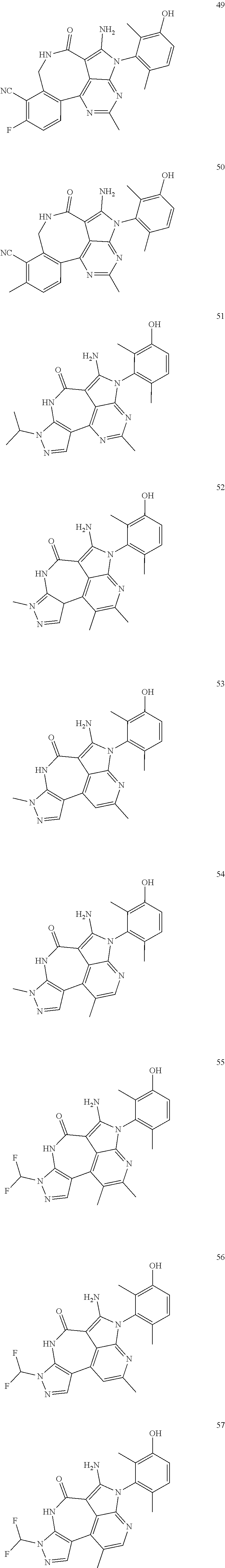

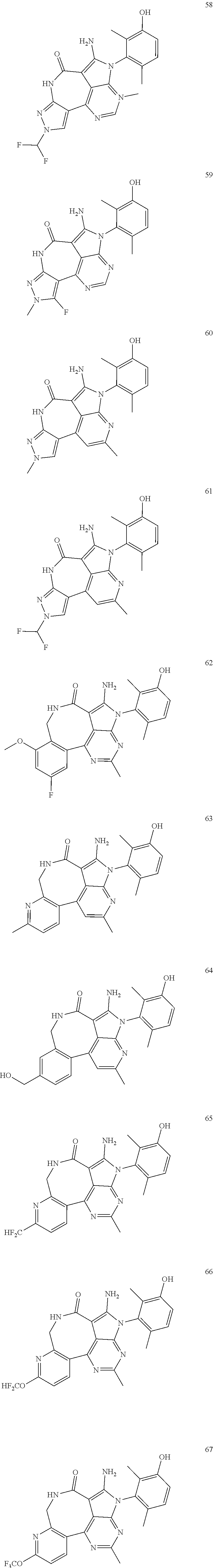

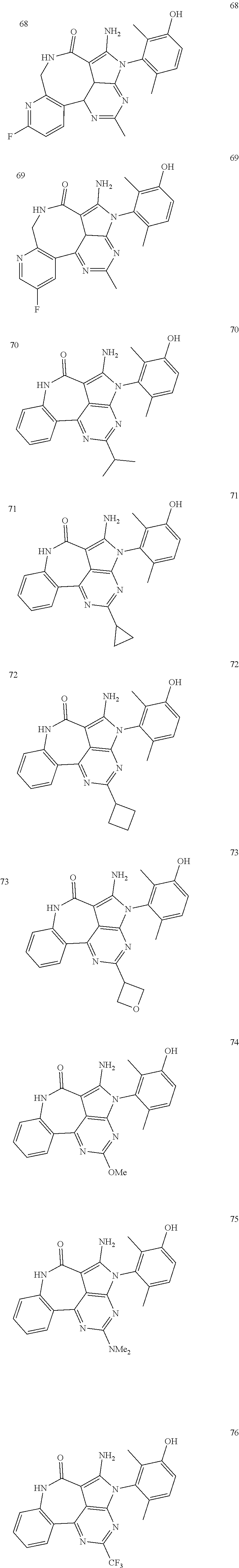

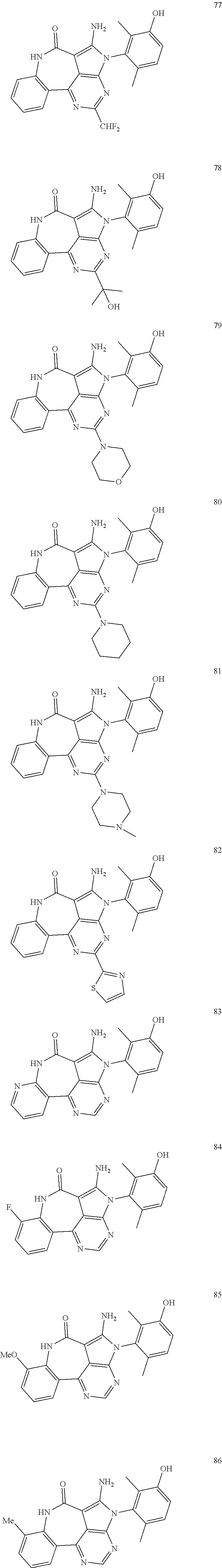

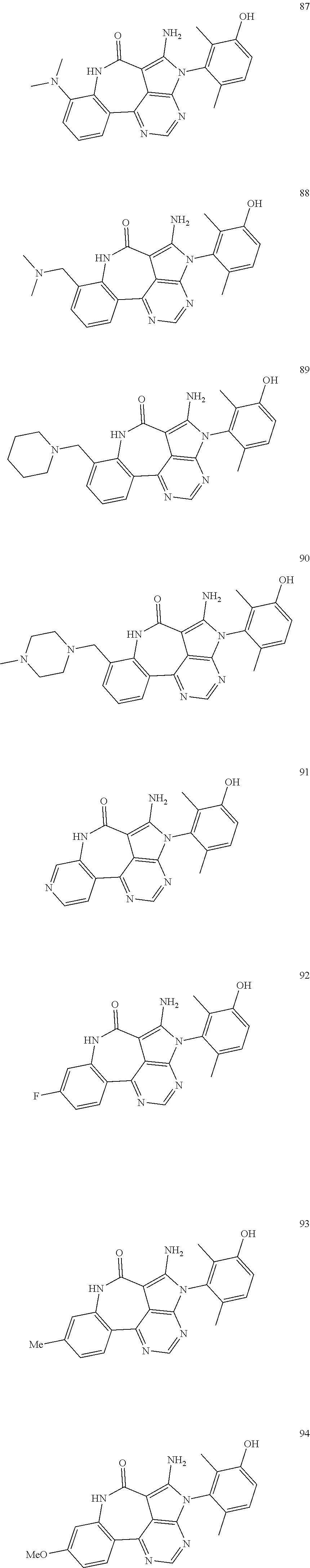

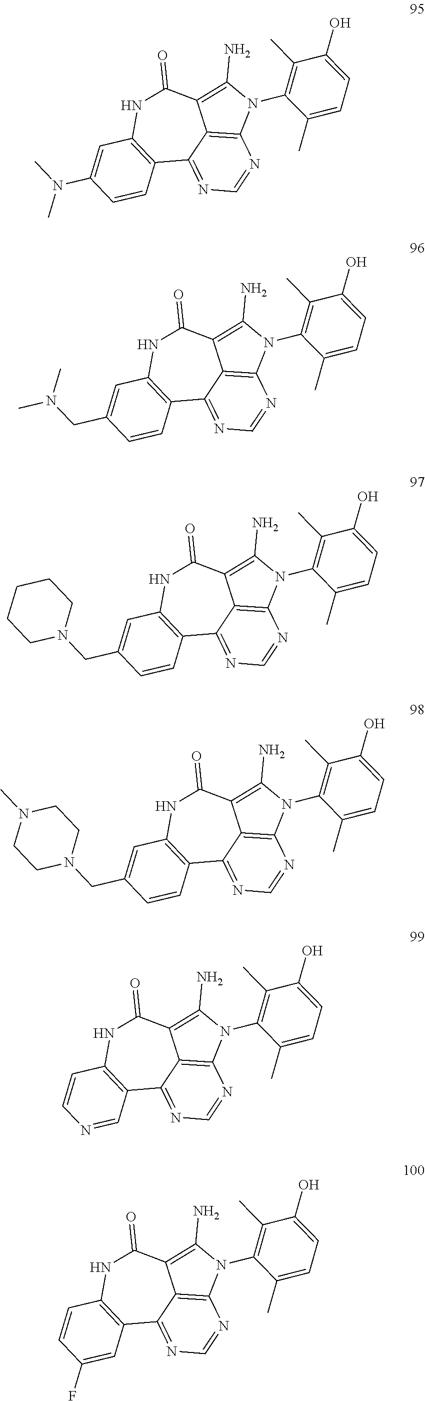 [SMILES: Cc1ccc(O)c(C)c1-n1c(N)c2c3c(ncnc31)-c1ccc(N(C)C)cc1NC2=O ; Cc1ccc(O)c(C)c1-n1c(N)c2c3c(ncnc31)-c1ccc(CN(C)C)cc1NC2=O ; Cc1ccc(O)c(C)c1-n1c(N)c2c3c(ncnc31)-c1ccc(CN3CCCCC3)cc1NC2=O ; Cc1ccc(O)c(C)c1-n1c(N)c2c3c(ncnc31)-c1ccc(CN3CCN(C)CC3)cc1NC2=O ; Cc1ccc(O)c(C)c1-n1c(N)c2c3c(ncnc31)-c1cnccc1NC2=O ; Cc1ccc(O)c(C)c1-n1c(N)c2c3c(ncnc31)-c1cc(F)ccc1NC2=O]

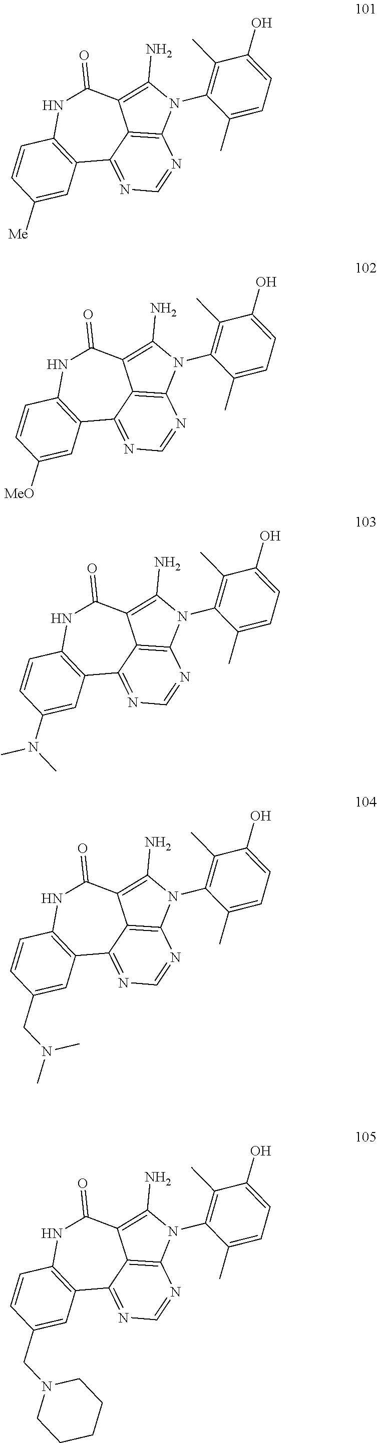 [SMILES: Cc1ccc2c(c1)-c1ncnc3c1c(c(N)n3-c1c(C)ccc(O)c1C)C(=O)N2 ; COc1ccc2c(c1)-c1ncnc3c1c(c(N)n3-c1c(C)ccc(O)c1C)C(=O)N2 ; Cc1ccc(O)c(C)c1-n1c(N)c2c3c(ncnc31)-c1cc(N(C)C)ccc1NC2=O ; Cc1ccc(O)c(C)c1-n1c(N)c2c3c(ncnc31)-c1cc(CN(C)C)ccc1NC2=O ; Cc1ccc(O)c(C)c1-n1c(N)c2c3c(ncnc31)-c1cc(CN3CCCCC3)ccc1NC2=O]

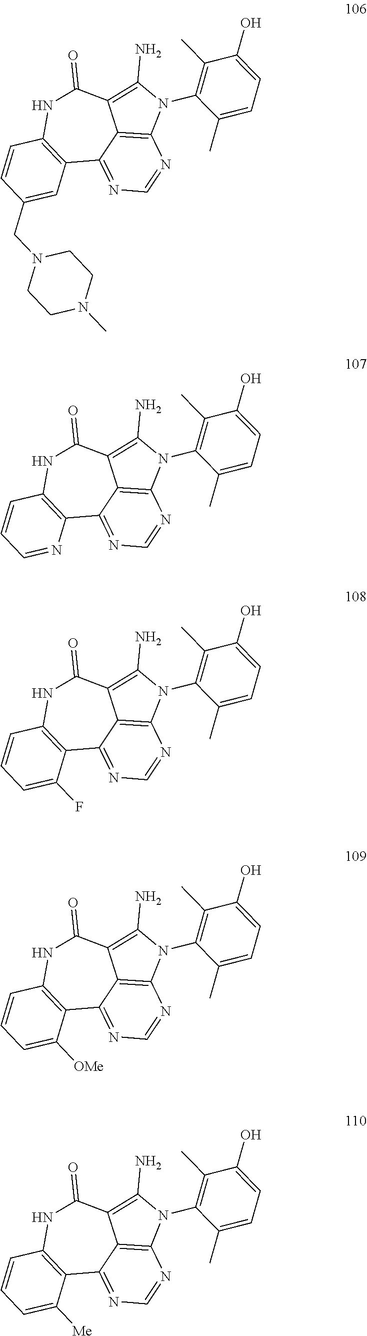 [SMILES: Cc1ccc(O)c(C)c1-n1c(N)c2c3c(ncnc31)-c1cc(CN3CCN(C)CC3)ccc1NC2=O ; Cc1ccc(O)c(C)c1-n1c(N)c2c3c(ncnc31)-c1ncccc1NC2=O ; Cc1ccc(O)c(C)c1-n1c(N)c2c3c(ncnc31)-c1c(F)cccc1NC2=O ; COc1cccc2c1-c1ncnc3c1c(c(N)n3-c1c(C)ccc(O)c1C)C(=O)N2 ; Cc1cccc2c1-c1ncnc3c1c(c(N)n3-c1c(C)ccc(O)c1C)C(=O)N2]

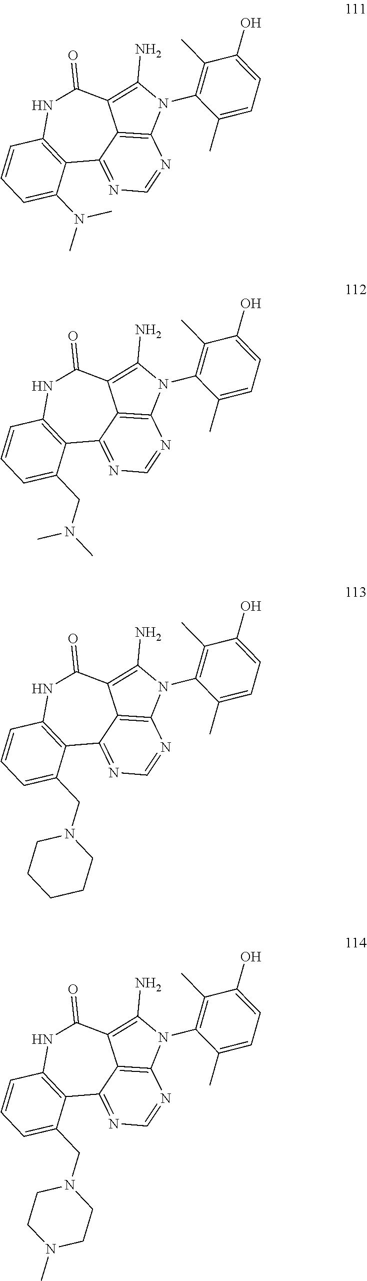 [SMILES: Cc1ccc(O)c(C)c1-n1c(N)c2c3c(ncnc31)-c1c(cccc1N(C)C)NC2=O ; Cc1ccc(O)c(C)c1-n1c(N)c2c3c(ncnc31)-c1c(CN(C)C)cccc1NC2=O ; Cc1ccc(O)c(C)c1-n1c(N)c2c3c(ncnc31)-c1c(CN3CCCCC3)cccc1NC2=O ; Cc1ccc(O)c(C)c1-n1c(N)c2c3c(ncnc31)-c1c(CN3CCN(C)CC3)cccc1NC2=O]

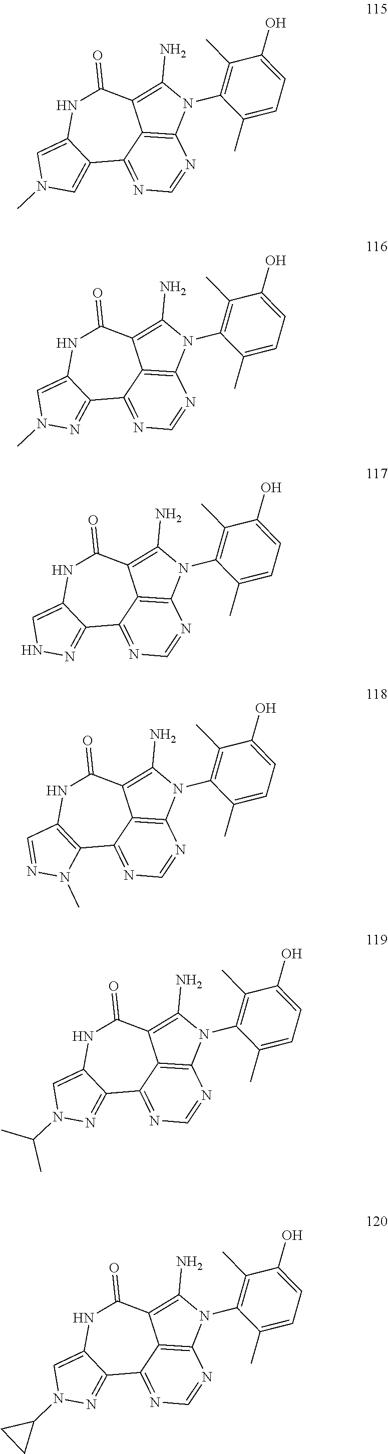 [SMILES: Cc1ccc(O)c(C)c1-n1c(N)c2c3c(ncnc31)-c1cn(C)cc1NC2=O ; Cc1ccc(O)c(C)c1-n1c(N)c2c3c(ncnc31)-c1nn(C)cc1NC2=O ; Cc1ccc(O)c(C)c1-n1c(N)c2c3c(ncnc31)-c1n[nH]cc1NC2=O ; Cc1ccc(O)c(C)c1-n1c(N)c2c3c(ncnc31)-c1c(cnn1C)NC2=O ; Cc1ccc(O)c(C)c1-n1c(N)c2c3c(ncnc31)-c1nn(C(C)C)cc1NC2=O ; Cc1ccc(O)c(C)c1-n1c(N)c2c3c(ncnc31)-c1nn(C3CC3)cc1NC2=O]

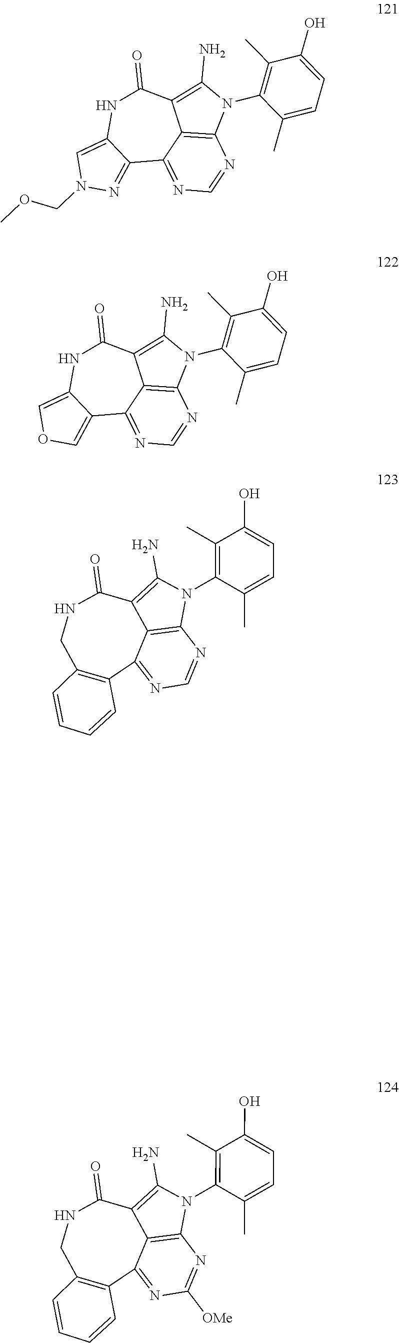 [SMILES: COCn1cc2c(n1)-c1ncnc3c1c(c(N)n3-c1c(C)ccc(O)c1C)C(=O)N2 ; Cc1ccc(O)c(C)c1-n1c(N)c2c3c(ncnc31)-c1cocc1NC2=O ; Cc1ccc(O)c(C)c1-n1c(N)c2c3c(ncnc31)-c1ccccc1CNC2=O ; COc1nc2c3c(c(N)n(-c4c(C)ccc(O)c4C)c3n1)C(=O)NCc1ccccc1-2]

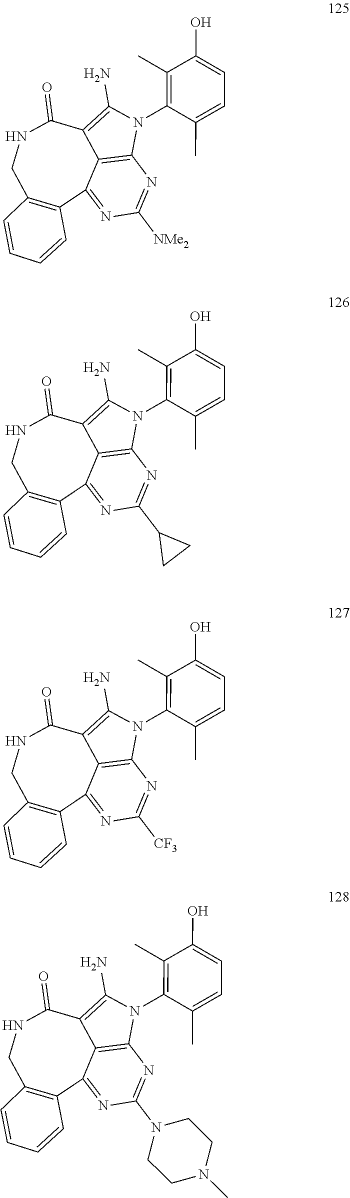 [SMILES: Cc1ccc(O)c(C)c1-n1c(N)c2c3c(nc(N(C)C)nc31)-c1ccccc1CNC2=O ; Cc1ccc(O)c(C)c1-n1c(N)c2c3c(nc(C4CC4)nc31)-c1ccccc1CNC2=O ; Cc1ccc(O)c(C)c1-n1c(N)c2c3c(nc(C(F)(F)F)nc31)-c1ccccc1CNC2=O ; Cc1ccc(O)c(C)c1-n1c(N)c2c3c(nc(N4CCN(C)CC4)nc31)-c1ccccc1CNC2=O]

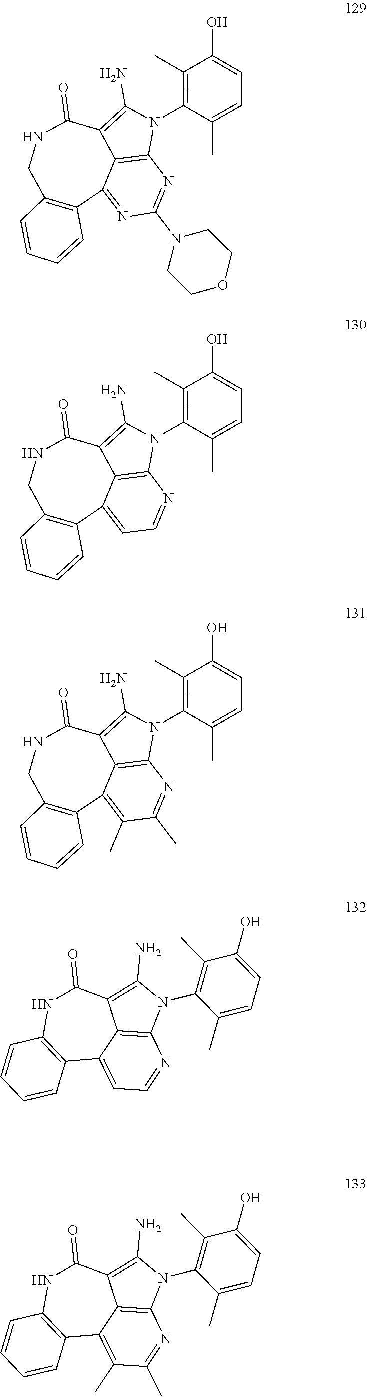 [SMILES: Cc1ccc(O)c(C)c1-n1c(N)c2c3c(nc(N4CCOCC4)nc31)-c1ccccc1CNC2=O ; Cc1ccc(O)c(C)c1-n1c(N)c2c3c(ccnc31)-c1ccccc1CNC2=O ; Cc1ccc(O)c(C)c1-n1c(N)c2c3c(c(C)c(C)nc31)-c1ccccc1CNC2=O ; Cc1ccc(O)c(C)c1-n1c(N)c2c3c(ccnc31)-c1ccccc1NC2=O ; Cc1ccc(O)c(C)c1-n1c(N)c2c3c(c(C)c(C)nc31)-c1ccccc1NC2=O]

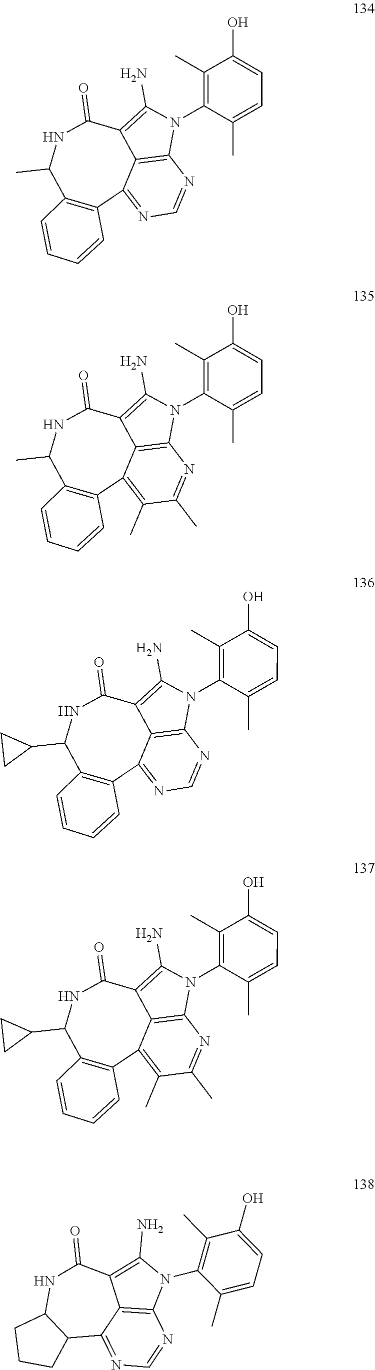 [SMILES: Cc1ccc(O)c(C)c1-n1c(N)c2c3c(ncnc31)-c1ccccc1C(C)NC2=O ; Cc1ccc(O)c(C)c1-n1c(N)c2c3c(c(C)c(C)nc31)-c1ccccc1C(C)NC2=O ; Cc1ccc(O)c(C)c1-n1c(N)c2c3c(ncnc31)-c1ccccc1C(C1CC1)NC2=O ; Cc1ccc(O)c(C)c1-n1c(N)c2c3c(c(C)c(C)nc31)-c1ccccc1C(C1CC1)NC2=O ; Cc1ccc(O)c(C)c1-n1c(N)c2c3c(ncnc31)C1CCCC1NC2=O]

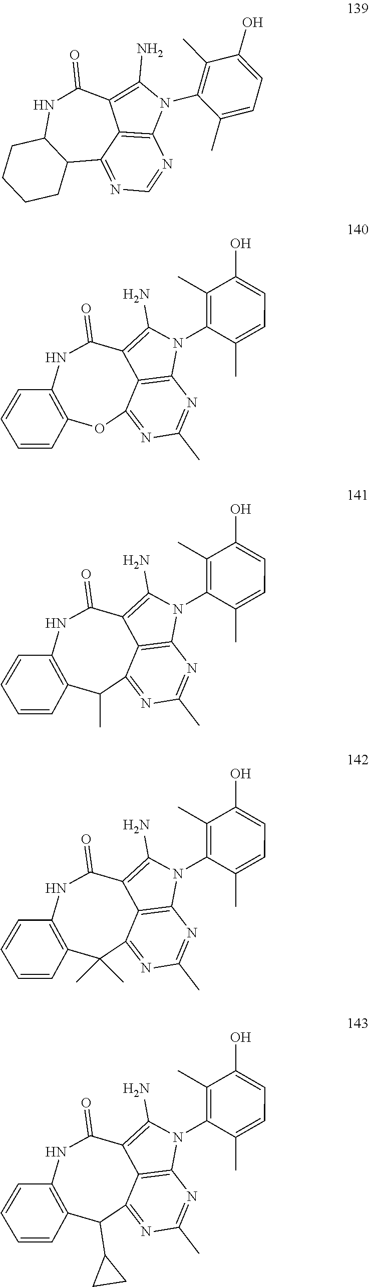 [SMILES: Cc1ccc(O)c(C)c1-n1c(N)c2c3c(ncnc31)C1CCCCC1NC2=O ; Cc1nc2c3c(c(N)n(-c4c(C)ccc(O)c4C)c3n1)C(=O)Nc1ccccc1O2 ; Cc1nc2c3c(c(N)n(-c4c(C)ccc(O)c4C)c3n1)C(=O)Nc1ccccc1C2C ; Cc1nc2c3c(c(N)n(-c4c(C)ccc(O)c4C)c3n1)C(=O)Nc1ccccc1C2(C)C ; Cc1nc2c3c(c(N)n(-c4c(C)ccc(O)c4C)c3n1)C(=O)Nc1ccccc1C2C1CC1]

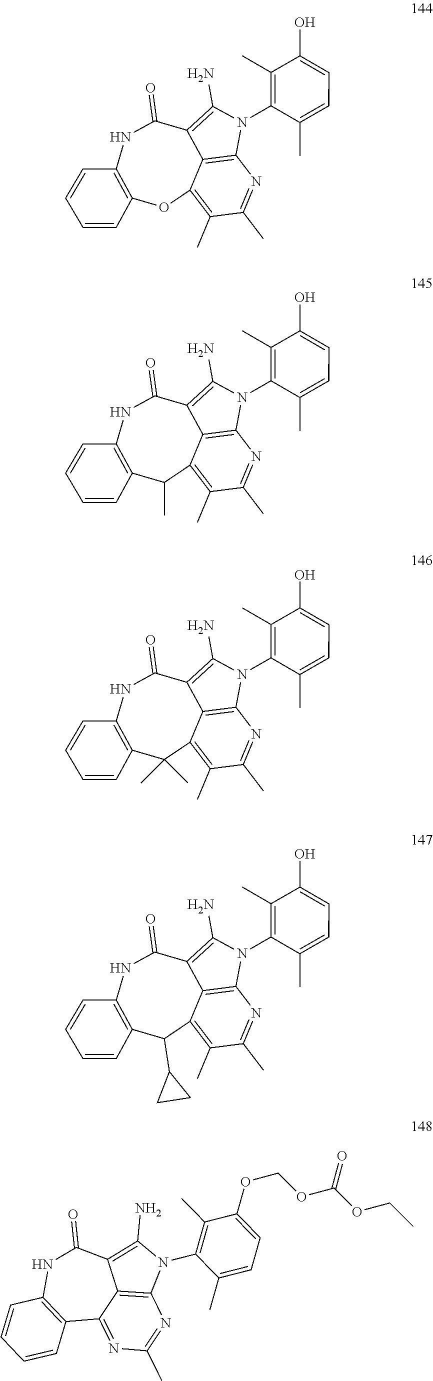 [SMILES: Cc1ccc(O)c(C)c1-n1c(N)c2c3c(c(C)c(C)nc31)Oc1ccccc1NC2=O ; Cc1ccc(O)c(C)c1-n1c(N)c2c3c(c(C)c(C)nc31)C(C)c1ccccc1NC2=O ; Cc1ccc(O)c(C)c1-n1c(N)c2c3c(c(C)c(C)nc31)C(C)(C)c1ccccc1NC2=O ; Cc1ccc(O)c(C)c1-n1c(N)c2c3c(c(C)c(C)nc31)C(C1CC1)c1ccccc1NC2=O ; CCOC(=O)OCOc1ccc(C)c(-n2c(N)c3c4c(nc(C)nc42)-c2ccccc2NC3=O)c1C]

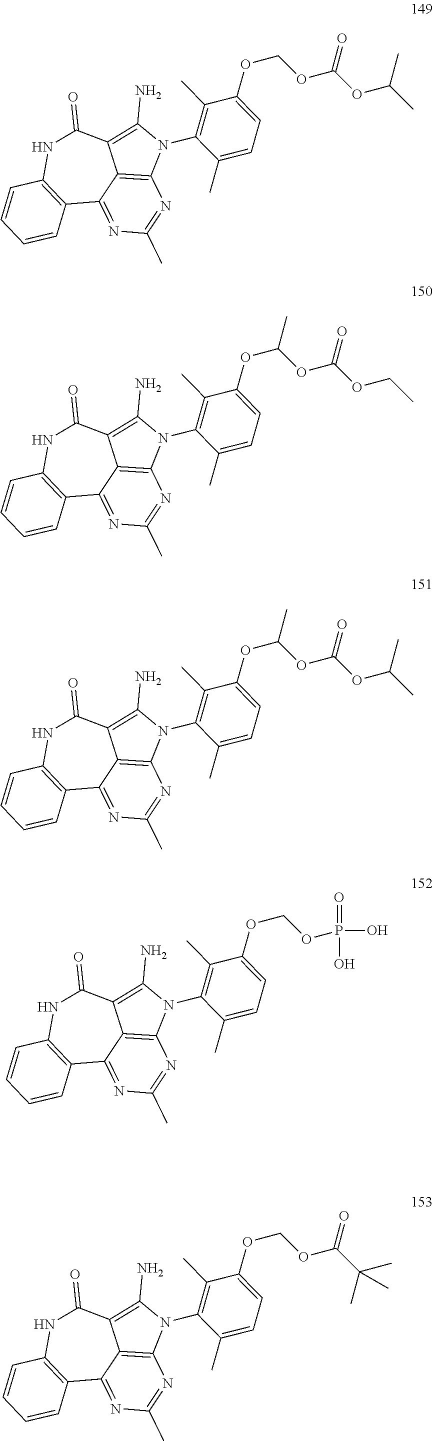 [SMILES: Cc1nc2c3c(c(N)n(-c4c(C)ccc(OCOC(=O)OC(C)C)c4C)c3n1)C(=O)Nc1ccccc1-2 ; CCOC(=O)OC(C)Oc1ccc(C)c(-n2c(N)c3c4c(nc(C)nc42)-c2ccccc2NC3=O)c1C ; Cc1nc2c3c(c(N)n(-c4c(C)ccc(OC(C)OC(=O)OC(C)C)c4C)c3n1)C(=O)Nc1ccccc1-2 ; Cc1nc2c3c(c(N)n(-c4c(C)ccc(OCOP(=O)(O)O)c4C)c3n1)C(=O)Nc1ccccc1-2 ; Cc1nc2c3c(c(N)n(-c4c(C)ccc(OCOC(=O)C(C)(C)C)c4C)c3n1)C(=O)Nc1ccccc1-2]

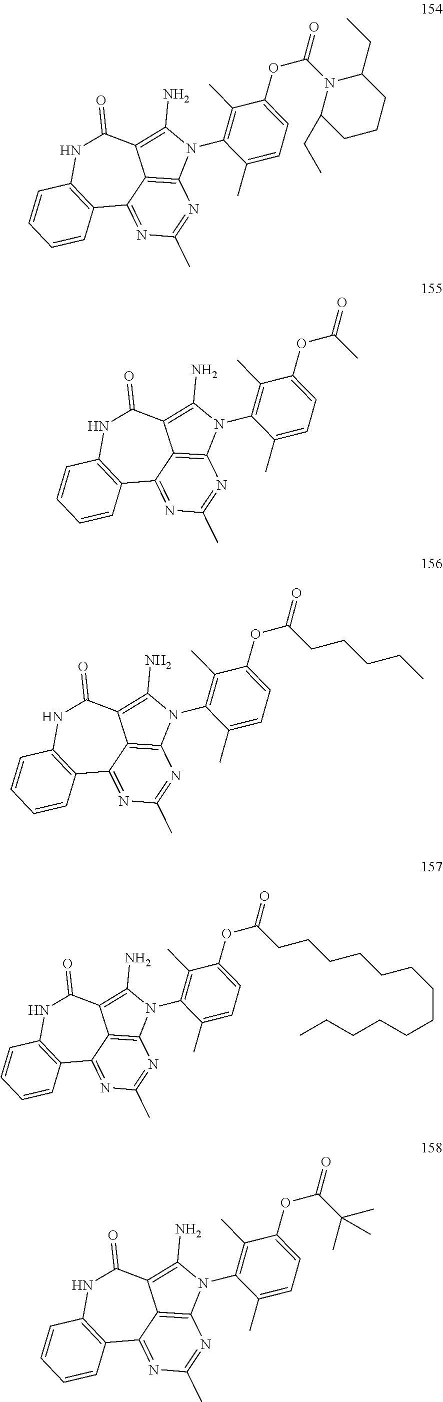 [SMILES: CCC1CCCC(CC)N1C(=O)Oc1ccc(C)c(-n2c(N)c3c4c(nc(C)nc42)-c2ccccc2NC3=O)c1C ; CC(=O)Oc1ccc(C)c(-n2c(N)c3c4c(nc(C)nc42)-c2ccccc2NC3=O)c1C ; CCCCCC(=O)Oc1ccc(C)c(-n2c(N)c3c4c(nc(C)nc42)-c2ccccc2NC3=O)c1C ; CCCCCCCCCCCCCC(=O)Oc1ccc(C)c(-n2c(N)c3c4c(nc(C)nc42)-c2ccccc2NC3=O)c1C ; Cc1nc2c3c(c(N)n(-c4c(C)ccc(OC(=O)C(C)(C)C)c4C)c3n1)C(=O)Nc1ccccc1-2]

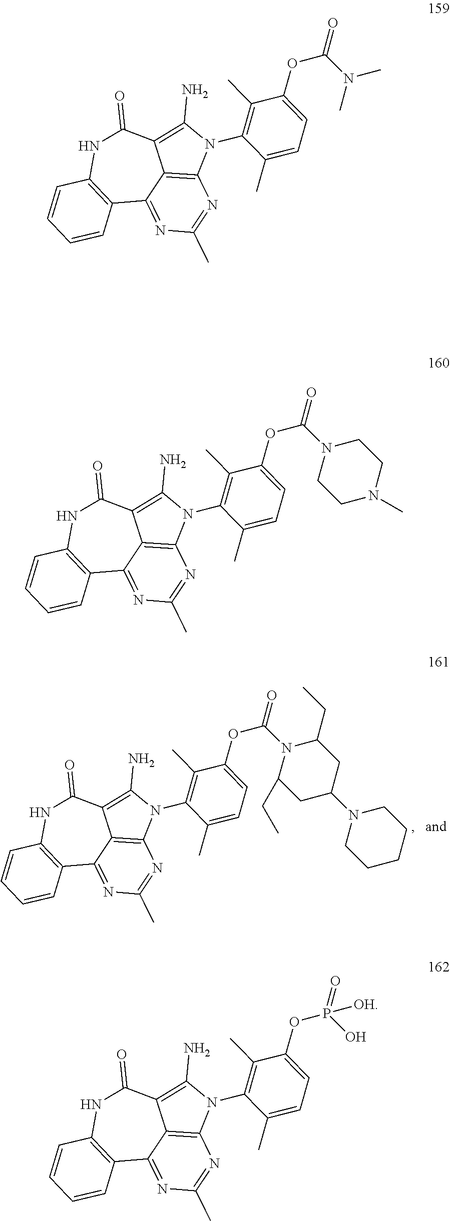 [SMILES: Cc1nc2c3c(c(N)n(-c4c(C)ccc(OC(=O)N(C)C)c4C)c3n1)C(=O)Nc1ccccc1-2 ; Cc1nc2c3c(c(N)n(-c4c(C)ccc(OC(=O)N5CCN(C)CC5)c4C)c3n1)C(=O)Nc1ccccc1-2 ; CCC1CC(N2CCCCC2)CC(CC)N1C(=O)Oc1ccc(C)c(-n2c(N)c3c4c(nc(C)nc42)-c2ccccc2NC3=O)c1C ; Cc1nc2c3c(c(N)n(-c4c(C)ccc(OP(=O)(O)O)c4C)c3n1)C(=O)Nc1ccccc1-2]

In another aspect, the present application provides a pharmaceutical composition comprising the compound of formula (I) or the pharmaceutically acceptable salt thereof of the present application and a pharmaceutically acceptable excipient.

In another aspect, the present application provides a method for treating diseases mediated by PKMYT1 in a mammal, comprising administering to a mammal, preferably a human, in need of such treatment a therapeutically effective amount of the compound of formula (I) or the pharmaceutically acceptable salt thereof, or the pharmaceutical composition comprising the same.

In another aspect, the present application provides use of the compound of formula (I) or the pharmaceutically acceptable salt thereof, or the pharmaceutical composition comprising the same in preparing a medicament for use in preventing or treating diseases mediated by PKMYT1.

In another aspect, the present application provides use of the compound of formula (I) or the pharmaceutically acceptable salt thereof, or the pharmaceutical composition comprising the same in preventing or treating diseases mediated by PKMYT1.

In another aspect, the present application provides the compound of formula (I) or the pharmaceutically acceptable salt thereof, or the pharmaceutical composition comprising the same for use in preventing or treating diseases mediated by PKMYT1.

In some embodiments, the diseases mediated by PKMYT1 are tumors. In some embodiments, the disease mediated by PKMYT1 is liver cancer.

### TERMINOLOGY AND DEFINITIONS

Unless otherwise stated, the terms used in the present disclosure have the following meanings, and the definitions of groups and terms described in the present disclosure, including their definitions as examples, exemplary definitions, preferred definitions, definitions documented in tables, definitions of specific compounds in the Examples, and the like, may be arbitrarily combined or incorporated with each other. A specific term without specifical definition should not be considered uncertain or unclear, and it should be understood in its ordinary meaning in the art. When referring to a trade name, it is intended to refer to its corresponding commercial product or its active ingredient.

Herein, “

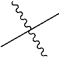

” represents a linking site.

Herein, the bond [symbol] depicted by a solid line and a dotted line represents a single bond or a double bond.

The term “tautomer” refers to functional isomers resulting from the rapid movement of a certain atom between two positions in the molecule. The compounds of the present disclosure may exhibit the tautomerism. Tautomeric compounds may exist in two or more interconvertible forms. Tautomers generally exist in equilibrium. Trying to separate a single tautomer usually leads to a mixture, the physicochemical properties of the single tautomer are consistent with those of the mixture of compounds. The position of the equilibrium depends on intramolecular chemical properties. For example, in many aliphatic aldehydes and ketones such as acetaldehyde, the keto-form predominates; whereas in phenol, the enol-form predominates. In the present disclosure, all tautomeric forms of the compound are included.

The term “stereoisomer” refers to isomers resulting from different spatial arrangements of the atoms in a molecule, including cis-trans-isomers, enantiomers, and diastereoisomers.

The compound of the present disclosure may have asymmetric atom(s) such as carbon atom(s), sulfur atom(s), nitrogen atom(s), and phosphorus atom(s), or asymmetric double bond(s), and thus the compound of the present disclosure may exist in the form of a particular geometric isomer or stereoisomer. The form of a particular geometric isomer or stereoisomer may be cis- and trans-isomers, E and Z geometric isomers, (−)- and (+)-enantiomers, (R)- and (S)-enantiomers, diastereoisomers, (D)-isomers, (L)-isomers, and racemic mixtures and other mixtures thereof, such as enantiomerically or diasteriomerically enriched mixtures, and all of the above isomers, as well as mixtures thereof, are encompassed within the definition scope of the compound of the present disclosure. Additional asymmetric carbon atom(s), asymmetric sulfur atom(s), asymmetric nitrogen atom(s), or asymmetric phosphorus atom(s) may be present in substituent(s) such as alkyl. All of these isomers and the mixtures thereof referred to in the substituents are also encompassed within the definition scope of the compound of the present disclosure. The compound with asymmetric atom(s) of the present disclosure can be separated in an enantiomerically pure form or in a racemic form. The enantiomerically pure form can be obtained by resolving racemic mixtures or by synthesis using chiral starting materials or chiral reagents.

The term “substituted” means that any one or more hydrogen atoms on a specific atom are replaced with substituent(s), as long as the valence of the specific atom is normal and the compound resulting from the substitution is stable. When the substituent is oxo (namely ═O), it means that two hydrogen atoms are substituted, and oxo is not available on an aromatic group.

The term “optional” or “optionally” means that the subsequently described event or circumstance may or may not occur. The description includes instances where the event or circumstance occurs and instances where the event or circumstance does not occur. For example, ethyl being “optionally” substituted with halogen means that the ethyl may be unsubstituted (CH₂CH₃), monosubstituted (CH₂CH₂F, CH₂CH₂Cl, or the like), polysubstituted (CHFCH₂F, CH₂CHF₂, CHFCH₂Cl, CH₂CHCl₂, or the like), or fully substituted (CF₂CF₃, CF₂CCl₃, CCl₂CCl₃, or the like). It will be understood by those skilled in the art that for any group containing one or more substituents, no substitution or substituting pattern that is sterically impossible to exist and/or cannot be synthesized will be introduced.

When any variable (e.g., n, R^{a}, or R^{b}) appears more than once in the composition or structure of a compound, the variable is independently defined in each case. For example, if a group is substituted with 2 R^{b}, the definition of each R^{b} is independent.

When the number of a linking group is 0, such as —(CH₂)₀—, it means that the linking group is a bond.

When one of variables is selected from the group consisting of a chemical bond or is absent, it means that the two groups which it links are linked directly. For example, when L in A-L-Z represents a bond, it means that the structure is actually A-Z.

Cₘ-Cₙ used herein means that the portion has an integer number of carbon atoms in the range of m-n. For example, “C₁-C₁₀” means that the group may have 1, 2, 3, 4, 5, 6, 7, 8, 9, or 10 carbon atoms.

The term “alkyl” refers to a hydrocarbyl group with a general formula of CₙH₂ₙ₊₁. The alkyl may be linear or branched. The term “C₁-C₁₀ alkyl” may be understood to represent a linear or branched saturated hydrocarbyl group having 1, 2, 3, 4, 5, 6, 7, 8, 9, or 10 carbon atoms. Specific examples of the alkyl include, but are not limited to, methyl, ethyl, propyl, butyl, pentyl, hexyl, isopropyl, isobutyl, sec-butyl, tert-butyl, isopentyl, 2-methylbutyl, 1-methylbutyl, 1-ethylpropyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, and the like. The term “C₁-C₆ alkyl” may be understood to represent an alkyl group having 1 to 6 carbon atoms. Specific examples include, but are not limited to, methyl, ethyl, n-propyl, isopropyl, n-butyl, isobutyl, sec-butyl, tert-butyl, n-pentyl, 1-methylbutyl, 2-methylbutyl, 3-methylbutyl, neopentyl, hexyl, 2-methylpentyl, and the like. The term “C₁-C₃ alkyl” may be understood to represent a linear or branched saturated alkyl group having 1 to 3 carbon atoms. The “C₁-C₁₀ alkyl” may include the range of “C₁-C₆ alkyl”, “C₁-C₃ alkyl”, or the like, and the “C₁-C₆ alkyl” may further include “C₁-C₃ alkyl”. The term “C₁-C₃ haloalkyl” refers to C₁-C₃ alkyl substituted with one or more halogens such as F, Cl, Br, or I, including monosubstituted, polysubstituted, or fully substituted.

The term “alkylene” refers to a saturated linear or branched aliphatic hydrocarbyl group having 2 residues derived from a parent alkane by the removal of two hydrogen atoms from the same carbon atom or two different carbon atoms, and it may be alkylene containing 1 to 12 (e.g., 1, 2, 3, 4, 5, 6, 7, 8, 9, 10, 11, and 12) carbon atoms, preferably alkylene containing 1, 2, 3, 4, 5, or 6 carbon atoms (i.e., C₁-C₆ alkylene), and more preferably alkylene containing 1, 2, or 3 carbon atoms (i.e., C₁-C₃ alkylene). Non-limiting examples of alkylene include, but are not limited to, methylene, —CH(CH₃)—, —CH₂CH₂—, —CH(CH₂CH₃)—, —CH₂CH(CH₃)—, —CH₂CH₂CH₂—, and the like.

The term “alkoxy” refers to a group derived from a linear or branched alcohol by loss of a hydrogen atom from hydroxy, and may be understood as “alkyloxy” or “alkyl-O—”. The term “C₁-C₁₀ alkoxy” may be understood as “C₁-C₁₀ alkyloxy” or “C₁-C₁₀ alkyl-O—”; the term “C₁-C₆ alkoxy” may be understood as “C₁-C₆ alkyloxy” or “C₁-C₆ alkyl-O—”. The “C₁-C₁₀ alkoxy” may include the ranges of “C₁-C₆ alkoxy”, “C₁-C₃ alkoxy”, and the like, and the “C₁-C₆ alkoxy” may further include “C₁-C₃ alkoxy”. The term “C₁-C₃ haloalkoxy” refers to C₁-C₃ haloalkyl-O—.

The term “alkenyl” refers to a linear or branched unsaturated aliphatic hydrocarbyl group consisting of carbon atoms and hydrogen atoms and having at least one double bond. The term “C₂-C₁₀ alkenyl” may be understood to represent a linear or branched unsaturated hydrocarbyl group comprising one or more double bonds and having 2, 3, 4, 5, 6, 7, 8, 9, or 10 carbon atoms, and the “C₂-C₁₀ alkenyl” is preferably “C₂-C₆ alkenyl”, further preferably “C₂-C₄ alkenyl”, and still further preferably C₂ or C₃ alkenyl. It may be understood that in the case that the alkenyl comprises more than one double bond, the double bonds can be isolated from one another or conjugated. Specific examples of the alkenyl includes, but is not limited to, vinyl, allyl, (E)-2-methylvinyl, (Z)-2-methylvinyl, (E)-but-2-enyl, (Z)-but-2-enyl, (E)-but-1-enyl, (Z)-but-1-enyl, isopropenyl, 2-methylprop-2-enyl, 1-methylprop-2-enyl, 2-methylprop-1-enyl, (E)-1-methylprop-1-enyl, (Z)-1-methylprop-1-enyl, and the like.

The term “alkynyl” refers to a linear or branched unsaturated aliphatic hydrocarbyl group consisting of carbon atoms and hydrogen atoms and having at least one triple bond. The term “C₂-C₁₀ alkynyl” may be understood to represent a linear or branched unsaturated hydrocarbyl group comprising one or more triple bonds and having 2, 3, 4, 5, 6, 7, 8, 9, or 10 carbon atoms. Examples of “C₂-C₁₀ alkynyl” include, but are not limited to, ethynyl (—C≡CH), propynyl (—C≡CCH₃ or —CH₂C≡CH), but-1-ynyl, but-2-ynyl, and but-3-ynyl. “C₂-C₁₀ alkynyl” may include “C₂-C₃ alkynyl”, and examples of “C₂-C₃ alkynyl” include ethynyl (—C≡CH), prop-1-ynyl (—C≡CCH₃), and prop-2-ynyl (—CH₂C≡CH).

The term “cycloalkyl” refers to a saturated carbon ring that exists in the form of a monocyclic ring, fused ring, bridged ring, spiro ring, or the like. Unless otherwise specified, the carbon ring is generally a 3- to 10-membered ring. The term “C₃-C₁₀ cycloalkyl” may be understood to represent a saturated monocyclic, fused, spiro, or bridged ring having 3-10 (3, 4, 5, 6, 7, 8, 9, or 10) carbon atoms. Specific examples of the cycloalkyl include, but are not limited to, cyclopropyl, cyclobutyl, cyclopentyl, cyclohexyl, cycloheptyl, cyclooctyl, cyclononyl, cyclodecyl, norbornyl (bicyclo[2.2.1]heptyl), bicyclo[2.2.2]octyl, adamantyl, spiro[4.5]decyl, and the like. The term “C₃-C₁₀ cycloalkyl” may include “C₃-C₆ cycloalkyl”, and the term “C₃-C₆ cycloalkyl” may be understood to represent a saturated monocyclic or bicyclic hydrocarbyl ring having 3-6 carbon atoms. Specific examples include, but are not limited to, cyclopropyl, cyclobutyl, cyclopentyl, cyclohexyl, and the like.

The term “cycloalkyloxy” may be understood as “cycloalkyl-O—”.

The term “heterocyclyl” or “heterocyclic ring” refers to a saturated or partially saturated (but not an aromatic heteroaryl on the whole) monocyclic, fused cyclic, spiro cyclic, or bridged cyclic group, and ring atoms of the group include 1-5 heteroatoms or heteroatom groups (i.e., heteroatom-containing groups). The “heteroatom or heteroatom group” includes, but is not limited to, a nitrogen atom (N), an oxygen atom (O), a sulfur atom (S), a phosphorus atom (P), a boron atom (B), —S(═O)₂—, —S(═O)—, —P(═O)₂—, —P(═O)—, —NH—, —S(═O)(═NH)—, —C(═O)NH—, —NHC(═O)NH—, and the like. The term “4-9 membered heterocyclyl” refers to a heterocyclyl group having 4, 5, 6, 7, 8, or 9 ring atoms, and ring atoms of the heterocyclyl group include 1, 2, 1-3, or 1-5 heteroatoms or heteroatom groups independently selected from the group consisting of those described above. “4-7 membered heterocyclyl” and “4-9 membered heterocyclyl” may each contain 1-3 (1, 2, or 3) heteroatoms independently selected from the group consisting of N, O, and S. “4-9 membered heterocyclyl” includes “4-7 membered heterocyclyl”, wherein specific examples of 4-membered heterocyclyl include, but are not limited to, azetidinyl and oxetanyl; specific examples of 5-membered heterocyclyl include, but are not limited to, tetrahydrofuranyl, dioxolyl, pyrrolidinyl, imidazolidinyl, pyrazolidinyl, pyrrolinyl, 4,5-dihydrooxazolyl, and 2,5-dihydro-1H-pyrrolyl; specific examples of 6-membered heterocyclyl include, but are not limited to, tetrahydropyranyl, piperidinyl, morpholinyl, dithianyl, thiomorpholinyl, piperazinyl, trithianyl, tetrahydropyridinyl, and 4H-[1,3,4]thiadiazinyl; and specific examples of 7-membered heterocyclyl include, but are not limited to, diazepanyl. The heterocyclyl may also be a bicyclic group, wherein specific examples of 5,5-membered bicyclic groups include, but are not limited to, hexahydrocyclopenta[c]pyrrol-2(1H)-yl; and specific examples of 5,6-membered bicyclic groups include, but are not limited to, hexahydropyrrolo[1,2-a]pyrazin-2(1H)-yl, 5,6,7,8-tetrahydro-[1,2,4]triazolo[4,3-a]pyrazinyl, and 5,6,7,8-tetrahydroimidazo[1,5-a]pyrazinyl. Optionally, the heterocyclyl may be a benzo-fused ring group of the 4-7 membered heterocyclyl described above. Specific examples include, but are not limited to, dihydroisoquinolyl and the like. “4-9 membered heterocyclyl” may include the ranges of “5-9 membered heterocyclyl”, “4-7 membered heterocyclyl”, “5-6 membered heterocyclyl”, “6-8 membered heterocyclyl”, “4-9 membered heterocycloalkyl”, “5-9 membered heterocycloalkyl”, “4-7 membered heterocycloalkyl”, “5-6 membered heterocycloalkyl”, “6-8 membered heterocycloalkyl”, and the like, and the “4-7 membered heterocyclyl” may further include the ranges of “4-6 membered heterocyclyl”, “5-6 membered heterocyclyl”, “4-7 membered heterocycloalkyl”, “4-6 membered heterocycloalkyl”, “5-6 membered heterocycloalkyl”, and the like. Although some bicyclic heterocyclyl herein comprise, in part, a benzene ring or a heteroaromatic ring, the heterocyclyl is still non-aromatic on the whole.

The term “heterocycloalkyl” refers to a saturated cyclic group that exists in the form of a monocyclic ring, fused ring, bridged ring, spiro ring, or the like, and ring atoms of the group include 1, 2, 1-3, or 1-5 heteroatoms or heteroatom groups (i.e., heteroatom-containing groups). The “heteroatom or heteroatom group” includes, but is not limited to, a nitrogen atom (N), an oxygen atom (O), a sulfur atom (S), a phosphorus atom (P), a boron atom (B), —S(═O)₂—, —S(═O)—, —NH—, —S(═O)(═NH)—, —C(═O)NH—, —NHC(═O)NH—, and the like. The term “4-9 membered heterocycloalkyl” refers to a heterocycloalkyl group with 4, 5, 6, 7, 8, or 9 ring atoms, and ring atoms of the group include 1-5 heteroatoms or heteroatom groups independently selected from the group consisting of those described above. “4-7 membered heterocycloalkyl” and “4-9 membered heterocycloalkyl” may each contain 1-3 (1, 2, or 3) heteroatoms independently selected from the group consisting of N, O, and S. “4-9 membered heterocycloalkyl” includes “4-7 membered heterocycloalkyl”, wherein specific examples of 4-membered heterocycloalkyl include, but are not limited to, azetidinyl, oxetanyl, and thietanyl; specific examples of 5-membered heterocycloalkyl include, but are not limited to, tetrahydrofuranyl, tetrahydrothienyl, pyrrolidinyl, isoxazolidinyl, oxazolidinyl, isothiazolidinyl, thiazolidinyl, imidazolidinyl, and tetrahydropyrazolyl; specific examples of 6-membered heterocycloalkyl include, but are not limited to, piperidinyl, tetrahydropyranyl, tetrahydrothiopyranyl, morpholinyl, piperazinyl, 1,4-thioxanyl, 1,4-dioxanyl, thiomorpholinyl, 1,3-dithianyl, and 1,4-dithianyl; and specific examples of 7-membered heterocycloalkyl include, but are not limited to, azepanyl, oxepanyl, and thiepanyl.

The term “aryl” refers to an aromatic all-carbon monocyclic or fused polycyclic group with a conjugated π-electron system. Aryl may have 6-20 carbon atoms, 6-14 carbon atoms, 6-12 carbon atoms, or 6-10 carbon atoms. The term “C₆-C₂₀ aryl” may be understood as an aryl group having 6-20 carbon atoms, particularly a ring having 6 carbon atoms (“C₆ aryl”), such as phenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl, or naphthyl, or a ring having 13 carbon atoms (“C₁₃ aryl”), such as fluorenyl, or a ring having 14 carbon atoms (“C₁₄ aryl”), such as anthryl. The term “C₆-C₁₀ aryl” may be understood as an aryl group having 6-10 carbon atoms, particularly a ring having 6 carbon atoms (“C₆ aryl”), such as phenyl, or a ring having 9 carbon atoms (“C₉ aryl”), such as indanyl or indenyl, or a ring having 10 carbon atoms (“C₁₀ aryl”), such as tetrahydronaphthyl, dihydronaphthyl, or naphthyl.

The term “heteroaryl” or “heteroaromatic ring” refers to an aromatic cyclic group having an aromatic monocyclic or fused polycyclic system, which contains at least one (1, 2, or 3) ring atom selected from the group consisting of N, O, and S, with the remaining ring atoms being C. The term “5-9 membered heteroaryl” may be understood to include an aromatic monocyclic or bicyclic ring system, which has 5, 6, 7, 8, or 9 ring atoms, particularly 5 or 6 or 9 ring atoms, and comprises 1-5, preferably 1-3 heteroatoms independently selected from the group consisting of N, O, and S. In particular, the heteroaryl is selected from the group consisting of thienyl, furanyl, pyrrolyl, oxazolyl, thiazolyl, imidazolyl, pyrazolyl, isoxazolyl, isothiazolyl, oxadiazolyl, triazolyl, thiadiazolyl, and the like, and the benzo derivatives thereof, such as benzofuranyl, benzothienyl, benzothiazolyl, benzoxazolyl, benzoisoxazolyl, benzimidazolyl, benzotriazolyl, indazolyl, indolyl, isoindolyl, and the like; and pyridyl, pyridazinyl, pyrimidinyl, pyrazinyl, triazinyl, and the like, and the benzo derivatives thereof, such as quinolyl, quinazolinyl, isoquinolyl, and the like; and azocinyl, indolizinyl, purinyl, and the like, and the benzo derivatives thereof; and cinnolinyl, phthalazinyl, quinazolinyl, quinoxalinyl, naphthyridinyl, pteridinyl, carbazolyl, acridinyl, phenazinyl, phenothiazinyl, phenoxazinyl, and the like. The term “5-6 membered heteroaryl” refers to an aromatic ring system, which has 5 or 6 ring atoms and comprises 1-3, preferably 1-2 heteroatoms independently selected from the group consisting of N, O, and S.

The term “halo” or “halogen” refers to fluorine, chlorine, bromine, or iodine.

The term “hydroxymethyl” refers to —CH₂OH.

The term “hydroxy” refers to the —OH group.

The term “cyano” refers to the —CN group.

The term “sulfydryl” refers to the —SH group.

The term “amino” refers to the —NH₂ group.

The term “nitro” refers to the —NO₂ group.

The term “therapeutically effective amount” refers to an amount of the compound of the present disclosure for (i) treating a specific disease, condition, or disorder; (ii) alleviating, ameliorating, or eliminating one or more symptoms of a specific disease, condition, or disorder; or (iii) delaying the onset of the one or more symptoms of the specific disease, condition, or disorder described herein.

The amount of the compound disclosed herein constituting the “therapeutically effective amount” varies dependently on the compound, the disease state and its severity, the administration regimen, and the age of the mammal to be treated, but can be determined routinely by those skilled in the art in accordance with their knowledge and the content of the present disclosure.

The term “pharmaceutically acceptable” is used herein for those compounds, materials, compositions, and/or dosage forms which are, within the scope of sound medical judgment, suitable for use in contact with the tissues of human beings and animals without excessive toxicity, irritation, allergic response, or other problems or complications, and commensurate with a reasonable benefit/risk ratio.

The term “pharmaceutically acceptable salt” refers to salts of pharmaceutically acceptable acids or bases, including salts formed from the compound and an inorganic or organic acid, and salts formed from the compound and an inorganic or organic base.

The term “pharmaceutical composition” refers to a mixture consisting of one or more of the compounds or the salts thereof of the present disclosure and a pharmaceutically acceptable excipient.

The pharmaceutical composition is intended to facilitate the administration of the compound of the present disclosure to an organism.

The term “pharmaceutically acceptable excipients” refers to those that do not have a significant irritating effect on an organism and do not impair the biological activity and properties of the active compound. Suitable excipients are well known to those skilled in the art, such as carbohydrate, wax, water-soluble and/or water-swellable polymers, hydrophilic or hydrophobic materials, gelatin, oil, solvent, water, and the like.

The word “comprise” and variations thereof such as “comprises” or “comprising” may be understood in an open, non-exclusive sense, i.e., “including but not limited to”.

The present disclosure also includes isotopically labeled compounds of the present disclosure which are identical to those documented herein but have one or more atoms replaced by atom(s) having an atomic mass or mass number different from the atomic mass or mass number usually found in nature. Examples of isotopes that can be incorporated into the compounds of the present disclosure include isotopes of hydrogen, carbon, nitrogen, oxygen, phosphorus, sulfur, fluorine, iodine and chlorine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ¹²³I, ¹²⁵I and ³⁶Cl, respectively, and the like.

Certain isotopically labeled compounds of the present disclosure (e.g., those labeled with ³H and ¹⁴C) can be used to analyze tissue distribution of compounds and/or substrates. Tritiated (i.e., ³H) and carbon-14 (i.e., ¹⁴C) isotopes are particularly preferred for their ease of preparation and detectability.

Positron emitting isotopes, such as ¹⁵O, ¹³N, ¹¹C, and ¹⁸F, can be used in positron emission tomography (PET) studies to determine substrate occupancy. Isotopically labeled compounds of the present disclosure can generally be prepared by following procedures analogous to those disclosed in the schemes and/or examples below while substituting a non-isotopically labeled reagent with an isotopically labeled reagent.

The pharmaceutical composition of the present disclosure can be prepared by combining the compound of the present disclosure with a suitable pharmaceutically acceptable excipient, and can be formulated, for example, into a solid, semi-solid, liquid, or gaseous formulation such as tablets, pills, capsules, powders, granules, ointments, emulsions, suspensions, suppositories, injections, inhalants, gels, microspheres, aerosols, and the like.

Typical routes of administration of the compound or the pharmaceutically acceptable salt thereof or the pharmaceutical composition thereof of the present disclosure include, but are not limited to, oral, rectal, local, inhalation, parenteral, sublingual, intravaginal, intranasal, intraocular, intraperitoneal, intramuscular, subcutaneous, and intravenous administration.

The pharmaceutical composition of the present disclosure can be manufactured by methods well-known in the art, such as conventional methods of mixing, dissolving, granulating, emulsifying, lyophilizing, and the like.

In some embodiments, the pharmaceutical composition is in an oral form. For oral administration, the pharmaceutical composition can be formulated by mixing the active compounds with pharmaceutically acceptable excipients well-known in the art. These excipients enable the compounds of the present disclosure to be formulated into tablets, pills, lozenges, dragees, capsules, liquids, gels, slurries, suspensions, and the like, for oral administration to patients.

A solid oral composition can be prepared by conventional mixing, filling, or tableting method. For example, it can be obtained by the following method: mixing the active compounds with solid excipients, optionally grinding the resulting mixture, adding additional suitable excipients if desired, and processing the mixture into granules to get the core parts of tablets or dragees. Suitable excipients include, but are not limited to: binders, diluents, disintegrants, lubricants, glidants, or flavoring agents, and the like.

The pharmaceutical compositions may also be suitable for parenteral administration, such as sterile solutions, suspensions, or lyophilized products in suitable unit dosage forms.

In all of the administration methods for the compound of general formula (I) described herein, the daily dose administered is from 0.01 mg/kg body weight to 200 mg/kg body weight, preferably from 0.05 mg/kg body weight to 50 mg/kg body weight, and more preferably from 0.1 mg/kg body weight to 30 mg/kg body weight, given in a single dose or divided doses.

The compounds of the present disclosure can be prepared by a variety of synthetic methods well known to those skilled in the art, including the specific embodiments listed below, embodiments formed by combinations thereof with other chemical synthetic methods, and equivalents thereof well-known to those skilled in the art. The preferred embodiments include, but are not limited to, the examples of the present disclosure.

The chemical reactions of the specific embodiments disclosed herein are conducted in a suitable solvent that must be suitable for the chemical changes in the present disclosure and the reagents and materials required thereby. In order to obtain the compounds of the present disclosure, it is sometimes necessary for those skilled in the art to modify or select synthesis steps or a reaction procedures based on the existing embodiments.

### DETAILED DESCRIPTION

The following abbreviations are used in the present disclosure: DCM for dichloromethane; LiHMDS for lithium bis(trimethylsilyl)amide; DMF for N,N-dimethylformamide; TsOH·H₂O for p-toluenesulfonic acid monohydrate; LiTMP for lithium 2,2,6,6-tetramethylpiperidine; B(OiPr)₃ for triisopropyl borate; B(OMe)₃ for trimethyl borate; NMP for N-methyl-2-pyrrolidone; DME for ethylene glycol dimethyl ether; KOAc for potassium acetate; AcOH for acetic acid; ^{t}BuOH for tert-butanol; NaO^{t}Bu for sodium tert-butoxide; ^{t}BuLi for tert-butyllithium; n-BuLi for n-butyllithium; dppf for 1,1′-bis(diphenylphosphino)ferrocene; Pd(dppf)Cl₂ for [1,1′-bis(diphenylphosphino)ferrocene]palladium dichloride; Pd(dppf)Cl₂·DCM for a [1,1′-bis(diphenylphosphino)ferrocene]palladium dichloride dichloromethane complex; Pd₂(dba)₃ for tris(dibenzylideneacetone)dipalladium; Pd(dba)₂ for bis(dibenzylideneacetone)palladium; rt for room temperature; min for minute; h for hour; Me for methyl; MeOH for methanol; EtOH for ethanol; ⁱPr for isopropyl; Boc for tert-butoxycarbonyl; (Boc)₂O for di-tert-butyl dicarbonate; EtOAc for ethyl acetate; THF for tetrahydrofuran; TEA/Et₃N for triethylamine; Xphos for 2-dicyclohexylphosphino-2′,4′,6′-triisopropylbiphenyl; Xphos Pd G1 for chloro(2-dicyclohexylphosphino-2′,4′,6′-triisopropyl-1,1′-biphenyl)[2-(2-aminoethylphenyl)]palladium(II); SPhos for 2-dicyclohexylphosphino-2′,6′-dimethoxybiphenyl; t-BuXPhos-Pd-G₃ for methanesulfonato(2-di-tert-butylphosphino-2′,4′,6′-triisopropyl-1,1′-biphenyl)(2′-amino-1,1′-biphenyl-2-yl)palladium(II); PPh₃ for triphenylphosphine; Pd(PPh₃)₄ for tetrakis(triphenylphosphine)palladium; meCgPPh for 1,3,5,7-tetramethyl-6-phenyl-2,4,8-trioxa-6-phosphaadamantane; dioxane for 1,4-dioxane; Ni-Raney for Raney nickel; DMAP for 4-dimethylaminopyridine; B₂Pin₂ for bis(pinacolato)diboron; Tf for trifluoromethanesulfonyl; DPPA for diphenylphosphoryl azide; Sn₂ⁿBu₆ for hexa-n-butylditin; Dess-Martin for Dess-Martin oxidizer; Et₃SiH for triethylsilane; Pd(OAc)₂ for palladium acetate; PCy₃ for tricyclohexylphosphine; MOMO for methoxymethoxy; PhNTf₂ for N-phenylbis(trifluoromethanesulfonyl)imide; DPPF for 1,1′-bis(diphenylphosphino)ferrocene; m-CPBA for m-chloroperoxybenzoic acid; TMSCN for trimethylsilyl cyanide; ACN for acetonitrile; NBS for n-bromosuccinimide; AcCl for acetyl chloride; DMAP for 4-dimethylaminopyridine; ^{t}BuONO for tert-butyl nitrite; cataCXium A Pd G₃ for mesylate[n-butyldi(1-adamantyl)phosphine](2-amino-1,1′-biphenyl-2-yl)palladium(II); TEMP for 2,2,6,6-tetramethylpiperidine; NIS for N-iodosuccinimide; DIAD for diisopropyl azodicarboxylate; TMSI for trimethylsilyl iodide; DCE for 1,2-dichloroethane; PhNTf₂ for N-phenylbis(trifluoromethanesulfonyl)imide; xantphos for 4,5-bis(diphenylphosphino)-9,9-dimethylxanthene; DIPEA for N,N-diisopropylethylamine; ATP for adenosine triphosphate; CDK1 for cyclin-dependent kinase 1; ADP for adenosine diphosphate; FBS for fetal bovine serum.

The present disclosure is described in detail below by way of examples, which, however, are not intended to disadvantageously limit the scope of the present disclosure in any way. Although the present disclosure has been described in detail herein and specific embodiments have also been disclosed, it will be apparent to those skilled in the art that various modifications can be made to the specific embodiments of the present disclosure without departing from the spirit and scope of the present disclosure. All reagents used in the present disclosure are commercially available and can be used without further purification.

Unless otherwise stated, the ratios expressed for mixed solvents are volume mixing ratios.

Unless otherwise stated, % refers to wt %.

Compounds are named either manually or by ChemDraw® software, and supplier's catalog names are given for commercially available compounds.

The structures of the compounds are determined by nuclear magnetic resonance (NMR) and/or mass spectrometry (MS). NMR shifts are given in 10⁻⁶ (ppm). The solvents for NMR determination are deuterated dimethyl sulfoxide, deuterated chloroform, deuterated methanol, and the like, and the internal standard is tetramethylsilane (TMS); “IC₅₀” refers to the half maximal inhibitory concentration, which is the concentration at which half of the maximal inhibitory effect is achieved.

Hereinafter, the eluent may be a mixed eluent formed by two or more solvents, and the ratio thereof is the volume ratio of each solvent.

### Example 1. Preparation of 5-amino-4-(3-hydroxy-2,6-dimethylphenyl)-4,7-dihydro-6H-1,3,4,7-tetraazadibenzo[cd,f]azulen-6-one (Compound 1)

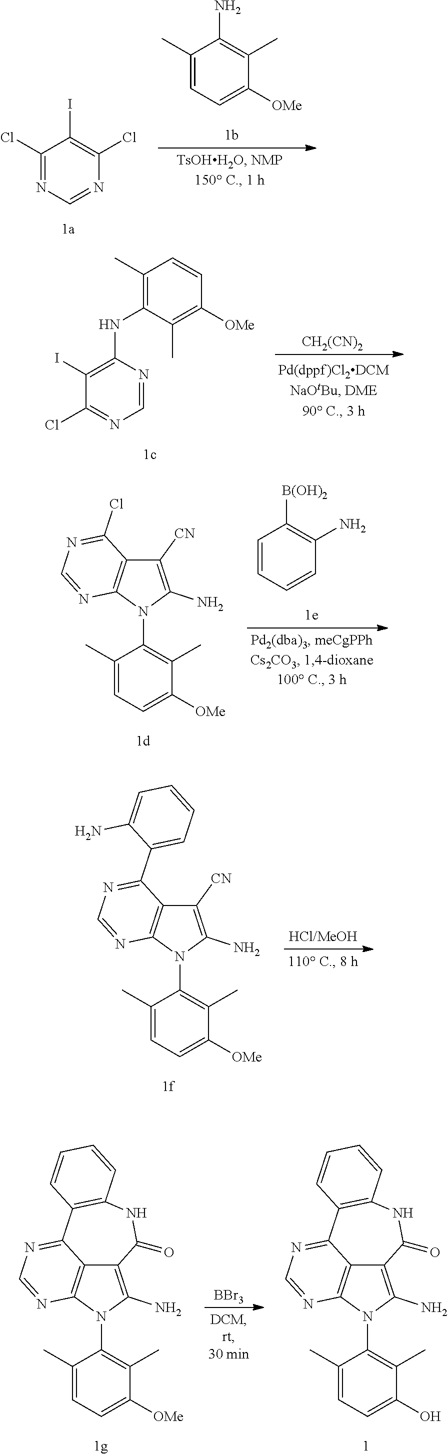

### Step 1: Synthesis of 6-chloro-5-iodo-N-(3-methoxy-2,6-dimethylphenyl)pyrimidin-4-amine (1c)

4,6-Dichloro-5-iodopyrimidine 1a (2.0 g, 7.3 mmol) was dissolved in anhydrous N-methyl-2-pyrrolidone (4 mL). 3-Methoxy-2,6-dimethylaniline 1b (2.3 g, 15 mmol) and p-toluenesulfonic acid monohydrate (14 mg, 73 μmol) were added. The mixture was reacted at 150° C. for 1 h. The reaction mixture was cooled to room temperature and then directly purified by reverse-phase column chromatography (C18, 0.05% aqueous ammonia:acetonitrile=20:1-1:20) to obtain compound 1c (1.5 g, yield: 53%). m/z (ESI): 390.1 [M+H]⁺.

### Step 2: Synthesis of 6-amino-4-chloro-7-(3-methoxy-2,6-dimethylphenyl)-7H-pyrrolo[2,3-d]pyrimidine-5-carbonitrile (1d)

Malononitrile (0.066 g, 1.0 mmol) was dissolved in anhydrous ethylene glycol dimethyl ether (2 mL), and sodium tert-butoxide (0.15 g, 1.5 mmol) was added. After the mixture was stirred at room temperature for 30 min, 1c (0.10 g, 0.25 mmol) and [1,1′-bis(diphenylphosphino)ferrocene]palladium dichloride dichloromethane complex (8.1 mg, 10 μmol) were added. The system was purged with nitrogen 3 times, and the mixture was reacted at 90° C. for 3 h under nitrogen atmosphere. The reaction mixture was cooled to room temperature and then filtered to remove the insoluble substance, and the filtrate was concentrated to dryness by rotary evaporation. The resulting residue was purified by reverse-phase column chromatography (C18, 0.05% aqueous ammonia:acetonitrile=20:1-1:20) to obtain compound 1d (62 mg, yield: 73%). m/z (ESI): 328.1 [M+H]⁺.

### Step 3: Synthesis of 6-amino-4-(2-aminophenyl)-7-(3-methoxy-2,6-dimethylphenyl)-7H-pyrrolo[2,3-d]pyrimidine-5-carbonitrile (1f)

Compound 1d (62 mg, 0.19 mmol) and 2-aminobenzeneboronic acid 1e (52 mg, 0.38 mmol) were dissolved in a mixed solution of 1,4-dioxane (3 mL)/water (0.3 mL) at room temperature, and then tris(dibenzylideneacetone)dipalladium (8.7 mg, 9.5 μmol), 1,3,5,7-tetramethyl-6-phenyl-2,4,8-trioxa-6-phosphaadamantane (11 mg, 38 μmol), and cesium carbonate (0.19 g, 0.57 mmol) were added. The system was purged with nitrogen, and then the mixture was reacted at 100° C. for 3 h. The reaction mixture was cooled to room temperature and then filtered to remove the insoluble substance, and the filtrate was concentrated to dryness by rotary evaporation. The resulting residue was purified by reverse-phase column chromatography (C18, 0.05% aqueous ammonia:acetonitrile=20:1-1:20) to obtain compound 1f (43 mg, yield: 59%). m/z (ESI): 385.4 [M+H]⁺.

### Step 4: Synthesis of 5-amino-4-(3-methoxy-2,6-dimethylphenyl)-4,7-dihydro-6H-1,3,4,7-tetraazadibenzo[cd,f]azulen-6-one (1g)

Compound 1f (43 mg, 0.11 mmol) was dissolved in a hydrochloric acid methanol (7 N, 10 mL) solution, and the mixture was reacted at 110° C. for 8 h in a sealed tube. The reaction mixture was cooled to room temperature and then distilled under reduced pressure to remove the solvent. The resulting residue was subjected to reverse-phase column chromatography (C18, 0.05% aqueous ammonia:acetonitrile=20:1-1:20) to obtain compound 1g (19 mg, yield: 45%). m/z (ESI): 386.3 [M+H]⁺.

### Step 5: Synthesis of 5-amino-4-(3-hydroxy-2,6-dimethylphenyl)-4,7-dihydro-6H-1,3,4,7-tetraazadibenzo[cd,f]azulen-6-one (Compound 1)

Compound 1g (19 mg, 49 μmol) was dissolved in anhydrous dichloromethane (2 mL) at room temperature. A solution of boron tribromide in dichloromethane (1 mol/L, 0.5 mL) was added, and the mixture was stirred for 30 min. The reaction mixture was quenched with methanol (1 mL) and then distilled under reduced pressure to remove the solvent. The resulting residue was purified by reverse-phase column chromatography (C18, 0.05% aqueous ammonia:acetonitrile=20:1-1:20) to obtain compound 1 (10 mg, yield: 55%) as a white solid. m/z (ESI): 372.4 [M+H]⁺.

¹H NMR (400 MHz, DMSO-d₆): δ 9.64 (s, 1H), 9.14 (s, 1H), 8.51 (d, J=8.0 Hz, 1H), 8.33 (s, 1H), 7.27 (t, J=8.0 Hz, 1H), 7.13-6.93 (m, 6H), 1.79 (s, 3H), 1.70 (s, 3H).

### Example 2. Preparation of 5-amino-4-(3-hydroxy-2,6-dimethylphenyl)-2-methyl-4,7-dihydro-6H-1,3,4,7-tetraazadibenzo[cd,f]azulen-6-one (Compound 2)

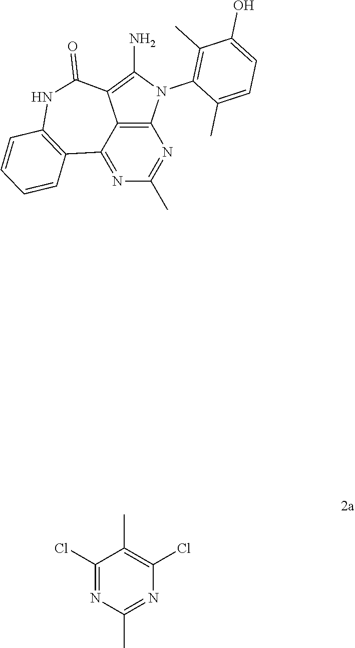

Compound 2 was prepared by replacing compound 1a with compound 2a and using a method similar to that in Example 1.

m/z (ESI): 386.3[M+H]⁺;

¹H NMR (400 MHz, DMSO-d₆): δ 9.60 (s, 1H), 9.07 (s, 1H), 8.52 (d, J=8.0 Hz, 1H), 7.25 (t, J=8.0 Hz, 1H), 7.09 (t, J=8.0 Hz, 1H), 8.33 (s, 1H), 7.01-6.89 (m, 4H), 2.44 (s, 3H), 1.79 (s, 3H), 1.70 (s, 3H).

### Example 3. Preparation of 2-amino-1-(3-hydroxy-2,6-dimethylphenyl)-11-methyl-4,5-dihydro-1,4,10,12-tetraazabenzo[4,5]cyclooctatetraeno[1,2,3-cd]inden-3(1H)-one (Compound 3)

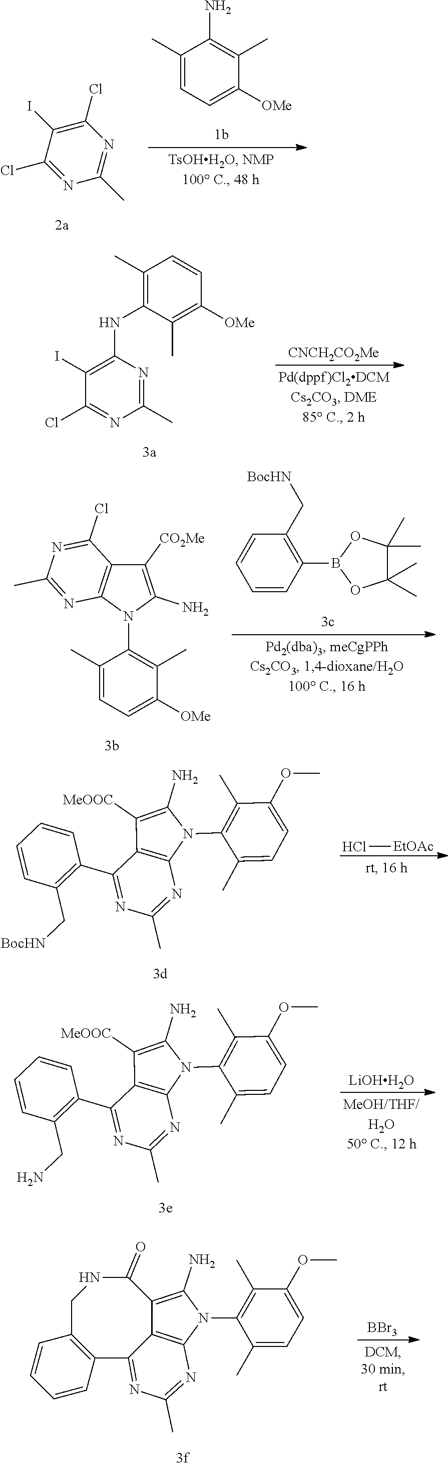

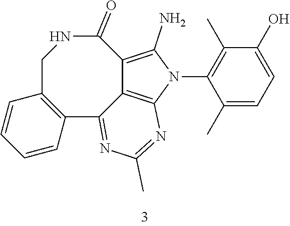 [SMILES: Cc1nc2c3c(c(N)n(-c4c(C)ccc(O)c4C)c3n1)C(=O)NCc1ccccc1-2]

### Step 1: Synthesis of 6-chloro-5-iodo-N-(3-methoxy-2,6-dimethylphenyl)-2-methylpyrimidin-4-amine (3a)

4,6-Dichloro-5-iodo-2-methylpyrimidine 2a (5.8 g, 20 mmol) and 3-methoxy-2,6-dimethylaniline 1b (6.0 g, 40 mmol) were dissolved in N-methyl-2-pyrrolidone (10 mL) at room temperature, and then p-toluenesulfonic acid monohydrate (0.19 g, 1.0 mmol) was added. The mixture was stirred at 100° C. for 48 h. After the reaction was cooled to room temperature, the reaction mixture was diluted with water (20 mL) and extracted with ethyl acetate (50 mL×3). The organic phases were combined, washed with saturated brine (50 mL), dried over anhydrous sodium sulfate, and filtered. The filtrate was concentrated. The resulting residue was purified by normal-phase column chromatography (petroleum ether:ethyl acetate=10:1-2:1) to obtain compound 3a (1.8 g, yield: 22%) as a white solid. m/z (ESI): 404.1 [M+H]⁺.

### Step 2: Synthesis of methyl 6-amino-4-chloro-7-(3-methoxy-2,6-dimethylphenyl)-2-methyl-7H-pyrrolo[2,3-d]pyrimidine-5-carboxylate (3b)

Methyl cyanoacetate (1.6 g, 16 mmol) was dissolved in anhydrous ethylene glycol dimethyl ether (20 mL). Cesium carbonate (7.8 g, 24 mmol) was added. After the mixture was stirred at room temperature for 30 min, compound 3a (1.6 g, 4 mmol) and [1,1′-bis(diphenylphosphino)ferrocene]palladium dichloride dichloromethane complex (0.33 g, 0.4 mmol) were added. The system was purged with nitrogen 3 times, and the mixture was reacted at 85° C. for 2 h under nitrogen atmosphere. After the reaction mixture was cooled to room temperature, saturated brine (20 mL) was added. The mixture was extracted three times with ethyl acetate (50 mL×3), and the organic phases were combined, washed with saturated brine (50 mL), dried over anhydrous sodium sulfate, and filtered. The filtrate was concentrated under reduced pressure. The resulting residue was purified by silica gel column chromatography (petroleum ether:ethyl acetate=10:1-1:2) to obtain the product compound 3b (0.79 g, yield: 53%). m/z (ESI): 375.2 [M+H]⁺.

### Step 3: Synthesis of methyl 6-amino-4-(2-(((tert-butoxycarbonyl)amino)methyl)phenyl)-7-(3-methoxy-2,6-dimethylphenyl)-2-methyl-7H-pyrrolo[2,3-d]pyrimidine-5-carboxylate (3d)

Compound 3b (90 mg, 0.24 mmol), compound 3c (0.12 g, 0.36 mmol), tris(dibenzylideneacetone)dipalladium (22 mg, 24 μmol), 1,3,5,7-tetramethyl-6-phenyl-2,4,8-trioxa-6-phosphaadamantane (28 mg, 96 μmol), and cesium carbonate (0.16 g, 0.48 mmol) were dissolved in 1,4-dioxane/water (2 mL/0.4 mL) at room temperature. The system was purged with nitrogen three times. The reaction mixture was then moved to 100° C. and reacted for 16 h. The reaction mixture was cooled to room temperature and then filtered to remove the insoluble substance, and saturated brine (10 mL) was added. The mixture was extracted three times with ethyl acetate (20 mL×3), and the organic phases were combined, washed with saturated brine (20 mL), dried over anhydrous sodium sulfate, filtered, and concentrated under reduced pressure. The resulting residue was purified by silica gel column chromatography (petroleum ether:ethyl acetate=5:1-3:1) to obtain the product compound 3d (110 mg, yield: 84%). m/z (ESI): 546.3 [M+H]⁺.

### Step 4: Synthesis of methyl 6-amino-4-(2-(aminomethyl)phenyl)-7-(3-methoxy-2,6-dimethylphenyl)-2-methyl-7H-pyrrolo[2,3-d]pyrimidine-5-carboxylate (3e)

Compound 3d (90 mg, 0.16 mmol) was dissolved in a solution of hydrochloric acid in ethyl acetate (4 M in EtOAc, 4 mL) at room temperature, and the mixture was reacted under these conditions for 16 h. After the reaction was completed, the reaction mixture was concentrated. The resulting solid residue, compound 3e, could be directly used in the next step without purification.

### Step 5: Synthesis of 2-amino-1-(3-methoxy-2,6-dimethylphenyl)-11-methyl-4,5-dihydro-1,4,10,12-tetraazabenzo[4,5]cyclooctatetraeno[1,2,3-cd]inden-3(1H)-one (3f)

Compound 3e (45 mg, 0.10 mmol) obtained in the previous step was dissolved in methanol/tetrahydrofuran/water (2 mL/2 mL/2 mL), and lithium hydroxide monohydrate (42 mg, 1.0 mmol) was then added. The reaction mixture was reacted at 50° C. for 12 h. The reaction mixture was concentrated. The resulting residue was purified by reverse-phase column chromatography (C18, 0.05% aqueous ammonia:acetonitrile=20:1-1:20) to obtain the target compound 3f (30 mg, yield: 72%). LC-MS: m/z (ESI): 414.2 [M+H]⁺.

### Step 6: Synthesis of 2-amino-1-(3-hydroxy-2,6-dimethylphenyl)-11-methyl-4,5-dihydro-1,4,10,12-tetraazabenzo[4,5]cyclooctatetraeno[1,2,3-cd]inden-3(1H)-one (3)

Compound 3f (30 mg, 72 μmol) was dissolved in dichloromethane (4 mL) at room temperature. A solution of boron tribromide (1 mol/L, 2 mL) in dichloromethane was added, and the mixture was stirred for 30 min. The reaction mixture was quenched with methanol (5 mL) and then concentrated. The resulting residue was purified by reverse-phase column chromatography (C18, 0.05% aqueous ammonia:acetonitrile=20:1-1:20) to obtain compound 3 (15 mg, yield: 51%). m/z (ESI): 400.2 [M+H]⁺.

¹H NMR (400 MHz, CD₃OD) δ 7.80-7.78 (m, 1H), 7.54-7.52 (m, 2H), 7.44-7.42 (m, 1H), 7.17-7.14 (m, 1H), 6.99 (d, J=8.4 Hz, 1H), 5.21-5.16 (m, 1H), 4.22-4.18 (m, 1H), 2.63 (s, 3H), 2.03-1.98 (m, 3H), 1.84-1.78 (m, 3H).

### Example 4. Preparation of 2-amino-1-(3-hydroxy-2,6-dimethylphenyl)-11-methyl-4,5-dihydro-1,4,6,10,12-pentaazabenzo[4,5]cyclooctatetraeno[1,2,3-cd]inden-3(1H)-one (Compound 4)

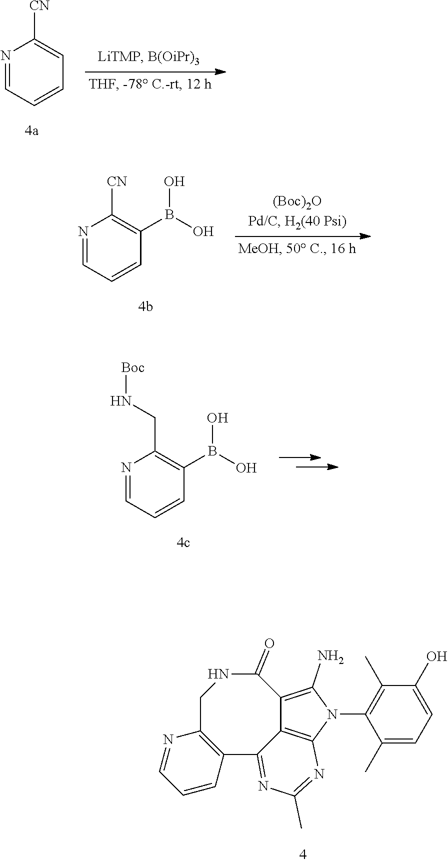 [SMILES: N#Cc1ccccn1 ; N#Cc1ncccc1B(O)O ; CC(C)(C)OC(=O)NCc1ncccc1B(O)O ; Cc1nc2c3c(c(N)n(-c4c(C)ccc(O)c4C)c3n1)C(=O)NCc1ncccc1-2]

### Step 1: Synthesis of (2-cyanopyridin-3-yl)boronic Acid (4b)

Lithium tetramethylpiperidine (28.4 mL, 1 M) was dissolved in anhydrous tetrahydrofuran at −78° C. Triisopropyl borate (2.4 g, 13 mmol) was added dropwise thereto, and a solution of 2-cyanopyridine (1.4 g, 13 mmol) in tetrahydrofuran (10 mL) was then added dropwise. After the dropwise addition was completed, the reaction was moved to room temperature and reacted for 12 h. A saturated sodium bicarbonate solution (10 mL) was added to quench the reaction, and the mixture was extracted 3 times with ethyl acetate (50 mL×3). The organic phases were combined, washed with a saturated aqueous sodium chloride solution (20 mL), dried over anhydrous sodium sulfate, filtered, and concentrated. The resulting solid was purified by normal-phase column chromatography (petroleum ether:ethyl acetate=1:1-0:1) to obtain the product compound 4b (1.5 g, yield: 77%). m/z (ESI): 149.2 [M+H]⁺.

### Step 2: Synthesis of (2-(((tert-butoxycarbonyl)amino)methyl)pyridin-3-yl)boronic Acid (4c)

Compound 4b (1.5 g, 10 mmol) was dissolved in anhydrous methanol (10 mL) at room temperature, and then di-tert-butyl dicarbonate (4.4 g, 20 mmol) and Pd/C (10%, 30 mg) were added. The system was purged with hydrogen. The mixture was reacted at 50° C. for 12 h under an oil bath at a pressure of 40 psi. The reaction mixture was cooled to room temperature and then filtered to remove the insoluble substance, and the filtrate was concentrated. The resulting residue was purified by silica gel column chromatography (petroleum ether:ethyl acetate=1:1-1:3) to obtain the product compound 4c (1.4 g, yield: 55%). m/z (ESI): 253.2 [M+H]⁺.

Compound 4 was prepared by replacing compound 3c with compound 4c and using a method similar to that in Example 3.

m/z (ESI): 401.2 [M+H]⁺;

¹H NMR (400 MHz, DMSO-d₆) δ 9.61 (d, J=8.4 Hz, 1H), 8.61 (dd, J=4.8, 1.6 Hz, 1H), 8.18-8.14 (m, 1H), 8.07 (t, J=7.0 Hz, 1H), 7.51 (dd, J=8.0, 4.8 Hz, 1H), 7.11-7.07 (m, 1H), 7.05 (s, 2H), 6.95 (d, J=8.0 Hz, 1H), 5.15-5.09 (m, 1H), 4.11-4.06 (m, 1H), 2.50 (m, 3H), 1.92-1.79 (m, 3H), 1.71-1.58 (m, 3H).

### Example 5. Preparation of 2-amino-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (Compound 5)

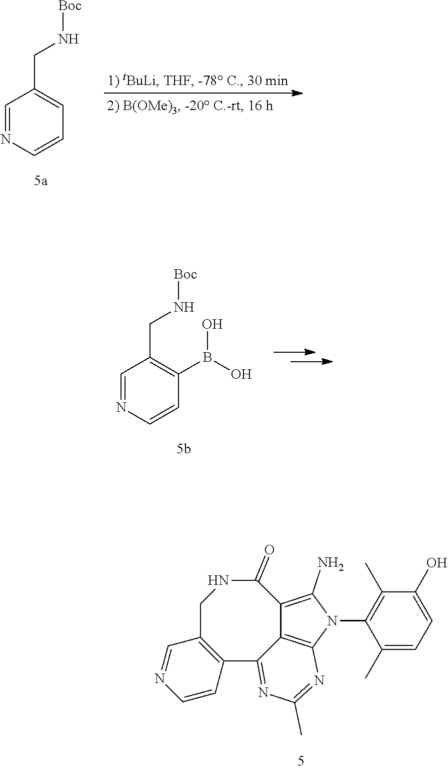 [SMILES: CC(C)(C)OC(=O)NCc1cccnc1 ; CC(C)(C)OC(=O)NCc1cnccc1B(O)O ; Cc1nc2c3c(c(N)n(-c4c(C)ccc(O)c4C)c3n1)C(=O)NCc1cnccc1-2]

### Step 1: Synthesis of (3-(((tert-butoxycarbonyl)amino)methyl)pyridin-4-yl)boronic Acid (5b)

Compound 5a (1.2 g, 5.8 mmol) was dissolved in anhydrous tetrahydrofuran (15 mL) at room temperature, and the system was purged with nitrogen three times. The reaction was placed at −78° C., and tert-butyllithium (11 mL, 1.3 mol/L in pentane solution) was slowly added dropwise to the system. After the dropwise addition was completed, the system was slowly warmed to −20° C. and stirred for 30 min. Trimethyl borate (2.4 g, 23 mmol) was slowly added dropwise to the system. After the dropwise addition was completed, the reaction mixture was moved to room temperature and reacted for 16 h. Under an ice bath, a saturated aqueous ammonium chloride solution was slowly added to the reaction mixture to quench the reaction. The mixture was extracted three times with a mixed solution of dichloromethane/methanol (3/1, 20 mL). The organic phases were combined, dried over anhydrous sodium sulfate, and filtered, and the filtrate was concentrated in vacuum to obtain compound 5b (1.2 g, yield: 54.53%), which was directly used in the next step. m/z (ESI): 253.2 [M+H]⁺.

Compound 5 was prepared by replacing compound 3c with compound 5b and using a method similar to that in Example 3.

m/z (ESI): 401.2 [M+H]⁺;

¹H NMR (400 MHz, CD₃OD) δ 8.67-8.65 (m, 1H), 8.56 (s, 1H), 7.83-7.81 (m, 1H), 7.17-7.11 (m, 1H), 6.97-6.95 (m, 1H), 5.24-5.20 (m, 1H), 4.32-4.28 (m, 1H), 2.60 (s, 3H), 2.00-1.93 (m, 3H), 1.81-1.73 (m, 3H).

### Example 6. Preparation of 2-amino-1-(3-hydroxy-2,6-dimethylphenyl)-11-methyl-4,5-dihydro-1,4,9,10,12-pentaazabenzo[4,5]cyclooctatetraeno[1,2,3-cd]inden-3(1H)-one (Compound 6)

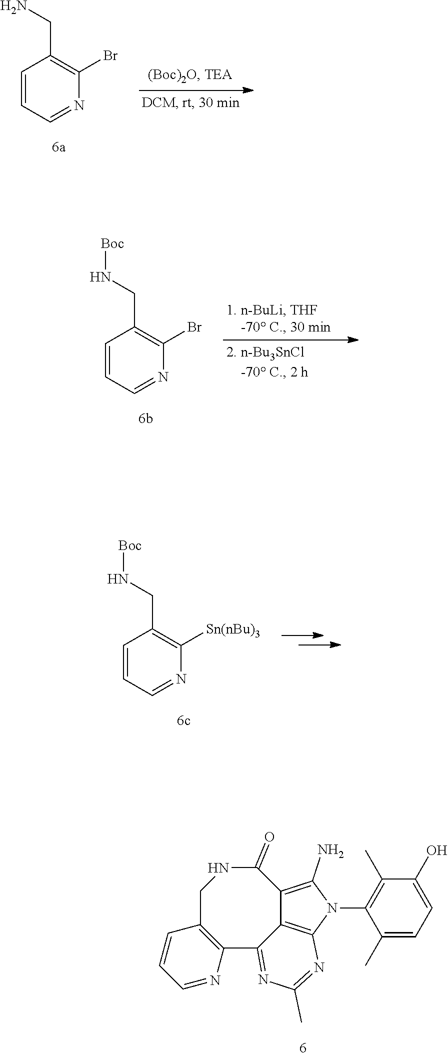

### Step 1: Synthesis of tert-butyl ((2-bromopyridin-3-yl)methyl)carbamate (6b)

Compound 6a (0.50 g, 2.7 mmol) was dissolved in dichloromethane (5 mL) at room temperature, and then di-tert-butyl dicarbonate (0.88 g, 4 mmol) and triethylamine (0.54 g, 5.4 mmol) were added. The mixture was stirred for 30 min and concentrated in vacuum, and the crude product was purified by silica gel column chromatography (petroleum ether:ethyl acetate=1:0-0:1) to obtain compound 6b (0.70 g, yield: 96%).

### Step 2: Synthesis of tert-butyl ((2-(tributyltin)pyridin-3-yl)methyl)carbamate (6c)

Compound 6b (0.36 g, 1.3 mmol) was dissolved in tetrahydrofuran (4 mL) at room temperature, and the system was purged with nitrogen three times. The reaction was then moved to −70° C. n-Butyllithium (1.1 mL, 2.5 mol/L in tetrahydrofuran solution) was slowly added dropwise to the system. After the dropwise addition was completed, the mixture was stirred at the temperature for 30 min. Tri-n-butyltin chloride (0.90 g, 2.8 mmol) was then added dropwise to the system. After the dropwise addition was completed, the mixture was stirred at −70° C. for another 2 h. A saturated potassium fluoride solution was added to the system to quench the reaction, and the mixture was stirred at room temperature for 1 h. The mixture was extracted three times with ethyl acetate (10 mL).

The organic phases were combined, washed once with saturated brine (10 mL), dried over anhydrous sodium sulfate, and concentrated in vacuum. The crude product was purified by silica gel column chromatography (petroleum ether:ethyl acetate=1:0-10:1) to obtain compound 6c (0.20 g, yield: 32%).

Compound 6 was prepared by replacing compound 3c with compound 6c and using a method similar to that in Example 3.

m/z (ESI): 401.2 [M+H]⁺.

¹H NMR (400 MHz, DMSO-d₆) δ 8.65-8.64 (m, 1H), 7.78 (t, J=7.2 Hz, 1H), 7.65 (dd, J=1.6, 7.6 Hz, 1H), 7.40 (dd, J=4.4, 7.6 Hz, 1H), 7.03 (dd, J=8.4, 13.6 Hz, 1H), 6.98 (br s, 2H), 6.89 (d, J=8.0 Hz, 1H), 6.33-6.13 (m, 1H), 4.92-4.86 (m, 1H), 4.03 (dd, J=6.4, 14.8 Hz, 1H), 2.47 (br s, 3H), 1.85-1.74 (m, 3H), 1.64-1.53 (m, 3H).

### Example 7. Preparation of 2-amino-1-(3-hydroxy-2,6-dimethylphenyl)-11-methyl-4,5-dihydro-1,4,8,10,12-pentaazabenzo[4,5]cyclooctatetraeno[1,2,3-cd]inden-3(1H)-one (Compound 7)

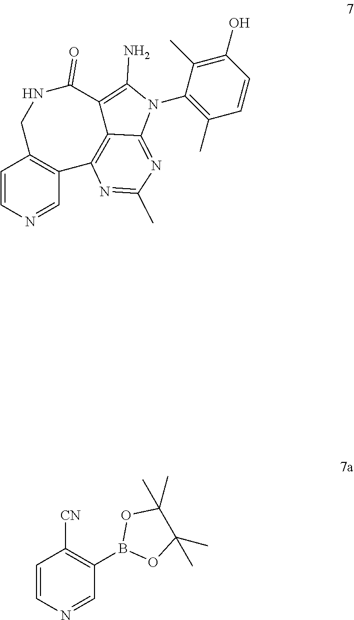 [SMILES: Cc1nc2c3c(c(N)n(-c4c(C)ccc(O)c4C)c3n1)C(=O)NCc1ccncc1-2 ; CC1(C)OB(c2cnccc2C#N)OC1(C)C]

Compound 7 was prepared by replacing compound 4b with compound 7a and using a method similar to that in Example 4.

m/z (ESI): 401.2 [M+H]⁺;

¹H NMR (400 MHz, DMSO-d₆) δ 8.65-8.64 (m, 1H), 7.78 (t, J=7.2 Hz, 1H), 7.65 (dd, J=1.6, 7.6 Hz, 1H), 7.40 (dd, J=4.4, 7.6 Hz, 1H), 7.03 (dd, J=8.4, 13.6 Hz, 1H), 6.98 (br s, 2H), 6.89 (d, J=8.0 Hz, 1H), 6.33-6.13 (m, 1H), 4.89 (ddd, J=3.2, 7.6, 14.8 Hz, 1H), 4.03 (dd, J=6.4, 14.8 Hz, 1H), 2.47 (br s, 3H), 1.85-1.74 (m, 3H), 1.64-1.53 (m, 3H).

### Example 8. Preparation of 2-amino-1-(3-hydroxy-2,6-dimethylphenyl)-7,10-dimethyl-1,4,5,7-tetrahydro-3H-1,4,6,7,9,11-hexaazacyclopenta[4,5]cyclooctatetraeno[1,2,3-cd]inden-3-one (Compound 8)

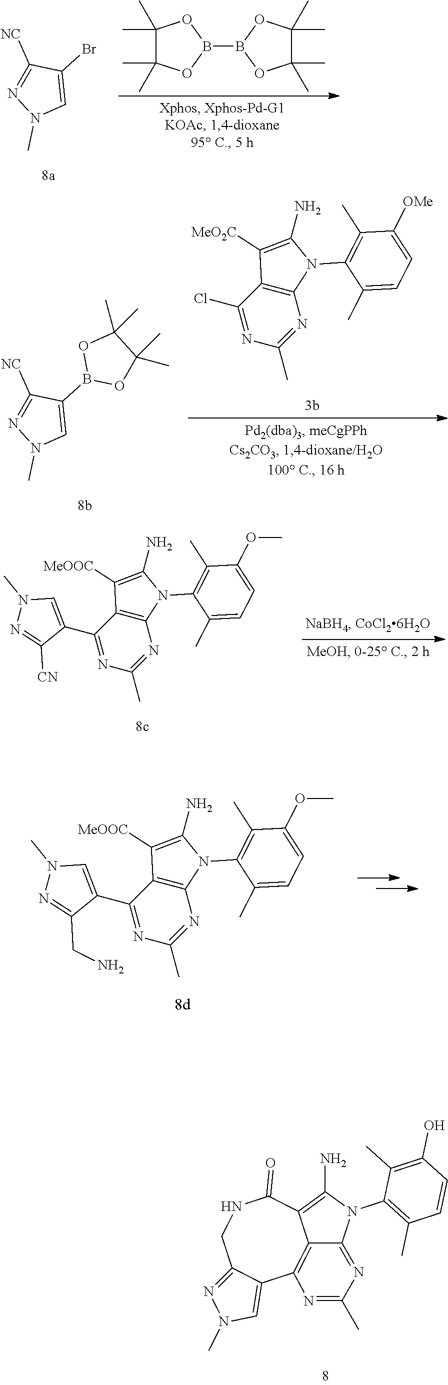

### Step 1: Synthesis of 1-methyl-4-(4,4,5,5-tetramethyl-1,3,2-dioxaborolan-2-yl)-1H-pyrazole-3-carbonitrile (8b)

Compound 8a (1.0 g, 5.4 mmol), bis(pinacolato)diboron (1.6 g, 6.4 mmol), 2-dicyclohexylphosphino-2′,4′,6′-triisopropylbiphenyl (0.23 g, 0.54 mmol), chloro(2-dicyclohexylphosphino-2′,4′,6′-triisopropyl-1,1′-biphenyl)[2-(2-aminoethylphenyl)]palladium(II) (0.40 g, 0.54 mmol), and potassium acetate (1.1 g, 11 mmol) were dissolved in 1,4-dioxane at room temperature. The system was purged with nitrogen 3 times, and the mixture was reacted at 95° C. for 5 h under nitrogen atmosphere. The reaction mixture was cooled to room temperature and filtered, and the solid was washed with ethyl acetate. The organic phase was washed with saturated brine (20 mL). The organic phases were combined, dried over anhydrous sodium sulfate, and filtered, and the filtrate was concentrated. The resulting residue was purified by silica gel column chromatography (petroleum ether:ethyl acetate=1:1-1:3) to obtain compound 8b (0.60, yield: 47%). m/z (ESI): 234.3 [M+H]⁺.

### Step 2: Synthesis of methyl 6-amino-4-(3-cyano-1-methyl-1H-pyrazol-4-yl)-7-(3-methoxy-2,6-dimethylphenyl)-2-methyl-7H-pyrrolo[2,3-d]pyrimidine-5-carboxylate (8c)

Compound 3b (0.37 g, 1.0 mmol), compound 8b (0.3 g, 1.3 mmol), bis(dibenzylideneacetone)palladium (91 mg, 99 μmol), 1,3,5,7-tetramethyl-6-phenyl-2,4,8-trioxa-6-phosphaadamantane (0.12 g, 0.40 mmol), and cesium carbonate (0.65 g, 2.0 mmol) were dissolved in 1,4-dioxane/water (4 mL/0.8 mL) at room temperature. The system was purged with nitrogen three times. The reaction mixture was then moved to 100° C. and reacted for 16 h. The reaction mixture was cooled to room temperature and then filtered to remove the insoluble substance, and the filtrate was concentrated to dryness by rotary evaporation. The resulting residue was subjected to column chromatography (petroleum ether:ethyl acetate=1:1-1:3) to obtain the product compound 8c (0.20 mg, yield: 44%). m/z (ESI): 446.2 [M+H]⁺.

### Step 3: Synthesis of methyl 6-amino-4-(3-(aminomethyl)-1-methyl-1H-pyrazol-4-yl)-7-(3-methoxy-2,6-dimethylphenyl)-2-methyl-7H-pyrrolo[2,3-d]pyrimidine-5-carboxylate (8d)

Compound 8c (0.20 g, 0.45 mmol) and cobalt chloride hexahydrate (0.53 g, 2.2 mmol) were dissolved in anhydrous methanol under an ice bath, and then sodium borohydride (85 mg, 2.2 mmol) was added. The mixture was then warmed to room temperature and reacted for 2 h. Water (10 mL) was added to the reaction mixture to quench the reaction. The mixture was extracted 3 times with ethyl acetate (15 mL×3), and the organic phases were combined, washed with a saturated aqueous sodium chloride solution (30 mL), dried over anhydrous sodium sulfate, and filtered. The filtrate was concentrated. The resulting solid could be used in the next step without purification. m/z (ESI): 450.2 [M+H]⁺.

Compound 8 was prepared by replacing compound 3e with compound 8d and using a method similar to that in Example 3.

m/z (ESI): 404.1 [M+H]⁺;

¹H NMR (400 MHz, DMSO-d₆) δ 9.58 (s, 1H), 8.07 (s, 1H), 7.83 (t, J=6.4 Hz, 1H), 7.08 (d, J=8.0 Hz, 1H), 6.94 (d, J=8.4 Hz, 1H), 6.86 (s, 2H), 4.68-4.45 (m, 1H), 4.10-3.92 (m, 1H), 3.87 (s, 3H), 2.40 (s, 3H), 1.90-1.54 (m, 6H).

### Example 9. Preparation of 2-amino-1-(3-hydroxy-2,6-dimethylphenyl)-7,11-dimethyl-4,5-dihydro-1,4,6,10,12-pentaazabenzo[4,5]cyclooctatetraeno[1,2,3-cd]inden-3(1H)-one (Compound 9)

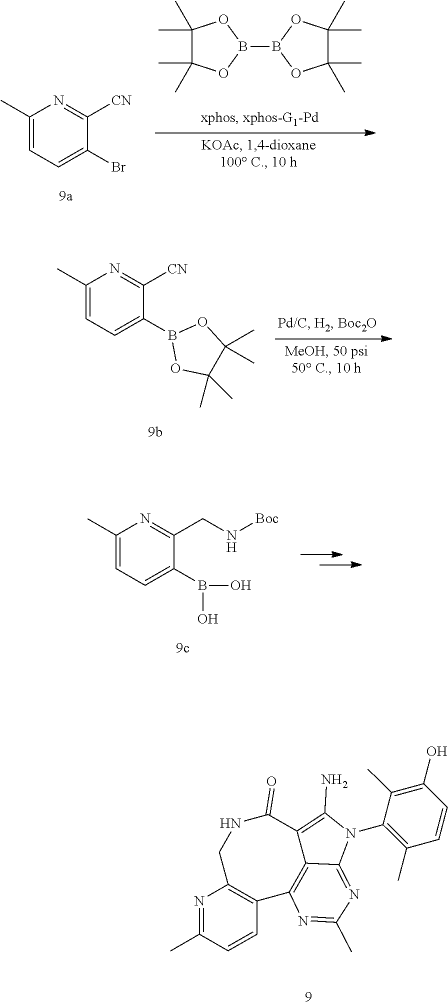

### Step 1: Synthesis of 6-methyl-3-(4,4,5,5-tetramethyl-1,3,2-dioxaborolan-2-yl)cyanopyridine (9b)

Compound 9a (1.0 g, 5.1 mmol), bis(pinacolato)diboron (1.9 g, 7.6 mmol), 2-dicyclohexylphosphino-2′,4′,6′-triisopropylbiphenyl (0.22 g, 0.51 mmol), chloro(2-dicyclohexylphosphino-2′,4′,6′-triisopropyl-1,1′-biphenyl)[2-(2-aminoethylphenyl)]palladium(II) (0.37 g, 0.51 mmol), and potassium acetate (1.0 g, 10 mmol) were dissolved in anhydrous 1,4-dioxane (20 mL) at room temperature. The system was purged with nitrogen 3 times, and the mixture was reacted at 100° C. for 10 h under nitrogen atmosphere. After the reaction mixture was cooled to room temperature, water (20 mL) was added to the reaction mixture to quench the reaction. The mixture was extracted 3 times with ethyl acetate (100 mL×3), and the organic phases were combined, washed with a saturated aqueous sodium chloride solution (30 mL), dried over anhydrous sodium sulfate, and filtered. The filtrate was concentrated. The resulting residue was subjected to column chromatography (petroleum ether:ethyl acetate=100:1-10:1) to obtain the product compound 9b (0.65 g, yield: 52%). m/z (ESI): 245.1 [M+H]⁺.

### Step 2: Synthesis of (2-(((tert-butoxycarbonyl)amino)methyl)-6-methylpyridin-3-yl)boronic Acid (9c)

Compound 9b (0.5 g, 2.0 mmol) was dissolved in anhydrous methanol (30 mL) at room temperature, and then di-tert-butyl dicarbonate (0.89 g, 4.1 mmol) and Pd/C (10%, 50 mg) were added. The system was purged with hydrogen. The reaction mixture was reacted at 50° C. for 10 h under an oil bath at a pressure of 50 psi. The reaction mixture was cooled to room temperature and then filtered to remove the insoluble substance, and the filtrate was concentrated. The resulting residue was purified by silica gel column chromatography (petroleum ether:ethyl acetate=100:1-1:1) to obtain the product compound 9c (0.12 g, yield: 22%). m/z (ESI): 267.2 [M+H]⁺.

Compound 9 was prepared by replacing compound 3c with compound 9c and using a method similar to that in Example 3.

m/z (ESI): 415.1 [M+H]⁺;

¹H NMR (400 MHz, CD₃OD) δ 8.13-8.11 (m, 1H), 7.43 (d, J=8.0 Hz, 1H), 7.16-7.11 (m, 1H), 6.96 (d, J=8.4 Hz, 1H), 5.32-5.28 (m, 1H), 4.22-4.18 (m, 1H), 2.64 (s, 3H), 2.59 (s, 3H), 2.00-1.94 (m, 3H), 1.79-1.74 (m, 3H).

### Example 10. Preparation of 2-amino-6-ethyl-1-(3-hydroxy-2,6-dimethylphenyl)-11-methyl-4,5-dihydro-1,4,10,12-tetraazabenzo[4,5]cyclooctatetraeno[1,2,3-cd]inden-3(1H)-one (Compound 10)

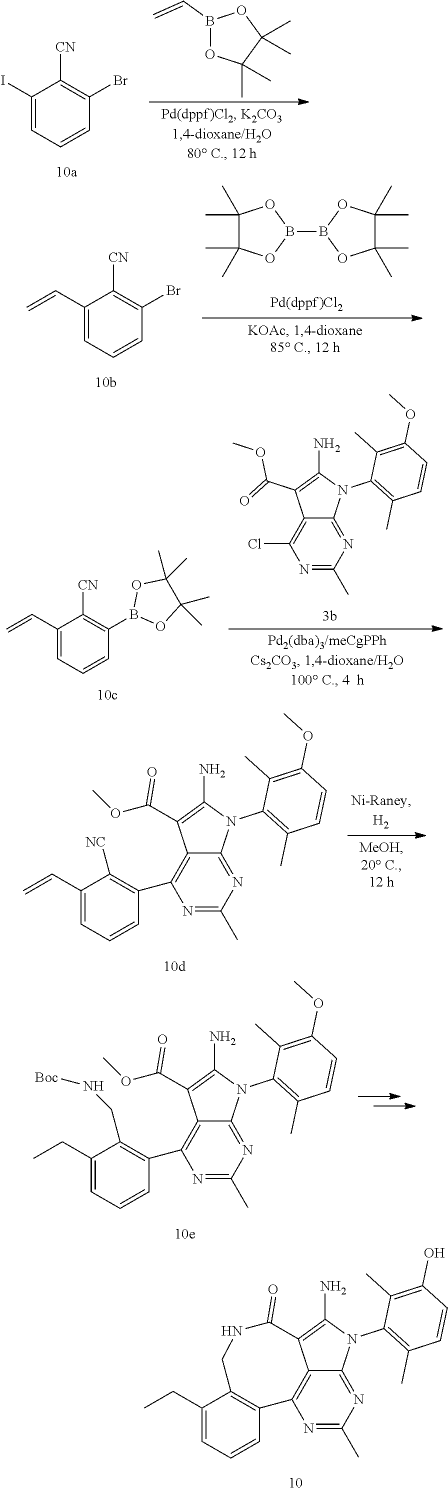

### Step 1: Synthesis of 2-bromo-6-vinylbenzonitrile (10b)

Compound 10a (2.0 g, 6.5 mmol) and vinyl borate pinacol ester (0.30 g, 1.5 mmol) were dissolved in a mixed solution of 1,4-dioxane (20 mL)/water (4 mL) at room temperature, and [1,1′-bis(diphenylphosphino)ferrocene]palladium dichloride (0.47 g, 0.65 mmol) and potassium carbonate (2.7 g, 19 mmol) were added. The system was purged with nitrogen 3 times, and then the mixture was reacted at 80° C. for 12 h. The reaction mixture was cooled to room temperature and then filtered to remove the insoluble substance, and the filtrate was concentrated. The resulting residue was purified by silica gel column chromatography (petroleum ether:ethyl acetate=100:0-99:1) to obtain the product compound 10b (0.90 g, yield: 67%).

### Step 2: Synthesis of 2-(4,4,5,5-tetramethyl-1,3,2-dioxaborolan-2-yl)-6-vinylbenzonitrile (10c)

Compound 10b (0.83 g, 4.0 mmol), bis(pinacolato)diboron (1.5 g, 6.0 mmol), [1,1′-bis(diphenylphosphino)ferrocene]palladium dichloride (0.29 g, 0.40 mmol), and potassium acetate (0.78 g, 8.0 mmol) were dissolved in 1,4-dioxane at room temperature. The system was purged with nitrogen 3 times, and the mixture was reacted at 85° C. for 12 h under nitrogen atmosphere. After the reaction mixture was cooled to room temperature, water (50 mL) was added to the reaction mixture to quench the reaction. The mixture was extracted 3 times with ethyl acetate (50 mL×3), and the organic phases were combined, washed with a saturated aqueous sodium chloride solution (30 mL), dried over anhydrous sodium sulfate, and filtered. The filtrate was concentrated. The resulting residue was purified by column chromatography (petroleum ether:ethyl acetate=100:1-20:1) to obtain the product compound 10c (0.95 g, yield: 93%).

### Step 3: Synthesis of methyl 6-amino-4-(2-cyano-3-vinylphenyl)-7-(3-methoxy-2,6-dimethylphenyl)-2-methyl-7H-pyrrolo[2,3-d]pyrimidine-5-carboxylate (10d)

Compound 3b (0.29 g, 0.78 mmol), compound 10c (0.40 g, 1.6 mmol), tris(dibenzylideneacetone)dipalladium (72 mg, 78 μmol), 1,3,5,7-tetramethyl-6-phenyl-2,4,8-trioxa-6-phosphaadamantane (92 mg, 0.32 mmol), and cesium carbonate (0.51 g, 1.6 mmol) were dissolved in 1,4-dioxane/water (2 mL/0.4 mL) at room temperature. The system was purged with nitrogen three times. The reaction mixture was then moved to 100° C. and reacted for 4 h. The reaction mixture was cooled to room temperature and then filtered to remove the insoluble substance, and saturated brine (10 mL) was added. The mixture was extracted three times with ethyl acetate (30 mL×3), and the organic phases were combined, washed with saturated brine (20 mL), dried over anhydrous sodium sulfate, and filtered. The filtrate was concentrated under reduced pressure. The resulting residue was purified by silica gel column chromatography (petroleum ether:ethyl acetate=10:1-1:1) to obtain the product compound 10d (0.25 g, yield: 65%). m/z (ESI): 468.2 [M+H]⁺.

### Step 4: Synthesis of methyl 6-amino-4-(2-(((tert-butoxycarbonyl)amino)methyl)-3-ethylphenyl)-7-(3-methoxy-2,6-dimethylphenyl)-2-methyl-7H-pyrrolo[2,3-d]pyrimidine-5-carboxylate (10e)

Compound 10d (0.25 g, 0.53 mmol) was dissolved in anhydrous methanol (10 mL) at room temperature, and then di-tert-butyl dicarbonate (0.17 g, 0.80 mmol) and Raney nickel (10 mg, 53 μmol) were added. The system was purged with hydrogen. The mixture was reacted at room temperature for 3 h at a pressure of 45 psi. The reaction mixture was filtered to remove the insoluble substance, and the filtrate was concentrated. The resulting residue was purified by silica gel column chromatography (petroleum ether:ethyl acetate=1:1-1:3) to obtain the product compound 10e (0.25 g, yield: 82%). m/z (ESI): 574.5 [M+H]⁺.

Compound 10 was prepared by replacing compound 3d with compound 10e and using a method similar to that in Example 3.

m/z (ESI): 428.2 [M+H]⁺;

¹H NMR (400 MHz, DMSO-d₆) δ 9.83-9.69 (m, 1H), 8.26 (d, J=6.4 Hz, 1H), 7.67-7.57 (m, 5H), 7.14 (dd, J=13.6, 8.4 Hz, 1H), 7.02 (d, J=8.4 Hz, 1H), 5.09-5.03 (m, 1H), 4.36-4.31 (m, 1H), 3.04-2.98 (m, 1H), 2.84-2.78 (m, 1H), 2.68 (s, 3H), 1.98-1.83 (m, 3H), 1.78-1.58 (m, 3H), 1.23 (t, J=7.6 Hz, 3H).

### Example 11. Preparation of 2-amino-1-(3-hydroxy-2,6-dimethylphenyl)-6-methoxy-11-methyl-4,5-dihydro-1,4,10,12-tetraazabenzo[4,5]cyclooctatetraeno[1,2,3-cd]inden-3(1H)-one (Compound 11)

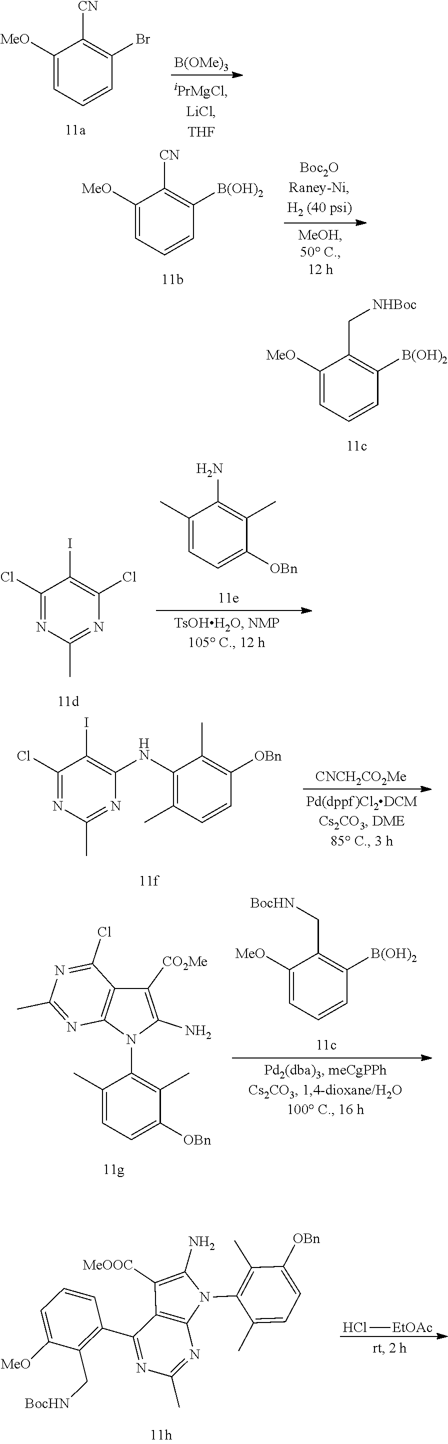

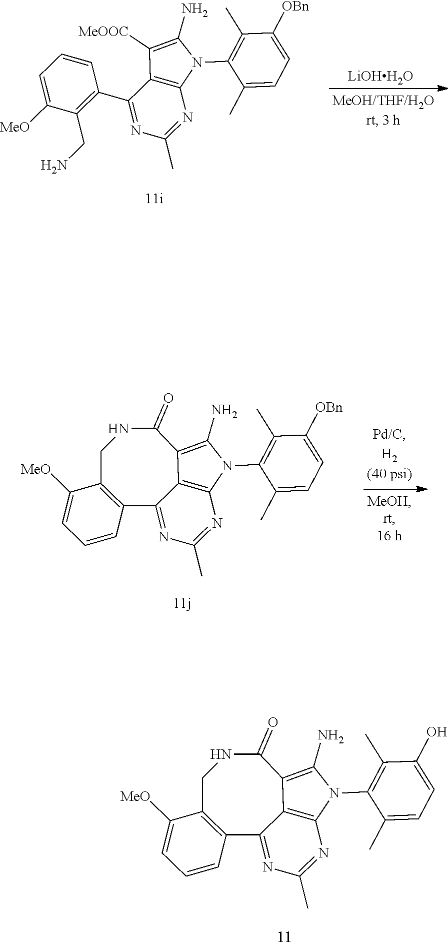 [SMILES: COC(=O)c1c(N)n(-c2c(C)ccc(OCc3ccccc3)c2C)c2nc(C)nc(-c3cccc(OC)c3CN)c12 ; "LiOH·H2O" ; COc1cccc2c1CNC(=O)c1c(N)n(-c3c(C)ccc(OCc4ccccc4)c3C)c3nc(C)nc-2c13 ; COc1cccc2c1CNC(=O)c1c(N)n(-c3c(C)ccc(O)c3C)c3nc(C)nc-2c13]

### Step 1: Synthesis of (2-cyano-3-methoxyphenyl)boronic Acid (11b)

Compound 11a (4 g, 19 mmol) was dissolved in anhydrous tetrahydrofuran, and a solution of isopropylmagnesium chloride-lithium chloride (16 mL, 1.3 M) in tetrahydrofuran was added dropwise at −70° C. After the dropwise addition was completed and the mixture was reacted at 0° C. for 1 h, trimethylborate (2.2 g, 21 mmol) was added dropwise at −70° C., and the mixture was moved to room temperature and reacted for 12 h. Water (20 mL) was added to the reaction mixture to quench the reaction. The mixture was extracted 3 times with ethyl acetate (100 mL×3), and the organic phases were combined, washed with a saturated aqueous sodium chloride solution (30 mL), dried over anhydrous sodium sulfate, filtered, and concentrated. The resulting residue was purified by column chromatography (petroleum ether:ethyl acetate=100:1-20:1) to obtain the product compound 11b (2.5 g, yield: 53%).

### Step 2: Synthesis of (2-(((tert-butoxycarbonyl)amino)methyl)-3-methoxyphenyl)boronic Acid (11c)

Compound 11b (2 g, 11 mmol) was dissolved in anhydrous methanol (10 mL) at room temperature, and then di-tert-butyl dicarbonate (4.9 g, 13 mmol) and Raney nickel (66 mg, 1.1 mmol) were added. The system was purged with hydrogen. The reaction mixture was reacted at 50° C. for 12 h under an oil bath at a pressure of 40 psi. The reaction mixture was cooled to room temperature and then filtered to remove the insoluble substance, and the filtrate was concentrated. The resulting residue was purified by silica gel column chromatography (petroleum ether:ethyl acetate=1:1-1:3) to obtain the product compound 11c (1.4 g, yield: 32%). m/z (ESI): 280.2 [M−H]⁻.

### Step 3: Synthesis of N-(3-(benzyloxy)-2,6-dimethylphenyl)-6-chloro-5-iodo-2-methylpyrimidin-4-amine (11f)

Compound 11d (5.0 g, 17 mmol) and compound 11e (4.7 g, 21 mmol) were dissolved in N-methyl-2-pyrrolidone (50 mL) at room temperature, and p-toluenesulfonic acid monohydrate (0.60 g, 3.5 mmol) was added. The mixture was then stirred at 105° C. for 12 h. After the reaction was cooled to room temperature, the reaction mixture was diluted with water (100 mL) and extracted with ethyl acetate (50 mL×3). The organic phases were combined, washed with saturated brine (50 mL), dried over anhydrous sodium sulfate, and filtered. The filtrate was concentrated. The resulting residue was purified by normal-phase column chromatography (petroleum ether:ethyl acetate=50:1) to obtain the target product compound 11f (1.2 g, yield: 14%). m/z (ESI): 480.1 [M+H]⁺.

### Step 4: Synthesis of methyl 6-amino-7-(3-(benzyloxy)-2,6-dimethylphenyl)-4-chloro-2-methyl-7H-pyrrolo[2,3-d]pyrimidine-5-carboxylate (11g)

Methyl cyanoacetate (0.50 g, 5.0 mmol) was dissolved in anhydrous ethylene glycol dimethyl ether (12 mL). Cesium carbonate (2.4 g, 7.4 mmol) was added. After the mixture was stirred at room temperature for 30 min, compound 11f (0.60 g, 1.2 mmol) and [1,1′-bis(diphenylphosphino)ferrocene]palladium dichloride dichloromethane complex (92 mg, 0.12 mmol) were added. The system was purged with nitrogen 3 times, and the mixture was reacted at 85° C. for 3 h under nitrogen atmosphere. The reaction mixture was cooled to room temperature and then filtered to remove the insoluble substance, and the filtrate was concentrated to dryness by rotary evaporation. The resulting residue was purified by reverse-phase column chromatography (C18, 0.05% aqueous ammonia:acetonitrile=20:1-1:20) to obtain compound 11g (0.21 g, yield: 35%). m/z (ESI): 451.1 [M+H]⁺.

### Step 5: Synthesis of methyl 6-amino-7-(3-(benzyloxy)-2,6-dimethylphenyl)-4-(2-(((tert-butoxycarbonyl)amino)methyl)-3-methoxyphenyl)-2-methyl-7H-pyrrolo[2,3-d]pyrimidine-5-carboxylate (11h)

Compound 11g (0.13 g, 0.36 mmol), compound 11c (0.10 g, 0.36 mmol), bis(dibenzylideneacetone)palladium (32 mg, 36 μmol), 1,3,5,7-tetramethyl-6-phenyl-2,4,8-trioxa-6-phosphaadamantane (42 mg, 0.14 mmol), and cesium carbonate (0.23 g, 0.71 mmol) were dissolved in 1,4-dioxane/water (5 mL/1 mL) at room temperature. The system was purged with nitrogen three times. The reaction mixture was then moved to 100° C. and reacted for 16 h. After the reaction was cooled to room temperature, the reaction mixture was diluted with water (10 mL) and extracted with ethyl acetate (20 mL×3). The organic phases were combined, washed with saturated brine (20 mL), dried over anhydrous sodium sulfate, and filtered. The filtrate was concentrated. The resulting residue was purified by normal-phase column chromatography (petroleum ether:ethyl acetate=1:1) to obtain the target product compound 11h (0.10 g, yield: 48%). m/z (ESI): 652.4 [M+H]⁺.

### Step 6: Synthesis of methyl 6-amino-4-(2-(aminomethyl)-3-methoxyphenyl)-7-(3-(benzyloxy)-2,6-dimethylphenyl)-2-methyl-7H-pyrrolo[2,3-d]pyrimidine-5-carboxylate (11i)

Compound 11h (50 mg, 87 μmol) was dissolved in a solution of hydrogen chloride in ethyl acetate (4 M in EtOAc, 4.8 mL) at room temperature, and the mixture was reacted under these conditions for 2 h. After the reaction was completed, the reaction mixture was concentrated. The resulting solid residue, compound 11i (40 mg), could be directly used in the next step without purification.

### Step 7: Synthesis of 2-amino-1-(3-(benzyloxy)-2,6-dimethylphenyl)-6-methoxy-11-methyl-4,5-dihydro-1,4,10,12-tetraazabenzo[4,5]cyclooctatetraeno[1,2,3-cd]inden-3(1H)-one (11j)

Compound 11i (40 mg, 84 μmol) obtained in the previous step was dissolved in methanol/tetrahydrofuran/water (2 mL/2 mL/2 mL), and lithium hydroxide monohydrate (10 mg, 0.42 mmol) was then added. The reaction mixture was reacted at room temperature for 3 h. The reaction mixture was concentrated. The resulting residue was purified by reverse-phase column chromatography (C18, 0.05% aqueous ammonia:acetonitrile=20:1-1:20) to obtain the target compound 11j (35 mg, yield: 89%). LC-MS: m/z (ESI): 520.3 [M+H]⁺.

### Step 8: Synthesis of 2-amino-1-(3-hydroxy-2,6-dimethylphenyl)-6-methoxy-11-methyl-4,5-dihydro-1,4,10,12-tetraazabenzo[4,5]cyclooctatetraeno[1,2,3-cd]inden-3(1H)-one (Compound 11)

Compound 11j (35 mg, 79 μmol) was dissolved in anhydrous methanol (5 mL) at room temperature, and then Pd/C (10%, 7.2 mg) was added. The system was purged with hydrogen. The reaction mixture was reacted at room temperature at a pressure of 40 psi for 12 h. The reaction mixture was filtered to remove the insoluble substance, and the filtrate was concentrated. The resulting residue was subjected to reverse-phase column chromatography (C18, 0.05% aqueous ammonia:acetonitrile=20:1-1:20) to obtain compound 11 (21 mg, yield: 62%). m/z (ESI): 430.1 [M+H]⁺;

¹H NMR (400 MHz, CDCl₃) δ 8.84-8.66 (m, 1H), 7.55-7.40 (m, 2H), 7.04-6.92 (m, 2H), 6.80-6.60 (m, 1H), 6.06-5.99 (m, 2H), 5.01 (br dd, J=8.8, 14.8 Hz, 1H), 4.65-4.55 (m, 1H), 3.95 (s, 3H), 2.82 (s, 3H), 2.02-1.88 (m, 3H), 1.84-1.70 (m, 3H).

### Example 12. Preparation of 5-amino-4-(3-hydroxy-2,6-dimethylphenyl)-2,8-dimethyl-7,8-dihydro-1,3,4,7,8,9-hexaazabenzo[cd]cyclopenta[f]azulen-6(4H)-one (Compound 12)

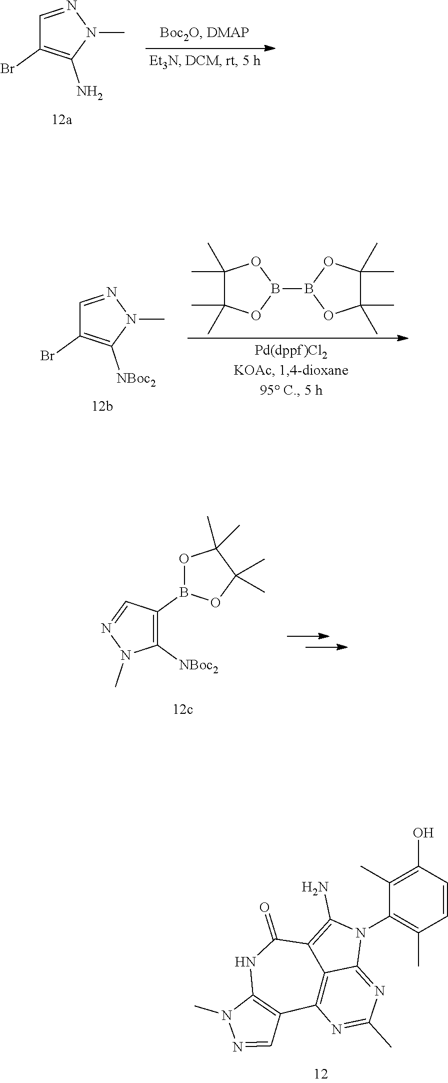

### Step 1: Synthesis of tert-butyl N-(4-bromo-2-methyl-pyrazol-3-yl)-N-tert-butoxycarbonyl-carbamate (12b)

Compound 12a (2.0 g, 11 mmol) was dissolved in an anhydrous dichloromethane solution at room temperature, and di-tert-butyl dicarbonate (3.7 g, 17 mmol), triethylamine (2.3 g, 23 mmol), and 4-dimethylaminopyridine (0.14 g, 1.1 mmol) were sequentially added. The mixture was reacted under the condition for 5 h, and the reaction was quenched with water (20 mL). The mixture was extracted 3 times with ethyl acetate (100 mL×3), and the organic phases were combined, washed with a saturated aqueous sodium chloride solution (100 mL), dried over anhydrous sodium sulfate, and filtered. The filtrate was concentrated, and the resulting residue was subjected to column chromatography (petroleum ether:ethyl acetate=1:0-3:1) to obtain the product compound 12b (3.1 g, yield: 73%).

### Step 2: Synthesis of tert-butyl N-tert-butoxycarbonyl-N-[2-methyl-4-(4,4,5,5-tetramethyl-1,3,2-dioxaborolan-2-yl)pyrazol-3-yl]carbamate (12c)

Compound 12b (3.0 g, 8.0 mmol), bis(pinacolato)diboron (3.0 g, 12 mmol), [1,1′-bis(diphenylphosphino)ferrocene]palladium dichloride (0.59 g, 0.80 mmol), and potassium acetate (1.6 g, 16 mmol) were dissolved in anhydrous 1,4-dioxane at room temperature. The system was purged with nitrogen 3 times, and the mixture was reacted at 95° C. for 5 h under nitrogen atmosphere. After the reaction mixture was cooled to room temperature, water (50 mL) was added to the reaction mixture to quench the reaction. The mixture was extracted 3 times with ethyl acetate (50 mL×3), and the organic phases were combined, washed with a saturated aqueous sodium chloride solution (30 mL), dried over anhydrous sodium sulfate, and filtered. The filtrate was concentrated. The resulting residue was purified by column chromatography (petroleum ether:ethyl acetate=100:1-20:1) to obtain the product compound 12c (1.8 g, yield: 53%). m/z (ESI): 424.2 [M+H]⁺.

Compound 12 was prepared by replacing compound 3c with compound 12c and using a method similar to that in Example 3.

m/z (ESI): 390.1 [M+H]⁺;

¹H NMR (400 MHz, DMSO-d₆) δ 7.70 (s, 1H), 7.08 (d, J=8.4 Hz, 1H), 6.94 (d, J=8.4 Hz, 1H), 6.91 (s, 2H), 3.74 (s, 3H), 2.35 (s, 3H), 1.80 (s, 3H), 1.71 (s, 3H).

### Example 13. Preparation of 2-amino-1-(3-hydroxy-2,6-dimethylphenyl)-6,9-dimethyl-4,6-dihydro-1,4,5,6,8,10-hexaazabenzo[cd]cyclopenta[f]azulen-3(1H)-one (Compound 13)

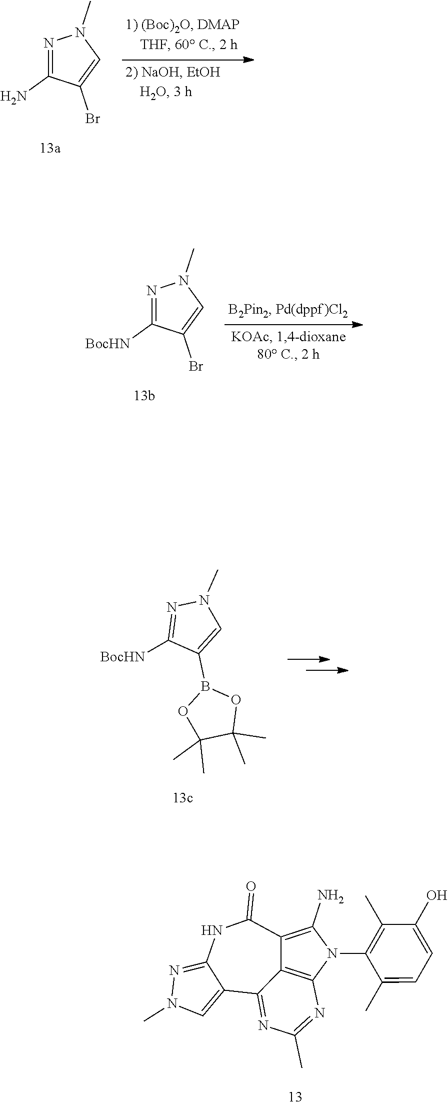

### Step 1: Synthesis of tert-butyl (4-bromo-1-methyl-1H-pyrazol-3-yl)carbamate (13b)

Compound 13a (0.3 g, 1.7 mmol) was dissolved in tetrahydrofuran (10 mL) at room temperature, and di-tert-butyl dicarbonate (2.2 g, 10 mmol) and 4-dimethylaminopyridine (20 mg, 0.2 mmol) were added to the system. The reaction mixture was moved to 60° C. and reacted for 2 h, and the mixture was concentrated in vacuum to remove the solvent. The crude product was dissolved in ethanol (10 mL), and a sodium hydroxide solution (2 mL, 20% aqueous solution) was added to the reaction. The mixture was reacted at room temperature for another 3 h and concentrated in vacuum to remove the solvent. The crude product was purified by reverse-phase column chromatography (C18, 0.05% aqueous ammonia:acetonitrile=20:1-1:20) to obtain compound 13b (0.32 g, yield: 67%). m/z (ESI): 220.0 [M+H−tBu]⁺.

### Step 2: Synthesis of tert-butyl (1-methyl-4-(4,4,5,5-tetramethyl-1,3,2-dioxaborolan-2-yl)-1H-pyrazol-3-yl)carbamate (13c)

Compound 13b (320 mg, 1.2 mmol), bis(pinacolato)diboron (0.61 g, 2.4 mmol), potassium acetate (0.35 g, 3.6 mmol), and [1,1′-bis(diphenylphosphino)ferrocene]palladium dichloride (90 mg, 0.12 mmol) were dissolved in dioxane (10 mL) at room temperature. The system was purged with nitrogen three times, and the mixture was heated to 80° C. and reacted for 2 h. The system was cooled to room temperature, and the reaction mixture was filtered and concentrated in vacuum. The resulting crude product could be directly used in the next step without purification.

Compound 13 was prepared by replacing compound 3c with compound 13c and using a method similar to that in Example 3.

m/z (ESI): 390.1 [M+H]⁺;

¹H NMR (400 MHz, DMSO-d₆) δ 9.60 (s, 1H), 9.54 (s, 1H), 8.11 (s, 1H), 7.07 (d, J=8.4 Hz, 1H), 6.93 (d, J=8.4 Hz, 1H), 6.89 (s, 2H), 3.74 (s, 3H), 2.35 (s, 3H), 1.78 (s, 3H), 1.69 (s, 3H).

### Example 14. Preparation of 2-amino-1-(3-hydroxy-2,6-dimethylphenyl)-9-methyl-6-(2,2,2-trifluoroethyl)-4,6-dihydro-1,4,5,6,8,10-hexaazabenzo[cd]cyclopenta[f]azulen-3(1H)-one (Compound 14)

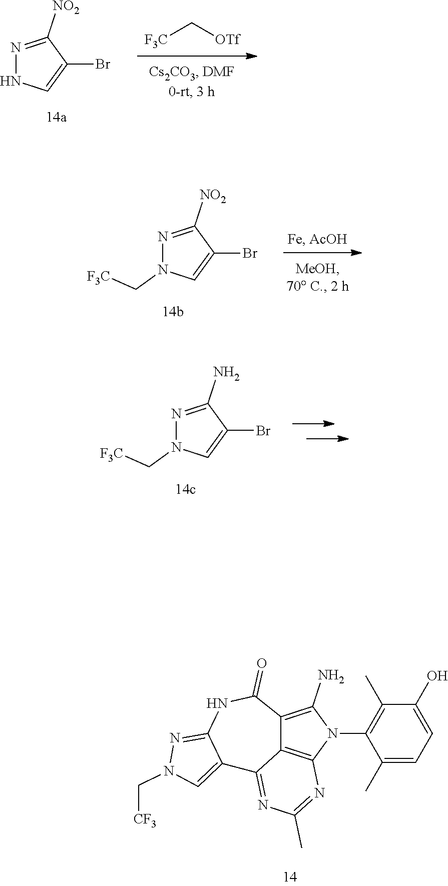 [SMILES: O=[N+]([O-])c1n[nH]cc1Br ; O=S(=O)(OCC(F)(F)F)C(F)(F)F ; O=[N+]([O-])c1nn(CC(F)(F)F)cc1Br ; Nc1nn(CC(F)(F)F)cc1Br ; Cc1nc2c3c(c(N)n(-c4c(C)ccc(O)c4C)c3n1)C(=O)Nc1nn(CC(F)(F)F)cc1-2]

### Step 1: Synthesis of 4-bromo-3-nitro-1-(2,2,2-trifluoroethyl)-1H-pyrazole (14b)

Compound 14a (2.0 g, 10 mmol) was dissolved in DMF (5 mL) under an ice bath. Cesium carbonate (6.8 g, 21 mmol) and 2,2,2-trifluoroethyl trifluoromethanesulfonate (1.9 g, 10 mmol) were then added. The mixture was warmed to room temperature and reacted for 3 h. Water (20 mL) was added to the reaction mixture to quench the reaction. The mixture was extracted 3 times with ethyl acetate (50 mL×3), and the organic phases were combined, washed with a saturated aqueous sodium chloride solution (30 mL), dried over anhydrous sodium sulfate, and filtered. The filtrate was concentrated. The resulting crude product could be directly used in the next step without purification. m/z (ESI): 273.7 [M+H]⁺.

### Step 2: Synthesis of 4-bromo-1-(2,2,2-trifluoroethyl)-1H-pyrazol-3-amine (14c)

Compound 14b (2.7 g, 9.7 mmol) was dissolved in methanol (20 mL). Iron powder (2.7 g, 49 mmol) and acetic acid (5.8 g, 97 mmol) were sequentially added. The mixture was warmed to 70° C. and reacted for 2 h. After the reaction mixture was cooled to room temperature, water (20 mL) was added to the reaction mixture to quench the reaction. The mixture was extracted 3 times with ethyl acetate (100 mL×3), and the organic phases were combined, washed with a saturated aqueous sodium chloride solution (50 mL), dried over anhydrous sodium sulfate, and filtered. The filtrate was concentrated. The resulting crude product was purified by silica gel column chromatography (petroleum ether:ethyl acetate=50:1-20:1) to obtain the target compound 14c (1.5 g, yield: 62%). m/z (ESI): 244.3 [M+H]⁺.

Compound 14 was prepared by replacing compound 13a with compound 14c and using a method similar to that in Example 13.

m/z (ESI): 458.2 [M+H]⁺.

¹H NMR (400 MHz, DMSO-d₆) δ 9.72 (s, 1H), 9.58 (s, 1H), 8.28 (s, 1H), 7.08 (d, J=8.3 Hz, 1H), 7.01-6.89 (m, 3H), 2.54 (s, 2H), 2.38 (s, 3H), 1.79 (s, 3H), 1.70 (s, 3H).

### Example 15. Preparation of 5-amino-9-fluoro-4-(3-hydroxy-2,6-dimethylphenyl)-2-methyl-4,7-dihydro-6H-1,3,4,7,10-pentaazadibenzo[cd,f]azulen-6-one (Compound 15)

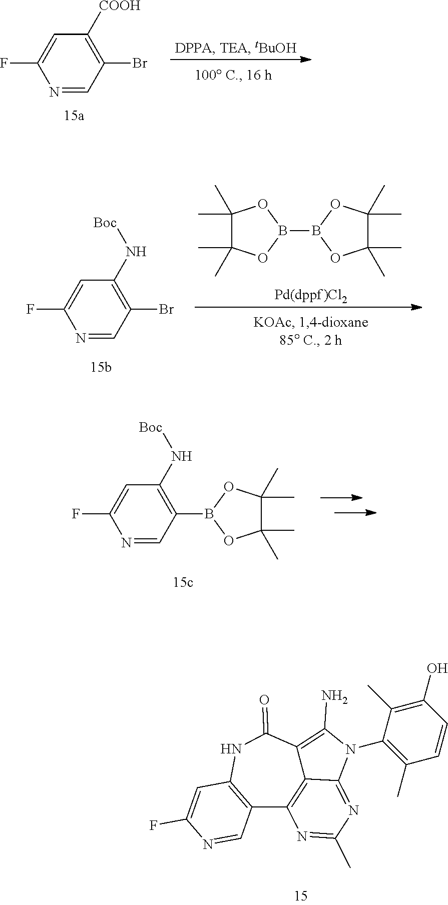

### Step 1: Synthesis of tert-butyl (5-bromo-2-fluoropyridin-4-yl)carbamate (15b)

Compound 15a (0.90 g, 4.1 mmol) was dissolved in tert-butanol. Diphenylphosphoryl azide (1.7 g, 6.1 mmol) and triethylamine (0.83 g, 1.1 mL, 8.2 mmol) were then added. The mixture was reacted at 100° C. for 16 h. The mixture was concentrated in vacuum to remove the solvent. The resulting crude product was purified by normal-phase column chromatography (petroleum ether:ethyl acetate=100:1-20:1) to obtain compound 15b (1.2 g, yield: 82%).

### Step 2: Synthesis of tert-butyl (2-fluoro-5-(4,4,5,5-tetramethyl-1,3,2-dioxaborolan-2-yl)pyridin-4-yl)carbamate (15c)

Compound 15b (1.0 g, 3.4 mmol), bis(pinacolato)diboron (1.7 g, 6.9 mmol), potassium acetate (1.0 g, 10 mmol), and [1,1′-bis(diphenylphosphino)ferrocene]palladium dichloride (0.25 g, 0.34 mmol) were dissolved in dioxane (10 mL) at room temperature. The system was purged with nitrogen three times, and the mixture was heated to 85° C. and reacted for 2 h. The system was cooled to room temperature, and the reaction mixture was filtered and concentrated in vacuum. The resulting crude product was purified by normal-phase column chromatography (petroleum ether:ethyl acetate=100:1-20:1) to obtain compound 15c (0.95 g, yield: 83%).

Compound 15 was prepared by replacing compound 3c with compound 15c and using a method similar to that in Example 3.

m/z (ESI): 405.1 [M+H]⁺;

¹H NMR (400 MHz, DMSO-d₆) δ 9.71 (s, 1H), 9.62 (s, 1H), 9.17 (s, 1H), 7.11 (s, 2H), 7.09 (d, J=8.0 Hz, 1H), 6.95 (d, J=8.4 Hz, 1H), 6.73 (s, 1H), 2.45 (s, 3H), 1.80 (s, 3H), 1.71 (s, 3H).

### Example 16. Preparation of 5-amino-8-fluoro-4-(3-hydroxy-2,6-dimethylphenyl)-2-methyl-4,7-dihydro-6H-1,3,4,7,10-pentaazadibenzo[cd,f]azulen-6-one (Compound 16)

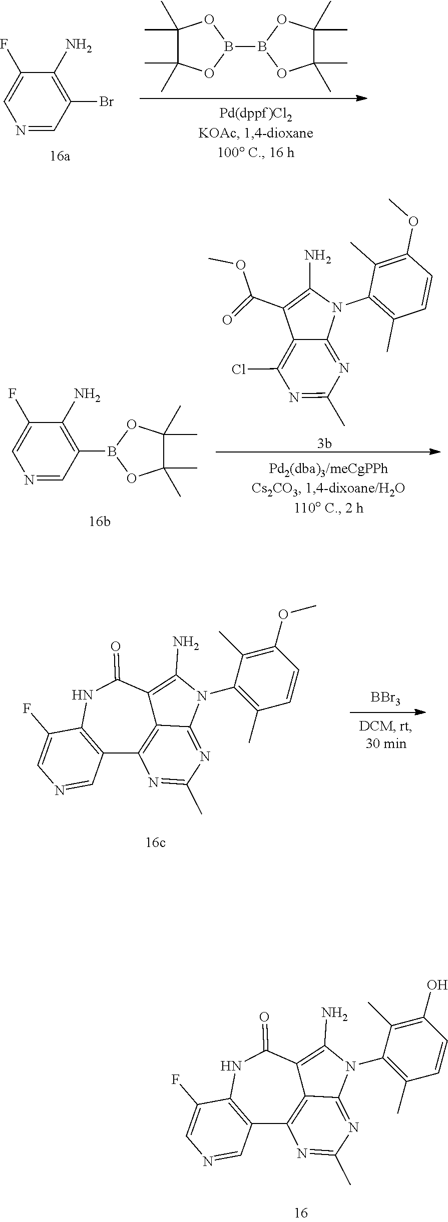

### Step 1: Synthesis of 3-fluoro-5-(4,4,5,5-tetramethyl-1,3,2-dioxaborolan-2-yl)pyridin-4-amine (16b)

Compound 16a (0.50 g, 2.6 mmol), bis(pinacolato)diboron (0.86 g, 3.4 mmol), potassium acetate (0.64 g, 6.5 mmol), and [1,1′-bis(diphenylphosphino)ferrocene]palladium dichloride (0.19 g, 0.26 mmol) were dissolved in dioxane (15 mL) at room temperature. The system was purged with nitrogen three times, and the mixture was heated to 100° C. and reacted for 16 h. The reaction was cooled to room temperature, and the reaction mixture was filtered. The filtrate was concentrated. The resulting crude product was purified by normal-phase column chromatography (petroleum ether:ethyl acetate=100:1-20:1) to obtain compound 16b (0.30 g, yield: 48%). m/z (ESI): 238.2 [M−H]⁻.

### Step 2: Synthesis of 5-amino-8-fluoro-4-(3-methoxy-2,6-dimethylphenyl)-2-methyl-4,7-dihydro-6H-1,3,4,7,10-pentaazadibenzo[cd,f]azulen-6-one (16c)

Compound 16b (0.20 g, 0.84 mmol), compound 3b (0.17 g, 0.47 mmol), bis(dibenzylideneacetone)palladium (43 mg, 47 μmol), 1,3,5,7-tetramethyl-6-phenyl-2,4,8-trioxa-6-phosphaadamantane (54 mg, 0.19 mmol), and cesium carbonate (0.30 g, 0.93 mmol) were dissolved in 1,4-dioxane/water (5 mL/1 mL) at room temperature. The system was purged with nitrogen three times. The reaction mixture was then moved to 110° C. and reacted for 2 h. After the reaction was cooled to room temperature, the reaction mixture was diluted with water (10 mL) and extracted with ethyl acetate (20 mL×3). The organic phases were combined, washed with saturated brine (20 mL), dried over anhydrous sodium sulfate, and filtered. The filtrate was concentrated. The resulting residue was purified by normal-phase column chromatography (petroleum ether:ethyl acetate=1:1) to obtain the target product compound 16c (0.15 g, yield: 77%). m/z (ESI): 451.2 [M+H]⁺.

### Step 3: Synthesis of 5-amino-8-fluoro-4-(3-hydroxy-2,6-dimethylphenyl)-2-methyl-4,7-dihydro-6H-1,3,4,7,10-pentaazadibenzo[cd,f]azulen-6-one (16)

Compound 16c (0.15 g, 0.36 mmol) was dissolved in dichloromethane (4 mL) at room temperature. A solution of boron tribromide (1 mol/L, 2 mL) in dichloromethane was added, and the mixture was stirred for 30 min. The reaction mixture was quenched with methanol (5 mL) and then concentrated. The resulting residue was purified by reverse-phase column chromatography (C18, 0.05% aqueous ammonia:acetonitrile=20:1-1:20) to obtain compound 16 (3.4 mg, yield: 2.4%). m/z (ESI): 405.1 [M+H]⁺;

¹H NMR (400 MHz, CDCl₃) δ 9.42 (s, 1H), 8.33 (d, J=2.8 Hz, 1H), 7.15 (d, J=9.2 Hz, 1H), 6.97 (d, J=7.6 Hz, 1H), 2.56 (s, 3H), 1.92 (s, 3H), 1.87 (s, 3H).

### Example 17. Preparation of 5-amino-4-(3-hydroxy-2,6-dimethylphenyl)-2-methyl-4,7-dihydro-6H-1,3,4,7,8-pentaazadibenzo[cd,f]azulen-6-one (Compound 17)

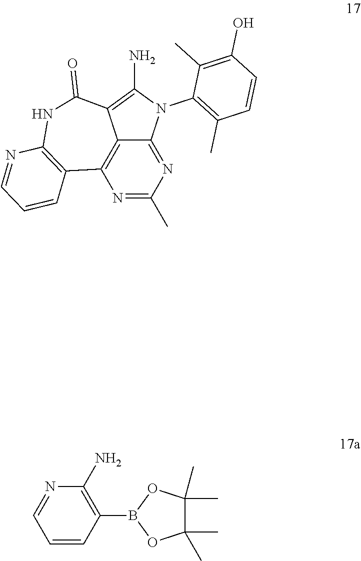 [SMILES: Cc1nc2c3c(c(N)n(-c4c(C)ccc(O)c4C)c3n1)C(=O)Nc1ncccc1-2 ; CC1(C)OB(c2cccnc2N)OC1(C)C]

Compound 17 was prepared by replacing compound 16b with compound 17a and using a method similar to that in Example 16.

m/z (ESI): 387.2 [M+H]⁺.

### Example 18. Preparation of 5-amino-4-(3-hydroxy-2,6-dimethylphenyl)-2-methyl-4,7-dihydro-6H-1,3,4,7,9-pentaazadibenzo[cd,f]azulen-6-one (Compound 18)

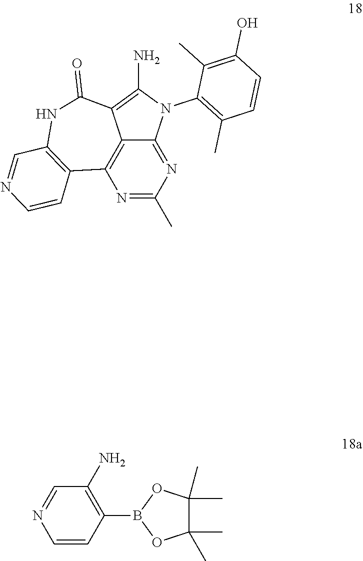 [SMILES: Cc1nc2c3c(c(N)n(-c4c(C)ccc(O)c4C)c3n1)C(=O)Nc1cnccc1-2 ; CC1(C)OB(c2ccncc2N)OC1(C)C]

Compound 18 was prepared by replacing compound 16b with compound 18a and using a method similar to that in Example 16.

m/z (ESI): 387.2 [M+H]⁺.

¹H NMR (400 MHz, DMSO-d₆) δ 9.68 (s, 1H), 9.31 (s, 1H), 8.36 (s, 1H), 8.23 (d, J=5.2 Hz, 1H), 8.14 (d, J=5.2 Hz, 1H), 7.09 (d, J=8.0 Hz, 1H), 7.07 (s, 2H), 6.95 (d, J=8.0 Hz, 1H), 2.46 (s, 3H), 1.79 (s, 3H), 1.71 (s, 3H).

### Example 19. Preparation of 5-amino-4-(3-hydroxy-2,6-dimethylphenyl)-2-methyl-4,7-dihydro-6H-1,3,4,7,10-pentaazadibenzo[cd,f]azulen-6-one (Compound 19)

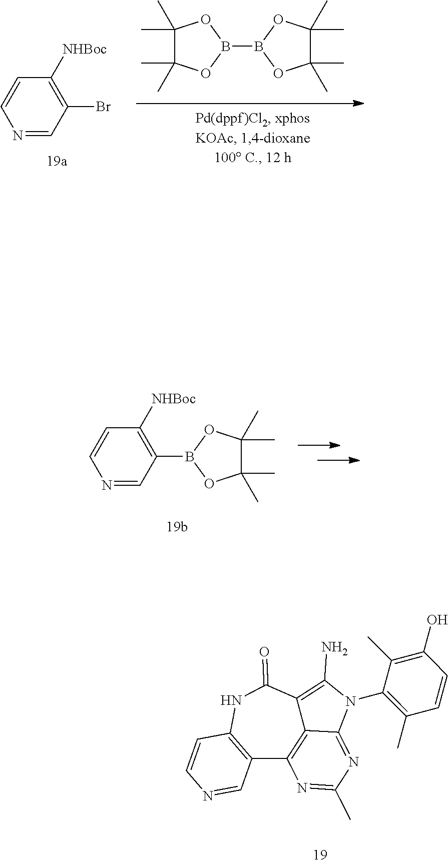

### Step 1: Synthesis of tert-butyl (3-(4,4,5,5-tetramethyl-1,3,2-dioxaborolan-2-yl)pyridin-4-yl)carbamate (19b)

Compound 19a (5.0 g, 18 mmol), bis(pinacolato)diboron (7.0 g, 27 mmol), potassium acetate (3.6 g, 37 mmol), [1,1′-bis(diphenylphosphino)ferrocene]palladium dichloride (1.3 g, 1.8 mmol), and 2-dicyclohexylphosphino-2′,4′,6′-triisopropylbiphenyl (0.87 g, 1.8 mmol) were dissolved in 1,4-dioxane (20 mL) at room temperature. The system was purged with nitrogen three times, and the mixture was heated to 100° C. and reacted for 12 h. The reaction was cooled to room temperature, and the reaction mixture was filtered. The filtrate was concentrated. The resulting crude product was sequentially recrystallized with acetonitrile and ethyl acetate to obtain compound 19b (2.0 g, yield: 34%). m/z (ESI): 319.2 [M−H]⁻.

Compound 19 was prepared by replacing compound 3c with compound 19b and using a method similar to that in Example 3.

m/z (ESI): 387.2 [M+H]⁺.

### Example 20. Preparation of 5-amino-4-(3-hydroxy-2,6-dimethylphenyl)-2-methyl-4,7-dihydro-6H-1,3,4,7,11-pentaazadibenzo[cd,f]azulen-6-one (Compound 20)

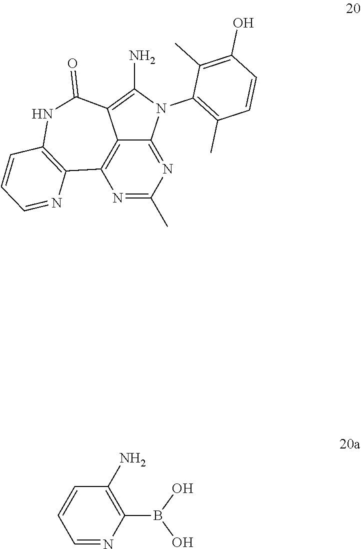 [SMILES: Cc1nc2c3c(c(N)n(-c4c(C)ccc(O)c4C)c3n1)C(=O)Nc1cccnc1-2 ; Nc1cccnc1B(O)O]

Compound 20 was prepared by replacing compound 16b with compound 20a and using a method similar to that in Example 16.

m/z (ESI): 387.2 [M+H]⁺;

¹H NMR (400 MHz, DMSO-d₆) δ 9.71 (s, 1H), 8.83-8.81 (m, 2H), 8.24 (dd, J=4.8, 1.6 Hz, 1H), 7.11-7.07 (m, 2H), 7.05 (s, 2H), 6.96 (d, J=8.4 Hz, 1H), 2.46 (s, 3H), 1.81 (s, 3H), 1.72 (s, 3H).

### Example 21. Preparation of 2-amino-1-(3-hydroxy-2,6-dimethylphenyl)-6,10-dimethyl-1,4,5,6-tetrahydro-3H-1,4,6,7,9,11-hexaazacyclopenta[4,5]cyclooctatetraeno[1,2,3-cd]inden-3-one (Compound 21)

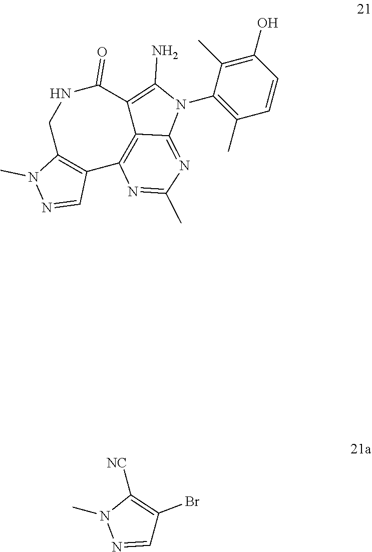

Compound 21 was prepared by replacing compound 8a with compound 21a and using a method similar to that in Example 8.

m/z (ESI): 404.1 [M+H]⁺.

¹H NMR (400 MHz, DMSO-d₆) δ 9.62 (s, 1H), 7.97-7.93 (m, 1H), 7.71 (s, 1H), 7.09 (d, J=8.0 Hz, 1H), 6.95-6.90 (m, 3H), 4.74 (brs, 1H), 4.28 (brs, 1H), 3.91 (s, 3H), 2.42 (s, 3H), 1.87-1.61 (m, 6H).

### Example 22. Preparation of 2-amino-8-fluoro-1-(3-hydroxy-2,6-dimethylphenyl)-11-methyl-4,5-dihydro-1,4,6,10,12-pentaazabenzo[4,5]cyclooctatetraeno[1,2,3-cd]inden-3(1H)-one (Compound 22)

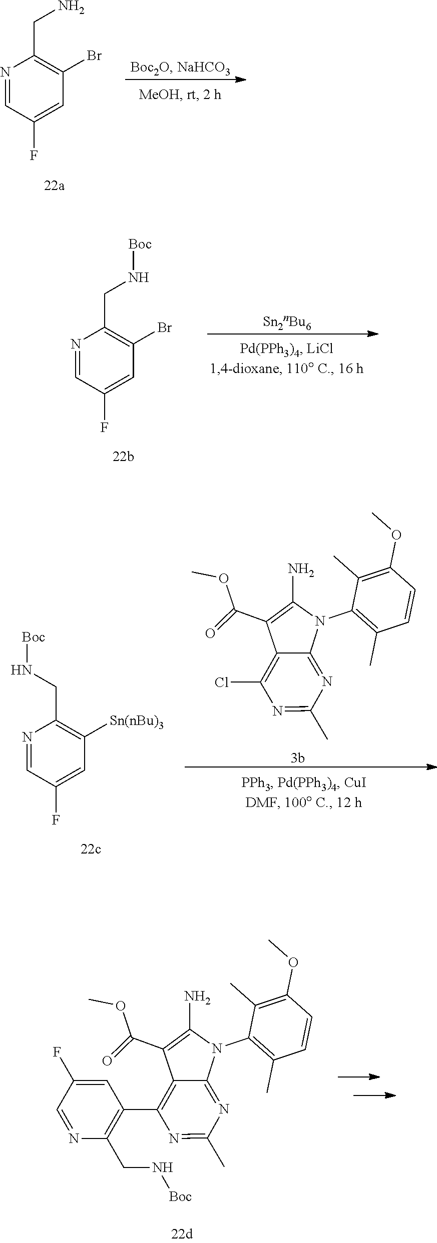

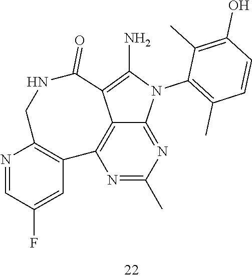 [SMILES: Cc1nc2c3c(c(N)n(-c4c(C)ccc(O)c4C)c3n1)C(=O)NCc1ncc(F)cc1-2]

### Step 1: Synthesis of tert-butyl ((3-bromo-5-fluoropyridin-2-yl)methyl)carbamate (22b)

At room temperature, compound 22a (1.0 g, 4.1 mmol) was dissolved in anhydrous methanol (10 mL), and then di-tert-butyl dicarbonate (0.90 g, 4.1 mmol) and sodium bicarbonate (0.70 g, 8.2 mmol) were added to the system. The mixture was reacted at room temperature for 2 h. Water (20 mL) was added to the reaction mixture to quench the reaction. The mixture was extracted 3 times with ethyl acetate (100 mL×3), and the organic phases were combined, washed with a saturated aqueous sodium chloride solution (50 mL), dried over anhydrous sodium sulfate, and filtered. The filtrate was concentrated. The resulting crude compound 22b could be directly used in the next step without purification. m/z (ESI): 251.0 [M+H−C₄H₉]⁺.

### Step 2: Synthesis of tert-butyl ((5-fluoro-3-(tri-n-butylstannyl)pyridin-2-yl)methyl)carbamate (22c)

Compound 22b (1.0 g, 3.3 mmol), tetrakis(triphenylphosphine)palladium (0.38 g, 0.33 mmol), hexa-n-butylditin (3.8 g, 6.6 mmol), and lithium chloride (0.42 g, 9.9 mmol) were dissolved in anhydrous 1,4-dioxane (15 mL). The system was purged with nitrogen three times, and the mixture was heated to 110° C. and reacted for 16 h. The system was cooled to room temperature, and water (20 mL) was added to the reaction mixture to quench the reaction. The mixture was extracted with ethyl acetate (50 mL×3), and the organic phases were combined, washed with a saturated aqueous sodium chloride solution (30 mL), dried over anhydrous sodium sulfate, and filtered. The filtrate was concentrated. The resulting residue was purified by column chromatography (petroleum ether:ethyl acetate=100:1-7:1) to obtain the product compound 22c (0.15 g, yield: 7%). m/z (ESI): 517.2 [M+H]⁺.

### Step 3: Synthesis of methyl 6-amino-4-(2-(((tert-butoxycarbonyl)amino)methyl)-5-fluoropyridin-3-yl)-7-(3-methoxy-2,6-dimethylphenyl)-2-methyl-7H-pyrrolo[2,3-d]pyrimidine-5-carboxylate (22d)

At room temperature, compound 22c (0.15 g, 0.28 mmol), compound 3b (73 mg, 0.19 mmol), tetrakis(triphenylphosphine)palladium (22 mg, 19 μmol), cuprous iodide (7.4 mg, 39 μmol), and triphenylphosphine (64 mg, 0.24 mmol) were dissolved in N,N-dimethylformamide (1 mL). The system was purged with nitrogen three times, and the reaction mixture was moved to 100° C. and reacted for 12 h. Water (30 mL) was added to the reaction mixture to quench the reaction. The mixture was extracted 3 times with ethyl acetate (100 mL×3), and the organic phases were combined, washed with a saturated aqueous sodium chloride solution (30 mL), dried over anhydrous sodium sulfate, and filtered. The filtrate was concentrated. The resulting residue was purified by column chromatography (petroleum ether:ethyl acetate=100:1-1:2) to obtain the product compound 22d (95 g, yield: 88%). m/z (ESI): 565.3 [M+H]⁺.

Compound 22 was prepared by replacing compound 3d with compound 22d and using a method similar to that in Example 3.

m/z (ESI): 419.1 [M+H]⁺;

¹H NMR (400 MHz, CD₃OD) δ 8.53 (d, J=2.8 Hz, 1H), 8.02 (dt, J=9.6, 2.4 Hz, 1H), 7.14 (dd, J=8.4, 13.6 Hz, 1H), 6.96 (d, J=8.4 Hz, 1H), 5.31 (dd, J=4.0, 14.4 Hz, 1H), 4.29 (d, J=14.8 Hz, 1H), 2.61 (s, 3H), 2.02-1.92 (m, 3H), 1.83-1.71 (m, 3H).

### Example 23. Preparation of 2-amino-1-(3-hydroxy-2,6-dimethylphenyl)-6-methoxy-11-methyl-4,5-dihydro-1,4,7,10,12-pentaazabenzo[4,5]cyclooctatetraeno[1,2,3-cd]inden-3(1H)-one (Compound 23)

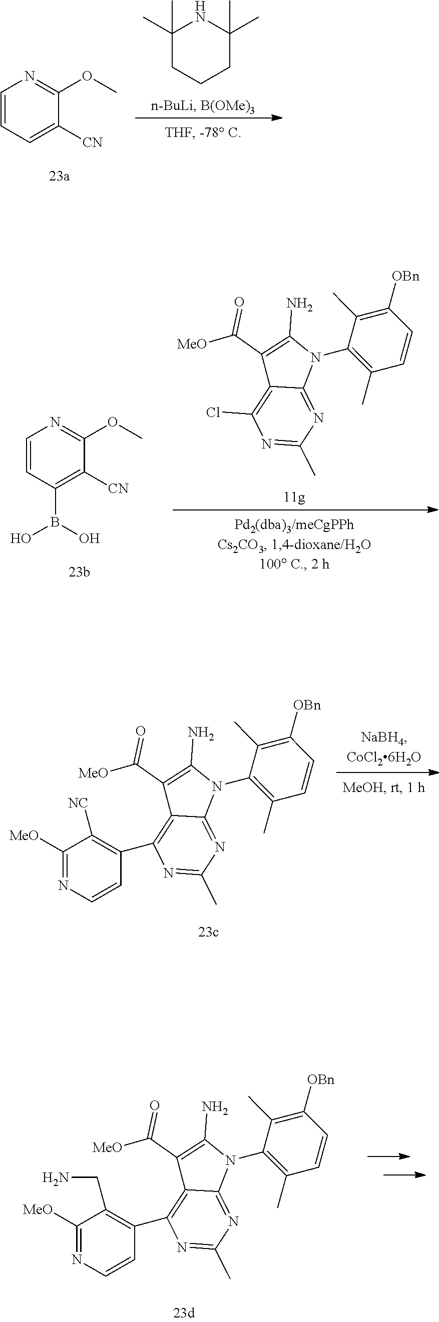

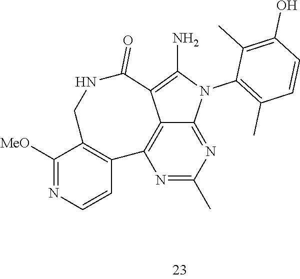 [SMILES: COc1nccc2c1CNC(=O)c1c(N)n(-c3c(C)ccc(O)c3C)c3nc(C)nc-2c13]

### Step 1: Synthesis of (3-cyano-2-methoxypyridin-4-yl)boronic Acid (23b)

At −30° C., n-butyllithium (2.5 M, 7.1 mmol, 2.8 mL) was added dropwise to a solution of 2,2,6,6-tetramethylpiperidine (1.1 g, 7.8 mmol) in tetrahydrofuran (10 mL). The mixture was warmed to 0° C. and reacted for 30 min. At −78° C., a solution of 3-cyano-2-methoxypyridine (23a, 0.50 g, 3.7 mmol) in tetrahydrofuran (5 mL) was added dropwise thereto. After stirring for 30 min, a solution of trimethyl borate (1.4 g, 7.5 mmol) in tetrahydrofuran was added dropwise thereto, and the mixture was reacted for another 30 min. The reaction was quenched with a saturated ammonium chloride solution at −78° C. The reaction mixture was concentrated, and formic acid was added dropwise to the solution to adjust the pH of the solution to 5. The mixture was extracted with ethyl acetate (50 mL×3). The organic phases were combined, washed with a saturated aqueous sodium chloride solution (30 mL), dried over anhydrous sodium sulfate, and filtered, and the filtrate was concentrated. The resulting residue, compound 23b (0.40 g, purity: 55%), could be directly used in the next step without purification.

### Step 2: Synthesis of methyl 6-amino-7-(3-benzyloxy-2,6-dimethylphenyl)-4-(3-cyano-2-methoxypyridin-4-yl)-2-methyl-7H-pyrrolo[2,3-d]pyrimidine-5-carboxylate (23c)

Compound 11g (0.20 g, 0.44 mmol), compound 23b (purity: 55%) (0.19 g, 0.58 mmol), tris(dibenzylideneacetone)dipalladium (19 mg, 44 μmol), 1,3,5,7-tetramethyl-6-phenyl-2,4,8-trioxa-6-phosphaadamantane (13 mg, 44 μmol), and cesium carbonate (0.29 g, 0.89 mmol) were dissolved in 1,4-dioxane/water (5 mL/1 mL) at room temperature. The system was purged with nitrogen three times. The reaction mixture was moved to 100° C. and reacted for 2 h. The reaction mixture was cooled to room temperature, filtered to remove the insoluble substance, diluted with water (10 mL), and extracted with ethyl acetate (20 mL×3). The organic phases were combined, washed with saturated brine (20 mL), dried over anhydrous sodium sulfate, and filtered. The filtrate was concentrated. The resulting residue was purified by normal-phase column chromatography (petroleum ether:ethyl acetate=1:1) to obtain the product compound 23c (0.14 g, yield: 54%). m/z (ESI): 549.2 [M+H]⁺.

### Step 3: Synthesis of methyl 6-amino-4-(3-(aminomethyl)-2-methoxypyridin-4-yl)-7-(3-(benzyloxy)-2,6-dimethylphenyl)-2-methyl-7H-pyrrolo[2,3-d]pyrimidine-5-carboxylate (23d)

Compound 23c (0.14 g, 0.24 mmol) and cobalt chloride hexahydrate (0.29 g, 1.2 mmol) were dissolved in anhydrous methanol under an ice bath, and then sodium borohydride (46 mg, 1.2 mmol) was added. The mixture was then warmed to room temperature and reacted for 1 h. A saturated aqueous ammonium chloride solution (10 mL) was added to the reaction mixture to quench the reaction. The mixture was extracted with ethyl acetate (15 mL×3), and the organic phases were combined, washed with a saturated aqueous sodium chloride solution (30 mL), dried over anhydrous sodium sulfate, and filtered. The filtrate was concentrated. The resulting solid could be directly used in the next step without purification. m/z (ESI): 553.2 [M+H]⁺.

Compound 23 was then prepared by replacing compound 11i with compound 23d and using a method similar to that in step 7 and step 8 of Example 11.

m/z (ESI): 431.1 [M+H]⁺;

¹H NMR (400 MHz, DMSO-d₆) δ 8.21 (d, J=5.2 Hz, 1H), 7.92 (t, J=6.8 Hz, 1H), 7.28 (dd, J=1.2, 5.2 Hz, 1H), 7.16-7.04 (m, 3H), 6.95 (d, J=8.0 Hz, 1H), 4.86-4.80 (m, 1H), 4.33-4.28 (m, 1H), 3.97 (s, 3H), 2.50 (s, 3H), 1.90-1.78 (m, 3H), 1.71-1.58 (m, 3H).

### Example 24. Preparation of 2-amino-1-(3-hydroxy-2,6-dimethylphenyl)-7-methoxy-11-methyl-4,5-dihydro-1,4,6,10,12-pentaazabenzo[4,5]cyclooctatetraeno[1,2,3-cd]inden-3(1H)-one (Compound 24)

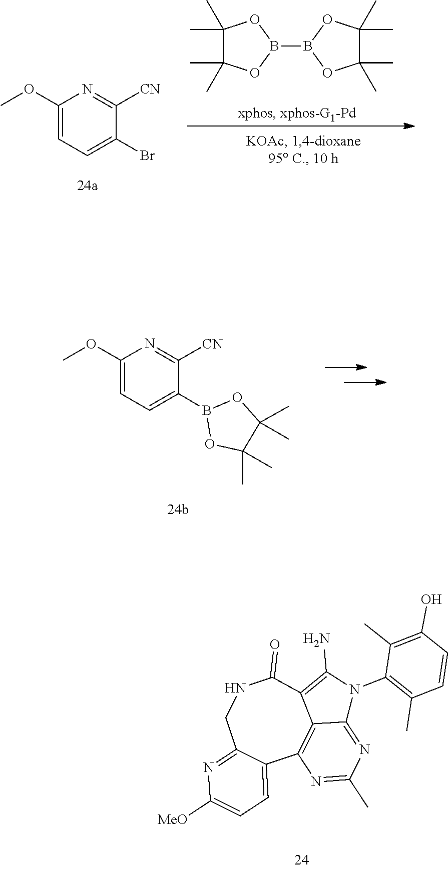

### Step 1: Synthesis of 6-methoxy-3-(4,4,5,5-tetramethyl-1,3,2-dioxaborolan-2-yl)cyanopyridine (24b)

Compound 24a (1.0 g, 4.7 mmol), bis(pinacolato)diboron (1.8 g, 7.0 mmol), 2-dicyclohexylphosphino-2′,4′,6′-triisopropylbiphenyl (0.20 g, 0.47 mmol), chloro(2-dicyclohexylphosphino-2′,4′,6′-triisopropyl-1,1′-biphenyl)[2-(2-aminoethylphenyl)]palladium(II) (0.35 g, 0.47 mmol), and potassium acetate (0.92 g, 9.4 mmol) were dissolved in anhydrous 1,4-dioxane (20 mL) at room temperature. The system was purged with nitrogen 3 times, and the mixture was reacted at 95° C. for 10 h under nitrogen atmosphere. After the reaction mixture was cooled to room temperature, water (20 mL) was added to the reaction mixture to quench the reaction. The mixture was extracted with ethyl acetate (100 mL×3), and the organic phases were combined, washed with a saturated aqueous sodium chloride solution (30 mL), dried over anhydrous sodium sulfate, and filtered. The filtrate was concentrated. The resulting residue was purified by column chromatography (petroleum ether:ethyl acetate=100:1-10:1) to obtain the product compound 24b (0.70 g, yield: 57%). m/z (ESI): 261.1 [M+H]⁺.

Compound 24 was then prepared by replacing compound 23b with compound 24b and using a method similar to that in Example 23.

m/z (ESI): 431.1 [M+H]⁺;

¹H NMR (400 MHz, DMSO-d₆) δ 8.11-8.02 (m, 2H), 7.11-7.06 (m, 1H), 6.98-6.92 (m, 4H), 5.07-5.00 (m, 1H), 3.97 (s, 3H), 3.94-3.90 (m, 1H), 2.49 (s, 3H), 1.90-1.81 (m, 3H), 1.70-1.61 (m, 3H).

### Example 25. Preparation of 2-amino-6-(difluoromethoxy)-1-(3-hydroxy-2,6-dimethylphenyl)-11-methyl-4,5-dihydro-1,4,10,12-tetraazabenzo[4,5]cyclooctatetraeno[1,2,3-cd]inden-3(1H)-one (Compound 25)

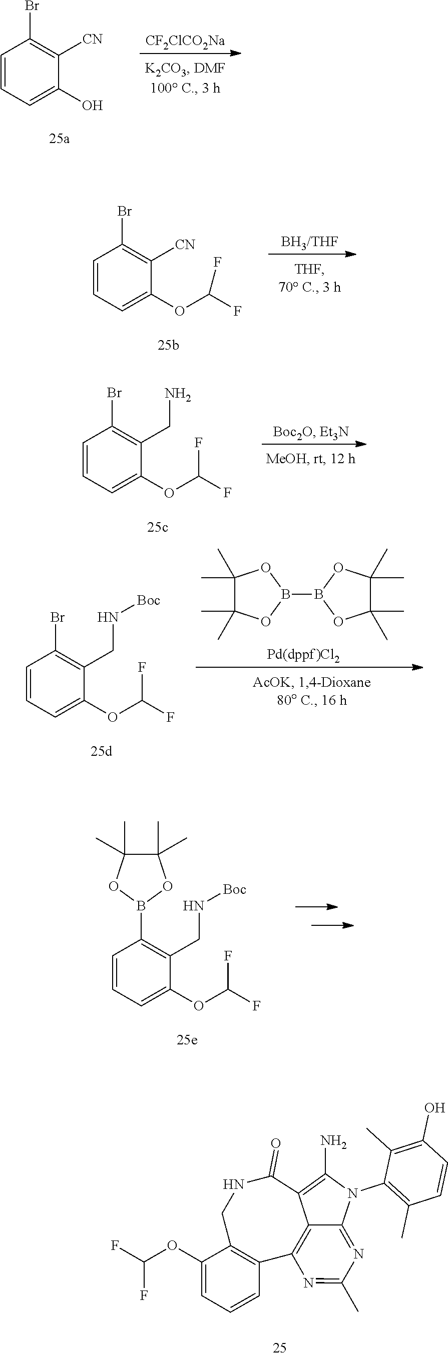

### Step 1: Synthesis of 2-bromo-6-(difluoromethoxy)benzonitrile (25b)

Compound 25a (2.0 g, 10 mmol) was dissolved in a mixed solution of N,N-dimethylformamide (18 mL) and water (2 mL). Sodium difluorochloroacetate (3.0 g, 20 mmol) and potassium carbonate (1.6 g, 12 mmol) were sequentially added. The mixture was warmed to 100° C. and reacted for 3 h. The reaction mixture was cooled to room temperature, filtered to remove the insoluble substance, diluted with water (10 mL), and extracted with ethyl acetate (50 mL×3). The organic phases were combined, washed with saturated brine (20 mL×3), dried over anhydrous sodium sulfate, and filtered. The filtrate was concentrated. The resulting residue was purified by normal-phase column chromatography (petroleum ether:ethyl acetate=100:1-10:1) to obtain the product compound 25b (1.0 g, yield: 40%). m/z (EI): 246.8 [M]⁺;

### Step 2: Synthesis of (2-bromo-6-(difluoromethoxy)phenyl)methylamine (25c)

Under an ice bath, compound 25b (1.0 g, 4.0 mmol) was dissolved in anhydrous tetrahydrofuran (30 mL). A solution of borane in tetrahydrofuran (1 M, 20 mL) was added dropwise. After the addition was completed, the mixture was warmed to 70° C. and reacted for 3 h. After the reaction was cooled to room temperature, methanol (20 mL) was added dropwise under an ice bath to quench the reaction. The reaction mixture was concentrated. The resulting product, compound 25c, could be directly used in the next step without purification.

### Step 3: Synthesis of tert-butyl (2-bromo-6-(difluoromethoxy)benzyl)carbamate (25d)

The product from the previous step, compound 25c (1.0 g, 4.0 mmol), was dissolved in anhydrous methanol. Triethylamine (0.60 g, 6.0 mmol) and di-tert-butyl dicarbonate (1.7 g, 8.0 mmol) were sequentially added. The mixture was reacted at room temperature for 12 h. The reaction mixture was concentrated. The resulting residue was purified by column chromatography (petroleum ether:ethyl acetate=100:1-10:1) to obtain the product compound 25d (1.2 g, yield: 85%). m/z (ESI): 296.0 [M−56+H]⁺;

### Step 4: Synthesis of tert-butyl (2-(difluoromethoxy)-6-(4,4,5,5-tetramethyl-1,3,2-dioxaborolan-2-yl)benzyl)carbamate (25e)

Compound 25d (0.50 g, 1.4 mmol), bis(pinacolato)diboron (0.54 g, 2.1 mmol), [1,1′-bis(diphenylphosphino)ferrocene]palladium dichloride (0.10 g, 0.14 mmol), and potassium acetate (0.27 g, 2.8 mmol) were dissolved in anhydrous 1,4-dioxane (15 mL). The system was purged with argon, and the mixture was reacted at 80° C. for 16 h under argon atmosphere. The reaction mixture was cooled to room temperature, then filtered to remove the insoluble substance, and washed with ethyl acetate (30 mL). The filtrate was concentrated. The resulting product, compound 25e, could be directly used in the next step without purification. m/z (ESI): 344.1 [M−56+H]⁺.

Compound 25 was then prepared by replacing compound 11c with compound 25e and using a method similar to that in step 5 to step 8 of Example 11.

m/z (ESI): 466.3 [M+H]⁺;

¹H NMR (400 MHz, DMSO-d₆) δ 9.62 (d, J=9.2 Hz, 1H), 7.84-7.82 (m, 1H), 7.62 (d, J=8.0 Hz, 1H), 7.53-7.51 (m, 1H), 7.32-7.28 (m, 2H), 7.10-7.07 (m, 3H), 6.96 (d, J=8.0 Hz, 1H), 4.87-4.80 (m, 1H), 4.45-4.31 (m, 1H), 2.54 (s, 3H), 1.91-1.82 (m, 3H), 1.70-1.61 (m, 3H).

### Example 26. Preparation of 2-amino-1-(3-hydroxy-2,6-dimethylphenyl)-11-methyl-6-(trifluoromethoxy)-4,5-dihydro-1,4,10,12-tetraazabenzo[4,5]cyclooctatetraeno[1,2,3-cd]inden-3(1H)-one (Compound 26)

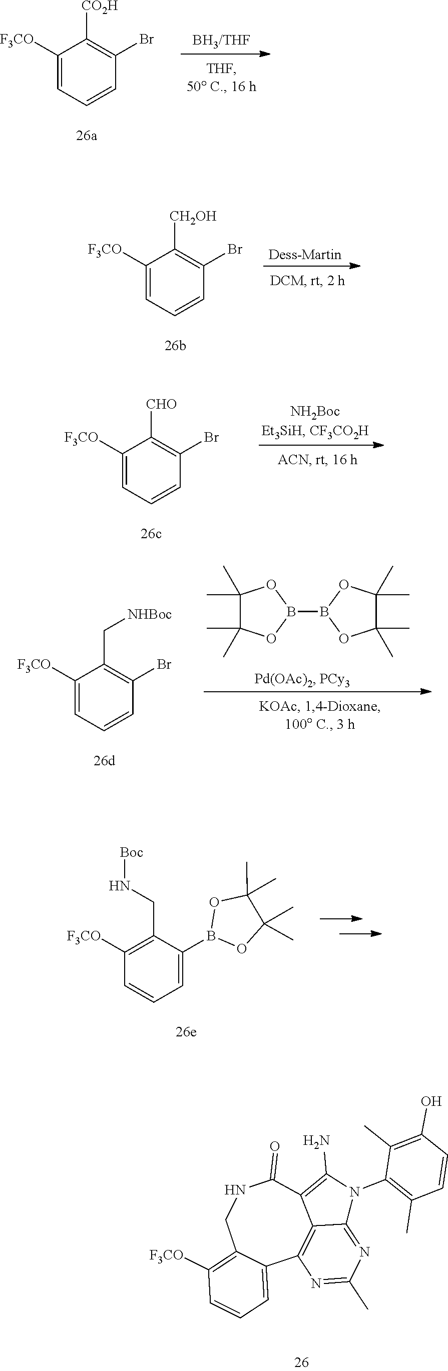

### Step 1: Synthesis of (2-bromo-6-(trifluoromethoxy)phenyl)methanol (26b)

Under an ice bath, compound 26a (1.0 g, 3.5 mmol) was dissolved in anhydrous tetrahydrofuran (20 mL). A solution of borane in tetrahydrofuran (1 M, 14 mL) was added dropwise. After the addition was completed, the mixture was warmed to 50° C. and reacted for 16 h. After the reaction was cooled to room temperature, 1 M hydrochloric acid solution (20 mL) was added dropwise under an ice bath to quench the reaction. The reaction mixture was concentrated, diluted with water (10 mL), and extracted with ethyl acetate (30 mL×3), and the organic phases were combined, washed with saturated brine (20 mL), dried over anhydrous sodium sulfate, and filtered. The filtrate was concentrated. The resulting product, compound 26b, could be directly used in the next step without purification.

### Step 2: Synthesis of 2-bromo-6-(trifluoromethoxy)benzaldehyde (26c)

Compound 26b (0.90 g, 3.3 mmol) was dissolved in anhydrous dichloromethane (10 mL). Dess-Martin oxidizer (2.1 g, 5.0 mmol) was added in portions. The mixture was reacted at room temperature for 2 h. A saturated aqueous sodium bicarbonate solution (10 mL) and sodium thiosulfate (10 mL) were sequentially added to the reaction mixture to quench the reaction, and the mixture was extracted with ethyl acetate (40 mL×3). The organic phases were combined, washed with saturated brine (20 mL), dried over anhydrous sodium sulfate, and filtered. The filtrate was concentrated. The resulting residue was purified by column chromatography (petroleum ether:ethyl acetate=10:1-5:1) to obtain the product compound 26c (0.42 g, yield: 44%). m/z (EI): 267.9 [M]⁺;

### Step 3: Synthesis of tert-butyl (2-bromo-6-(trifluoromethoxy)benzyl)carbamate (26d)

Compound 26c (0.40 g, 1.5 mmol) was dissolved in anhydrous acetonitrile. tert-Butyl carbamate (0.53 g, 4.5 mmol), triethylsilane (0.52 g, 4.5 mmol), and trifluoroacetic acid (0.34 g, 3.0 mmol) were sequentially added. The mixture was reacted at room temperature for 16 h. The reaction mixture was concentrated, diluted with water (10 mL), and extracted with ethyl acetate (30 mL×3). The organic phases were combined, washed with saturated brine (20 mL), dried over anhydrous sodium sulfate, and filtered. The filtrate was concentrated. The resulting residue was purified by column chromatography (petroleum ether:ethyl acetate=100:1-10:1) to obtain the product compound 26d (0.24 g, yield: 44%). m/z (ESI): 313.9 [M-56+H]⁺;

### Step 4: Synthesis of tert-butyl (2-(4,4,5,5-tetramethyl-1,3,2-dioxaborolan-2-yl)-6-(trifluoromethoxy)benzyl)carbamate (26e)

Compound 26d (37 mg, 0.10 mmol), bis(pinacolato)diboron (31 mg, 0.12 mmol), palladium acetate (4.5 mg, 20 μmol), tricyclohexylphosphine (5.6 mg, 20 μmol), and potassium acetate (20 mg, 0.20 mmol) were dissolved in anhydrous 1,4-dioxane (2 mL). The system was purged with argon, and the mixture was reacted at 100° C. for 3 h under argon atmosphere. The reaction mixture was cooled to room temperature, and then filtered to remove the insoluble substance. The resulting product, compound 26e, could be directly used in the next step without purification.

Compound 26 was then prepared by replacing compound 3c with compound 26e and using a method similar to that in step 3 to step 6 of Example 3.

m/z (ESI): 484.4 [M+H]⁺;

¹H NMR (400 MHz, DMSO-d₆) δ 9.62 (brs, 1H), 7.96 (t, J=7.2 Hz, 1H), 7.75 (dt, J=8.0, 1.2 Hz, 1H), 7.58 (t, J=8.0 Hz, 1H), 7.49-7.47 (m, 1H), 7.13-7.07 (m, 3H), H), 6.95 (d, J=8.0 Hz, 1H), 4.94-4.88 (m, 1H), 4.38-4.32 (m, 1H), 2.50 (s, 3H), 1.90-1.81 (m, 3H), 1.69-1.60 (m, 3H).

### Example 27. Preparation of 2-amino-1-(3-hydroxy-2,6-dimethylphenyl)-11-methyl-3-oxo-1,3,4,5-tetrahydro-1,4,10,12-tetraazabenzo[4,5]cyclooctatetraeno[1,2,3-cd]indene-6-carbonitrile (Compound 27)

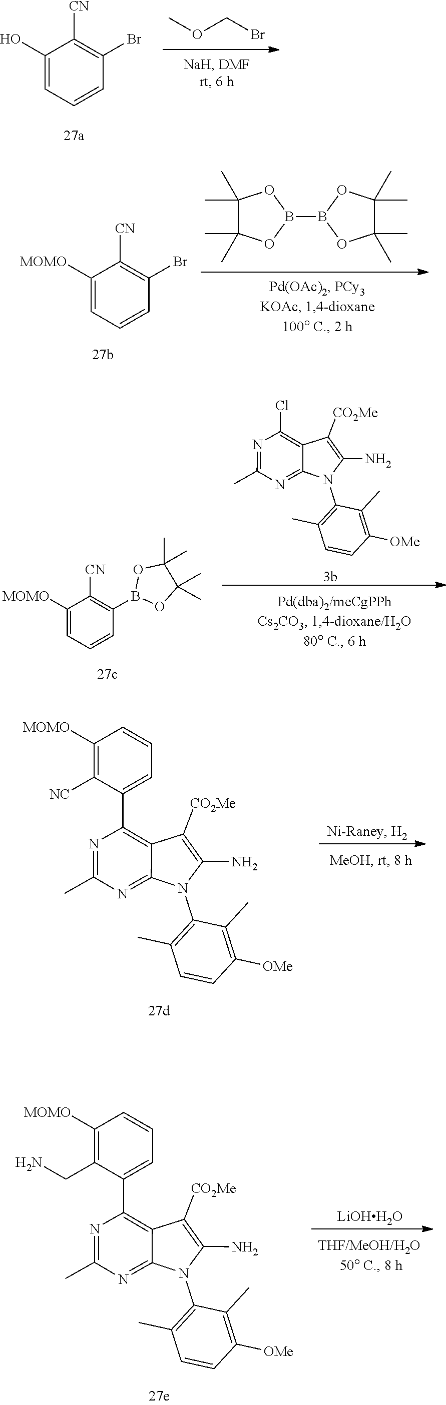

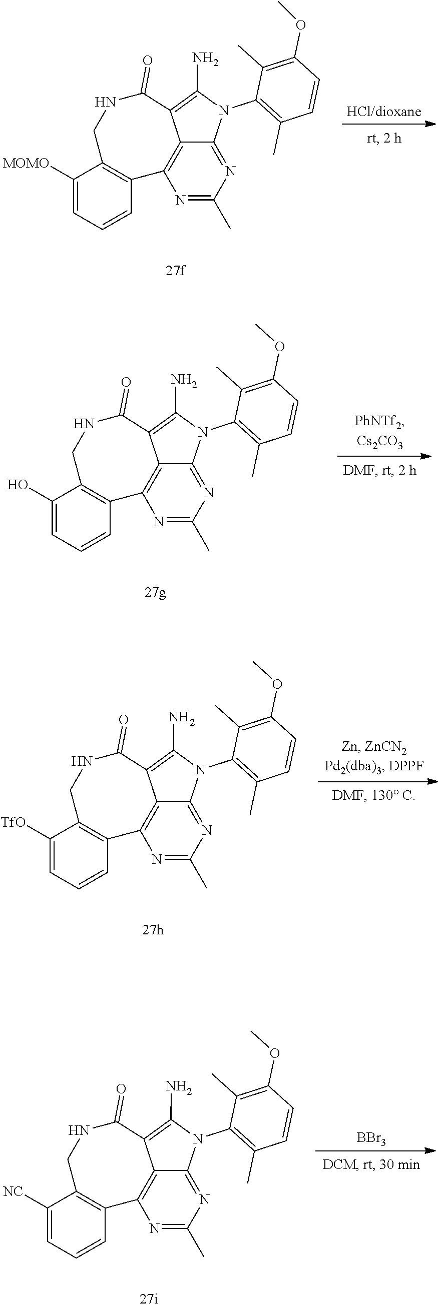

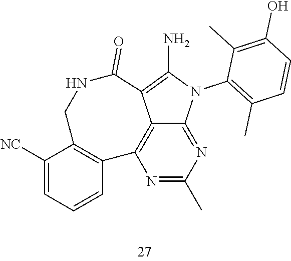 [SMILES: Cc1nc2c3c(c(N)n(-c4c(C)ccc(O)c4C)c3n1)C(=O)NCc1c(C#N)cccc1-2]

### Step 1: Synthesis of 2-bromo-6-(methoxymethoxy)benzonitrile (27b)

Compound 27a (1.0 g, 5.1 mmol) was dissolved in anhydrous N,N-dimethylformamide under an ice bath. Sodium hydride (60%, 0.61 g, 15 mmol) was then added. After 30 min of reaction, bromomethyl methyl ether (1.3 g, 10 mmol) was then added. The mixture was warmed to room temperature and reacted for 6 h. The mixture was diluted with water (10 mL), and extracted with ethyl acetate (100 mL×3). The organic phases were combined, washed with saturated brine (50 mL×3), dried over anhydrous sodium sulfate, and filtered. The filtrate was concentrated. The resulting residue was purified by normal-phase column chromatography (petroleum ether:ethyl acetate=100:1-10:1) to obtain the product compound 27b (1.2 g, yield: 99%).

### Step 2: Synthesis of 2-(methoxymethoxy)-6-(4,4,5,5-tetramethyl-1,3,2-dioxaborolan-2-yl)benzonitrile (27c)

Compound 27b (0.12 g, 0.5 mmol), bis(pinacolato)diboron (0.15 g, 0.60 mmol), palladium acetate (22 mg, 0.10 mmol), tricyclohexylphosphine (28 mg, 0.10 mmol), and potassium acetate (98 mg, 1.0 mmol) were dissolved in anhydrous 1,4-dioxane (5 mL). The system was purged with argon, and the mixture was reacted at 100° C. for 2 h under argon atmosphere. The reaction mixture was cooled to room temperature, and then filtered to remove the insoluble substance. The resulting product, compound 27c, could be directly used in the next step without purification. m/z (ESI): 290.1 [M+H]⁺.

### Step 3: Synthesis of methyl 6-amino-4-(2-cyano-3-(methoxymethoxy)phenyl)-7-(3-methoxy-2,6-dimethylphenyl)-2-methyl-7H-pyrrolo[2,3-d]pyrimidine-5-carboxylate (27d)

Compound 3b (80 mg, 0.21 mmol), compound 27c (0.12 g, 0.43 mmol), bis(dibenzylideneacetone)palladium (24 mg, 43 μmol), 1,3,5,7-tetramethyl-6-phenyl-2,4,8-trioxa-6-phosphaadamantane (12 mg, 43 μmol), and cesium carbonate (0.14 g, 0.43 mmol) were dissolved in 1,4-dioxane/water (5 mL/0.5 mL) at room temperature. The system was purged with nitrogen three times. The reaction mixture was moved to 80° C. and reacted for 6 h. The reaction mixture was cooled to room temperature and then filtered to remove the insoluble substance, and the filtrate was concentrated. The resulting residue was purified by reverse-phase column chromatography (C18, 0.05% aqueous ammonia:acetonitrile=20:1-1:20) to obtain the target compound 27d (82 mg, yield: 76%). m/z (ESI): 502.2 [M+H]⁺.

### Step 4: Synthesis of methyl 6-amino-4-(2-(aminomethyl)-3-(methoxymethoxy)phenyl)-7-(3-methoxy-2,6-dimethylphenyl)-2-methyl-7H-pyrrolo[2,3-d]pyrimidine-5-carboxylate (27e)

Compound 27d (50 mg, 0.10 mmol) was dissolved in anhydrous methanol (10 mL) at room temperature, and then Raney nickel (10 mg, 53 μmol) was added. The system was purged with hydrogen. The mixture was reacted at room temperature for 8 h at a pressure of 45 psi. The reaction mixture was filtered to remove the insoluble substance, and the filtrate was concentrated. The resulting residue was purified by reverse-phase column chromatography (C18, 0.05% aqueous ammonia:acetonitrile=20:1-1:20) to obtain the target compound 27e (50 mg, yield: 99%). m/z (ESI): 506.2 [M+H]⁺.

### Step 5: Synthesis of 2-amino-1-(3-methoxy-2,6-dimethylphenyl)-6-(methoxymethoxy)-11-methyl-4,5-dihydro-1,4,10,12-tetraazabenzo[4,5]cyclooctatetraeno[1,2,3-cd]inden-3(1H)-one (27f)

Compound 27e (50 mg, 0.10 mmol) was dissolved in methanol/tetrahydrofuran/water (2 mL/2 mL/2 mL), and lithium hydroxide monohydrate (42 mg, 1.0 mmol) was then added. The reaction mixture was reacted at 50° C. under an oil bath for 8 h. The reaction mixture was concentrated. The resulting residue was purified by reverse-phase column chromatography (C18, 0.05% aqueous ammonia:acetonitrile=20:1-1:20) to obtain the target compound 27f (47 mg, yield: 99%). m/z (ESI): 474.2 [M+H]⁺.

### Step 6: Synthesis of 2-amino-6-hydroxy-1-(3-methoxy-2,6-dimethylphenyl)-11-methyl-4,5-dihydro-1,4,10,12-tetraazabenzo[4,5]cyclooctatetraeno[1,2,3-cd]inden-3(1H)-one (27g)

At room temperature, compound 27f (47 mg, 0.10 mmol) was dissolved in a solution of hydrochloric acid in 1,4-dioxane (4 mL). The mixture was reacted under the condition for 2 h. After the reaction was completed, the reaction mixture was concentrated. The resulting residue was purified by reverse-phase column chromatography (C18, 0.05% aqueous ammonia:acetonitrile=20:1-1:20) to obtain the target compound 27g (40 mg, yield: 93%). m/z (ESI): 430.2 [M+H]⁺.

### Step 7: Synthesis of Intermediate 27h

Compound 27g (20 mg, 46 μmol) was dissolved in anhydrous N,N-dimethylformamide (5 mL). Cesium carbonate (23 mg, 70 μmol) and N-phenylbis(trifluoromethanesulfonyl)imide (22 mg, 60 μmol) were sequentially added. The mixture was reacted at room temperature for 2 h. The reaction mixture was directly purified by reverse-phase column chromatography (C18, 0.05% aqueous ammonia:acetonitrile=20:1-1:20) to obtain the target compound 27h (22 mg, yield: 84%). m/z (ESI): 562.1 [M+H]⁺.

### Step 8: Synthesis of 2-amino-1-(3-methoxy-2,6-dimethylphenyl)-11-methyl-3-oxo-1,3,4,5-tetrahydro-1,4,10,12-tetraazabenzo[4,5]cyclooctatetraeno[1,2,3-cd]indene-6-carbonitrile (27i)

Compound 27h (20 mg, 36 μmol) was dissolved in anhydrous N,N-dimethylformamide (5 mL). Zinc (1.2 mg, 18 μmol), zinc cyanide (4.2 mg, 36 μmol), tris(dibenzylideneacetone)dipalladium (3.2 mg, 3.6 μmol), and 1,1′-bis(diphenylphosphino)ferrocene (2.0 mg, 3.6 μmol) were sequentially added. The system was purged with nitrogen three times, and the reaction mixture was moved to 130° C. and reacted for 6 h. The reaction mixture was cooled to room temperature and then directly purified by reverse-phase column chromatography (C18, 0.05% aqueous ammonia:acetonitrile=20:1-1:20) to obtain the target compound 27i (12 mg, yield: 76%).

### Step 9: Synthesis of 2-amino-1-(3-hydroxy-2,6-dimethylphenyl)-11-methyl-3-oxo-1,3,4,5-tetrahydro-1,4,10,12-tetraazabenzo[4,5]cyclooctatetraeno[1,2,3-cd]indene-6-carbonitrile (27)

Compound 27i (12 mg, 27 μmol) was dissolved in dichloromethane (5 mL) at room temperature. A solution of boron tribromide (1 mol/L, 2 mL) in dichloromethane was added, and the mixture was stirred for 30 min. The reaction mixture was quenched with methanol (5 mL) and then concentrated. The resulting residue was purified by reverse-phase column chromatography (C18, 0.05% aqueous ammonia:acetonitrile=20:1-1:20) to obtain compound 27 (9.6 mg, yield: 84%). m/z (ESI): 425.3 [M+H]⁺;

¹H NMR (400 MHz, DMSO-d₆) δ 9.62 (d, J=9.2 Hz, 1H), 8.12 (t, J=6.8 Hz, 1H) 8.07 (d, J=8.0 Hz, 1H), 7.97 (d, J=8.0 Hz, 1H), 7.66 (t, J=8.0 Hz, 1H), 7.13-7.07 (m, 3H), 6.96 (d, J=8.0 Hz, 1H), 5.11-5.06 (m, 1H), 4.39-4.33 (m, 1H), 1.90-1.81 (m, 3H), 1.70-1.61 (m, 3H).

### Example 28. Preparation of 2-amino-7-cyclopropyl-1-(3-hydroxy-2,6-dimethylphenyl)-11-methyl-4,5-dihydro-1,4,6,10,12-pentaazabenzo[4,5]cyclooctatetraeno[1,2,3-cd]inden-3(1H)-one (Compound 28)

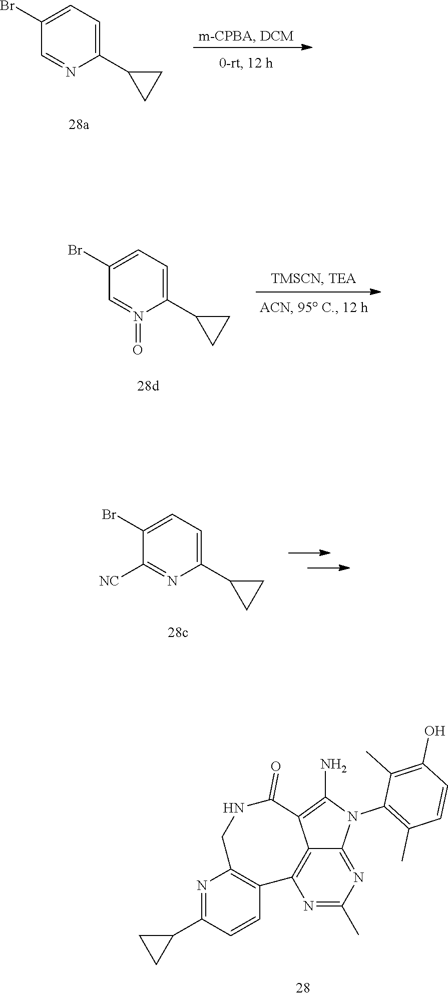 [SMILES: Brc1ccc(C2CC2)nc1 ; [O-][n+]1cc(Br)ccc1C1CC1 ; N#Cc1nc(C2CC2)ccc1Br ; Cc1nc2c3c(c(N)n(-c4c(C)ccc(O)c4C)c3n1)C(=O)NCc1nc(C3CC3)ccc1-2]

### Step 1: Synthesis of 5-bromo-2-cyclopropylpyridine-1-oxide (28b)

Compound 28a (2.5 g, 12.6 mmol) was dissolved in anhydrous dichloromethane under an ice bath. m-Chloroperoxybenzoic acid (2.6 g, 12.6 mmol) was then added. The mixture was warmed to room temperature and reacted for 12 h. A saturated aqueous ammonium chloride solution (30 mL) was added to the reaction mixture to quench the reaction. The pH was adjusted to 11 with 1 M NaOH solution. The mixture was extracted with ethyl acetate (30 mL×3), and the organic phases were combined, washed with saturated brine (20 mL), dried over anhydrous sodium sulfate, and filtered. The filtrate was concentrated, and the resulting residue, compound 28b, could be directly used in the next step without purification. m/z (ESI): 214.0 [M+H]⁺;

### Step 2: Synthesis of 3-bromo-6-cyclopropylcyanopyridine (28c)

Compound 28b (1.5 g, 7.0 mmol) was dissolved in anhydrous acetonitrile (5 mL). Triethylamine (2.1 g, 21 mmol) and trimethylsilyl cyanide (2.8 g, 28 mmol) were sequentially added. The mixture was warmed to 95° C. and reacted for 12 h. A saturated aqueous ammonium chloride solution (30 mL) was added to the reaction mixture to quench the reaction. The pH was adjusted to 11 with 1 M NaOH solution. The mixture was extracted with ethyl acetate (30 mL×3), and the organic phases were combined, washed with saturated brine (20 mL), dried over anhydrous sodium sulfate, and filtered. The filtrate was concentrated. The resulting residue was purified by column chromatography (petroleum ether:ethyl acetate=100:1-10:1) to obtain the product compound 28c (1.0 g, yield: 64%). m/z (ESI): 223.0 [M+H]⁺;

Compound 28 was then prepared by replacing compound 8a with compound 28c and using a method similar to that in Example 8.

m/z (ESI): 441.2 [M+H]⁺;

¹H NMR (400 MHz, DMSO-d₆) δ 9.59 (d, J=5.6 Hz, 1H), 8.03-7.95 (m, 2H), 7.35 (d, J=8.0 Hz, 1H), 7.10-7.07 (m, 1H), 6.98-6.94 (m, 3H), 5.06-5.00 (m, 1H), 3.98-3.93 (m, 1H), 2.48 (s, 3H), 2.21-2.15 (m, 1H), 1.89-1.80 (m, 3H), 1.70-1.61 (m, 3H), 1.05-1.00 (m, 4H).

### Example 29. Preparation of 2-amino-1-(3-hydroxy-2,6-dimethylphenyl)-11-methyl-7-(trifluoromethyl)-4,5-dihydro-1,4,6,10,12-pentaazabenzo[4,5]cyclooctatetraeno[1,2,3-cd]inden-3(1H)-one (Compound 29)

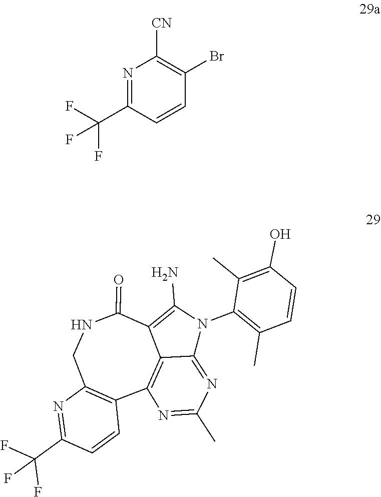 [SMILES: N#Cc1nc(C(F)(F)F)ccc1Br ; Cc1nc2c3c(c(N)n(-c4c(C)ccc(O)c4C)c3n1)C(=O)NCc1nc(C(F)(F)F)ccc1-2]

Compound 29 was prepared by replacing compound 8a with compound 29a and using a method similar to that in Example 8.

m/z (ESI): 469.1 [M+H]⁺;

¹H NMR (400 MHz, DMSO-d₆) δ 9.65 (d, J=6.0 Hz, 1H), 8.44 (d, J=8.4 Hz, 1H), 8.14-8.11 (m, 1H), 8.01 (d, J=8.4 Hz, 1H), 7.16-7.10 (m, 3H), 6.96 (d, J=8.0 Hz, 1H), 5.26-5.20 (m, 1H), 4.19-4.13 (m, 1H), 2.52 (s, 3H), 1.90-1.81 (m, 3H), 1.70-1.62 (m, 3H).

### Example 30. Preparation of 2-amino-7-fluoro-1-(3-hydroxy-2,6-dimethylphenyl)-6-methoxy-11-methyl-4,5-dihydro-1,4,10,12-tetraazabenzo[4,5]cyclooctatetraeno[1,2,3-cd]inden-3(1H)-one (Compound 30)

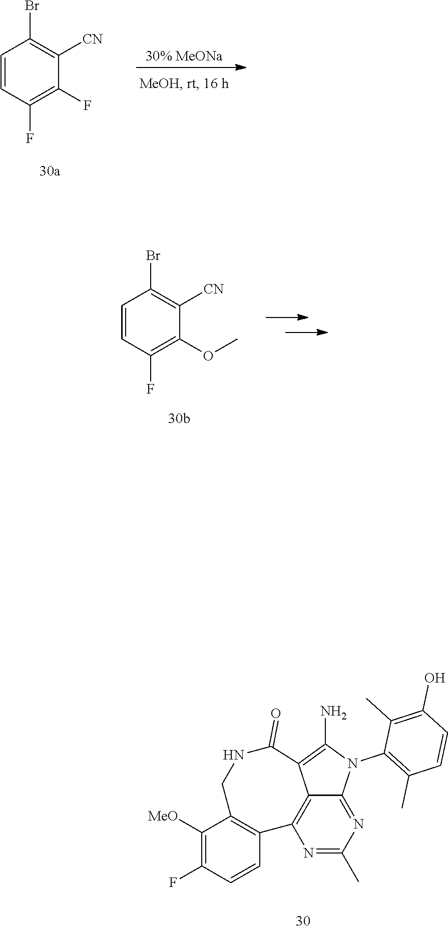 [SMILES: N#Cc1c(Br)ccc(F)c1F ; COc1c(F)ccc(Br)c1C#N ; COc1c(F)ccc2c1CNC(=O)c1c(N)n(-c3c(C)ccc(O)c3C)c3nc(C)nc-2c13]

### Step 1: Synthesis of 6-bromo-3-fluoro-2-methoxybenzonitrile (30b)

At room temperature, compound 30a (1.0 g, 4.6 mmol) was dissolved in anhydrous methanol. Sodium methoxide (30%, 1.0 g, 5.5 mmol) was then added. The mixture was reacted at room temperature for 16 h. A saturated aqueous ammonium chloride solution was added to quench the reaction. The reaction mixture was concentrated to remove methanol, and extracted with ethyl acetate (30 mL×3), and the organic phases were combined, washed with saturated brine (20 mL), dried over anhydrous sodium sulfate, and filtered. The filtrate was concentrated. The resulting product, compound 30b, could be directly used in the next step without purification. m/z (EI): 228.9 [M]⁺;

Compound 30 was then prepared by replacing compound 25b with compound 30b and using a method similar to that in Example 25.

m/z (ESI): 448.1 [M+H]⁺;

¹H NMR (400 MHz, CD₃OD) δ 7.48-7.46 (m, 1H), 7.31-7.29 (m, 1H), 7.14-7.10 (m, 1H), 6.95 (d, J=8.4 Hz, 1H), 5.40-5.29 (m, 1H), 4.98-4.87 (m, 1H), 4.07 (s, 3H), 2.58 (s, 3H), 1.99-1.93 (m, 3H), 1.79-1.74 (m, 3H).

### Example 31. Preparation of 2-amino-7-cyclopropoxy-1-(3-hydroxy-2,6-dimethylphenyl)-11-methyl-4,5-dihydro-1,4,6,10,12-pentaazabenzo[4,5]cyclooctatetraeno[1,2,3-cd]inden-3(1H)-one (Compound 31)

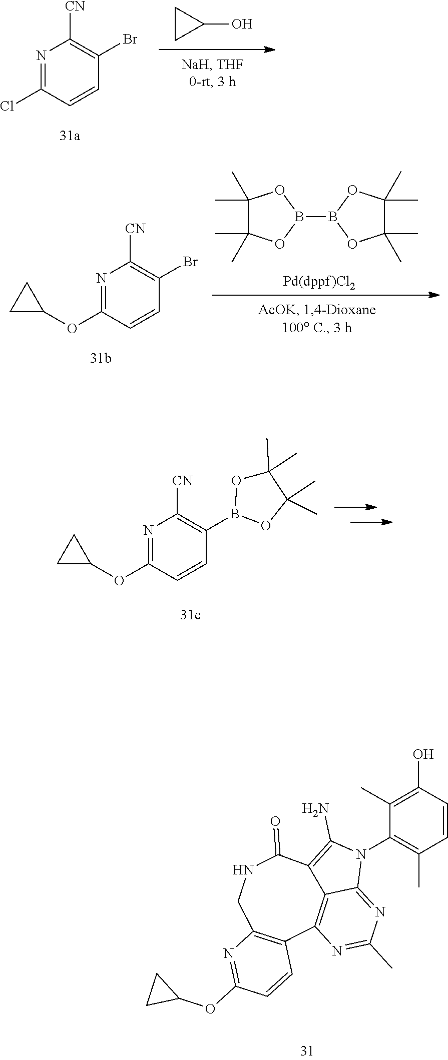

### Step 1: Synthesis of 3-bromo-6-cyclopropoxycyanopyridine (31b)

Cyclopropanol (0.64 g, 11 mmol) was dissolved in tetrahydrofuran (15 mL) under an ice bath. Sodium hydride (60%, 0.76 g, 19 mmol) was added, and the mixture was reacted under the condition for 30 min. Compound 31a (2.0 g, 9.2 mmol) was then added. The mixture was warmed to room temperature and reacted for another 3 h. A saturated aqueous ammonium chloride solution (20 mL) was added to quench the reaction, and the mixture was extracted with ethyl acetate (100 mL×3). The organic phases were combined, washed with saturated brine (50 mL), dried over anhydrous sodium sulfate, and filtered. The filtrate was concentrated. The resulting residue was purified by column chromatography (petroleum ether:ethyl acetate=10:1-5:1) to obtain the product compound 31b (1.6 g, yield: 72%). m/z (ESI): 238.9 [M+H]⁺;

### Step 2: Synthesis of 6-cyclopropoxy-3-(4,4,5,5-tetramethyl-1,3,2-dioxaborolan-2-yl)cyanopyridine (31c)

Compound 31b (1.0 g, 4.2 mmol), bis(pinacolato)diboron (1.6 g, 6.3 mmol), [1,1′-bis(diphenylphosphino)ferrocene]palladium dichloride (0.31 g, 0.42 mmol), and potassium acetate (1.0 g, 10 mmol) were dissolved in anhydrous 1,4-dioxane (10 mL). The system was purged with argon, and the mixture was reacted at 100° C. for 3 h under argon atmosphere. The reaction mixture was cooled to room temperature, then filtered to remove the insoluble substance, and washed with ethyl acetate (30 mL). The filtrate was concentrated. The resulting residue was purified by column chromatography (petroleum ether:ethyl acetate=5:1-2:1) to obtain the product compound 31c (0.73 g, yield: 61%). m/z (ESI): 205.1 [M−C₆H₁₀+H]⁺.

Compound 31 was then prepared by replacing compound 8b with compound 31c and using a method similar to that in Example 8.

m/z (ESI): 457.2 [M+H]⁺;

¹H NMR (400 MHz, DMSO-d₆) δ 9.60 (d, J=7.2 Hz, 1H), 8.13 (d, J=8.8 Hz, 1H), 8.02 (t, J=7.2 Hz, 1H), 7.12-7.10 (m, 1H), 7.07-6.93 (m, 4H), 5.05-5.00 (m, 1H), 4.33-4.29 (m, 1H), 3.96-3.90 (m, 1H), 2.48 (s, 3H), 1.89-1.80 (m, 3H), 1.70-1.61 (m, 3H), 0.85-0.73 (m, 2H).

### Example 32. Preparation of 2-amino-1-(3-hydroxy-2,6-dimethylphenyl)-7-(hydroxymethyl)-11-methyl-4,5-dihydro-1,4,6,10,12-pentaazabenzo[4,5]cyclooctatetraeno[1,2,3-cd]inden-3(1H)-one (Compound 32)

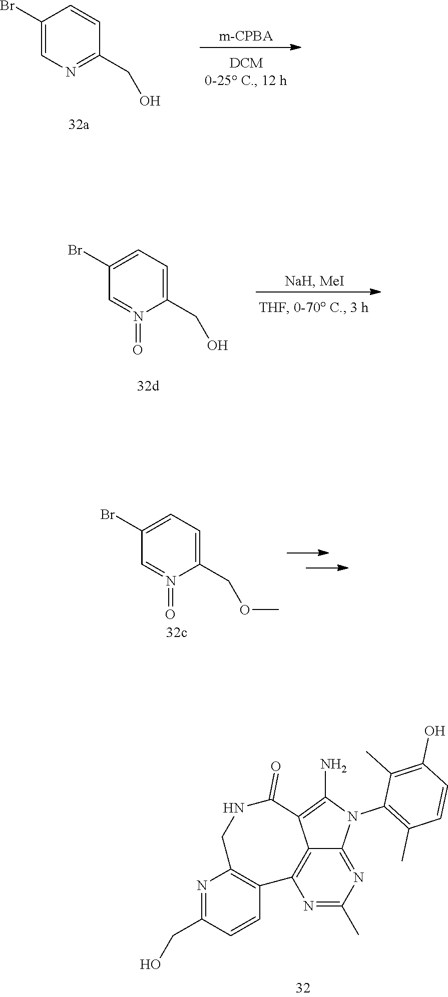 [SMILES: OCc1ccc(Br)cn1 ; [O-][n+]1cc(Br)ccc1CO ; COCc1ccc(Br)c[n+]1[O-] ; Cc1nc2c3c(c(N)n(-c4c(C)ccc(O)c4C)c3n1)C(=O)NCc1nc(CO)ccc1-2]

### Step 1: Synthesis of 5-bromo-2-(hydroxymethyl)pyridine-1-oxide (32b)

Compound 32a (5.0 g, 27 mmol) was dissolved in anhydrous dichloromethane under an ice bath. m-Chloroperoxybenzoic acid (8.1 g, 40 mmol) was then added. The mixture was warmed to room temperature and reacted for 12 h. The reaction mixture was filtered. The filter cake was washed with dichloromethane (20 mL), and the filtrate was concentrated. The resulting residue was purified by column chromatography (petroleum ether:ethyl acetate=10:1-1:2) to obtain the product compound 32b (2.2 g, yield: 40%). m/z (ESI): 187.9 [M−OH+H]⁺;

### Step 2: Synthesis of 5-bromo-2-(methoxymethyl)pyridine-1-oxide (32c)

Compound 32b (1.2 g, 5.9 mmol) was dissolved in anhydrous tetrahydrofuran (15 mL) under an ice bath. Sodium hydride (60%, 0.48 g, 12 mmol) was added. The mixture was warmed to room temperature and reacted for 1 h. Under an ice bath, iodomethane (1.3 g, 8.8 mmol) was added. After the addition was completed, the mixture was warmed to 70° C. and reacted for 3 h. A saturated aqueous ammonium chloride solution (20 mL) was added to quench the reaction, and the mixture was extracted with ethyl acetate (100 mL×3). The organic phases were combined, washed with saturated brine (50 mL), dried over anhydrous sodium sulfate, and filtered. The filtrate was concentrated. The resulting residue was purified by column chromatography (petroleum ether:ethyl acetate=10:1-1:1) to obtain the product compound 32c (0.7 g, yield: 54%).

Compound 32 was prepared by replacing compound 28b with compound 32c and using a method similar to that in Example 28.

m/z (ESI): 431.1 [M+H]⁺;

¹H NMR (400 MHz, DMSO-d₆) δ 9.73 (s, 1H), 8.19 (d, J=8.0 Hz, 1H), 8.03-8.01 (m, 1H), 7.59 (d, J=8.0 Hz, 1H), 7.12-7.03 (m, 3H), 6.96 (d, J=8.0 Hz, 1H), 5.5 (brs, 1H), 5.10-5.06 (m, 1H), 4.65 (s, 2H), 4.06-4.00 (m, 1H), 2.54 (m, 3H), 1.89-1.81 (m, 3H), 1.70-1.61 (m, 3H).

### Example 33. Preparation of 5-amino-8-cyclopropyl-4-(3-hydroxy-2,6-dimethylphenyl)-2-methyl-7,8-dihydro-1,3,4,7,8,9-hexaazabenzo[cd]cyclopenta[f]azulen-6(4H)-one (Compound 33)

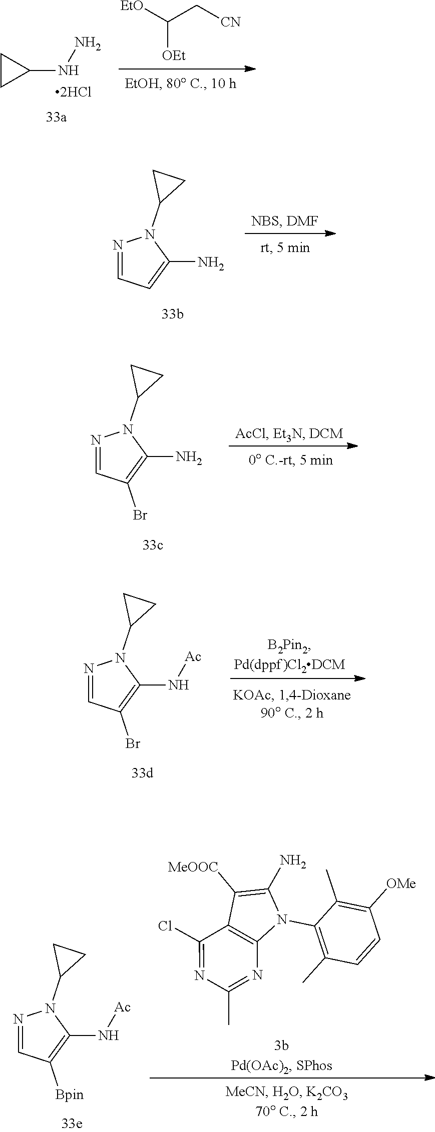

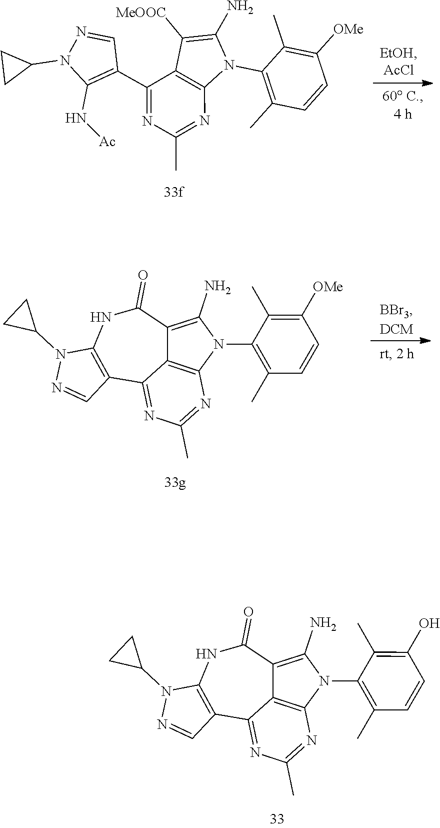 [SMILES: COC(=O)c1c(N)n(-c2c(C)ccc(OC)c2C)c2nc(C)nc(-c3cnn(C4CC4)c3NC(C)=O)c12 ; COc1ccc(C)c(-n2c(N)c3c4c(nc(C)nc42)-c2cnn(C4CC4)c2NC3=O)c1C ; Cc1nc2c3c(c(N)n(-c4c(C)ccc(O)c4C)c3n1)C(=O)Nc1c-2cnn1C1CC1]

### Step 1: Synthesis of 1-cyclopropyl-1H-pyrazol-5-amine (33b)

Cyclopropyl hydrazine dihydrochloride 33a (0.96 g, 6.3 mmol) and 3,3-diethoxypropionitrile (0.9 g, 6.3 mmol) were dissolved in ethanol (15 mL). The mixture was moved to 80° C. and reacted for 10 h. The reaction mixture was cooled to room temperature and concentrated in vacuum. The resulting residue was purified by silica gel column chromatography (dichloromethane:methanol=10:1) to obtain the target product compound 33b (0.28 g, yield: 36%). m/z (ESI): 124.1 [M+H]⁺.

### Step 2: Synthesis of 4-bromo-1-cyclopropyl-1H-pyrazol-5-amine (33c)

Compound 33b (0.28 g, 2.3 mmol) was dissolved in anhydrous N,N-dimethylformamide (5 mL). N-Bromosuccinimide (0.41 g, 2.3 mmol) was added, and the mixture was stirred at room temperature for 5 min. The resulting reaction mixture was purified by reverse-phase column chromatography (C18, 0.05% aqueous ammonia:acetonitrile=20:1-1:20) to obtain the product compound 33c (0.40 g, yield: 79%). m/z (ESI): 202.1 [M+H]⁺.

### Step 3: Synthesis of N-(4-bromo-1-cyclopropyl-1H-pyrazol-5-yl)acetamide (33d)

Under an ice bath, compound 33c (0.40 g, 2.0 mmol) and triethylamine (0.40 g, 4 mmol) were dissolved in dichloromethane (5 mL). Acetyl chloride (0.24 g, 3 mmol) was slowly added dropwise. The mixture was warmed to room temperature and reacted for 5 min. A saturated aqueous sodium bicarbonate solution (5 mL) was added to quench the reaction. The mixture was extracted with dichloromethane (10 mL×3), and the organic phases were combined, dried over anhydrous sodium sulfate, and concentrated under reduced pressure. The resulting residue was purified by reverse-phase column chromatography (C18, 0.05% aqueous ammonia:acetonitrile=20:1-1:20) to obtain the product compound 33d (0.31 g, yield: 64%). m/z (ESI): 244.2 [M+H]⁺.

### Step 4: Synthesis of N-(1-cyclopropyl-4-(4,4,5,5-tetramethyl-1,3,2-dioxaborolan-2-yl)-1H-pyrazol-5-yl)acetamide (33e)

Compound 33d (0.10 g, 0.41 mmol), bis(pinacolato)diboron (0.21 g, 0.82 mmol), potassium acetate (0.12 g, 1.2 mmol), and [1,1′-bis(diphenylphosphino)ferrocene]palladium dichloride dichloromethane complex (32 mg, 0.04 mmol) were dissolved in anhydrous dioxane (5 mL) at room temperature. The system was purged with nitrogen three times, and the reaction mixture was moved to 90° C. and reacted for 2 h. The reaction mixture was cooled to room temperature and then filtered to remove the insoluble substance. The filtrate was concentrated to dryness by rotary evaporation, and the resulting crude product could be directly used in the next step.

### Step 5: Synthesis of methyl 4-(5-acetylamino-1-cyclopropyl-1H-pyrazol-4-yl)-6-amino-7-(3-methoxy-2,6-dimethylphenyl)-2-methyl-7H-pyrrolo[2,3-d]pyrimidine-5-carboxylate (33f)

At room temperature, the crude product of compound 33e was dissolved in acetonitrile/water (5 mL/1 mL). 3b (75 mg, 0.20 mmol), palladium acetate (4.5 mg, 0.02 mmol), 2-dicyclohexylphosphino-2′,6′-dimethoxybiphenyl (16 mg, 0.04 mmol), and potassium carbonate (83 mg, 0.6 mmol) were added under stirring. The reaction mixture was moved to 70° C. and reacted for 2 h. The reaction mixture was cooled to room temperature and then filtered, and the filtrate was concentrated to dryness by rotary evaporation. The resulting crude product was purified by reverse-phase column chromatography (C18, 0.05% aqueous ammonia:acetonitrile=20:1-1:20) to obtain the product compound 33f (50 mg, yield: 25%). m/z (ESI): 504.6 [M+H]⁺.

### Step 6: Synthesis of 5-amino-8-cyclopropyl-4-(3-methoxy-2,6-dimethylphenyl)-2-methyl-7,8-dihydro-1,3,4,7,8,9-hexaazabenzo[cd]cyclopenta[f]azulen-6(4H)-one (33g)

Under an ice bath, acetyl chloride (5 mL) was slowly added dropwise to ethanol (10 mL). Compound 33f (50 mg, 99 μmol) was then added. The reaction mixture was moved to 60° C. and reacted for 4 h. The solvent was removed by distillation under reduced pressure. The pH was adjusted to neutral with aqueous ammonia (25% aqueous ammonia solution). The mixture was purified by reverse-phase column chromatography (C18, 0.05% aqueous ammonia:acetonitrile=20:1-1:20) to obtain the target product compound 33g (41 mg, yield: 96%). m/z (ESI): 430.2 [M+H]⁺.

### Step 7: Synthesis of 5-amino-8-cyclopropyl-4-(3-hydroxy-2,6-dimethylphenyl)-2-methyl-7,8-dihydro-1,3,4,7,8,9-hexaazabenzo[cd]cyclopenta[f]azulen-6(4H)-one (33)

Compound 33g (13 mg, 30 μmol) was dissolved in anhydrous dichloromethane (5 mL). Boron tribromide (1.0 mL, 1 mol/L in dichloromethane solution) was slowly added dropwise under an ice bath. The mixture was reacted at room temperature for 2 h. Methanol (5 mL) was added to quench the reaction. The solvent was removed by distillation under reduced pressure. The pH was adjusted to neutral with aqueous ammonia (25% aqueous ammonia solution). The mixture was purified by reverse-phase column chromatography (C18, 0.05% aqueous ammonia:acetonitrile=20:1-1:20) to obtain compound 33 (10 mg, yield: 79%).

m/z (ESI): 416.2 [M+H]⁺.

¹H NMR (400 MHz, DMSO-d₆) δ 9.59 (s, 1H), 9.53 (s, 1H), 8.11 (s, 1H), 7.07 (d, J=8.3 Hz, 1H), 6.98-6.86 (m, 3H), 3.63 (tt, J=7.5, 3.9 Hz, 1H), 2.36 (s, 3H), 1.78 (s, 3H), 1.69 (s, 3H), 1.11-1.03 (m, 2H), 0.95-0.90 (m, 2H).

### Example 34. Preparation of 2-amino-1-(3-hydroxy-2,6-dimethylphenyl)-5,7,9-trimethyl-4,5-dihydro-1,4,5,6,8,10-hexaazabenzo[cd]cyclopenta[f]azulen-3(1H)-one (Compound 34)

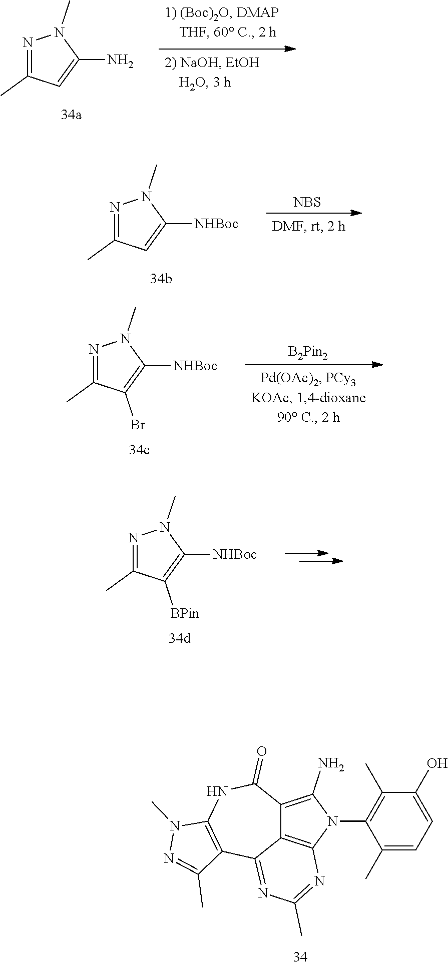

### Step 1: Synthesis of tert-butyl (1,3-dimethyl-1H-pyrazol-5-yl)carbamate (34b)

Compound 34a (1.0 g, 9.0 mmol) was dissolved in tetrahydrofuran (10 mL) at room temperature, and di-tert-butyl dicarbonate (3.0 g, 13.5 mmol) and 4-dimethylaminopyridine (0.11 g, 0.9 mmol) were added to the system. The mixture was reacted at 60° C. for 2 h and concentrated in vacuum to remove the solvent. The crude product was dissolved in ethanol (10 mL), and a sodium hydroxide solution (3 mL, 20% aqueous solution) was added to the reaction. The mixture was reacted at room temperature for another 3 h and concentrated in vacuum to remove the solvent. The crude product was purified by reverse-phase column chromatography (C18, 0.05% aqueous ammonia:acetonitrile=20:1-1:20) to obtain compound 34b (1.6 g, yield: 82%). m/z (ESI): 212.1[M+H]⁺.

### Step 2: Synthesis of tert-butyl (4-bromo-1,3-dimethyl-1H-pyrazol-5-yl)carbamate (34c)

Compound 34b (1.6 g, 7.6 mmol) was dissolved in N,N-dimethylformamide (15 mL). N-Bromosuccinimide (1.6 g, 9.1 mmol) was then added. The mixture was reacted at room temperature for 2 h. The mixture was concentrated in vacuum to remove the solvent. The crude product was purified by reverse-phase column chromatography (C18, 0.05% aqueous ammonia:acetonitrile=20:1-1:20) to obtain compound 34c (2.0 g, yield: 91%). m/z (ESI): 290.0 [M+H]⁺.

### Step 3: Synthesis of tert-butyl (1,3-dimethyl-4-(4,4,5,5-tetramethyl-1,3,2-dioxaborolan-2-yl)-1H-pyrazol-5-yl)carbamate (34d)

Compound 34c (0.12 g, 6.9 mmol), bis(pinacolato)diboron (2.3 g, 9.0 mmol), bis(dibenzylideneacetone)palladium (0.39 g, 0.69 mmol), tricyclohexylphosphine (0.39 g, 1.4 mmol), and potassium acetate (1.4 g, 14 mmol) were dissolved in anhydrous 1,4-dioxane (10 mL). The system was purged with argon, and the mixture was reacted at 90° C. for 2 h under argon atmosphere. The reaction mixture was cooled to room temperature and then filtered to remove the insoluble substance. The filtrate was concentrated. The resulting crude product, compound 34d, could be directly used in the next step without purification.

Compound 34 was then prepared by replacing compound 3c with compound 34d and using a method similar to that in step 3 to step 6 of Example 3.

m/z (ESI): 404.3 [M+H]⁺.

¹H NMR (400 MHz, DMSO-d₆) δ 9.58 (s, 1H), 9.11 (s, 1H), 7.08 (d, J=8.0 Hz, 1H), 6.94 (d, J=8.0 Hz, 1H), 6.88 (s, 2H), 3.67 (s, 3H), 2.44 (s, 3H), 2.36 (s, 3H), 1.79 (s, 3H), 1.70 (s, 3H).

### Example 35. Preparation of 2-amino-6-ethyl-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (Compound 35)

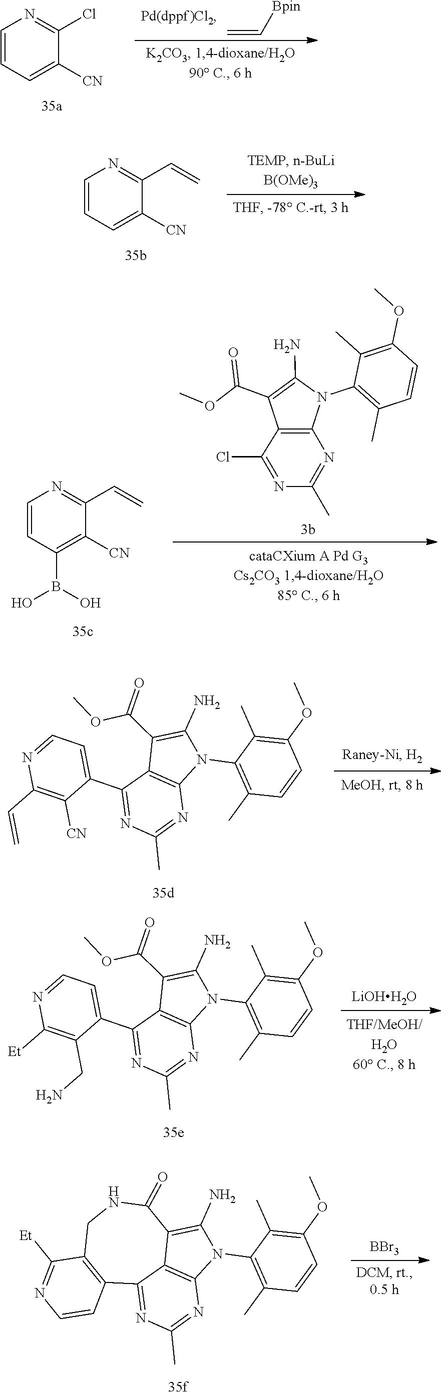

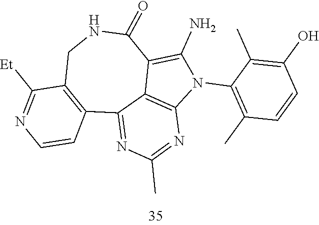 [SMILES: CCc1nccc2c1CNC(=O)c1c(N)n(-c3c(C)ccc(O)c3C)c3nc(C)nc-2c13]

### Step 1: 2-vinylnicotinonitrile (35b)

At room temperature, compound 35a (2.0 g, 14 mmol), vinylboronic acid pinacol ester (3.3 g, 22 mmol), [1,1′-bis(diphenylphosphino)ferrocene]palladium(II) dichloride (1.0 g, 1.4 mmol), and potassium carbonate (4.0 g, 29 mmol) were dissolved in 1,4-dioxane/water (10 mL/1 mL). The system was purged with nitrogen 3 times, and the reaction mixture was moved to 90° C. and reacted for 6 h. The reaction mixture was cooled to room temperature and then filtered to remove the insoluble substance, and the filtrate was concentrated. The resulting residue was purified by reverse-phase column chromatography (C18, 0.05% aqueous ammonia:acetonitrile=20:1-1:20) to obtain the target compound 35b (1.7 g, yield: 92%). m/z (ESI): 131.2 [M+H]⁺.

### Step 2: (3-cyano-2-vinylpyridin-4-yl)boronic Acid (35c)

2,2,6,6-Tetramethylpiperidine (0.23 g, 1.6 mmol) was dissolved in tetrahydrofuran (3 mL). The system was purged with argon and cooled to −78° C. n-Butyllithium (1.6 M, 1.0 mL) was slowly added dropwise under the condition. After the addition was completed, the mixture was warmed to 0° C. and stirred for 30 min. A solution of compound 35b (0.10 g, 0.76 mmol) in tetrahydrofuran (3 mL) was slowly added dropwise at −78° C. After the addition was completed, the mixture was stirred for another 30 min. A solution of trimethyl borate (0.16 g, 1.5 mmol) in tetrahydrofuran (3 mL) was slowly added dropwise at −78° C. After the addition was completed, the mixture was stirred for another 30 min. The reaction mixture was warmed to room temperature and reacted for 3 h. The pH was adjusted to 3-4 with 6 M hydrochloric acid. The mixture was extracted with ethyl acetate, and the organic phases were combined, washed with saturated brine, dried over anhydrous sodium sulfate, and filtered. The filtrate was concentrated, and the resulting crude compound 35c could be directly used in the next step without purification.

### Step 3: methyl 6-amino-4-(3-cyano-2-vinylpyridin-4-yl)-7-(3-methoxy-2,6-dimethylphenyl)-2-methyl-7H-pyrrolo[2,3-d]pyrimidine-5-carboxylate (35d)

At room temperature, compound 35c (45 mg, 0.26 mmol), compound 3b (32 mg, 85 μmol), mesylate[n-butyldi(1-adamantyl)phosphine](2-amino-1,1′-biphenyl-2-yl)palladium(II) (6.2 mg, 8.5 μmol), and cesium carbonate (56 mg, 0.17 mmol) were dissolved in 1,4-dioxane/water (3 mL/0.3 mL). The system was purged with nitrogen 3 times, and the mixture was reacted at 85° C. for 6 h under nitrogen atmosphere. The reaction mixture was cooled to room temperature and then filtered to remove the insoluble substance, and the filtrate was concentrated. The resulting residue was purified by reverse-phase column chromatography (C18, 0.05% aqueous ammonia:acetonitrile=20:1-1:20) to obtain compound 35d (35 mg, yield: 87%). m/z (ESI): 469.2 [M+H]⁺.

### Step 4: methyl 6-amino-4-(3-(aminomethyl)-2-ethylpyridin-4-yl)-7-(3-methoxy-2,6-dimethylphenyl)-2-methyl-7H-pyrrolo[2,3-d]pyrimidine-5-carboxylate (35e)

Compound 35d (35 mg, 74.71 μmol) was dissolved in anhydrous methanol (10 mL) at room temperature, and then Raney nickel (10 mg, 53 μmol) was added. The system was purged with hydrogen. The mixture was reacted at room temperature for 8 h. The reaction mixture was filtered to remove the insoluble substance, and the filtrate was concentrated. The resulting residue was purified by reverse-phase column chromatography (C18, 0.05% aqueous ammonia:acetonitrile=20:1-1:20) to obtain compound 35e (10 mg, yield: 28%). m/z (ESI): 475.3 [M+H]⁺.

### Step 5: 2-amino-6-ethyl-1-(3-methoxy-2,6-dimethylphenyl)-11-methyl-4,5-dihydro-1,4,7,10,12-pentaazabenzo[4,5]cyclooctatetraeno[1,2,3-cd]inden-3(1H)-one (35f)

Compound 35e (10 mg, 21 μmol) was dissolved in methanol/tetrahydrofuran/water (1 mL/5 mL/1 mL), and lithium hydroxide monohydrate (9.0 mg, 0.21 mmol) was added. The mixture was reacted at 60° C. for 8 h. The reaction mixture was concentrated. The resulting residue was purified by reverse-phase column chromatography (C18, 0.05% aqueous ammonia:acetonitrile=20:1-1:20) to obtain compound 35f (8 mg, yield: 85%). m/z (ESI): 443.2 [M+H]⁺.

### Step 6: 2-amino-6-ethyl-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (35)

Compound 35f (8 mg, 18 μmol) was dissolved in anhydrous dichloromethane (3 mL) at room temperature. A solution of boron tribromide (1 mol/L, 2 mL) in dichloromethane was added, and the mixture was reacted for 30 min under the condition. The reaction was quenched with methanol (5 mL). The reaction mixture was concentrated, adjusted to pH=14 with aqueous ammonia, and purified by reverse-phase column chromatography (C18, 0.05% aqueous ammonia:acetonitrile=20:1-1:20) to obtain compound 35 (5 mg, yield: 64%).

m/z (ESI): 429.3 [M+H]⁺;

¹H NMR (400 MHz, DMSO-d₆) δ 9.60 (d, J=9.6 Hz, 1H), 8.55 (d, J=4.8 Hz, 1H), 8.08 (t, J=7.2 Hz, 1H), 7.50 (d, J=4.8 Hz, 1H), 7.15-7.07 (m, 3H), 6.95 (d, J=8.4 Hz, 1H), 5.06-5.00 (m, 1H), 4.32-4.26 (m, 1H), 3.13-2.93 (m, 2H), 2.50 (m, 3H), 1.90-1.81 (m, 3H), 1.69-1.60 (m, 3H), 1.27 (t, J=7.2 Hz, 3H).

### Example 36. Preparation of 2-amino-1-(3-hydroxy-2,6-dimethylphenyl)-6,11-dimethyl-4,5-dihydro-1,4,7,10,12-pentaazabenzo[4,5]cyclooctatetraeno[1,2,3-cd]inden-3(1H)-one (Compound 36)

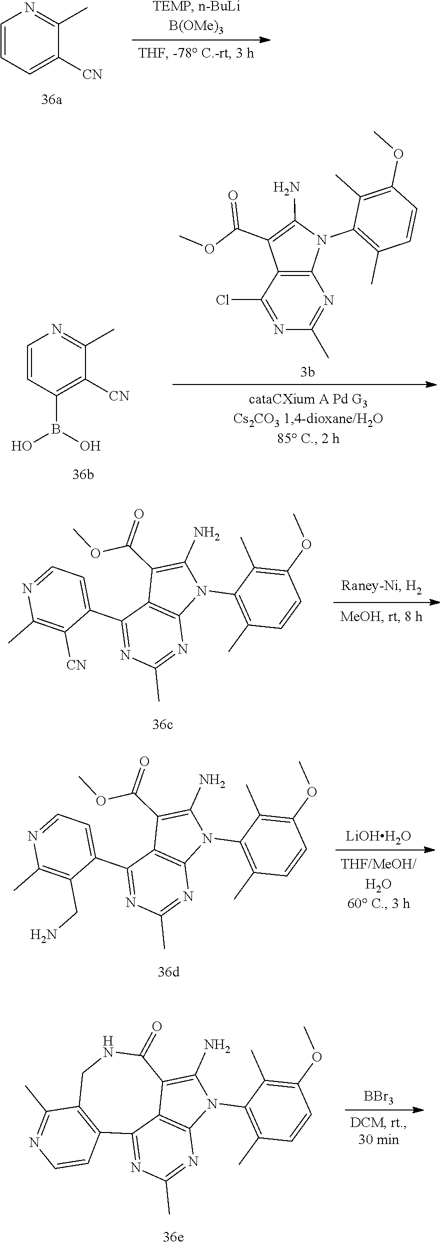

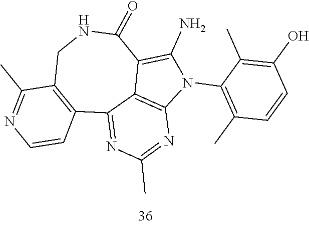 [SMILES: Cc1nc2c3c(c(N)n(-c4c(C)ccc(O)c4C)c3n1)C(=O)NCc1c-2ccnc1C]

### Step 1: (3-cyano-2-methylpyridin-4-yl)boronic Acid (36b)

2,2,6,6-Tetramethylpiperidine (2.2 g, 15 mmol) was dissolved in tetrahydrofuran (10 mL). The system was purged with argon and cooled to −78° C. n-Butyllithium (1.6 M, 9.5 mL) was slowly added dropwise. After the addition was completed, the mixture was warmed to 0° C. and stirred for 30 min. A solution of compound 36a (0.90 g, 7.6 mmol) in tetrahydrofuran (10 mL) was slowly added dropwise at −78° C. After the addition was completed, the mixture was stirred for 30 min. A solution of trimethyl borate (2.4 g, 23 mmol) in tetrahydrofuran (10 mL) was slowly added dropwise at −78° C. After the addition was completed, the mixture was stirred for another 30 min. The mixture was warmed to room temperature and reacted for 3 h. The pH was adjusted to 3-4 with 6 M hydrochloric acid. The mixture was extracted with ethyl acetate, and the organic phases were combined, washed with saturated brine, dried over anhydrous sodium sulfate, and filtered. The filtrate was concentrated, and the resulting crude compound 36b could be directly used in the next step without purification.

### Step 2: methyl 6-amino-4-(3-cyano-2-methylpyridin-4-yl)-7-(3-methoxy-2,6-dimethylphenyl)-2-methyl-7H-pyrrolo[2,3-d]pyrimidine-5-carboxylate (36c)

At room temperature, compound 36b (39 mg, 0.24 mmol), compound 3b (30 mg, 80 μmol), mesylate[n-butyldi(1-adamantyl)phosphine](2-amino-1,1′-biphenyl-2-yl)palladium(II) (5.8 mg, 8.0 μmol), and cesium carbonate (52 mg, 0.16 mmol) were dissolved in 1,4-dioxane/water (5 mL/0.5 mL). The system was purged with nitrogen 3 times, and the mixture was moved to 85° C. and reacted for 2 h. The reaction mixture was cooled to room temperature and then filtered to remove the insoluble substance, and the filtrate was concentrated. The resulting residue was purified by reverse-phase column chromatography (C18, 0.05% aqueous ammonia:acetonitrile=20:1-1:20) to obtain compound 36c (30 mg, yield: 82%). m/z (ESI): 457.2 [M+H]⁺.

### Step 3: methyl 6-amino-4-(3-(aminomethyl)-2-methylpyridin-4-yl)-7-(3-methoxy-2,6-dimethylphenyl)-2-methyl-7H-pyrrolo[2,3-d]pyrimidine-5-carboxylate (36d)

Compound 36c (30 mg, 66 μmol) was dissolved in anhydrous methanol (15 mL) at room temperature, and then Raney nickel (10 mg) was added. The system was purged with hydrogen. The mixture was reacted at room temperature for 8 h. The reaction mixture was filtered to remove the insoluble substance, and the filtrate was concentrated. The resulting residue was purified by reverse-phase column chromatography (C18, 0.05% aqueous ammonia:acetonitrile=20:1-1:20) to obtain compound 36d (10 mg, yield: 33%). m/z (ESI): 461.2 [M+H]⁺.

### Step 4: 2-amino-1-(3-methoxy-2,6-dimethylphenyl)-6,11-dimethyl-4,5-dihydro-1,4,7,10,12-pentaazabenzo[4,5]cyclooctatetraeno[1,2,3-cd]inden-3(1H)-one (36e)

At room temperature, compound 36d (5 mg, 11 μmol) was dissolved in tetrahydrofuran (5 mL) and methanol (2 mL). Lithium hydroxide monohydrate (4.6 mg, 0.11 mmol) and water (1 mL) were then added. The mixture was reacted at 60° C. for 3 h. The reaction was cooled to room temperature, and the pH was adjusted to 6-7 with 1 M hydrochloric acid. The mixture was concentrated under reduced pressure, and the resulting residue was purified by reverse-phase column chromatography (C18, 0.05% aqueous ammonia:acetonitrile=20:1-1:20) to obtain compound 36e (2 mg, yield: 42%). m/z (ESI): 429.2 [M+H]⁺.

### Step 5: 2-amino-1-(3-hydroxy-2,6-dimethylphenyl)-6,11-dimethyl-4,5-dihydro-1,4,7,10,12-pentaazabenzo[4,5]cyclooctatetraeno[1,2,3-cd]inden-3(1H)-one (36)

At room temperature, compound 36e (2 mg, 4.7 μmol) was dissolved in anhydrous dichloromethane (3 mL). A solution of boron tribromide in dichloromethane (1 mL, 1 M) was added. The mixture was reacted at room temperature for 30 min. The reaction was quenched with methanol (1 mL). The reaction mixture was concentrated, adjusted to pH=13-14 with aqueous ammonia, and purified by reverse-phase column chromatography (C18, 0.05% aqueous ammonia:acetonitrile=20:1-1:20) to obtain compound 36 (1 mg, yield: 51%).

m/z (ESI): 415.3 [M+H]⁺.

¹H NMR (400 MHz, DMSO-d₆) δ 9.63 (brs, 1H), 8.47 (d, J=5.2 Hz, 1H), 8.18 (t, J=6.8 Hz, 1H), 7.49 (dd, J=5.2, 1.2 Hz, 1H), 7.15-7.07 (m, 3H), 6.96 (d, J=8.4 Hz, 1H), 5.06-5.00 (m, 1H), 4.28-4.22 (m, 1H), 2.67 (s, 1H), 2.52 (s, 3H), 1.90-1.81 (m, 3H), 1.69-1.60 (m, 3H).

### Example 37. Preparation of 5-amino-4-(3-hydroxy-2,6-dimethylphenyl)-2-methyl-4,7-dihydro-6H-8-thia-1,3,4,7,9-pentaazabenzo[cd]cyclopenta[f]azulen-6-one (Compound 37)

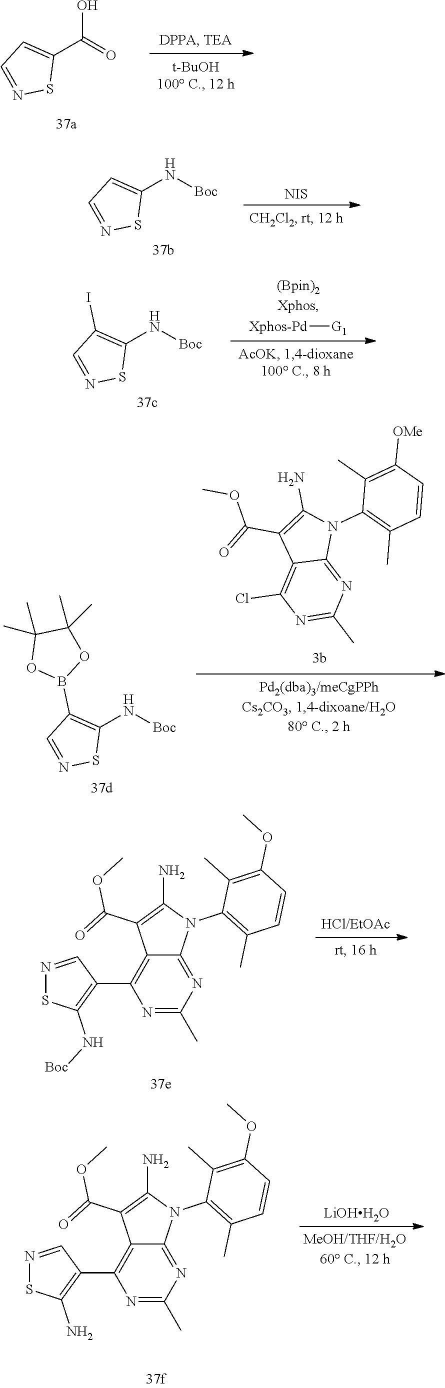

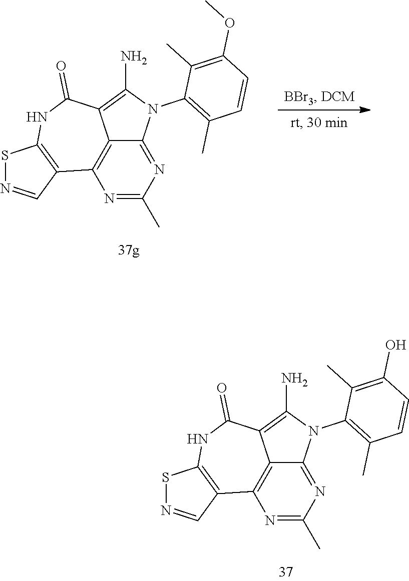

### Step 1: tert-butyl isothiazol-5-ylcarbamate (37b)

At room temperature, isothiazole-5-carboxylic acid (37a, 1.0 g, 7.7 mmol), diphenylphosphoryl azide (2.1 g, 7.7 mmol), and triethylamine (0.78 g, 7.7 mmol) were dissolved in tert-butanol. The mixture was warmed to 100° C. and reacted for 12 h. The reaction mixture was cooled to room temperature and concentrated in vacuum to remove tert-butanol. The residue was dissolved in water and extracted 3 times with ethyl acetate (100 mL×3). The organic phases were combined, washed with saturated brine (50 mL), dried over anhydrous sodium sulfate, and filtered. The filtrate was concentrated. The resulting residue was purified by column chromatography (petroleum ether:ethyl acetate=10:1-1:1) to obtain the product compound 37b (0.55 g, yield: 36%). m/z (ESI): 201.0 [M+H]⁺;

### Step 2: tert-butyl (4-iodoisothiazol-5-yl)carbamate (37c)

Compound 37b (0.40 g, 2.0 mmol) was dissolved in anhydrous dichloromethane (10 mL). N-Iodosuccinimide (0.54 g, 2.4 mmol) was added, and the mixture was reacted at room temperature for 12 h. A saturated aqueous sodium chloride solution (10 mL) was added to the reaction mixture to quench the reaction, and the mixture was extracted 3 times with dichloromethane (20 mL×3). The organic phases were combined, washed with saturated brine (15 mL), dried over anhydrous sodium sulfate, and filtered. The filtrate was concentrated. The resulting residue was purified by normal-phase column chromatography (petroleum ether:ethyl acetate=10:1-1:1) to obtain the product compound 37c (0.61 g, yield: 94%). m/z (ESI): 326.9 [M+H]⁺;

### Step 3: tert-butyl (4-(4,4,5,5-tetramethyl-1,3,2-dioxaborolan-2-yl)isothiazol-5-yl)carbamate (37d)

Compound 37c (0.40 g, 1.2 mmol), bis(pinacolato)diboron (0.47 g, 1.8 mmol), 2-dicyclohexylphosphino-2′,4′,6′-triisopropylbiphenyl (57 mg, 0.12 mmol), chloro(2-dicyclohexylphosphino-2′,4′,6′-triisopropyl-1,1′-biphenyl)[2-(2-aminoethylphenyl)]palladium(II) (89 mg, 0.12 mmol), and potassium acetate (0.24 g, 2.4 mmol) were dissolved in anhydrous 1,4-dioxane (10 mL). The system was purged with nitrogen 3 times, and the mixture was reacted at 100° C. for 8 h under nitrogen atmosphere. The reaction mixture was cooled to room temperature and then filtered. The filtrate was concentrated. The resulting residue, compound 37d, could be directly used in the next step without purification.

### Step 4: methyl 6-amino-4-(5-((tert-butoxycarbonyl)amino)isothiazol-4-yl)-7-(3-methoxy-2,6-dimethylphenyl)-2-methyl-7H-pyrrolo[2,3-d]pyrimidine-5-carboxylate (37e)

The crude product 37d (0.30 g, 0.92 mmol) from the previous step, compound 3b (0.34 g, 0.92 mmol), tris(dibenzylideneacetone)dipalladium (84 mg, 92 μmol), 1,3,5,7-tetramethyl-6-phenyl-2,4,8-trioxa-6-phosphaadamantane (54 mg, 0.19 mmol), and cesium carbonate (0.90 g, 2.8 mmol) were dissolved in 1,4-dioxane/water (5 mL/1 mL). The system was purged with nitrogen 3 times. The reaction mixture was then moved to 80° C. and reacted for 2 h. After the reaction was cooled to room temperature, the reaction mixture was diluted with water (5 mL) and extracted with ethyl acetate (20 mL×3). The organic phases were combined, washed with saturated brine (20 mL), dried over anhydrous sodium sulfate, and filtered. The filtrate was concentrated. The resulting residue was purified by normal-phase column chromatography (petroleum ether:ethyl acetate=10:1-1:2) to obtain the product compound 37e (90 mg, yield: 18%). m/z (ESI): 539.3 [M+H]⁺.

### Step 5: methyl 6-amino-4-(5-aminoisothiazol-4-yl)-7-(3-methoxy-2,6-dimethylphenyl)-2-methyl-7H-pyrrolo[2,3-d]pyrimidine-5-carboxylate (37f)

Compound 37e (90 mg, 0.17 mmol) was dissolved in a solution of hydrochloric acid in ethyl acetate (2 M in EtOAc, 3 mL). The mixture was reacted at room temperature for 16 h. After the reaction was completed, the reaction mixture was concentrated. The resulting crude product, compound 37f, could be directly used in the next step without purification.

### Step 6: 5-amino-4-(3-methoxy-2,6-dimethylphenyl)-2-methyl-4,7-dihydro-6H-8-thia-1,3,4,7,9-pentaazabenzo[cd]cyclopenta[f]azulen-6-one (37g)

Compound 37f (70 mg, 0.15 mmol) obtained in the previous step was dissolved in methanol/tetrahydrofuran/water (2 mL/2 mL/2 mL), and lithium hydroxide monohydrate (63 mg, 1.5 mmol) was then added. The mixture was reacted at 60° C. for 12 h. The reaction mixture was concentrated. The resulting residue was directly purified by reverse-phase column chromatography (C18, 0.05% aqueous ammonia:acetonitrile=20:1-1:20) to obtain compound 37g (40 mg, yield: 67%). m/z (ESI): 407.3 [M+H]⁺.

### Step 7: 5-amino-4-(3-hydroxy-2,6-dimethylphenyl)-2-methyl-4,7-dihydro-6H-8-thia-1,3,4,7,9-pentaazabenzo[cd]cyclopenta[f]azulen-6-one (37)

Compound 37g (30 mg, 74 μmol) was dissolved in anhydrous dichloromethane (4 mL). A solution of boron tribromide (1 mol/L, 2 mL) in dichloromethane was added, and the mixture was reacted at room temperature for 30 min. The reaction mixture was quenched with methanol (5 mL), concentrated, adjusted to pH=13-14 with aqueous ammonia, and purified by reverse-phase column chromatography (C18, 0.05% aqueous ammonia:acetonitrile=20:1-1:20) to obtain compound 37 (12 mg, yield: 41%).

m/z (ESI): 393.1 [M+H]⁺.

¹H NMR (400 MHz, DMSO-d₆) δ 8.62 (s, 1H), 7.25-7.19 (m, 1H), 7.08 (d, J=8.4 Hz, 1H), 6.98-6.91 (m, 3H), 2.38 (s, 3H), 1.79 (s, 3H), 1.70 (s, 3H).

### Example 38. Preparation of 5-amino-4-(3-hydroxy-2,6-dimethylphenyl)-2-methyl-4,7-dihydro-6H-8-thia-1,3,4,7,10-pentaazabenzo[cd]cyclopenta[f]azulen-6-one (Compound 38)

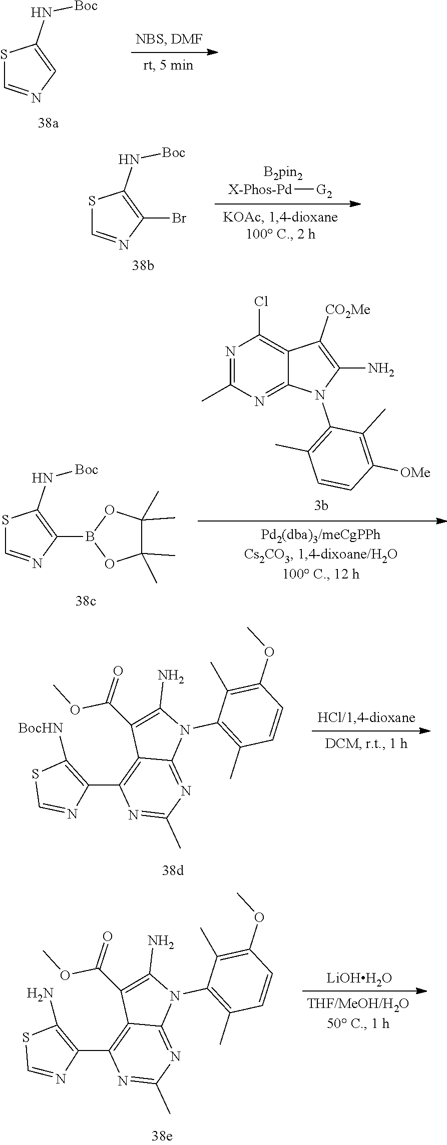

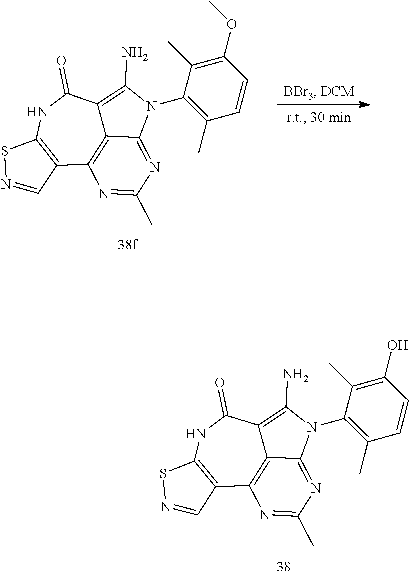

### Step 1: tert-butyl (4-bromothiazol-5-yl)carbamate (38b)

Compound 38a (0.45 g, 2.3 mmol) was dissolved in anhydrous N,N-dimethylformamide (10 mL). N-Bromosuccinimide (0.36 g, 2.0 mmol) was added. The mixture was reacted at room temperature for 5 min. The reaction mixture was directly purified by reverse-phase column chromatography (C18, 0.05% aqueous ammonia:acetonitrile=20:1-1:20) to obtain the product compound 38b (0.55 g, yield: 88%). m/z (ESI): 279.1 [M+H]⁺.

### Step 2: tert-butyl [4-(4,4,5,5-tetramethyl-1,3,2-dioxaborolan-2-yl)thiazol-5-yl]carbamate (38c)

Compound 38b (0.25 g, 0.90 mmol), 4,4,5,5-tetramethyl-2-(4,4,5,5-tetramethyl-1,3,2-dioxaborolan-2-yl)-1,3,2-dioxaborolane (0.46 g, 1.8 mmol), chloro(2-dicyclohexylphosphino-2′,4′,6′-triisopropyl-1,1′-biphenyl)[2-(2′-amino-1,1′-biphenyl)]palladium(II) (0.14 g, 0.18 mmol), and potassium acetate (0.26 g, 2.7 mmol) were dissolved in 1,4-dioxane (5 mL). The system was purged with nitrogen 3 times. The mixture was reacted at 100° C. for 2 h under nitrogen atmosphere. The reaction mixture was cooled to room temperature and then filtered to remove the insoluble substance. The filtrate was concentrated. The resulting crude product, compound 38c, could be directly used in the next step without purification. m/z (ESI): 327.2 [M+H]⁺.

### Step 3: methyl 6-amino-4-(5-((tert-butoxycarbonyl)amino)thiazol-4-yl)-7-(3-methoxy-2,6-dimethylphenyl)-2-methyl-7H-pyrrolo[2,3-d]pyrimidine-5-carboxylate (38d)

The crude compound 38c (0.26 g, 0.80 mmol) from the previous step, compound 3b (0.10 g, 0.27 mmol), tris(dibenzylideneacetone)dipalladium (25 mg, 27 μmol), 1,3,5,7-tetramethyl-6-phenyl-2,4,8-trioxa-6-phosphaadamantane (16 mg, 53 μmol), and cesium carbonate (0.26 g, 0.80 mmol) were dissolved in 1,4-dioxane and water (5 mL/0.5 mL). The system was purged with nitrogen three times. The mixture was reacted at 100° C. for 12 h under nitrogen atmosphere. The reaction mixture was cooled to room temperature and then filtered to remove the insoluble substance, and the filtrate was concentrated. The resulting residue was purified by reverse-phase column chromatography (C18, 0.05% aqueous ammonia:acetonitrile=20:1-1:20) to obtain the product compound 38d (19 mg, yield: 14%). m/z (ESI): 539.2 [M+H]⁺.

### Step 4: methyl 6-amino-4-(5-aminothiazol-4-yl)-7-(3-methoxy-2,6-dimethylphenyl)-2-methyl-7H-pyrrolo[2,3-d]pyrimidine-5-carboxylate (38e)

Compound 38d (19 mg, 35 μmol) was dissolved in anhydrous dichloromethane (3 mL). Under an ice bath, a solution of hydrogen chloride in dioxane (3 mL, 4 M) was added to the reaction mixture. The mixture was warmed to room temperature and reacted for 1 h. The reaction mixture was concentrated to remove the solvent. The pH was adjusted to neutral with aqueous ammonia (25% aqueous ammonia solution). The mixture was purified by reverse-phase column chromatography (C18, 0.05% aqueous ammonia:acetonitrile=20:1-1:20) to obtain compound 38e (15 mg, yield: 97%). m/z (ESI): 439.3 [M+H]⁺.

### Step 5: 5-amino-4-(3-methoxy-2,6-dimethylphenyl)-2-methyl-4,7-dihydro-6H-8-thia-1,3,4,7,10-pentaazabenzo[cd]cyclopenta[f]azulen-6-one (38f)

Compound 38e (15 mg, 35 μmol) was dissolved in a mixed solution of tetrahydrofuran (2 mL), methanol (2 mL), and water (2 mL). Lithium hydroxide monohydrate (43 mg, 1.0 mmol) was added. The mixture was moved to 50° C. and reacted for 1 h. The reaction mixture was cooled to room temperature and then filtered to remove the insoluble substance. The filtrate was concentrated, and the resulting crude product was purified by reverse-phase column chromatography (C18, 0.05% aqueous ammonia:acetonitrile=20:1-1:20) to obtain compound 38f (8 mg, yield: 58%). m/z (ESI): 407.2 [M+H]⁺.

### Step 6: 5-amino-4-(3-hydroxy-2,6-dimethylphenyl)-2-methyl-4,7-dihydro-6H-8-thia-1,3,4,7,10-pentaazabenzo[cd]cyclopenta[f]azulen-6-one (38)

Compound 38f (8 mg, 20 μmol) was dissolved in anhydrous dichloromethane (3 mL). A solution of boron tribromide (0.1 mL, 1 mol/L) in dichloromethane was slowly added dropwise under an ice bath. The mixture was warmed to room temperature and reacted for 2 h. The reaction was quenched with methanol (2 mL). The reaction mixture was concentrated to remove the solvent. The pH was adjusted to neutral with aqueous ammonia (25% aqueous ammonia solution). The mixture was purified by reverse-phase column chromatography (C18, 0.05% aqueous ammonia:acetonitrile=20:1-1:20) to obtain compound 38 (3.8 mg, yield: 49%). m/z (ESI): 393.4 [M+H]⁺.

¹H NMR (400 MHz, DMSO-d₆) δ 10.11 (s, 1H), 9.60 (s, 1H), 8.47 (s, 1H), 7.08 (d, J=8.3 Hz, 1H), 6.94 (d, J=8.3 Hz, 1H), 6.85 (s, 2H), 2.37 (s, 3H), 1.79 (s, 3H), 1.71 (s, 3H).

### Example 39. Preparation of 2-amino-6-(difluoromethoxy)-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (Compound 39)

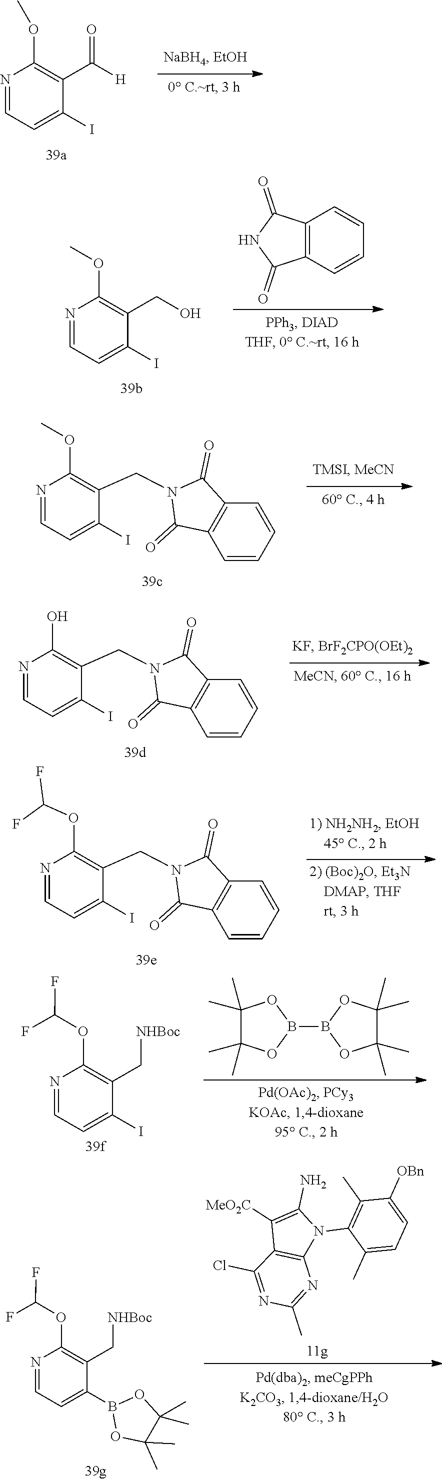

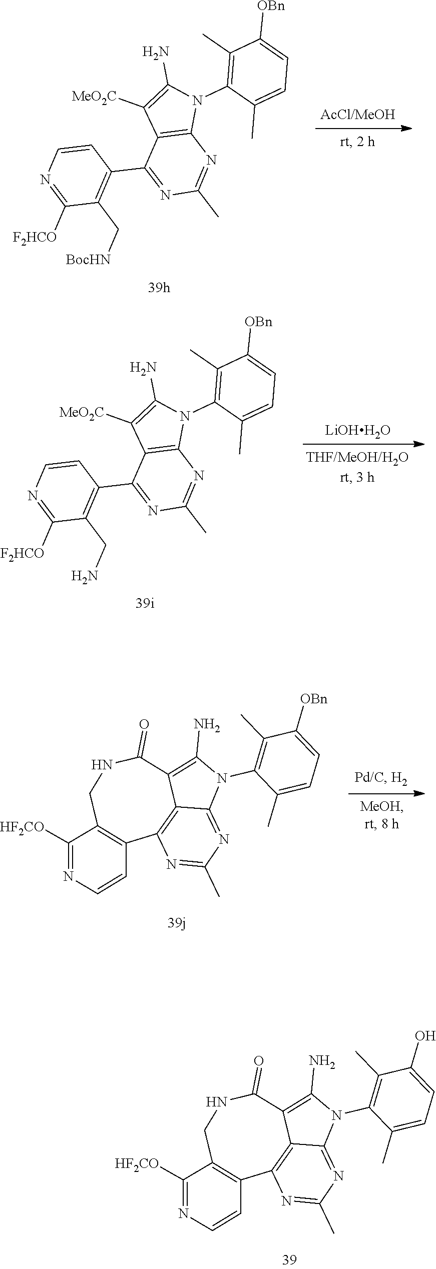

### Step 1: (4-iodo-2-methoxypyridin-3-yl)methanol (39b)

Compound 39a (10 g, 38 mmol) was dissolved in anhydrous ethanol (120 mL). Sodium borohydride (2.1 g, 57 mmol) was slowly added dropwise to the reaction mixture under an ice bath. The mixture was moved to room temperature and reacted for 3 h. A small amount of water (10 mL) was slowly added dropwise to the reaction system to quench the reaction. The reaction mixture was concentrated to remove ethanol. The mixture was extracted with ethyl acetate (200 mL×3), and the organic phases were combined, washed with saturated brine (200 mL), dried over anhydrous sodium sulfate, and filtered. The filtrate was concentrated to obtain the product compound 39b (9.6 g, yield: 95%). m/z (ESI): 266.0 [M+H]⁺.

### Step 2: 2-((4-iodo-2-methoxypyridin-3-yl)methyl)isoindoline-1,3-dione (39c)

Compound 39b (6.0 g, 23 mmol) was dissolved in anhydrous tetrahydrofuran (80 mL). Phthalimide (4.0 g, 27 mmol) and triphenylphosphine (12 g, 45 mmol) were sequentially added. The system was purged with nitrogen 3 times and then cooled to 0° C. under nitrogen atmosphere. Diisopropyl azodicarboxylate (9.2 g, 45 mmol) was slowly added dropwise. The mixture was warmed to room temperature and reacted for 16 h. The reaction mixture was concentrated, and the residue was dissolved in water. The mixture was extracted with ethyl acetate (60 mL×3), and the organic phases were combined, washed with saturated brine (60 mL), dried over anhydrous sodium sulfate, and filtered. The filtrate was concentrated, and the resulting residue was purified by normal-phase column chromatography (petroleum ether:ethyl acetate=4:1-3:2) to obtain compound 39c (6.0 g, yield: 67%). m/z (ESI): 394.9 [M+H]⁺.

### Step 3: 2-((2-hydroxy-4-iodopyridin-3-yl)methyl)isoindoline-1,3-dione (39d)

Compound 39c (6.0 g, 15 mmol) was dissolved in anhydrous acetonitrile (80 mL). Trimethylsilyl iodide (12 g, 60 mmol) was added, and the mixture was warmed to 60° C. and reacted for 4 h. A saturated aqueous sodium thiosulfate solution (60 mL) was added to quench the reaction. The reaction mixture was concentrated to remove acetonitrile and extracted 3 times with ethyl acetate (60 mL×3), and the organic phases were combined, washed with saturated brine (60 mL), dried over anhydrous sodium sulfate, and filtered. The filtrate was concentrated, and the resulting residue was purified by normal-phase column chromatography (dichloromethane:methanol=10:1-10:2) to obtain compound 39d (4.7 g, yield: 80%). m/z (ESI): 380.9 [M+H]⁺.

### Step 4: 2-((2-(difluoromethoxy)-4-iodopyridin-3-yl)methyl)isoindoline-1,3-dione (39e)

Compound 39d (4.7 g, 12 mmol) was dissolved in anhydrous acetonitrile (60 mL). Diethyl (bromodifluoromethyl)phosphonate (4.9 g, 18 mmol) and potassium fluoride (1.5 g, 24 mmol) were added sequentially, and the mixture was warmed to 60° C. and reacted for 16 h. The reaction mixture was concentrated to remove acetonitrile, and the residue was dissolved in water (30 mL). The mixture was extracted with ethyl acetate (60 mL×3), and the organic phases were combined, washed with saturated brine (60 mL), dried over anhydrous sodium sulfate, and filtered. The filtrate was concentrated, and the resulting residue was subjected to normal-phase column chromatography (petroleum ether:ethyl acetate=4:1-3:2) to obtain compound 39e (2.8 g, yield: 53%). m/z (ESI): 430.9 [M+H]⁺.

### Step 5: tert-butyl ((2-(difluoromethoxy)-4-iodopyridin-3-yl)methyl)carbamate (39f)

Compound 39e (2.8 g, 6.5 mmol) was dissolved in ethanol (30 mL). Hydrazine hydrate (85%, 1.1 g, 33 mmol) was added, and the mixture was warmed to 45° C. and reacted for 2 h. The reaction was cooled to room temperature, and then di-tert-butyl dicarbonate (2.0 g, 6.5 mmol), triethylamine (0.21 g, 20 mmol), and 4-dimethylaminopyridine (1.4 g, 6.5 mmol) were sequentially added. The mixture was reacted at room temperature for another 3 h. The reaction mixture was concentrated to remove the organic solvent, and the residue was dissolved in water (30 mL). The mixture was extracted with ethyl acetate (50 mL×3), and the organic phases were combined, washed with saturated brine (30 mL), dried over anhydrous sodium sulfate, and filtered. The filtrate was concentrated, and the resulting residue was subjected to normal-phase column chromatography (petroleum ether:ethyl acetate=10:1-5:1) to obtain compound 39f (1.7 g, yield: 65%).

### Step 6: tert-butyl ((2-(difluoromethoxy)-4-(4,4,5,5-tetramethyl-1,3,2-dioxaborolan-2-yl)pyridin-3-yl)methyl)carbamate (39g)

Compound 39f (40 mg, 0.10 mmol), bis(pinacolato)diboron (31 mg, 0.12 mmol), palladium acetate (4.5 mg, 20 μmol), tricyclohexylphosphine (5.6 mg, 20 μmol), and potassium acetate (29 mg, 0.30 mmol) were dissolved in anhydrous 1,4-dioxane (2 mL). The system was purged with argon, and the mixture was reacted at 95° C. for 2 h under argon atmosphere. The reaction mixture was cooled to room temperature and then filtered to remove the insoluble substance. The filtrate was concentrated. The resulting crude product, compound 39g, could be directly used in the next step without purification. m/z (ESI): 401.4 [M+H]⁺.

### Step 7: methyl 6-amino-7-(3-(benzyloxy)-2,6-dimethylphenyl)-4-(3-(((tert-butoxycarbonyl)amino)methyl)-2-(difluoromethoxy)pyridin-4-yl)-2-methyl-7H-pyrrolo[2,3-d]pyrimidine-5-carboxylate (39h)

The crude product compound 39g (40 mg, 0.1 mmol) from the previous step, compound 11g (40 mg, 88 μmol), bis(dibenzylideneacetone)palladium (5.0 mg, 8.8 μmol), 1,3,5,7-tetramethyl-6-phenyl-2,4,8-trioxa-6-phosphaadamantane (5.2 mg, 18 μmol), and potassium carbonate (0.29 g, 0.89 mmol) were dissolved in 1,4-dioxane/water (5 mL/1 mL). The system was purged with nitrogen 3 times. The reaction mixture was moved to 80° C. and reacted for 3 h. The reaction mixture was cooled to room temperature and then filtered to remove the insoluble substance, and the filtrate was concentrated. The resulting residue was purified by reverse-phase column chromatography (C18, 0.05% aqueous ammonia:acetonitrile=20:1-1:20) to obtain compound 39h (12 mg, yield: 20%). m/z (ESI): 689.8 [M+H]⁺.

### Step 8: methyl 6-amino-4-(3-(aminomethyl)-2-(difluoromethoxy)pyridin-4-yl)-7-(3-(benzyloxy)-2,6-dimethylphenyl)-2-methyl-7H-pyrrolo[2,3-d]pyrimidine-5-carboxylate (39i)

Compound 39h (12 mg, 17 μmol) was dissolved in anhydrous methanol (5 mL). Acetyl chloride (5 mL) was then slowly added dropwise to the solution under an ice bath. After the addition was completed, the mixture was moved to room temperature and reacted for 2 h. After the reaction was completed, the reaction mixture was concentrated. The resulting crude product, compound 39i (8 mg), could be directly used in the next step without purification. m/z (ESI): 589.8 [M+H]⁺.

### Step 9: 2-amino-1-(3-(benzyloxy)-2,6-dimethylphenyl)-6-(difluoromethoxy)-11-methyl-4,5-dihydro-1,4,7,10,12-pentaazabenzo[4,5]cyclooctatetraeno[1,2,3-cd]inden-3(1H)-one (39j)

Compound 39i (8 mg, 14 μmol) obtained in the previous step was dissolved in methanol/tetrahydrofuran/water (5 mL/5 mL/5 mL), and lithium hydroxide monohydrate (5.7 mg, 0.14 mmol) was then added. The mixture was reacted at room temperature for 3 h. The reaction mixture was concentrated. The resulting residue was purified by reverse-phase column chromatography (C18, 0.05% aqueous ammonia:acetonitrile=20:1-1:20) to obtain the target compound 39j (5.1 mg, yield: 67%). m/z (ESI): 557.6 [M+H]⁺.

### Step 10: 2-amino-6-(difluoromethoxy)-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (39)

Compound 39j (5.1 mg, 79 μmol) was dissolved in anhydrous methanol (5 mL), and then Pd/C (10%, 1.1 mg) was added. The system was purged with hydrogen. The mixture was reacted at room temperature for 8 h. The reaction mixture was filtered to remove the insoluble substance, and the filtrate was concentrated. The resulting residue was subjected to reverse-phase column chromatography (C18, 0.05% aqueous ammonia:acetonitrile=20:1-1:20) to obtain compound 39 (2.8 mg, yield: 65%).

m/z (ESI): 467.4 [M+H]⁺.

¹H NMR (400 MHz, DMSO-d₆) δ 9.65 (s, 1H), 8.32 (d, J=5.3 Hz, 1H), 8.02 (t, J=7.0 Hz, 1H), 7.81 (t, J=72.6 Hz, 1H), 7.59 (d, J=5.3 Hz, 1H), 7.21 (s, 2H), 7.13 (dd, J=12.0, 8.2 Hz, 1H), 6.98 (d, J=8.2 Hz, 1H), 5.00-4.88 (m, 1H), 4.40-4.26 (m, 1H), 2.53 (s, 3H), 1.94-1.81 (m, 3H), 1.74-1.63 (m, 3H).

### Example 40. Preparation of 2-amino-6-(fluoromethoxy)-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (Compound 40)

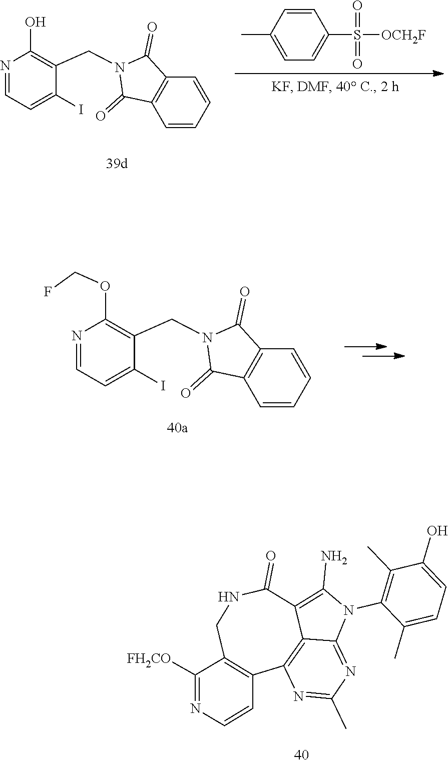

### Step 1: 2-((2-(fluoromethoxy)-4-iodopyridin-3-yl)methyl)isoindoline-1,3-dione (40a)

At room temperature, compound 39d (2.0 g, 5.3 mmol) was dissolved in anhydrous N,N-dimethylformamide (20 mL). Fluoromethyl 4-methylbenzenesulfonate (4.3 g, 21 mmol) and potassium fluoride (1.2 g, 21 mmol) were sequentially added. The mixture was warmed to 40° C. and reacted for 2 h. The reaction mixture was cooled to room temperature and filtered. The filtrate was diluted with water (20 mL) and extracted 3 times with ethyl acetate (50 mL×3). The organic phases were combined, washed 3 times with saturated brine (20 mL×3), dried over anhydrous sodium sulfate, and filtered. The filtrate was concentrated, and the resulting residue was purified by normal-phase column chromatography (petroleum ether:ethyl acetate=10:1-2:1) to obtain compound 40a (0.18 g, yield: 8.3%). m/z (ESI): 413.1 [M+H]⁺.

Compound 40 was then prepared by replacing compound 39e with compound 40a and using a method similar to that in step 5 to step 10 of Example 39.

m/z (ESI): 449.4 [M+H]⁺;

¹H NMR (400 MHz, DMSO-d₆) δ 8.27 (d, J=5.6 Hz, 1H), 7.96 (t, J=6.8 Hz, 1H), 7.46 (d, J=4.8 Hz, 1H), 7.13-7.05 (m, 3H), 6.94 (d, J=8.4 Hz, 1H), 6.16 (td, J=52.8, 2.4 Hz, 2H), 4.92-4.87 (m, 1H), 4.38-4.32 (m, 1H), 2.53 (s, 3H), 1.89-1.80 (m, 3H), 1.69-1.60 (m, 3H).

### Example 41. Preparation of 2-amino-1-(3-hydroxy-2,6-dimethylphenyl)-11-methyl-6-(trifluoromethoxy)-4,5-dihydro-1,4,7,10,12-pentaazabenzo[4,5]cyclooctatetraeno[1,2,3-cd]inden-3(1H)-one (Compound 41)

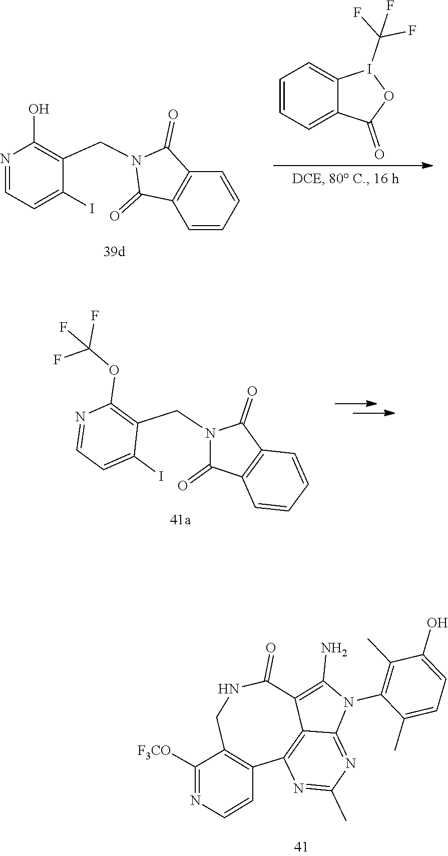 [SMILES: O=C1c2ccccc2C(=O)N1Cc1c(I)ccnc1O ; O=C1OI(C(F)(F)F)c2ccccc21 ; O=C1c2ccccc2C(=O)N1Cc1c(I)ccnc1OC(F)(F)F ; Cc1nc2c3c(c(N)n(-c4c(C)ccc(O)c4C)c3n1)C(=O)NCc1c-2ccnc1OC(F)(F)F]

### Step 1: 2-((4-iodo-2-(trifluoromethoxy)pyridin-3-yl)methyl)isoindoline-1,3-dione (41a)

Compound 39d (1.4 g, 3.7 mmol) was dissolved in 1,2-dichloroethane (20 mL). 1-(Trifluoromethyl)-1,2-benziodoxol-3(1H)-one (1.7 g, 5.2 mmol) was then added. The mixture was warmed to 80° C. and reacted for 16 h. The reaction mixture was concentrated to remove the solvent. The resulting residue was directly purified by reverse-phase column chromatography (C18, 0.05% formic acid:acetonitrile=20:1-1:20) to obtain compound 41a (0.17 g, yield: 12%). m/z (ESI): 448.9 [M+H]⁺.

Compound 41 was then prepared by replacing compound 39e with compound 41a and using a method similar to that in step 5 to step 10 of Example 39.

m/z (ESI): 485.3 [M+H]⁺;

¹H NMR (400 MHz, DMSO-d₆) δ 8.38 (d, J=5.2 Hz, 1H), 8.17-8.13 (m, 1H), 7.69 (d, J=6.8 Hz, 1H), 7.25-7.15 (m, 3H), 7.12-7.08 (m, 1H), 6.96 (d, J=8.4 Hz, 1H), 5.00-4.93 (m, 1H), 4.33-4.27 (m, 1H), 2.53 (s, 3H), 1.90-1.81 (m, 3H), 1.70-1.61 (m, 3H).

### Example 42. Preparation of 5-amino-8-(difluoromethyl)-4-(3-hydroxy-2,6-dimethylphenyl)-2-methyl-7,8-dihydro-1,3,4,7,8,9-hexaazabenzo[cd]cyclopenta[f]azulen-6(4H)-one (Compound 42)

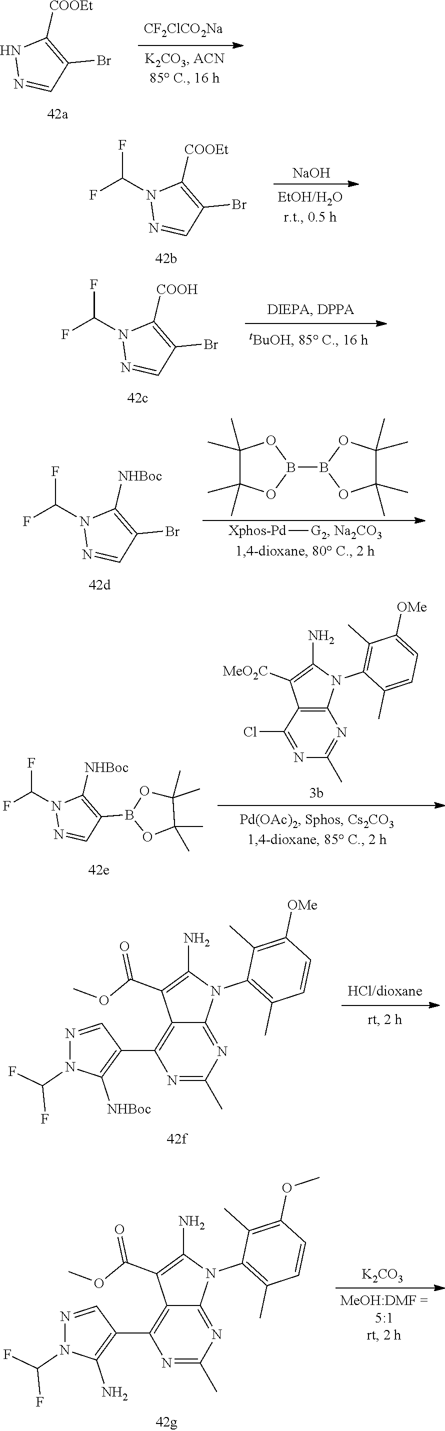

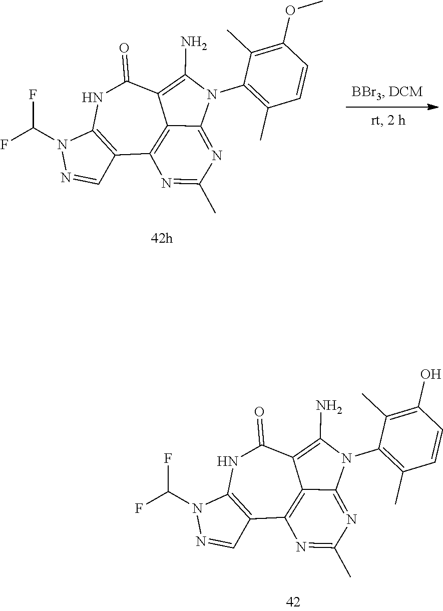 [SMILES: COc1ccc(C)c(-n2c(N)c3c4c(nc(C)nc42)-c2cnn(C(F)F)c2NC3=O)c1C ; Cc1nc2c3c(c(N)n(-c4c(C)ccc(O)c4C)c3n1)C(=O)Nc1c-2cnn1C(F)F]

### Step 1: ethyl 4-bromo-1-(difluoromethyl)-1H-pyrazole-5-carboxylate (42b)

Compound 42a (5.0 g, 23 mmol) was dissolved in anhydrous acetonitrile (4 mL). Sodium difluorochloroacetate (7.1 g, 46 mmol) and potassium carbonate (6.3 g, 46 mmol) were sequentially added. The mixture was heated to 85° C. and reacted for 16 h. The reaction mixture was cooled to room temperature and then filtered, and the filtrate was concentrated. The resulting residue was purified by normal-phase column chromatography (petroleum ether:ethyl acetate=10:1-5:1) to obtain the target compound 42b (0.68 g, yield: 11%). m/z (ESI): 269.0 [M+H]⁺.

### Step 2: 4-bromo-1-(difluoromethyl)-1H-pyrazole-5-carboxylic Acid (42c)

Compound 42b (0.48 g, 1.8 mmol) was dissolved in EtOH:H₂O=1:1 (4 mL). Sodium hydroxide (0.14 g, 3.6 mmol) was added. The mixture was reacted at room temperature for 0.5 h. After the reaction was completed, the pH was adjusted to 2-3 with 1 N hydrochloric acid. The mixture was extracted with ethyl acetate (20 mL×3), and the organic phases were combined, washed with saturated brine (20 mL), dried over anhydrous sodium sulfate, and filtered. The filtrate was concentrated, and the resulting crude product, compound 42c, could be directly used in the next step without purification. m/z (ESI): 240.9 [M+1]⁺.

### Step 3: tert-butyl (4-bromo-1-(difluoromethyl)-1H-pyrazol-5-yl)carbamate (42d)

Compound 42c (0.38 g, 1.6 mmol) was dissolved in tert-butanol (8 mL). N,N-Diisopropylethylamine (0.31 g, 2.4 mmol) and diphenylphosphoryl azide (0.65 g, 2.4 mmol) were sequentially added. The mixture was warmed to 85° C. and reacted for 16 h. The reaction mixture was cooled to room temperature and then filtered, and the filtrate was concentrated. The resulting residue was purified by normal-phase column chromatography (petroleum ether:ethyl acetate=10:1-1:1) to obtain compound 42d (0.17 g, yield: 34%). m/z (ESI): 312.1 [M+1]⁺.

### Step 4: tert-butyl (1-(difluoromethyl)-4-(4,4,5,5-tetramethyl-1,3,2-dioxaborolan-2-yl)-1H-pyrazol-5-yl)carbamate (42e)

Compound 42d (0.50 g, 1.6 mmol), 4,4,5,5-tetramethyl-2-(4,4,5,5-tetramethyl-1,3,2-dioxaborolan-2-yl)-1,3,2-dioxaborolane (0.81 g, 3.2 mmol), chloro(2-dicyclohexylphosphino-2′,4′,6′-triisopropyl-1,1′-biphenyl)[2-(2′-amino-1,1′-biphenyl)]palladium(II) (0.25 g, 0.32 mmol), and sodium carbonate (0.33 g, 3.2 mmol) were dissolved in 1,4-dioxane (10 mL). The system was purged with nitrogen 3 times. The mixture was then reacted at 80° C. for 2 h under nitrogen atmosphere. The reaction mixture was cooled to room temperature and then filtered. The filtrate was concentrated. The resulting residue, compound 42e, could be directly used in the next step without purification. m/z (ESI): 360.4 [M+H]⁺.

### Step 5: methyl 6-amino-4-(5-((tert-butoxycarbonyl)amino)-1-(difluoromethyl)-1H-pyrazol-4-yl)-7-(3-methoxy-2,6-dimethylphenyl)-2-methyl-7H-pyrrolo[2,3-d]pyrimidine-5-carboxylate (42f)

Compound 42e (0.35 g, 0.97 mmol) obtained in the previous step, compound 3b (0.33 g, 0.88 mmol), palladium acetate (42 mg, 0.19 mmol), 2-dicyclohexylphosphino-2′,6′-dimethoxybiphenyl (77 mg, 0.19 mmol), and cesium carbonate (0.61 g, 1.9 mmol) were dissolved in 1,4-dioxane/water (5 mL/0.5 mL). The system was purged with nitrogen 3 times. The mixture was then reacted at 85° C. for 2 h under nitrogen atmosphere. The reaction mixture was cooled to room temperature and then filtered, and the filtrate was concentrated. The resulting residue was purified by reverse-phase column chromatography (C18, 0.05% formic acid:acetonitrile=20:1-1:20) to obtain compound 42f (0.30 g, yield: 60%). m/z (ESI): 572.3 [M+H]⁺.

### Step 6: methyl 6-amino-4-(5-amino-1-(difluoromethyl)-1H-pyrazol-4-yl)-7-(3-methoxy-2,6-dimethylphenyl)-2-methyl-7H-pyrrolo[2,3-d]pyrimidine-5-carboxylate (42g)

Compound 42f (0.30 g, 0.52 mmol) was dissolved in 1,4-dioxane (5 mL). A solution of hydrochloric acid in dioxane (4 mol/L, 2 mL) was then slowly added dropwise. The mixture was reacted at room temperature for 2 h. After the reaction was completed, the reaction mixture was concentrated. The resulting residue was purified by reverse-phase column chromatography (C18, 0.05% formic acid:acetonitrile=20:1-1:20) to obtain compound 42g (0.16 g, yield: 65%). m/z (ESI): 472.3 [M+H]⁺.

### Step 7: 5-amino-8-(difluoromethyl)-4-(3-methoxy-2,6-dimethylphenyl)-2-methyl-7,8-dihydro-1,3,4,7,8,9-hexaazabenzo[cd]cyclopenta[f]azulen-6(4H)-one (42h)

Compound 42g (40 mg, 85 μmol) was dissolved in N,N-dimethylformamide:methanol=1:5 (2 mL). Potassium carbonate (24 mg, 0.17 mol) was then added. The mixture was reacted at room temperature for 2 h. After the reaction was completed, the reaction mixture was concentrated. The resulting residue was purified by reverse-phase column chromatography (C18, 0.05% formic acid:acetonitrile=20:1-1:20) to obtain compound 42h (30 mg, yield: 80%). m/z (ESI): 440.3 [M+H]⁺.

### Step 8: 5-amino-8-(difluoromethyl)-4-(3-hydroxy-2,6-dimethylphenyl)-2-methyl-7,8-dihydro-1,3,4,7,8,9-hexaazabenzo[cd]cyclopenta[f]azulen-6(4H)-one (42)

Compound 42h (15 mg, 34 μmol) was dissolved in dichloromethane. A solution of boron tribromide in dichloromethane (2 mL, 2 mol/L) was then added. The mixture was reacted at room temperature for 2 h. After the reaction was completed, methanol (5 mL) was slowly added dropwise to the reaction mixture to quench the reaction under an ice bath. The pH was then adjusted to 14 with aqueous ammonia. The reaction mixture was concentrated. The resulting residue was purified by reverse-phase column chromatography (C18, 0.05% formic acid:acetonitrile=20:1-1:20) to obtain compound 42 (2.3 mg, yield: 16%).

m/z (ESI): 426.3 [M+H]⁺.

¹H NMR (400 MHz, CD₃OD) δ 8.05 (s, 1H), 7.66-7.37 (m, 1H), 7.02 (d, J=8.4 Hz, 1H), 6.85 (d, J=8.4 Hz, 1H), 2.38 (s, 3H), 1.79 (s, 3H), 1.74 (s, 3H).

### Example 43. Preparation of 5-amino-4-(3-hydroxy-2,6-dimethylphenyl)-8-(2-methoxyethyl)-2-methyl-7,8-dihydro-1,3,4,7,8,9-hexaazabenzo[cd]cyclopenta[f]azulen-6(4H)-one (Compound 43)

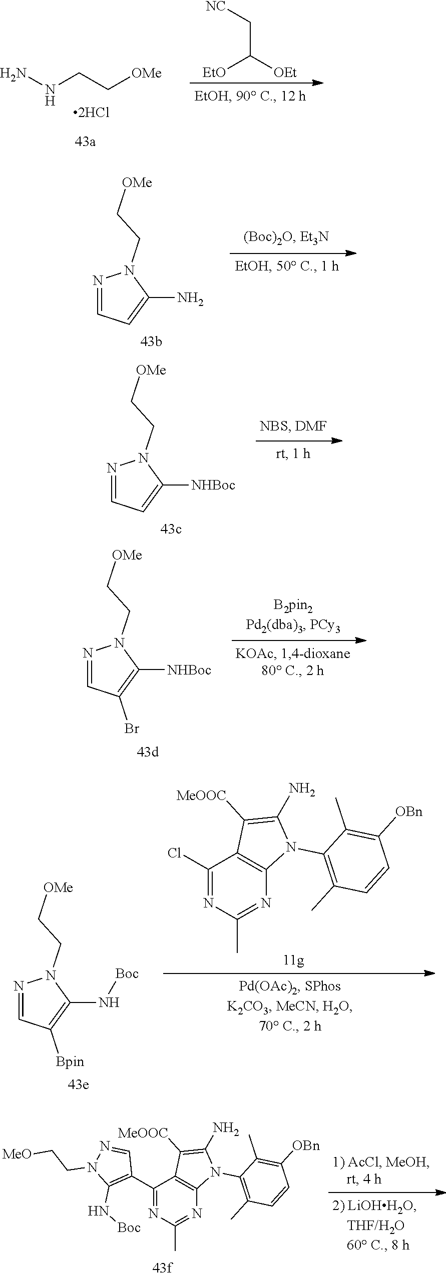

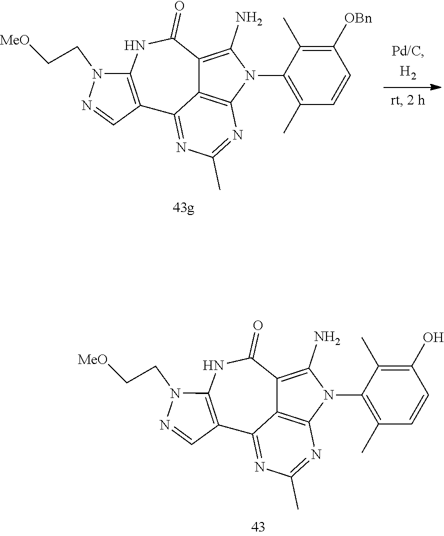 [SMILES: COCCn1ncc2c1NC(=O)c1c(N)n(-c3c(C)ccc(OCc4ccccc4)c3C)c3nc(C)nc-2c13 ; COCCn1ncc2c1NC(=O)c1c(N)n(-c3c(C)ccc(O)c3C)c3nc(C)nc-2c13]

### Step 1: 1-(2-methoxyethyl)-1H-pyrazol-5-amine (43b)

Compound 43a (1.0 g, 6.1 mmol) was dissolved in anhydrous ethanol (20 mL). Cyanoacetaldehyde diethyl acetal (0.87 g, 6.1 mmol) was then added. The mixture was reacted at 90° C. for 12 h. The mixture was concentrated in vacuum to remove the solvent. The resulting crude compound 43b could be directly used in the next step without purification.

### Step 2: tert-butyl (1-(2-methoxyethyl)-1H-pyrazol-5-yl)carbamate (43c)

The crude compound 43b obtained in the previous step was dissolved in ethanol (20 mL). Di-tert-butyl dicarbonate (1.3 g, 61 mmol) and triethylamine (6.1 g, 61 mmol) were sequentially added. The mixture was reacted at 50° C. for 1 h. The mixture was concentrated in vacuum to remove the solvent. The resulting residue was purified by reverse-phase column chromatography (C18, 0.05% aqueous ammonia:acetonitrile=20:1-1:20) to obtain compound 43c (0.99 g, two-step yield: 67%). m/z (ESI): 242.2 [M+H]⁺.

### Step 3: tert-butyl (4-bromo-1-(2-methoxyethyl)-1H-pyrazol-5-yl)carbamate (43d)

Compound 43c (0.99 g, 4.1 mmol) was dissolved in anhydrous N,N-dimethylformamide (5 mL). N-Bromosuccinimide (0.73 g, 4.1 mmol) was then added. The mixture was reacted at room temperature for 1 h. The reaction mixture was directly purified by reverse-phase column chromatography (C18, 0.05% aqueous ammonia:acetonitrile=20:1-1:20) to obtain the target compound 43d (1.1 g, yield: 87%). m/z (ESI): 320.2 [M+H]⁺.

### Step 4: tert-butyl (1-(2-methoxyethyl)-4-(4,4,5,5-tetramethyl-1,3,2-dioxaborolan-2-yl)-1H-pyrazol-5-yl)carbamate (43e)

Compound 43d (0.60 g, 1.9 mmol), bis(pinacolato)diboron (0.97 g, 3.8 mmol), tricyclohexylphosphine (0.11 g, 0.38 mmol), tris(dibenzylideneacetone)dipalladium (0.18 g, 0.2 mmol), and potassium acetate (0.56 g, 5.7 mmol) were dissolved in anhydrous 1,4-dioxane. The system was purged with argon 3 times. The mixture was then reacted at 80° C. for 2 h under nitrogen atmosphere. The reaction mixture was cooled to room temperature and then filtered to remove the insoluble substance. The filtrate was concentrated. The resulting crude compound 43e could be directly used in the next step without purification.

### Step 5: methyl 6-amino-7-(3-(benzyloxy)-2,6-dimethylphenyl)-4-(5-((tert-butoxycarbonyl)amino)-1-(2-methoxyethyl)-1H-pyrazol-4-yl)-2-methyl-7H-pyrrolo[2,3-d]pyrimidine-5-carboxylate (43f)

The crude compound 43e (0.70 g, 1.9 mmol) obtained in the previous step, compound 11g (0.13 g, 0.28 mmol), palladium acetate (6.3 mg, 28 μmol), 2-dicyclohexylphosphino-2′,6′-dimethoxybiphenyl (23 mg, 56 μmol), and potassium carbonate (0.12 g, 0.84 mmol) were dissolved in acetonitrile and water (10 mL/1 mL). The system was purged with argon 3 times. The mixture was then reacted at 70° C. under an oil bath for 2 h. The reaction mixture was cooled to room temperature and then filtered to remove the insoluble substance. The filtrate was concentrated. The resulting residue was purified by reverse-phase column chromatography (C18, 0.05% aqueous ammonia:acetonitrile=20:1-1:20) to obtain compound 43f (20 mg, yield: 11%). m/z (ESI): 656.8 [M+H]⁺.

### Step 6: 5-amino-4-(3-(benzyloxy)-2,6-dimethylphenyl)-8-(2-methoxyethyl)-2-methyl-7,8-dihydro-1,3,4,7,8,9-hexaazabenzo[cd]cyclopenta[f]azulen-6(4H)-one (43g)

Compound 43f (20 mg, 31 μmol) was dissolved in anhydrous methanol under an ice bath. Acetyl chloride (5 mL) was then slowly added dropwise. After the addition was completed, the mixture was warmed to room temperature and reacted for 4 h. The mixture was concentrated in vacuum to remove the solvent. The solid residue was then dissolved in tetrahydrofuran/water (5 mL/5 mL). Lithium hydroxide monohydrate (84 mg, 2 mmol) was then added. The mixture was reacted at 60° C. for 8 h. The mixture was concentrated in vacuum to remove the solvent. The resulting residue was purified by reverse-phase column chromatography (C18, 0.05% aqueous ammonia:acetonitrile=20:1-1:20) to obtain compound 43g (12 mg, yield: 75%). m/z (ESI): 524.6 [M+H]⁺.

### Step 7: preparation of 5-amino-4-(3-hydroxy-2,6-dimethylphenyl)-8-(2-methoxyethyl)-2-methyl-7,8-dihydro-1,3,4,7,8,9-hexaazabenzo[cd]cyclopenta[f]azulen-6(4H)-one (Compound 43)

Compound 43g (12 mg, 23 μmol) was dissolved in methanol (5 mL). Palladium/carbon (10 mg, 10% Pd, containing approximately 55% water) was then added. The system was purged with hydrogen 3 times. The mixture was reacted at room temperature for 2 h. The reaction mixture was filtered, and the filtrate was concentrated. The resulting residue was purified by reverse-phase column chromatography (C18, 0.05% aqueous ammonia:acetonitrile=20:1-1:20) to obtain compound 43 (7 mg, yield: 70%). m/z (ESI): 434.4 [M+H]⁺.

¹H NMR (400 MHz, DMSO-d₆) δ 9.62 (s, 1H), 9.53 (s, 1H), 8.10 (s, 1H), 7.08 (d, J=8.3 Hz, 1H), 7.00-6.84 (m, 3H), 4.14 (t, J=5.2 Hz, 2H), 3.66 (t, J=5.2 Hz, 2H), 3.26 (s, 3H), 2.36 (s, 3H), 1.79 (s, 3H), 1.70 (s, 3H).

### Example 44. Preparation of 5-amino-4-(3-hydroxy-2,6-dimethylphenyl)-1,2,9-trimethyl-7,9-dihydro-3,4,7,9,10-pentaazabenzo[cd]cyclopenta[f]azulen-6(4H)-one (Compound 44)

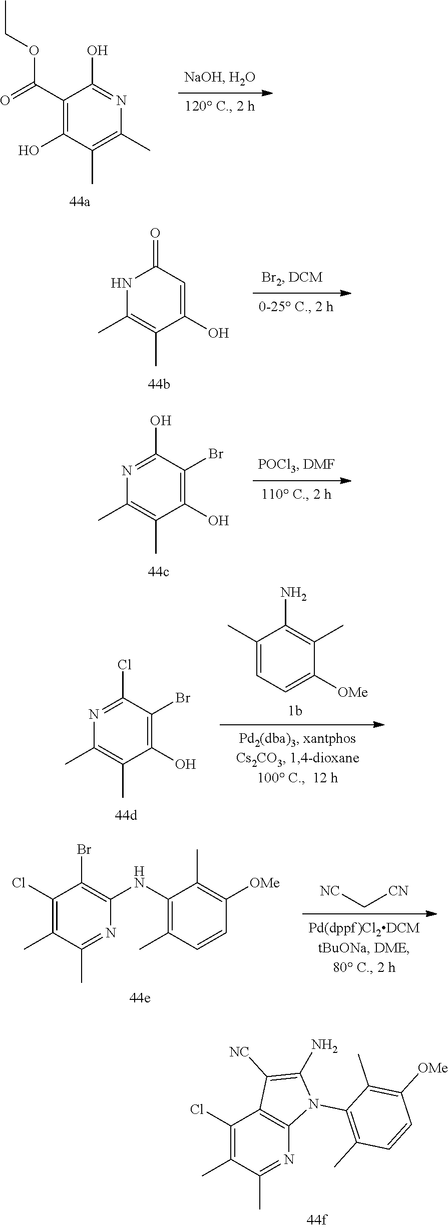

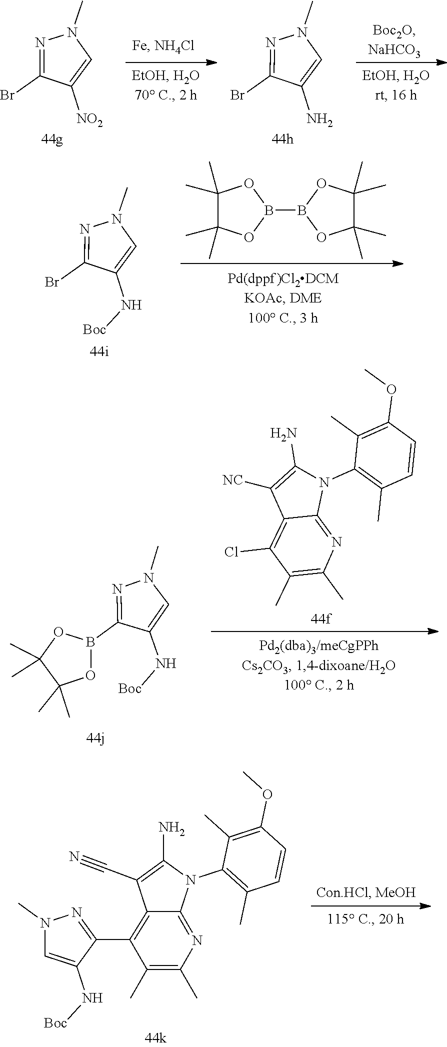

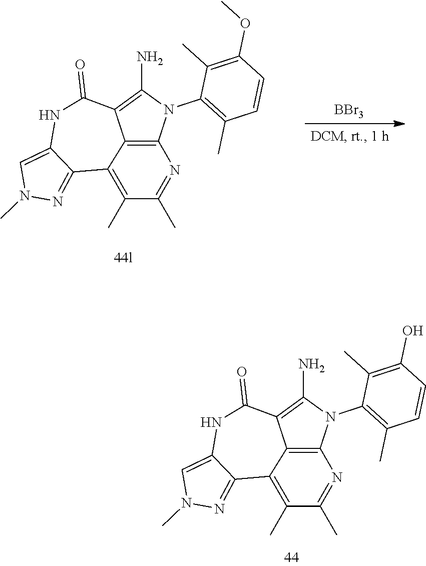 [SMILES: COc1ccc(C)c(-n2c(N)c3c4c(c(C)c(C)nc42)-c2nn(C)cc2NC3=O)c1C ; "BBr3" ; Cc1ccc(O)c(C)c1-n1c(N)c2c3c(c(C)c(C)nc31)-c1nn(C)cc1NC2=O]

### Step 1: 4-hydroxy-5,6-dimethylpyridin-2(1H)-one (44b)

Compound 44a (30 g, 142 mmol) was dissolved in water (300 mL). Sodium hydroxide (57 g, 1.4 mol) was then added. The mixture was then warmed to 120° C. and reacted for 2 h. After the reaction was cooled to room temperature, the reaction mixture was neutralized to pH=7 with 6 N hydrochloric acid. The mixture was filtered, and the filter cake was washed with water and dried to obtain compound 44b (16 g, yield: 80%). m/z (ESI): 140.0 [M+H]⁺.

### Step 2: 3-bromo-5,6-dimethylpyridine-2,4-diol (44c)

Compound 44b (11.5 g, 82.6 mmol) was dissolved in anhydrous dichloromethane (50 mL). Liquid bromine (13.2 g, 82.6 mmol) was then slowly added dropwise under an ice bath. After the addition was completed, the mixture was warmed to room temperature and reacted for 2 h. The reaction was quenched with a saturated aqueous sodium thiosulfate solution (10 mL). The mixture was filtered, and the filter cake was washed with dichloromethane. The solid was dried to obtain the product compound 44c (13 g, yield: 73%). m/z (ESI): 217.9 [M+H]⁺.

### Step 3: 3-bromo-2,4-dichloro-5,6-dimethylpyridine (44d)

Compound 44c (9.9 g, 45 mmol) was dissolved in phosphorus oxychloride (6 mL). N,N-Dimethylformamide (0.16 g, 2.3 mmol) was then added dropwise. The mixture was warmed to 110° C. and reacted for 2 h. The reaction mixture was concentrated to remove most of the phosphorus oxychloride. Water (100 mL) was then added, and the mixture was stirred for 30 min and extracted with ethyl acetate (200 mL×3). The organic phases were combined, washed with saturated brine (100 mL), dried over anhydrous sodium sulfate, and filtered. The filtrate was concentrated, and the resulting residue was purified by normal-phase column chromatography (petroleum ether:ethyl acetate=10:1-1:1) to obtain compound 44d (7.1 g, yield: 62%). m/z (ESI): 253.8 [M+H]⁺.

### Step 4: 3-bromo-4-chloro-N-(3-methoxy-2,6-dimethylphenyl)-5,6-dimethylpyridin-2-amine (44e)

Compound 44d (1.0 g, 3.9 mmol), 3-methoxy-2,6-dimethylaniline (1b, 0.88 g, 5.9 mmol), 4,5-bis(diphenylphosphino)-9,9-dimethylxanthene (0.23 g, 0.39 mmol), tris(dibenzylideneacetone)dipalladium (0.36 g, 0.39 mmol), and cesium carbonate (2.6 g, 7.9 mmol) were dissolved in anhydrous 1,4-dioxane. The system was purged with nitrogen 3 times. The mixture was then reacted at 100° C. for 12 h under nitrogen atmosphere. The reaction mixture was cooled to room temperature and then filtered to remove the insoluble substance, and the filtrate was concentrated to dryness by rotary evaporation. The resulting residue was purified by reverse-phase column chromatography (C18, 0.05% aqueous ammonia:acetonitrile=20:1-1:20) to obtain compound 44e (0.25 g, yield: 17%). m/z (ESI): 369.0 [M+H]⁺.

### Step 5: 2-amino-4-chloro-1-(3-methoxy-2,6-dimethylphenyl)-5,6-dimethyl-1H-pyrrolo[2,3-b]pyridine-3-carbonitrile (44f)

Malononitrile (0.18 g, 2.7 mmol) was dissolved in anhydrous ethylene glycol dimethyl ether (12 mL). Sodium tert-butoxide (0.39 g, 4.1 mmol) was added. After the mixture was stirred at room temperature for 30 min, compound 44e (0.25 g, 0.67 mmol) and [1,1′-bis(diphenylphosphino)ferrocene]palladium dichloride dichloromethane complex (54 mg, 67 μmol) were added. The system was purged with nitrogen 3 times, and the mixture was reacted at 80° C. for 2 h under nitrogen atmosphere. The reaction mixture was cooled to room temperature and then filtered to remove the insoluble substance, and the filtrate was concentrated to dryness by rotary evaporation. The resulting residue was purified by reverse-phase column chromatography (C18, 0.05% aqueous ammonia:acetonitrile=20:1-1:20) to obtain compound 44f (0.12 g, yield: 46%). m/z (ESI): 355.2 [M+H]⁺.

### Step 6: 3-bromo-1-methyl-1H-pyrazol-4-amine (44h)

Compound 44g (1.0 g, 4.8 mmol) and ammonium chloride (1.6 g, 29 mmol) were dissolved in a mixed solution of ethanol (15 mL) and water (15 mL) at room temperature. Iron powder (1.6 g, 29 mmol) was then added. The mixture was warmed to 70° C., reacted for 2 h, filtered to remove the insoluble substance, and washed with ethanol. The filtrate was concentrated, and the resulting crude product, compound 44h, could be directly used in the next step without purification. m/z (ESI): 175.9 [M+H]⁺.

### Step 7: tert-butyl (3-bromo-1-methyl-1H-pyrazol-4-yl)carbamate (44i)

Compound 44h (0.85 g, 4.8 mmol) and sodium bicarbonate (3.3 g, 39 mmol) were dissolved in a mixed solution of ethanol (25 mL) and water (25 mL). Di-tert-butyl dicarbonate (1.6 g, 7.2 mmol) was then added. The mixture was reacted at room temperature for 16 h. The reaction mixture was concentrated to remove ethanol and then extracted with ethyl acetate (50 mL×3). The organic phases were combined, washed with saturated brine (20 mL), dried over anhydrous sodium sulfate, and filtered. The filtrate was concentrated, and the resulting residue was purified by normal-phase column chromatography (petroleum ether:ethyl acetate=10:1-3:1) to obtain the product compound 44i (0.70 g, yield: 52%). m/z (ESI): 275.9 [M+H]⁺.

### Step 8: tert-butyl (1-methyl-3-(4,4,5,5-tetramethyl-1,3,2-dioxaborolan-2-yl)-1H-pyrazol-4-yl)carbamate (44j)

Compound 44i (0.25 g, 0.91 mmol), bis(pinacolato)diboron (0.25 g, 1.0 mmol), [1,1′-bis(diphenylphosphino)ferrocene]palladium dichloride dichloromethane complex (67 mg, 91 μmol), and potassium acetate (0.18 g, 1.8 mmol) were dissolved in anhydrous ethylene glycol dimethyl ether (10 mL) at room temperature. The system was purged with nitrogen 3 times, and the mixture was reacted at 100° C. for 3 h under nitrogen atmosphere. The reaction mixture was cooled to room temperature and then filtered. The filtrate was concentrated. The resulting residue, compound 44j, could be directly used in the next step without purification.

### Step 9: tert-butyl (3-(2-amino-3-cyano-1-(3-methoxy-2,6-dimethylphenyl)-5,6-dimethyl-1H-pyrrolo[2,3-b]pyridin-4-yl)-1-methyl-1H-pyrazol-4-yl)carbamate (44k)

The crude product compound 44j (0.25 g, 0.77 mmol) from the previous step, compound 44f (90 mg, 0.26 mmol), tris(dibenzylideneacetone)dipalladium (24 mg, 26 μmol), 1,3,5,7-tetramethyl-6-phenyl-2,4,8-trioxa-6-phosphaadamantane (30 mg, 0.10 mmol), and cesium carbonate (0.17 g, 0.52 mmol) were dissolved in 1,4-dioxane/water (5 mL/1 mL) at room temperature. The system was purged with nitrogen 3 times. The reaction mixture was then moved to 100° C. and reacted for 2 h. After the reaction was cooled to room temperature, the reaction mixture was diluted with water (10 mL) and extracted with ethyl acetate (20 mL×3). The organic phases were combined, washed with saturated brine (20 mL), dried over anhydrous sodium sulfate, and filtered. The filtrate was concentrated. The resulting residue was purified by normal-phase column chromatography (petroleum ether:ethyl acetate=10:1-4:1) to obtain the target product compound 44k (70 mg, yield: 52%). m/z (ESI): 516.3 [M+H]⁺.

### Step 10: 5-amino-4-(3-methoxy-2,6-dimethylphenyl)-1,2,9-trimethyl-7,9-dihydro-3,4,7,9,10-pentaazabenzo[cd]cyclopenta[f]azulen-6(4H)-one (44l)

Compound 44k (70 mg, 0.14 mmol) was dissolved in methanol (5 mL) at room temperature. Concentrated hydrochloric acid (5 mL) was then added. The reaction mixture was reacted at 115° C. in a sealed tube for 20 h and concentrated under reduced pressure to remove the solvent. The resulting residue was purified by reverse-phase column chromatography (C18, 0.05% aqueous ammonia:acetonitrile=20:1-1:20) to obtain the target compound 44l (3 mg, yield: 5%). m/z (ESI): 417.4 [M+H]⁺.

### Step 11: 5-amino-4-(3-hydroxy-2,6-dimethylphenyl)-1,2,9-trimethyl-7,9-dihydro-3,4,7,9,10-pentaazabenzo[cd]cyclopenta[f]azulen-6(4H)-one (44)

Compound 44l (3.0 mg, 7.2 μmol) was dissolved in dichloromethane (3 mL) at room temperature. Boron tribromide (180 mg, 72 μmol) was then added, and the mixture was stirred for 1 h. The reaction mixture was quenched with methanol (5 mL) and then concentrated. The resulting residue was purified by reverse-phase column chromatography (C18, 0.05% aqueous ammonia:acetonitrile=20:1-1:20) to obtain compound 44 (2.3 mg, yield: 79%).

m/z (ESI): 403.4 [M+H]⁺.

¹H NMR (400 MHz, DMSO-d₆) δ 9.49 (s, 1H), 8.94 (s, 1H), 7.29 (s, 1H), 7.05 (d, J=8.0 Hz, 1H), 6.90 (d, J=8.0 Hz, 1H), 6.76 (s, 2H), 3.79 (s, 3H), 2.57 (s, 3H), 2.27 (s, 3H), 1.78 (s, 3H), 1.69 (s, 3H).

### Example 45. Preparation of 2-amino-1-(3-hydroxy-2,6-dimethylphenyl)-11-methyl-3-oxo-1,3,4,5-tetrahydro-1,4,7,10,12-pentaazabenzo[4,5]cyclooctatetraeno[1,2,3-cd]indene-6-carbonitrile (Compound 45)

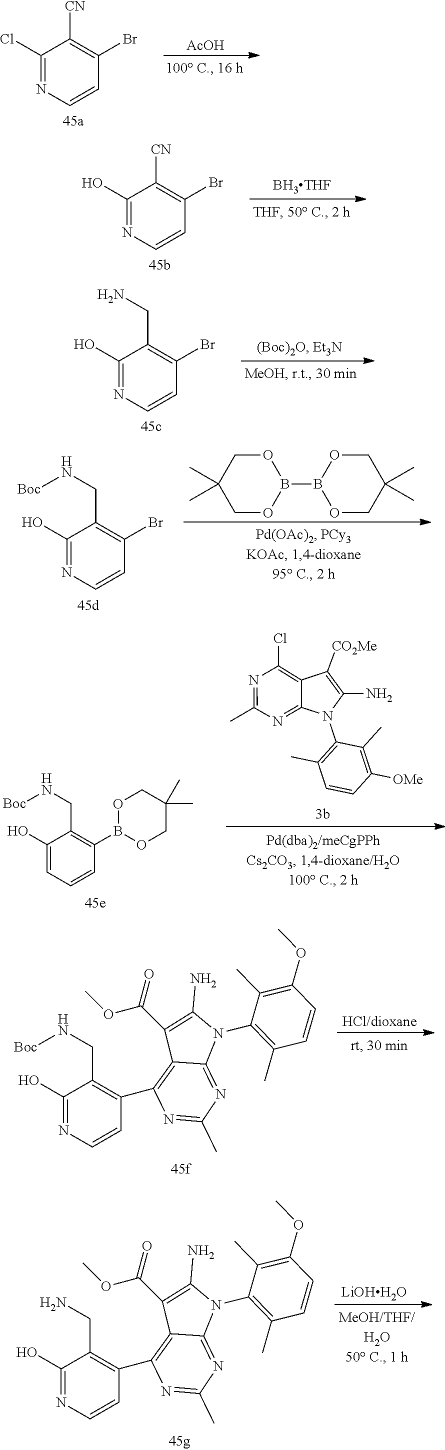

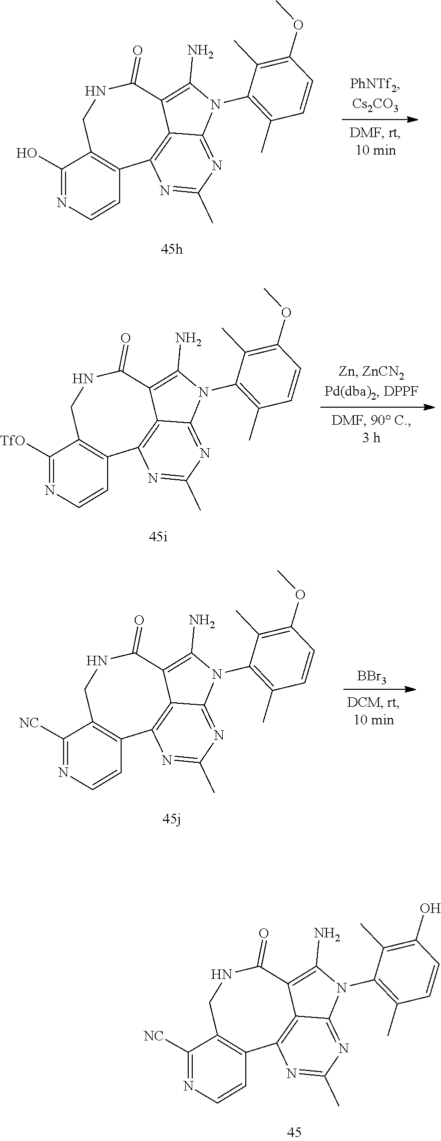

### Step 1: 4-bromo-2-hydroxynicotinonitrile (45b)

Compound 45a (2.0 g, 9.2 mmol) was dissolved in glacial acetic acid (30 mL). The mixture was heated to 100° C. and reacted for 16 h. The reaction mixture was cooled to room temperature and then filtered to remove the insoluble substance, and the filtrate was concentrated. The resulting residue was purified by reverse-phase column chromatography (C18, 0.05% formic acid:acetonitrile=20:1-1:20) to obtain compound 45b (1.5 g, yield: 82%). m/z (ESI): 199.0 [M+H]⁺.

### Step 2: 3-(aminomethyl)-4-bromopyridine-2-ol (45c)

Compound 45b (1.5 g, 7.5 mmol) was dissolved in anhydrous tetrahydrofuran (30 mL). A solution of borane-tetrahydrofuran complex in tetrahydrofuran (1 mol/L, 15 mL) was then added. The system was purged with nitrogen 3 times. The reaction mixture was moved to 50° C. and reacted for 2 h. After the reaction was cooled to room temperature, diluted hydrochloric acid (1 mol/L, 50 mL) was added. The mixture was stirred at room temperature for 20 min. The organic phase was separated and discarded. The aqueous phase was concentrated, and the resulting crude product, compound 45c, could be directly used in the next step without purification. m/z (ESI): 203.0 [M+H]⁺.

### Step 3: tert-butyl ((4-bromo-2-hydroxypyridin-3-yl)methyl)carbamate (45d)

Compound 45c (1.5 g, 7.5 mmol) was dissolved in anhydrous methanol (30 mL). Triethylamine (1.5 g, 15 mmol) and di-tert-butyl dicarbonate (3.2 g, 15 mmol) were then added. The mixture was reacted at room temperature for 30 min. The reaction mixture was concentrated. The resulting residue was purified by reverse-phase column chromatography (C18, 0.05% aqueous ammonia:acetonitrile=20:1-1:20) to obtain compound 45d (0.88 g, yield: 40%). m/z (ESI): 303.0 [M+H]⁺.

### Step 4: tert-butyl ((4-(5,5-dimethyl-1,3,2-dioxaborinan-2-yl)-2-hydroxypyridin-3-yl)methyl)carbamate (45e)

Compound 45d (0.15 g, 0.5 mmol), bis(neopentyl glycolato)diboron (0.17 g, 0.75 mmol), palladium acetate (22 mg, 0.10 mmol), tricyclohexylphosphine (28 mg, 0.10 mmol), and potassium acetate (98 mg, 1.0 mmol) were dissolved in anhydrous 1,4-dioxane (5 mL). The system was purged with argon, and the mixture was reacted at 95° C. for 2 h under argon atmosphere. The reaction mixture was cooled to room temperature, and then filtered to remove the insoluble substance. The resulting product, compound 45e, could be directly used in the next step without purification. m/z (ESI): 337.2 [M+H]⁺.

### Step 5: methyl 6-amino-4-(3-(((tert-butoxycarbonyl)amino)methyl)-2-hydroxypyridin-4-yl)-7-(3-methoxy-2,6-dimethylphenyl)-2-methyl-7H-pyrrolo[2,3-d]pyrimidine-5-carboxylate (45f)

Compound 45e (0.11 g, 0.31 mmol), compound 3b (0.10 g, 0.28 mmol), bis(dibenzylideneacetone)palladium (24 mg, 43 μmol), 1,3,5,7-tetramethyl-6-phenyl-2,4,8-trioxa-6-phosphaadamantane (12 mg, 43 μmol), and cesium carbonate (0.14 g, 0.43 mmol) were dissolved in 1,4-dioxane/water (5 mL/0.5 mL). The system was purged with nitrogen 3 times. The reaction mixture was moved to 100° C. and reacted for 2 h. The reaction mixture was cooled to room temperature and then filtered to remove the insoluble substance, and the filtrate was concentrated. The resulting residue was purified by reverse-phase column chromatography (C18, 0.05% aqueous ammonia:acetonitrile=20:1-1:20) to obtain compound 45f (42 mg, yield: 24%). m/z (ESI): 563.3 [M+H]⁺.

### Step 6: methyl 6-amino-4-(3-(aminomethyl)-2-hydroxypyridin-4-yl)-7-(3-methoxy-2,6-dimethylphenyl)-2-methyl-7H-pyrrolo[2,3-d]pyrimidine-5-carboxylate (45g)

Compound 45f (42 mg, 75 μmol) was dissolved in a solution of hydrogen chloride in dioxane (4 mol/L, 2 mL), and the mixture was reacted at room temperature for 30 min. After the reaction was completed, the reaction mixture was concentrated. The resulting product, compound 45g, could be directly used in the next step without purification. m/z (ESI): 463.2 [M+H]⁺.

### Step 7: 2-amino-6-hydroxy-1-(3-methoxy-2,6-dimethylphenyl)-11-methyl-4,5-dihydro-1,4,7,10,12-pentaazabenzo[4,5]cyclooctatetraeno[1,2,3-cd]inden-3(1H)-one (45h)

Compound 45g (35 mg, 75 μmol) was dissolved in methanol/tetrahydrofuran/water (2 mL/2 mL/2 mL), and lithium hydroxide monohydrate (42 mg, 1.0 mmol) was added. The reaction mixture was transferred to 50° C. and reacted for 1 h. The reaction mixture was concentrated. The resulting residue was purified by reverse-phase column chromatography (C18, 0.05% aqueous ammonia:acetonitrile=20:1-1:20) to obtain compound 45h (25 mg, yield: 78%). m/z (ESI): 431.2 [M+H]⁺.

### Step 8: 2-amino-1-(3-methoxy-2,6-dimethylphenyl)-11-methyl-3-oxo-1,3,4,5-tetrahydro-1,4,7,10,12-pentaazabenzo[4,5]cyclooctatetraeno[1,2,3-cd]inden-6-yl trifluoromethanesulfonate (45i)

Compound 45h (5.0 mg, 12 μmol) was dissolved in anhydrous N,N-dimethylformamide (5 mL). Cesium carbonate (4.2 mg, 13 μmol) and N-phenylbis(trifluoromethanesulfonyl)imide (4.4 mg, 12 μmol) were sequentially added. The mixture was reacted at room temperature for 10 min. The reaction mixture was directly purified by reverse-phase column chromatography (C18, 0.05% aqueous ammonia:acetonitrile=20:1-1:20) to obtain the target compound 45i (5.8 mg, yield: 89%). m/z (ESI): 563.2 [M+H]⁺.

### Step 9: 2-amino-1-(3-methoxy-2,6-dimethylphenyl)-11-methyl-3-oxo-1,3,4,5-tetrahydro-1,4,7,10,12-pentaazabenzo[4,5]cyclooctatetraeno[1,2,3-cd]indene-6-carbonitrile (45j)

Compound 45i (5.0 mg, 8.9 μmol) was dissolved in anhydrous N,N-dimethylformamide (5 mL). Zinc (0.6 mg, 8.9 μmol), zinc cyanide (1.0 mg, 8.9 μmol), tris(dibenzylideneacetone)dipalladium (0.8 mg, 0.89 μmol), and 1,1′-bis(diphenylphosphino)ferrocene (0.20 mg, 0.17 μmol) were sequentially added. The system was purged with nitrogen 3 times, and the reaction mixture was moved to 90° C. and reacted for 3 h. The reaction mixture was cooled to room temperature and then directly purified by reverse-phase column chromatography (C18, 0.05% aqueous ammonia:acetonitrile=20:1-1:20) to obtain the target compound 45j (3.2 mg, yield: 82%).

### Step 10: 2-amino-1-(3-hydroxy-2,6-dimethylphenyl)-11-methyl-3-oxo-1,3,4,5-tetrahydro-1,4,7,10,12-pentaazabenzo[4,5]cyclooctatetraeno[1,2,3-cd]indene-6-carbonitrile (45)

Compound 45j (3.2 mg, 7.3 μmol) was dissolved in dichloromethane (5 mL) at room temperature. A solution of boron tribromide (1 mol/L, 2 mL) in dichloromethane was added, and the mixture was stirred for 10 min. The reaction mixture was quenched with methanol (5 mL) and then concentrated. The resulting residue was purified by reverse-phase column chromatography (C18, 0.05% aqueous ammonia:acetonitrile=20:1-1:20) to obtain compound 45 (1.0 mg, yield: 32%). m/z (ESI): 426.4 [M+H]⁺.

¹H NMR (400 MHz, DMSO-d₆) δ 9.62 (d, J=8.4 Hz, 1H), 8.81 (d, J=4.8 Hz, 1H), 8.23 (t, J=6.8 Hz, 1H), 8.03 (dd, J=5.2, 1.2 Hz, 1H), 7.24 (brs, 2H), 7.11 (dd, J=12.0, 8.0 Hz, 1H), 6.96 (d, J=8.0 Hz, 1H), 5.24-5.18 (m, 1H), 4.45-4.39 (m, 1H), 2.51 (s, 3H), 1.90-1.81 (m, 3H), 1.70-1.61 (m, 3H).

### Example 46. Preparation of 2-amino-6-cyclopropyl-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (Compound 46)

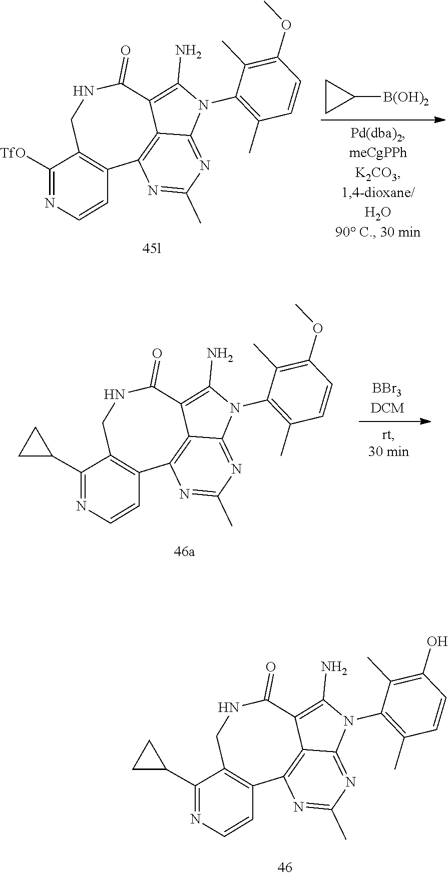

### Step 1: 2-amino-6-cyclopropyl-1-(3-methoxy-2,6-dimethylphenyl)-11-methyl-4,5-dihydro-1,4,7,10,12-pentaazabenzo[4,5]cyclooctatetraeno[1,2,3-cd]inden-3(1H)-one (46a)

Compound 45i (8.6 mg, 15 μmol), cyclopropylboronic acid (13 mg, 0.15 mmol), bis(dibenzylideneacetone)palladium (0.87 mg, 1.5 mol), 1,3,5,7-tetramethyl-6-phenyl-2,4,8-trioxa-6-phosphaadamantane (0.89 mg, 3.1 μmol), and potassium carbonate (6.3 mg, 46 μmol) were dissolved in 1,4-dioxane/water (5 mL/0.5 mL). The system was purged with nitrogen 3 times. The reaction mixture was then moved to 90° C. and reacted for 0.5 h. The reaction mixture was cooled to room temperature and then filtered, and the filtrate was concentrated. The resulting residue was purified by reverse-phase column chromatography (C18, 0.05% aqueous ammonia:acetonitrile=20:1-1:20) to obtain compound 46a (3.6 mg, yield: 52%). m/z (ESI): 455.4 [M+H]⁺.

### Step 2: 2-amino-6-cyclopropyl-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (Compound 46)

Compound 46a (3.6 mg, 7.3 μmol) was dissolved in anhydrous dichloromethane (5 mL). A solution of boron tribromide (1 mol/L, 2 mL) in dichloromethane was added, and the mixture was reacted at room temperature for 30 min. The reaction mixture was quenched with methanol (5 mL), adjusted to pH=14 with aqueous ammonia, and then concentrated. The resulting residue was purified by reverse-phase column chromatography (C18, 0.05% aqueous ammonia:acetonitrile=20:1-1:20) to obtain compound 46 (2.2 mg, yield: 63%).

m/z (ESI): 441.4 [M+H]⁺;

¹H NMR (400 MHz, DMSO-d₆) δ 9.61 (d, J=9.6 Hz, 1H), 8.45 (d, J=5.2 Hz, 1H), 8.12-8.08 (m, 1H), 7.44-7.40 (m, 1H), 7.21-7.08 (m, 3H), 6.96 (d, J=8.4 Hz, 1H), 5.16-5.06 (m, 1H), 4.52-4.48 (m, 1H), 2.53 (s, 3H), 2.05-1.95 (m, 1H), 1.91-1.82 (m, 3H), 1.70-1.61 (m, 3H), 0.90-0.78 (m, 4H).

### Example 47. Preparation of 2-amino-6-ethoxy-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (Compound 47)

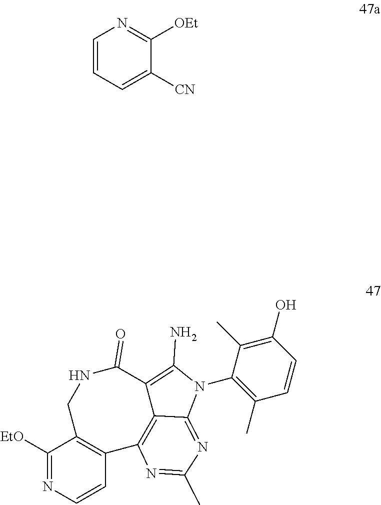 [SMILES: CCOc1ncccc1C#N ; CCOc1nccc2c1CNC(=O)c1c(N)n(-c3c(C)ccc(O)c3C)c3nc(C)nc-2c13]

Compound 47 was prepared by replacing compound 23a with compound 47a and using a method similar to that in Example 23.

m/z (ESI): 445.4 [M+H]⁺;

¹H NMR (400 MHz, DMSO-d₆) δ 9.60 (brs, 1H), 8.18 (d, J=5.2 Hz, 1H), 7.92 (t, J=6.8 Hz, 1H), 7.24 (dd, J=8.4, 1.2 Hz, 1H), 7.12-7.08 (m, 3H), 6.95 (d, J=8.4 Hz, 1H), 4.84-4.78 (m, 1H), 4.40 (q, J=6.8 Hz, 2H), 4.33-4.28 (m, 1H), 2.53 (s, 3H), 1.89-1.80 (m, 3H), 1.69-1.60 (m, 3H), 1.41 (t, J=6.8 Hz, 3H).

### Example 48. Preparation of 2-amino-6-hydroxy-1-(3-hydroxy-2,6-dimethylphenyl)-11-methyl-4,5-dihydro-1,4,7,10,12-pentaazabenzo[4,5]cyclooctatetraeno[1,2,3-cd]inden-3(1H)-one (Compound 48)

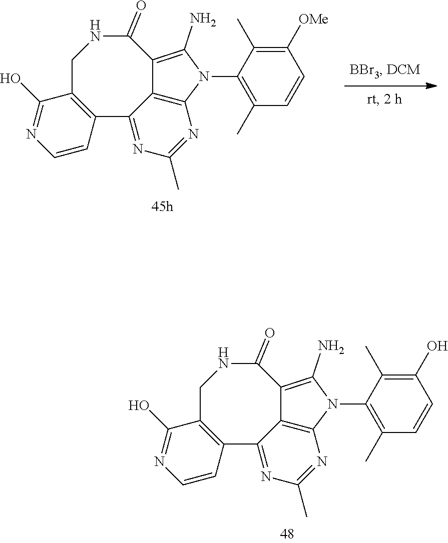 [SMILES: COc1ccc(C)c(-n2c(N)c3c4c(nc(C)nc42)-c2ccnc(O)c2CNC3=O)c1C ; Cc1nc2c3c(c(N)n(-c4c(C)ccc(O)c4C)c3n1)C(=O)NCc1c-2ccnc1O]

Compound 45h (5.0 mg, 12 μmol) was dissolved in anhydrous dichloromethane (5 mL). Boron tribromide (0.50 mL, 1 mol/L dichloromethane solution) was slowly added dropwise under an ice bath. The mixture was reacted at room temperature for 2 h. Methanol (5 mL) was added to quench the reaction. The solvent was removed by distillation under reduced pressure. The pH was adjusted to neutral with aqueous ammonia (25% aqueous ammonia solution). The mixture was purified by reverse-phase column chromatography (C18, 0.05% aqueous ammonia:acetonitrile=20:1-1:20) to obtain compound 48 (4 mg, yield: 80%). m/z (ESI): 417.4 [M+H]⁺.

¹H NMR (400 MHz, DMSO-d₆) δ 7.69 (t, J=6.8 Hz, 1H), 7.41 (d, J=6.8 Hz, 1H), 7.14-7.02 (m, 3H), 6.94 (d, J=8.2 Hz, 1H), 6.47 (m, 1H), 4.68-4.46 (m, 1H), 4.35-4.17 (m, 1H), 2.48 (s, 3H), 1.89-1.75 (m, 3H), 1.72-1.55 (m, 3H).

### Example 49. Preparation of 2-amino-7-fluoro-1-(3-hydroxy-2,6-dimethylphenyl)-11-methyl-3-oxo-1,3,4,5-tetrahydro-1,4,10,12-tetraazabenzo[4,5]cyclooctatetraeno[1,2,3-cd]inden-6-carbonitrile (Compound 49)

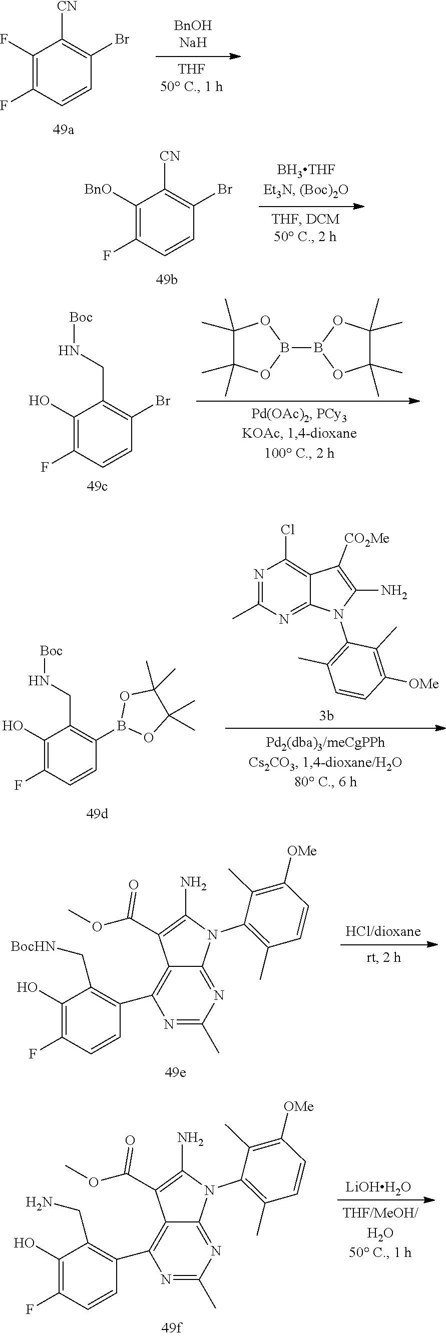

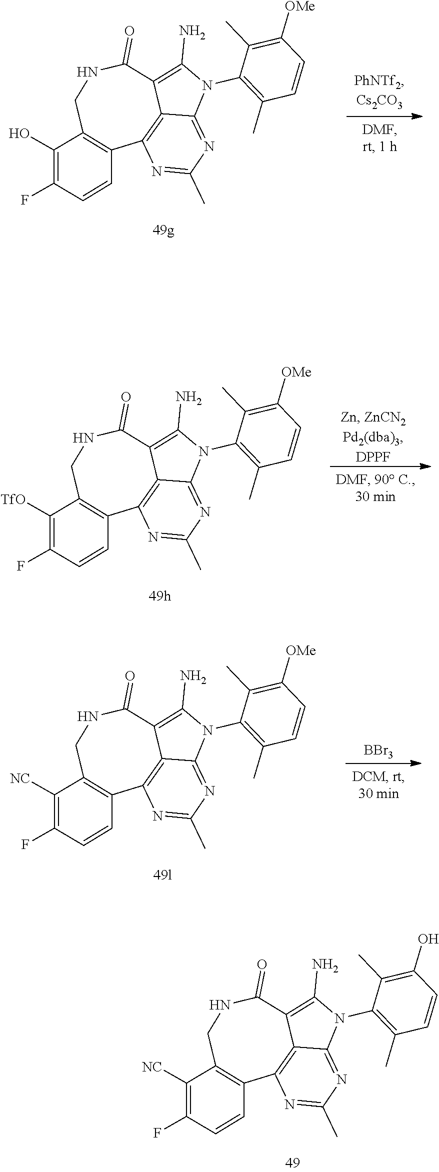

### Step 1: 2-(benzyloxy)-6-bromo-3-fluorobenzonitrile (49b)

Benzyl alcohol (0.25 g, 2.3 mmol) was dissolved in anhydrous N,N-dimethylformamide (3 mL) under an ice bath. Sodium hydride (60%, 0.12 g, 3.0 mmol) was then added. After the mixture was reacted for 30 min, compound 49a (0.50 g, 2.3 mmol) was added. The mixture was then warmed to 50° C. and reacted for 1 h. Water (10 mL) was added to the reaction system to quench the reaction. The mixture was extracted with ethyl acetate (50 mL×3). The organic phases were combined, washed with saturated brine (30 mL×3), dried over anhydrous sodium sulfate, and filtered. The filtrate was concentrated, and the resulting residue was purified by reverse-phase column chromatography (C18, 0.05% aqueous ammonia:acetonitrile=20:1-1:20) to obtain the product compound 49b (0.51 g, yield: 72%).

### Step 2: tert-butyl (6-bromo-3-fluoro-2-hydroxybenzyl)carbamate (49c)

Compound 49b (0.20 g, 0.65 mmol) was dissolved in anhydrous tetrahydrofuran (5 mL) at room temperature. A solution of borane-tetrahydrofuran complex in tetrahydrofuran (3.3 mL, 1 M, 3.27 mmol) was then added. The system was purged with nitrogen 3 times. The reaction mixture was moved to 50° C. and reacted for 2 h. After the reaction was cooled to room temperature, 1 N hydrochloric acid was added to quench the reaction. The mixture was stirred for 30 min and extracted with ethyl acetate (50 mL×3). The organic phases were combined, concentrated to dryness by rotary evaporation, and dissolved in dichloromethane (10 mL). Triethylamine (0.28 g, 2.7 mmol) and di-tert-butyl dicarbonate (0.40 g, 1.8 mmol) were added at room temperature. The mixture was stirred at room temperature for 30 min. The mixture was filtered, and the filtrate was concentrated. The resulting residue was purified by reverse-phase column chromatography (C18, 0.05% aqueous ammonia:acetonitrile=20:1-1:20) to obtain the target compound 49c (0.12 g, yield: 44%). m/z (ESI): 320.2 [M+H]⁺.

### Step 3: tert-butyl (3-fluoro-2-hydroxy-6-(4,4,5,5-tetramethyl-1,3,2-dioxaborolan-2-yl)benzyl)carbamate (49d)

Compound 49c (50 mg, 0.16 mmol), bis(pinacolato)diboron (79 mg, 0.31 mmol), palladium acetate (3.5 mg, 16 μmol), tricyclohexylphosphine (4.4 mg, 16 μmol), and potassium acetate (46 mg, 0.47 mmol) were dissolved in anhydrous 1,4-dioxane (5 mL). The system was purged with argon, and the mixture was reacted at 100° C. for 2 h under argon atmosphere. The reaction mixture was cooled to room temperature, and then filtered to remove the insoluble substance. The resulting product, 49d, could be directly used in the next step without purification. m/z (ESI): 368.2 [M+H]⁺.

### Step 4: methyl 6-amino-4-(2-(((tert-butoxycarbonyl)amino)methyl)-4-fluoro-3-hydroxyphenyl)-7-(3-methoxy-2,6-dimethylphenyl)-2-methyl-7H-pyrrolo[2,3-d]pyrimidine-5-carboxylate (49e)

Compound 49d (67 mg, 0.18 mmol), compound 3b (45 mg, 0.12 mmol), tris(dibenzylideneacetone)dipalladium (11 mg, 12 μmol), 1,3,5,7-tetramethyl-6-phenyl-2,4,8-trioxa-6-phosphaadamantane (3.5 mg, 12 μmol), and cesium carbonate (117 mg, 0.36 mmol) were dissolved in 1,4-dioxane/water (5 mL/0.5 mL) at room temperature. The system was purged with nitrogen 3 times. The reaction mixture was moved to 80° C. and reacted for 6 h. The reaction mixture was cooled to room temperature and then filtered to remove the insoluble substance, and the filtrate was concentrated. The resulting residue was purified by reverse-phase column chromatography (C18, 0.05% aqueous ammonia:acetonitrile=20:1-1:20) to obtain the target compound 49e (31 mg, yield: 45%). m/z (ESI): 580.2 [M+H]⁺.

### Step 5: methyl 6-amino-4-(2-(aminomethyl)-4-fluoro-3-hydroxyphenyl)-7-(3-methoxy-2,6-dimethylphenyl)-2-methyl-7H-pyrrolo[2,3-d]pyrimidine-5-carboxylate (49f)

At room temperature, compound 49e (25 mg, 43 μmol) was dissolved in a solution of hydrochloric acid in 1,4-dioxane (2 mL). The mixture was reacted under the condition for 2 h. After the reaction was completed, the reaction mixture was concentrated. The resulting product, 49f, could be directly used in the next step without purification. m/z (ESI): 480.2 [M+H]⁺.

### Step 6: 2-amino-7-fluoro-6-hydroxy-1-(3-methoxy-2,6-dimethylphenyl)-11-methyl-4,5-dihydro-1,4,10,12-tetraazabenzo[4,5]cyclooctatetraeno[1,2,3-cd]inden-3(1H)-one (49g)

Compound 49f (21 mg, 43 μmol) was dissolved in methanol/tetrahydrofuran/water (2 mL/2 mL/2 mL), and lithium hydroxide monohydrate (52 mg, 1.2 mmol) was then added. The reaction mixture was reacted at 50° C. under an oil bath for 1 h. The reaction mixture was concentrated. The resulting residue was purified by reverse-phase column chromatography (C18, 0.05% aqueous ammonia:acetonitrile=20:1-1:20) to obtain the target compound 49g (19 mg, yield: 97%). m/z (ESI): 448.2 [M+H]⁺.

### Step 7: 2-amino-7-fluoro-1-(3-methoxy-2,6-dimethylphenyl)-11-methyl-3-oxo-1,3,4,5-tetrahydro-1,4,10,12-tetraazabenzo[4,5]cyclooctatetraeno[1,2,3-cd]inden-6-yl trifluoromethanesulfonate (49h)

Compound 49g (19 mg, 42 μmol) was dissolved in anhydrous N,N-dimethylformamide (5 mL). Cesium carbonate (28 mg, 85 μmol) and N-phenylbis(trifluoromethanesulfonyl)imide (23 mg, 64 μmol) were sequentially added. The mixture was reacted at room temperature for 1 h. The reaction mixture was directly purified by reverse-phase column chromatography (C18, 0.05% aqueous ammonia:acetonitrile=20:1-1:20) to obtain the target compound 49h (22 mg, yield: 88%). m/z (ESI): 580.2 [M+H]⁺.

### Step 8: 2-amino-7-fluoro-1-(3-methoxy-2,6-dimethylphenyl)-11-methyl-3-oxo-1,3,4,5-tetrahydro-1,4,10,12-tetraazabenzo[4,5]cyclooctatetraeno[1,2,3-cd]indene-6-carbonitrile (49i)

Compound 49h (10 mg, 17 μmol) was dissolved in anhydrous N,N-dimethylformamide (3 mL). Zinc (0.6 mg, 8.7 μmol), zinc cyanide (2 mg, 17 μmol), tris(dibenzylideneacetone)dipalladium (1.6 mg, 1.8 μmol), and 1,1′-bis(diphenylphosphino)ferrocene (1 mg, 1.8 μmol) were sequentially added. The system was purged with nitrogen 3 times, and the reaction mixture was moved to 90° C. and reacted for 30 min. The reaction mixture was cooled to room temperature and then directly purified by reverse-phase column chromatography (C18, 0.05% aqueous ammonia:acetonitrile=20:1-1:20) to obtain the target compound 49i (6 mg, yield: 76%). m/z (ESI): 457.2 [M+H]⁺.

### Step 9: 2-amino-7-fluoro-1-(3-methoxy-2,6-dimethylphenyl)-11-methyl-3-oxo-1,3,4,5-tetrahydro-1,4,10,12-tetraazabenzo[4,5]cyclooctatetraeno[1,2,3-cd]indene-6-carbonitrile (49)

Compound 49i (6 mg, 14 μmol) was dissolved in dichloromethane (3 mL) at room temperature. A solution of boron tribromide (1 mol/L, 1 mL) in dichloromethane was added, and the mixture was stirred for 30 min. The reaction mixture was quenched with methanol (5 mL), adjusted to pH=14 with aqueous ammonia, and then concentrated. The resulting residue was purified by reverse-phase column chromatography (C18, 0.05% aqueous ammonia:acetonitrile=20:1-1:20) to obtain compound 49 (4.5 mg, yield: 71%).

m/z (ESI): 443.3 [M+H]⁺.

¹H NMR (400 MHz, DMSO-d₆) δ 9.60 (s, 1H), 8.16-8.13 (m, 2H), 7.63 (t, J=8.8 Hz, 1H), 7.13-7.07 (m, 3H), 6.95 (d, J=8.0 Hz, 1H), 5.20-5.10 (m, 1H), 4.35-4.29 (m, 1H), 2.49 (s, 3H), 1.90-1.81 (m, 3H), 1.70-1.61 (m, 3H).

### TEST EXAMPLES FOR BIOLOGICAL ACTIVITY AND RELATED PROPERTIES

### Test Example 1: Inhibition Assay for PKMYT1 Enzyme Activity

Experimental principle: After co-incubation of PKMYT1 with the compound, the substrate CDK1 was phosphorylated under the action of ATP. ADP produced by the reaction was quantified using an ADP-Glo™ assay kit from Promega to reflect the enzyme activity.

Experimental instrument: Echo550 liquid handler (Labcyte Inc.); Envision microplate reader (Perkin Elmer Inc.); 5810R centrifuge (Eppendorf Inc.).

### Materials:

| Reagent | Brand | Catalog No. |
|---|---|---|
| MYT1, Active | SignalChem | M67-10G-10 |
| CDK1, Unactive | SignalChem | C22-14G-50 |
| Kinase Dilution Buffer IX | SignalChem | K29-09-20 |
| Kinase Assay Buffer III | SignalChem | K03-09-20 |
| ATP | Promega | V915B |
| ADP-Glo kinase detection kit | Promega | V9101 |
| 384-well plate | Perkin Elmer | 6007290 |

Experimental method: Using the Echo liquid handler, the test compound was diluted to different concentrations in dimethyl sulfoxide (DMSO) and transferred to a 384-well plate. Thus, the final concentration of the compound in the reaction system started at 1 μM and was subjected to a 3-fold serial dilution, with the final DMSO concentration maintained at 1%. 50 nL/well of the test compound solution was added. 3 μL/well of MYT1 diluted with Kinase Dilution Buffer was then added. After incubation for 15 min, 2 μL/well of a reaction mixture solution consisting of the substrate CDK1 diluted with Kinase Dilution Buffer and ATP diluted with Kinase Assay Buffer III was then added to initiate the enzyme reaction. The final concentration of the enzyme MYT1 was 2 ng/μL, the final concentration of the ATP was 25 μM, and the final concentration of the substrate CDK1 was 20 ng/μL. After the mixture was reacted for 40 min, 5 μL/well of ADP-Glo reagent was added, and the mixture was incubated for 40 min. 10 μL/well of the kinase reaction detection reagent was then added. After incubation for 30 min, the fluorescence signal values were measured using an Envision microplate reader.

### Data Analysis:

The % compound inhibition was calculated and fitted using XLfit software to obtain the IC₅₀ value of the compound.

A blank group and a DMSO group were set up in the experiment. The reaction system of the blank group was 1% DMSO and the reaction mixture solution, and the compound inhibition rate was considered to be 100% under this condition. The reaction system of the DMSO group was 1% DMSO, PKMYT1 (2 nM/μL), and the reaction mixture solution, and the compound inhibition rate was considered to be 0% under this condition.

$$\% Compound inhibition = ( 100 - 100 \times ( experimental well - blank well ) / ( DMSO group - blank well ) ) \%$$

Herein, the experimental well, blank well, and DMSO group refer to the fluorescence signal values recorded from the experimental group, blank group, and DMSO group, respectively.

The inhibitory activity of the compounds of the present application on PKMYT1 was determined by the above assay, and the IC₅₀ values obtained are as shown in Table 1.

TABLE 1
IC₅₀ values of compounds for PKMYT1 inhibition activity

| Compound No. | IC₅₀ (nM) |
|---|---|
| 1 | 8.8 |
| 2 | 5.7 |

### Test Example 2: Assay on Inhibition of Tumor Cell Proliferation by Compounds

Experimental principle: The compound was incubated with tumor cells for 7 days, and ATP in viable cells was quantified using the CTG kit from Promega to evaluate the effect of the compound on tumor cell proliferation.

Experimental instrument: Envision microplate reader (Perkin Elmer Inc.); 5810R centrifuge (Eppendorf Inc.); automatic cell counter (Countstar Inc.).

### Materials:

| Reagent | Brand | Catalog No. |
|---|---|---|
| HCC1569 cell line | ATCC | CRL-2330 |
| RPMI Medium 1640 | Gibco | A10491-01 |
| FBS | Gibco | 10099-141C |
| 0.25% Trypsin-EDTA | Gibco | 25200-172 |
| PBS | HyClone | SH30256.01 |
| 96-well plate | Corning | 3610 |
| CelltiterGlo assay kit (CTG) | Promega | G7573 |

Experimental method: The cultured cells were resuspended after being rinsed with PBS (for adherent cells) and digested with 0.25% Trypsin-EDTA, followed by cell counting to determine cell density and viability. 3000 HCC1569 cells/well were diluted with RPMI Medium 1640 containing 10% FBS, added to a 96-well plate (90 μL/well), and cultured in an incubator at 37° C. with 5% CO₂ for 24 h. The test compound was diluted to different concentrations in dimethyl sulfoxide (DMSO) and added to a 96-well plate. Thus, the final concentration of the compound in the reaction system started at 25 μM and was subjected to a 4-fold serial dilution, with the final DMSO concentration maintained at 0.25%. The compound and cells were further incubated at 37° C. with 5% CO₂ for 7 days. 50 μL/well of CTG was then added to measure the inhibitory effect of the compound on tumor cell growth, and the inhibition rate and half-maximal inhibitory concentration (IC₅₀) were calculated.

### Data Analysis:

The % compound inhibition was calculated and fitted using XLfit software to obtain the IC₅₀ of the compound.

Blank wells and DMSO wells were set up. The blank well contained 100 μL of RPMI Medium 1640 with 10% FBS, where the inhibition rate of the compound on tumor cell growth was considered 100%. The DMSO well contained 0.25% DMSO added to the cell well, where the inhibition rate of the compound on tumor cell growth was considered 0%.

$$\% Compound inhibition = ( 100 \times ( DMSO well - experimental well ) / ( DMSO well - blank well ) ) \%$$

The inhibition of tumor cell growth by the compounds of the present application was determined by the above assay, and the IC₅₀ values obtained are as shown in Table 2 below.

TABLE 2
IC₅₀ of compounds for inhibition of HCC1569 cell growth

| Compound No. | IC₅₀ (nM) |
|---|---|
| 1 | 42 |
| 2 | 22 |
| 3 | 293 |
| 4 | 240 |
| 5 | 539 |
| 8 | 768 |
| 9 | 210 |
| 11 | 59.2 |
| 12 | 9.2 |
| 13 | 27.3 |
| 14 | 254 |
| 16 | 264 |
| 17 | 24.6 |
| 18 | 11 |
| 19 | 50 |
| 20 | 25.5 |
| 23 | 24.9 |
| 27 | 131 |
| 28 | 70 |
| 29 | 562 |
| 30 | 308 |
| 31 | 297 |
| 32 | 798 |
| 33 | 49 |
| 34 | 9.1 |
| 35 | 40.5 |
| 36 | 63.8 |
| 37 | 33.8 |
| 38 | 92 |
| 39 | 81.9 |
| 40 | 87.6 |
| 41 | 86.1 |
| 42 | 46.3 |
| 43 | 291.3 |
| 44 | 55.2 |
| 45 | 194 |
| 46 | 113 |
| 47 | 32 |
| 49 | 20.9 |

1. A compound of formula (I) or a pharmaceutically acceptable salt thereof, 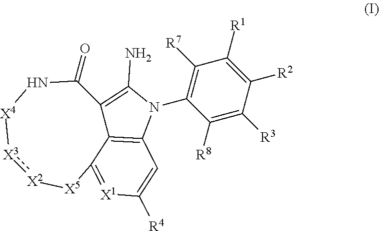
wherein
X¹ is selected from the group consisting of N and CR⁹; [symbol]
represents a single bond or a double bond;
X² is selected from the group consisting of CR⁵R⁶, NR⁵, CR⁵, and N;
X³ is selected from the group consisting of CR⁵′R⁶′, NR⁵′, CR⁵′, and N;
X⁴ and X⁵ are independently selected from the group consisting of (C(R¹⁰)₂)ₙ, NR¹⁰, and O;
R¹ and R³ are independently selected from the group consisting of hydrogen, hydroxy, amino, nitro, halogen, cyano, C₁-C₆ alkyl, C₁-C₆ alkoxy, C₃-C₉ cycloalkyl-O—, 4-9 membered heterocyclyl-O—, C₁-C₆ alkyl-C(O)O—, C₃-C₉ cycloalkyl-C(O)O—, 4-9 membered heterocyclyl-C(O)O—, P(O)(OH)₂O, and NH₂C(O)O—, wherein the C₁-C₆ alkyl, C₁-C₆ alkoxy, C₃-C₉ cycloalkyl-O—, 4-9 membered heterocyclyl-O—, C₁-C₆ alkyl-C(O)O—, C₃-C₉ cycloalkyl-C(O)O—, 4-9 membered heterocyclyl-C(O)O—, P(O)(OH)₂O, or NH₂C(O)O— is optionally substituted with one or more R¹¹;
R² is selected from the group consisting of hydrogen, cyano, halogen, C₁-C₆ alkyl, C₃-C₉ cycloalkyl, 4-9 membered heterocyclyl, C₆-C₁₀ aryl, 5-9 membered heteroaryl, C(═O)H, C₂-C₆ alkenyl, and C₂-C₆ alkynyl, wherein the C₁-C₆ alkyl, C₃-C₉ cycloalkyl, 4-9 membered heterocyclyl, C₆-C₁₀ aryl, 5-9 membered heteroaryl, C(═O)H, C₂-C₆ alkenyl, or C₂-C₆ alkynyl is optionally substituted with one or more R^{a};
or R¹ and R², together with the atoms linked thereto, form a 5-8 membered heterocyclic ring or 5-9 membered heteroaromatic ring;
R⁴, R⁶, R⁶′, and R¹⁰ are independently selected from the group consisting of hydrogen, deuterium, amino, hydroxy, sulfydryl, halogen, cyano, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, and —C(═O)NH₂, wherein the amino, hydroxy, sulfydryl, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, or —C(═O)NH₂ is optionally substituted with one or more R^{a};
R⁵ and R⁵′, together with the atoms linked thereto, form a C₃-C₉ saturated or partially saturated carbon ring, a C₆-C₁₀ aromatic ring, a 5-8 membered heterocyclic ring, or a 5-9 membered heteroaromatic ring, wherein the C₃-C₉ saturated or partially saturated carbon ring, C₆-C₁₀ aromatic ring, 5-8 membered heterocyclic ring, or 5-9 membered heteroaromatic ring is optionally substituted with one or more R^{a};
R⁷ and R⁸ are independently selected from the group consisting of hydrogen, halogen, and C₁-C₆ alkyl, wherein the C₁-C₆ alkyl is optionally substituted with deuterium;
R⁹ is selected from the group consisting of hydrogen, hydroxy, amino, cyano, halogen, C₁-C₆ alkyl, C₃-C₆ cycloalkyl, C₂-C₆ alkenyl, C₂-C₆ alkynyl, and C(═O)H, wherein the hydroxy, amino, C₁-C₆ alkyl, C₃-C₆ cycloalkyl, C₂-C₆ alkenyl, C₂-C₆ alkynyl, or C(═O)H is optionally substituted with one or more R^{a};
R¹¹ is selected from the group consisting of C₁-C₁₀ alkyl, C₃-C₉ cycloalkyl, 4-9 membered heterocyclyl, C₁-C₆ alkoxy, C₃-C₉ cycloalkyl-O—, 4-9 membered heterocyclyl-O—, C₁-C₆ alkyl-C(O)O—, C₃-C₉ cycloalkyl-C(O)O—, 4-9 membered heterocyclyl-C(O)O—, P(O)(OH)₂O, NH₂C(O)O—, and C₁-C₆ alkyl-OC(O)O—;
n is selected from the group consisting of 0, 1, and 2;
R^{a} is independently selected from the group consisting of deuterium, halogen, oxo, hydroxy, amino, cyano, C₁-C₆ alkyl, C₃-C₆ cycloalkyl, 4-7 membered heterocyclyl, C₆-C₁₀ aryl, and 5-9 membered heteroaryl, wherein the hydroxy, amino, C₁-C₆ alkyl, C₃-C₆ cycloalkyl, 4-7 membered heterocyclyl, C₆-C₁₀ aryl, or 5-9 membered heteroaryl is optionally substituted with one or more R^{b};
R^{b} is independently selected from the group consisting of halogen, hydroxy, amino, cyano, C₁-C₃ alkyl, NH(C₁-C₃ alkyl), N(C₁-C₃ alkyl)₂, C₃-C₆ cycloalkyl, 4-7 membered heterocyclyl, C₁-C₆ alkoxy, C₆-C₁₀ aryl, and 5-9 membered heteroaryl, wherein the C₁-C₃ alkyl, C₃-C₆ cycloalkyl, 4-7 membered heterocyclyl, C₁-C₆ alkoxy, C₆-C₁₀ aryl, or 5-9 membered heteroaryl is optionally substituted with one or more R^{c};
R^{c} is independently selected from the group consisting of halogen, hydroxy, amino, cyano, and C₁-C₃ alkyl.

2. The compound of formula (I) or the pharmaceutically acceptable salt thereof according to claim 1, wherein X² is selected from the group consisting of CR⁵R⁶, NR⁵, and CR⁵; or X² is selected from the group consisting of CR⁵R⁶ and CR⁵; or X² is CR⁵R⁶; or X² is CR⁵.

3. The compound of formula (I) or the pharmaceutically acceptable salt thereof according to claim 1, wherein X³ is selected from the group consisting of CR⁵′R⁶′, NR⁵′, and CR⁵′; or X³ is selected from the group consisting of CR⁵′R⁶′ and CR⁵′; or X³ is CR⁵′R⁶′ or X³ is CR⁵′.

4. The compound of formula (I) or the pharmaceutically acceptable salt thereof according to claim 1, wherein X⁴ and X⁵ are independently selected from the group consisting of a bond, C(R¹⁰)₂, and O; or X⁴ is a bond, —CH₂—, —CH(CH₃)—, or 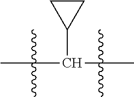 [SMILES: *C(*)C1CC1]
or X⁵ is a bond, —O—, —CH(CH₃)—, —C(CH₃)₂—, or 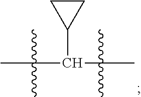 [SMILES: *C(*)C1CC1]
or X⁴ and X⁵ are both bonds; or X⁴ is —CH₂— and X⁵ is a bond.

5. The compound of formula (I) or the pharmaceutically acceptable salt thereof according to claim 1, wherein R¹ and R³ are independently selected from the group consisting of hydrogen and hydroxy; or R¹ is hydroxy and R³ is hydrogen.

6. The compound of formula (I) or the pharmaceutically acceptable salt thereof according to claim 1, wherein R² is hydrogen.

7. The compound of formula (I) or the pharmaceutically acceptable salt thereof according to claim 1, wherein R⁴, R⁶, R⁶′, and R¹⁰ are independently selected from the group consisting of hydrogen, amino, hydroxy, sulfydryl, halogen, cyano, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, and —C(═O)NH₂, wherein the amino, hydroxy, sulfydryl, C₁-C₆ alkyl, C₃-C₉ cycloalkyl, 4-9 membered heterocyclyl, C₆-C₁₀ aryl, 5-9 membered heteroaryl, C₂-C₆ alkenyl, C₂-C₆ alkynyl, or —C(═O)NH₂ is optionally substituted with one or more R^{a}; or
R⁴, R⁶, R⁶′, and R¹⁰ are independently selected from the group consisting of hydrogen, amino, hydroxy, C₁-C₆ alkyl, C₃-C₉ cycloalkyl, 4-9 membered heterocyclyl, C₆-C₁₀ aryl, and 5-9 membered heteroaryl, wherein the amino, hydroxy, C₁-C₆ alkyl, C₃-C₉ cycloalkyl, 4-9 membered heterocyclyl, C₆-C₁₀ aryl, or 5-9 membered heteroaryl is optionally substituted with one or more R^{a}, or R⁴, R⁶, R⁶′, and R¹⁰ are independently selected from the group consisting of hydrogen, amino, hydroxy, C₁-C₃ alkyl, C₃-C₆ cycloalkyl, 4-6 membered heterocyclyl, and 5-6 membered heteroaryl, wherein the amino, hydroxy, C₁-C₃ alkyl, C₃-C₆ cycloalkyl, 4-6 membered heterocyclyl, or 5-6 membered heteroaryl is optionally substituted with one or more R^{a}; or
R⁴ is selected from the group consisting of hydrogen, amino, hydroxy, C₁-C₆ alkyl, C₃-C₉ cycloalkyl, 4-9 membered heterocyclyl, C₆-C₁₀ aryl, and 5-9 membered heteroaryl, wherein the amino, hydroxy, C₁-C₆ alkyl, C₃-C₉ cycloalkyl, 4-9 membered heterocyclyl, C₆-C₁₀ aryl, or 5-9 membered heteroaryl is optionally substituted with one or more R^{a}; or
R⁴ is selected from the group consisting of —H, —CH₃, —CH(CH₃)₂, —OCH₃, —N(CH₃)₂, —CF₃, —CHF₂, —C(CH₃)₂(OH), 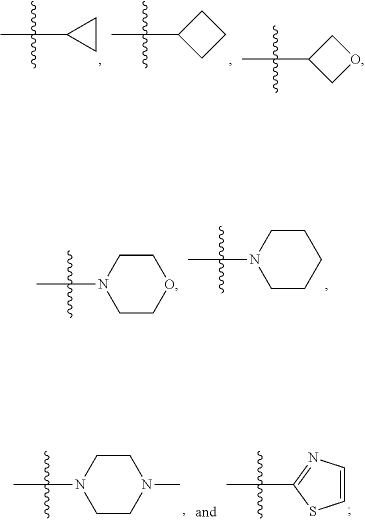
or
R⁴ is selected from the group consisting of H and methyl; or
each R¹⁰ is independently selected from the group consisting of hydrogen, C₁-C₆ alkyl, and C₃-C₆ cycloalkyl.

8. The compound of formula (I) or the pharmaceutically acceptable salt thereof according to claim 1, wherein R⁵ and R⁵′, together with the atoms linked thereto, form a C₄-C₇ saturated or partially saturated carbon ring, a benzene ring, a 5-6 heterocyclic ring, or a 5-6 membered heteroaromatic ring, wherein the C₄-C₇ saturated or partially saturated carbon ring, benzene ring, 5-6 heterocyclic ring, or 5-6 membered heteroaromatic ring is optionally substituted with one or more R^{a}, or R⁵ and R⁵′, together with the atoms linked thereto, form a benzene ring or a 5-6 membered heteroaromatic ring, wherein the benzene ring or 5-6 membered heteroaromatic ring is optionally substituted with one or more R^{a}; or
X² is CR⁵, X³ is CR⁵′, and R⁵ and R⁵′, together with the atoms linked thereto, form a benzene ring or a 5-6 membered heteroaromatic ring, wherein the benzene ring or 5-6 membered heteroaromatic ring is optionally substituted with one or more R^{a}; or
X² is CR⁵, X³ is CR⁵′, and R⁵ and R⁵′, together with the atoms linked thereto, form a benzene ring, a pyridine ring, a pyrazole ring, a thiazole ring, an isothiazole ring, a pyrrole ring, or a furan ring, wherein the benzene ring, pyridine ring, pyrazole ring, thiazole ring, isothiazole ring, pyrrole ring, or furan ring is optionally substituted with one or more R^{a}.

9. The compound of formula (I) or the pharmaceutically acceptable salt thereof according to claim 1, wherein R⁷ and R⁸ are independently selected from the group consisting of halogen and C₁-C₆ alkyl; or R⁷ and R⁸ are independently selected from the group consisting of C₁-C₃ alkyl; or R⁷ and R⁸ are both methyl.

10. The compound of formula (I) or the pharmaceutically acceptable salt thereof according to claim 1, wherein X¹ is N; or X¹ is CR⁹, and
R⁹ is selected from the group consisting of hydrogen, hydroxy, amino, halogen, and C₁-C₃ alkyl, wherein the hydroxy, amino, and C₁-C₃ alkyl are each optionally substituted with one or more R^{a}; or R⁹ is selected from the group consisting of hydrogen and methyl.

11. The compound of formula (I) or the pharmaceutically acceptable salt thereof according to claim 1, wherein n is selected from the group consisting of 0 and 1.

12. The compound of formula (I) or the pharmaceutically acceptable salt thereof according to claim 1, wherein R^{a} is independently selected from the group consisting of halogen, oxo, hydroxy, amino, cyano, C₁-C₆ alkyl, C₃-C₆ cycloalkyl, 4-7 membered heterocyclyl, C₆-C₁₀ aryl, and 5-9 membered heteroaryl, wherein the hydroxy, amino, C₁-C₆ alkyl, C₃-C₆ cycloalkyl, 4-7 membered heterocyclyl, C₆-C₁₀ aryl, or 5-9 membered heteroaryl is optionally substituted with one or more R^{b}; or R^{a} is independently selected from the group consisting of cyano, halogen, hydroxy, amino, C₁-C₆ alkyl, and C₃-C₆ cycloalkyl, wherein the hydroxy, amino, C₁-C₆ alkyl, and C₃-C₆ cycloalkyl are each optionally substituted with one or more R^{b}; or R^{a} is independently selected from the group consisting of halogen, hydroxy, cyano, C₁-C₆ alkyl, and C₃-C₆ cycloalkyl, wherein the hydroxy, C₁-C₆ alkyl, and C₃-C₆ cycloalkyl are each optionally substituted with one or more R^{b}; or R^{a} is independently selected from the group consisting of cyano, halogen, C₁-C₃ alkyl, C₁-C₃ alkoxy, C₁-C₃ haloalkyl, C₁-C₃ haloalkoxy, C₁-C₃ alkoxy-C₁-C₃ alkylene, C₃-C₆ cycloalkyl, C₃-C₆ cycloalkyloxy, and hydroxymethyl; or R^{a} is independently selected from the group consisting of cyano, halogen, C₁-C₃ alkyl, C₁-C₃ alkoxy, and C₁-C₃ haloalkyl; or R^{a} is independently selected from the group consisting of —OH, —CH₃, —C₂H₅, —OCH₃, —OCH₂CH₃, —CH₂CF₃, —F, —OCF₂H, —OCF₃, —CN, —CF₃, —CH₂OH, —OCH₂F, —CHF₂, —CH₂CH₂OCH₃, —CH(CH₃)₂, —N(CH₃)₂, —CH₂N(CH₃)₂, —CH₂OCH₃,

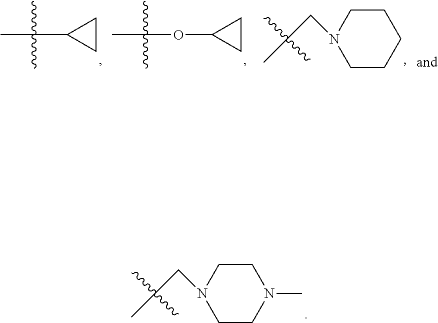

13. The compound of formula (I) or the pharmaceutically acceptable salt thereof according to claim 1, wherein R^{b} is independently selected from the group consisting of halogen, hydroxy, amino, cyano, C₁-C₃ alkyl, NH(C₁-C₃ alkyl), N(C₁-C₃ alkyl)₂, C₃-C₆ cycloalkyl, 4-7 membered heterocyclyl, C₁-C₆ alkoxy, C₆-C₁₀ aryl, and 5-9 membered heteroaryl, wherein the C₃-C₆ cycloalkyl, 4-7 membered heterocyclyl, C₁-C₆ alkoxy, C₆-C₁₀ aryl, or 5-9 membered heteroaryl is optionally substituted with one or more R^{c}; or R^{b} is independently selected from the group consisting of halogen, hydroxy, amino, cyano, NH(C₁-C₃ alkyl), N(C₁-C₃ alkyl)₂, C₃-C₆ cycloalkyl, 4-7 membered heterocyclyl, C₁-C₆ alkoxy, C₆-C₁₀ aryl, and 5-9 membered heteroaryl, wherein the C₃-C₆ cycloalkyl, 4-7 membered heterocyclyl, C₁-C₆ alkoxy, C₆-C₁₀ aryl, or 5-9 membered heteroaryl is optionally substituted with one or more R^{c}; or R^{b} is independently selected from the group consisting of amino, NH(C₁-C₃ alkyl), N(C₁-C₃ alkyl)₂, C₃-C₆ cycloalkyl, 4-7 membered heterocyclyl, and C₁-C₆ alkoxy, wherein the C₃-C₆ cycloalkyl, 4-7 membered heterocyclyl, and C₁-C₆ alkoxy are each optionally substituted with one or more R^{c}; or R^{b} is independently selected from the group consisting of halogen, hydroxy, C₁-C₃ alkyl, and C₃-C₆ cycloalkyl, wherein the hydroxy, C₁-C₃ alkyl, and C₃-C₆ cycloalkyl are each optionally substituted with one or more R^{c}; or R^{b} is independently selected from the group consisting of halogen, hydroxy, C₁-C₃ alkyl, N(C₁-C₃ alkyl)₂, C₃-C₆ cycloalkyl, 4-7 membered heterocyclyl, and C₁-C₆ alkoxy, wherein the C₁-C₃ alkyl, C₃-C₆ cycloalkyl, 4-7 membered heterocyclyl, and C₁-C₆ alkoxy are each optionally substituted with one or more R^{c}.

14. The compound of formula (I) or the pharmaceutically acceptable salt thereof according to claim 1, wherein R^{c} is independently selected from the group consisting of halogen and C₁-C₃ alkyl; or R^{c} is selected from the group consisting of C₁-C₃ alkyl; or R^{c} is independently halogen.

15. The compound of formula (I) or the pharmaceutically acceptable salt thereof according to claim 1, selected from the group consisting of a compound of formula (II) or a pharmaceutically acceptable salt thereof, 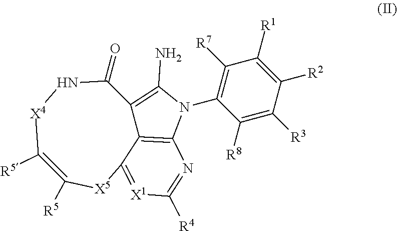
wherein R⁵ and R⁵′, together with the atoms linked thereto, form a C₆-C₁₀ aromatic ring or a 5-9 membered heteroaromatic ring, wherein the C₆-C₁₀ aromatic ring or 5-9 membered heteroaromatic ring is optionally substituted with one or more R^{a}; R¹, R², R³, R⁴, R⁷, R⁸, X¹, X⁴, X⁵, and R^{a} are as defined in claim 1.

16. The compound of formula (I) or the pharmaceutically acceptable salt thereof according to claim 1, selected from the group consisting of a compound of formula (III) or a pharmaceutically acceptable salt thereof, 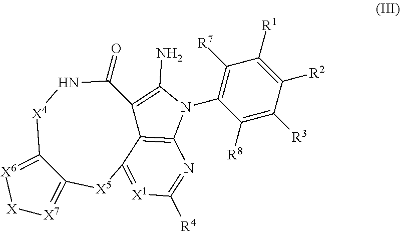
wherein X⁶ and X⁷ are independently selected from the group consisting of CH and N, wherein the CH is optionally substituted with halogen, C₁-C₃ alkyl, C₁-C₃ alkoxy, C₁-C₃ haloalkyl, and C₁-C₃ haloalkoxy; X is selected from the group consisting of NR^{a} and O; R¹, R², R³, R⁴, R⁷, R⁸, X¹, X⁴, X⁵, and R^{a} are as defined in claim 1.

17. The compound of formula (I) or the pharmaceutically acceptable salt thereof according to claim 1, selected from the group consisting of the following compounds or pharmaceutically acceptable salts thereof:

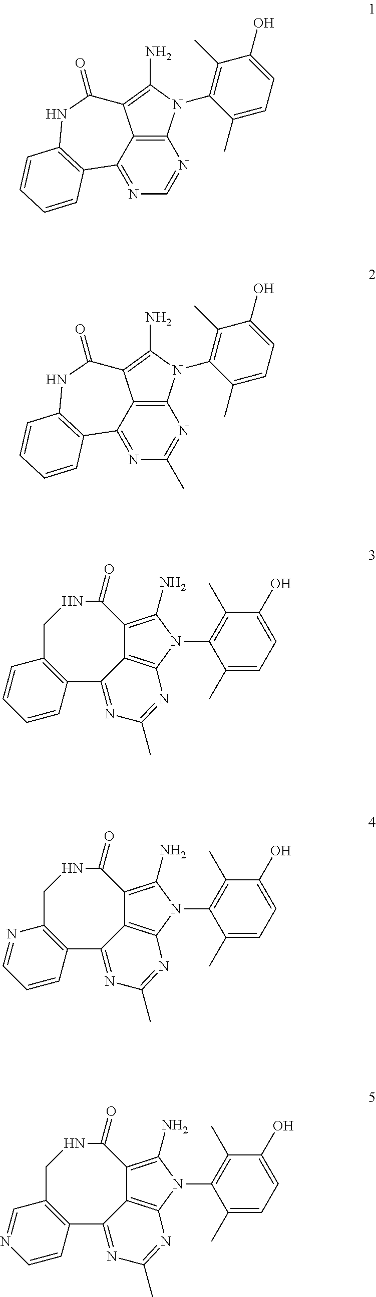 [SMILES: Cc1ccc(O)c(C)c1-n1c(N)c2c3c(ncnc31)-c1ccccc1NC2=O ; Cc1nc2c3c(c(N)n(-c4c(C)ccc(O)c4C)c3n1)C(=O)Nc1ccccc1-2 ; Cc1nc2c3c(c(N)n(-c4c(C)ccc(O)c4C)c3n1)C(=O)NCc1ccccc1-2 ; Cc1nc2c3c(c(N)n(-c4c(C)ccc(O)c4C)c3n1)C(=O)NCc1ncccc1-2 ; Cc1nc2c3c(c(N)n(-c4c(C)ccc(O)c4C)c3n1)C(=O)NCc1cnccc1-2]

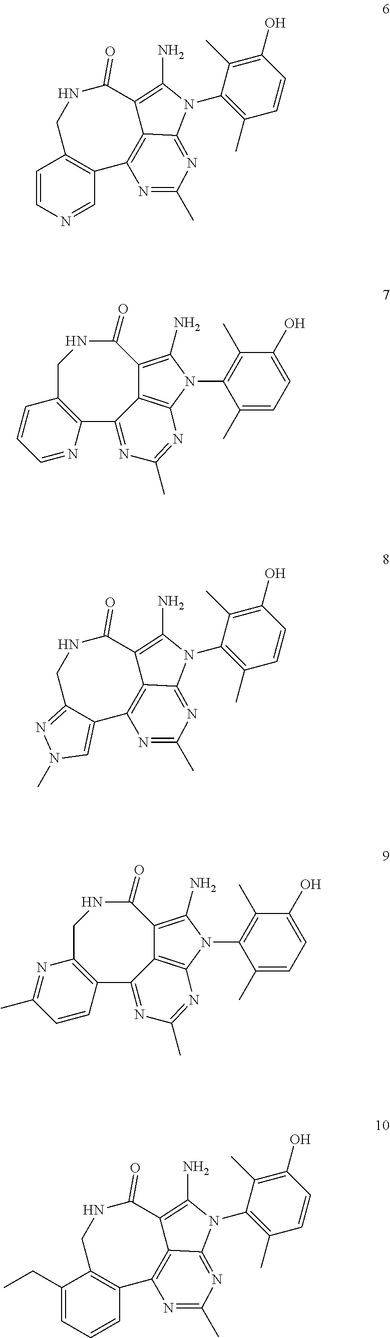 [SMILES: Cc1nc2c3c(c(N)n(-c4c(C)ccc(O)c4C)c3n1)C(=O)NCc1ccncc1-2 ; Cc1nc2c3c(c(N)n(-c4c(C)ccc(O)c4C)c3n1)C(=O)NCc1cccnc1-2 ; Cc1nc2c3c(c(N)n(-c4c(C)ccc(O)c4C)c3n1)C(=O)NCc1nn(C)cc1-2 ; Cc1ccc2c(n1)CNC(=O)c1c(N)n(-c3c(C)ccc(O)c3C)c3nc(C)nc-2c13 ; CCc1cccc2c1CNC(=O)c1c(N)n(-c3c(C)ccc(O)c3C)c3nc(C)nc-2c13]

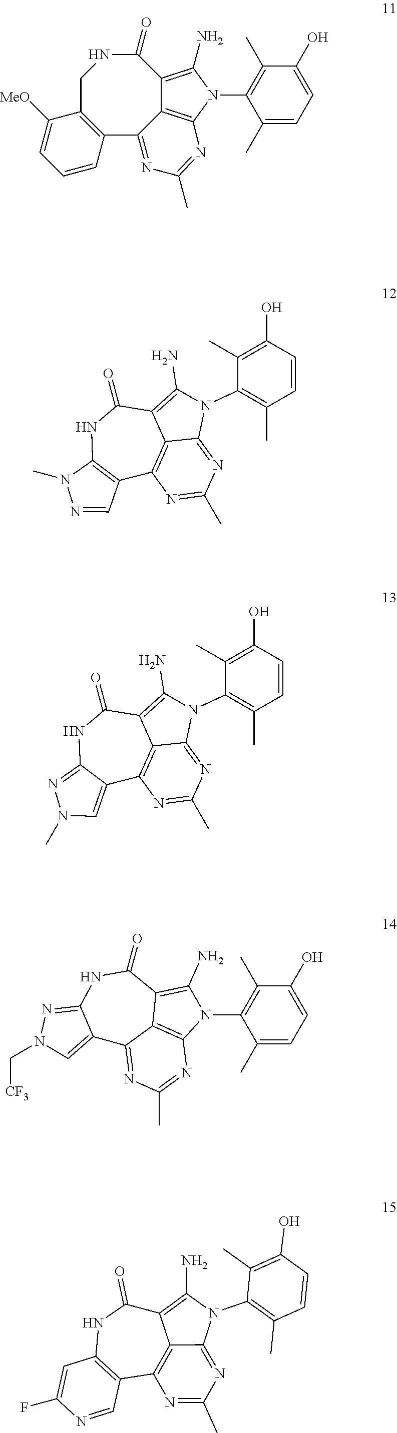 [SMILES: COc1cccc2c1CNC(=O)c1c(N)n(-c3c(C)ccc(O)c3C)c3nc(C)nc-2c13 ; Cc1nc2c3c(c(N)n(-c4c(C)ccc(O)c4C)c3n1)C(=O)Nc1c-2cnn1C ; Cc1nc2c3c(c(N)n(-c4c(C)ccc(O)c4C)c3n1)C(=O)Nc1nn(C)cc1-2 ; Cc1nc2c3c(c(N)n(-c4c(C)ccc(O)c4C)c3n1)C(=O)Nc1nn(CC(F)(F)F)cc1-2 ; Cc1nc2c3c(c(N)n(-c4c(C)ccc(O)c4C)c3n1)C(=O)Nc1cc(F)ncc1-2]

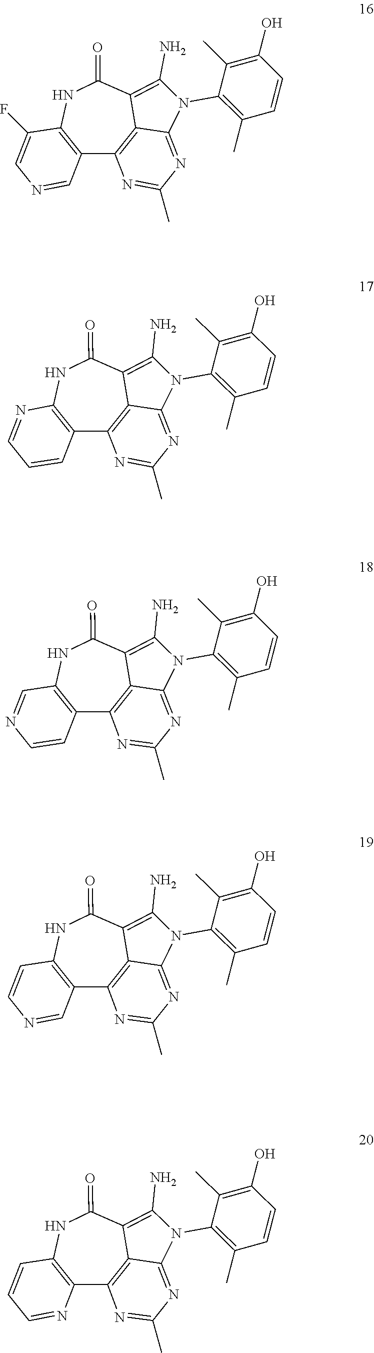 [SMILES: Cc1nc2c3c(c(N)n(-c4c(C)ccc(O)c4C)c3n1)C(=O)Nc1c(F)cncc1-2 ; Cc1nc2c3c(c(N)n(-c4c(C)ccc(O)c4C)c3n1)C(=O)Nc1ncccc1-2 ; Cc1nc2c3c(c(N)n(-c4c(C)ccc(O)c4C)c3n1)C(=O)Nc1cnccc1-2 ; Cc1nc2c3c(c(N)n(-c4c(C)ccc(O)c4C)c3n1)C(=O)Nc1ccncc1-2 ; Cc1nc2c3c(c(N)n(-c4c(C)ccc(O)c4C)c3n1)C(=O)Nc1cccnc1-2]

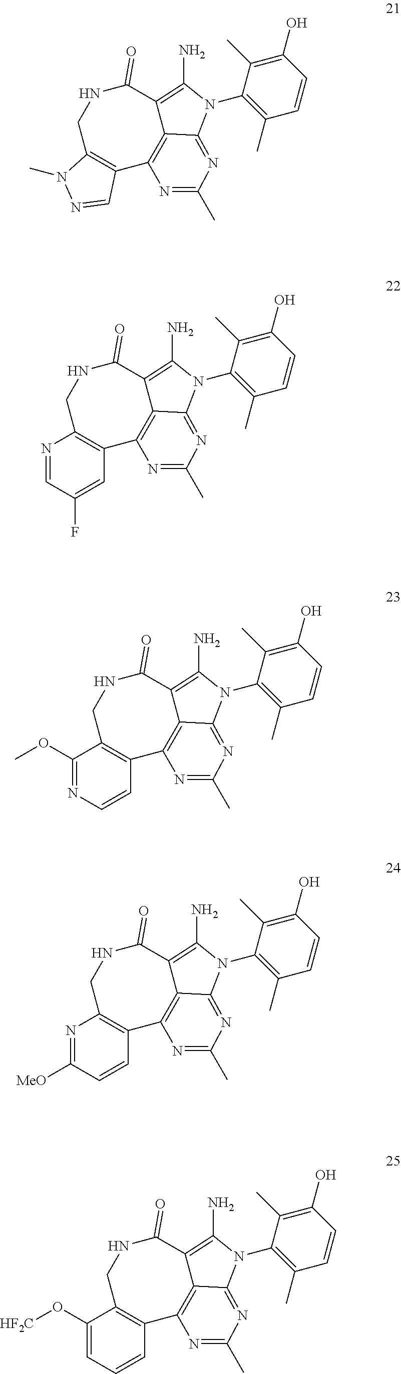

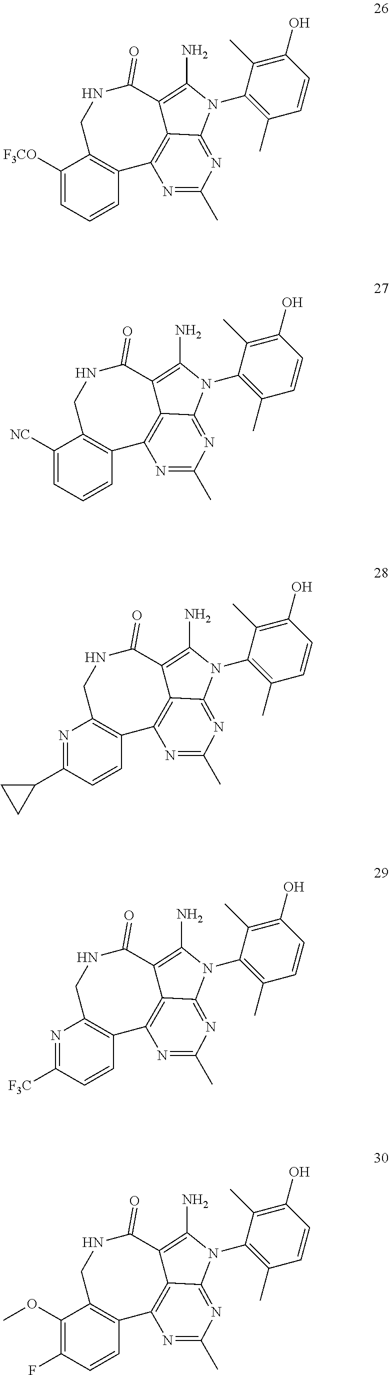 [SMILES: Cc1nc2c3c(c(N)n(-c4c(C)ccc(O)c4C)c3n1)C(=O)NCc1c(OC(F)(F)F)cccc1-2 ; Cc1nc2c3c(c(N)n(-c4c(C)ccc(O)c4C)c3n1)C(=O)NCc1c(C#N)cccc1-2 ; Cc1nc2c3c(c(N)n(-c4c(C)ccc(O)c4C)c3n1)C(=O)NCc1nc(C3CC3)ccc1-2 ; Cc1nc2c3c(c(N)n(-c4c(C)ccc(O)c4C)c3n1)C(=O)NCc1nc(C(F)(F)F)ccc1-2 ; COc1c(F)ccc2c1CNC(=O)c1c(N)n(-c3c(C)ccc(O)c3C)c3nc(C)nc-2c13]

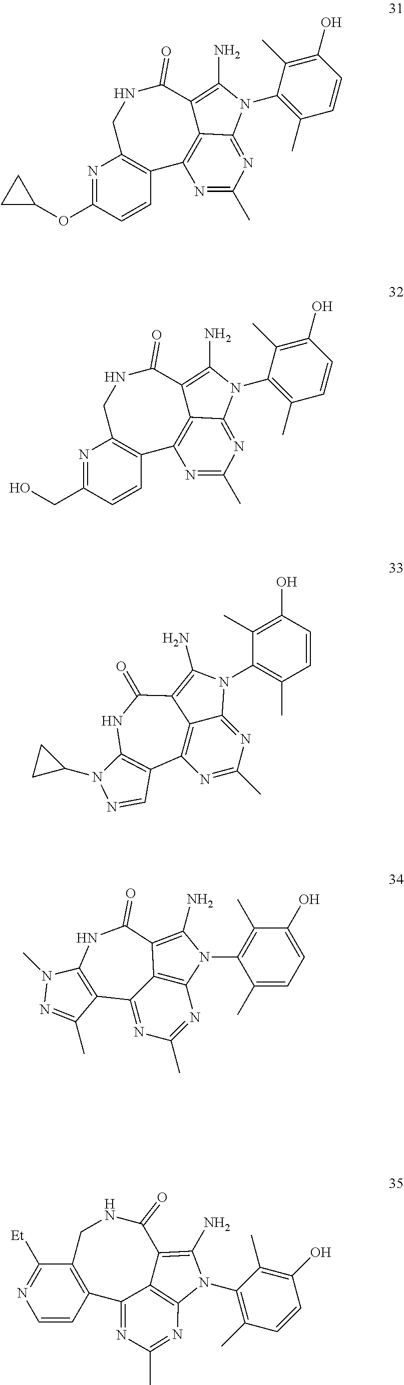 [SMILES: Cc1nc2c3c(c(N)n(-c4c(C)ccc(O)c4C)c3n1)C(=O)NCc1nc(OC3CC3)ccc1-2 ; Cc1nc2c3c(c(N)n(-c4c(C)ccc(O)c4C)c3n1)C(=O)NCc1nc(CO)ccc1-2 ; Cc1nc2c3c(c(N)n(-c4c(C)ccc(O)c4C)c3n1)C(=O)Nc1c-2cnn1C1CC1 ; Cc1nc2c3c(c(N)n(-c4c(C)ccc(O)c4C)c3n1)C(=O)Nc1c-2c(C)nn1C ; CCc1nccc2c1CNC(=O)c1c(N)n(-c3c(C)ccc(O)c3C)c3nc(C)nc-2c13]

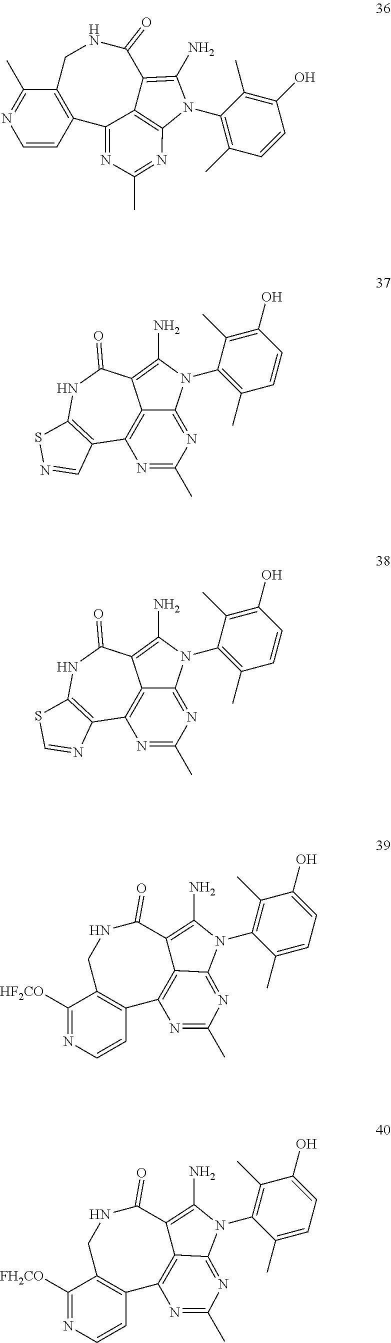

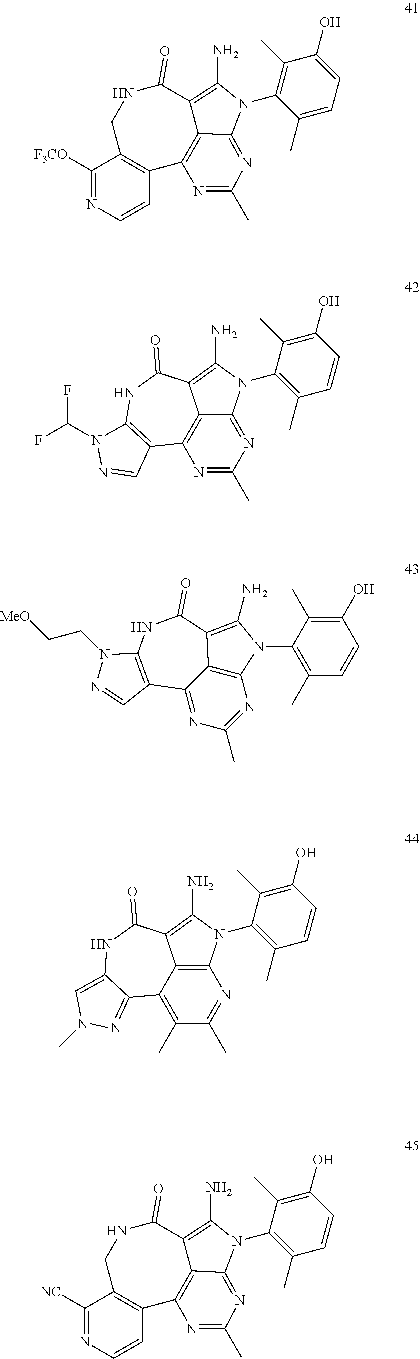 [SMILES: Cc1nc2c3c(c(N)n(-c4c(C)ccc(O)c4C)c3n1)C(=O)NCc1c-2ccnc1OC(F)(F)F ; Cc1nc2c3c(c(N)n(-c4c(C)ccc(O)c4C)c3n1)C(=O)Nc1c-2cnn1C(F)F ; COCCn1ncc2c1NC(=O)c1c(N)n(-c3c(C)ccc(O)c3C)c3nc(C)nc-2c13 ; Cc1ccc(O)c(C)c1-n1c(N)c2c3c(c(C)c(C)nc31)-c1nn(C)cc1NC2=O ; Cc1nc2c3c(c(N)n(-c4c(C)ccc(O)c4C)c3n1)C(=O)NCc1c-2ccnc1C#N]

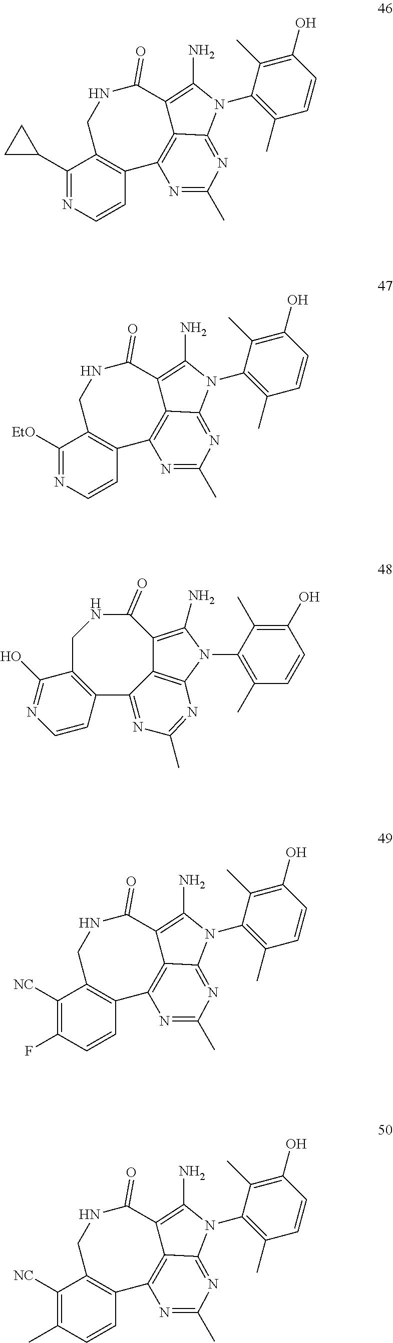 [SMILES: Cc1nc2c3c(c(N)n(-c4c(C)ccc(O)c4C)c3n1)C(=O)NCc1c-2ccnc1C1CC1 ; CCOc1nccc2c1CNC(=O)c1c(N)n(-c3c(C)ccc(O)c3C)c3nc(C)nc-2c13 ; Cc1nc2c3c(c(N)n(-c4c(C)ccc(O)c4C)c3n1)C(=O)NCc1c-2ccnc1O ; Cc1nc2c3c(c(N)n(-c4c(C)ccc(O)c4C)c3n1)C(=O)NCc1c-2ccc(F)c1C#N ; Cc1nc2c3c(c(N)n(-c4c(C)ccc(O)c4C)c3n1)C(=O)NCc1c-2ccc(C)c1C#N]

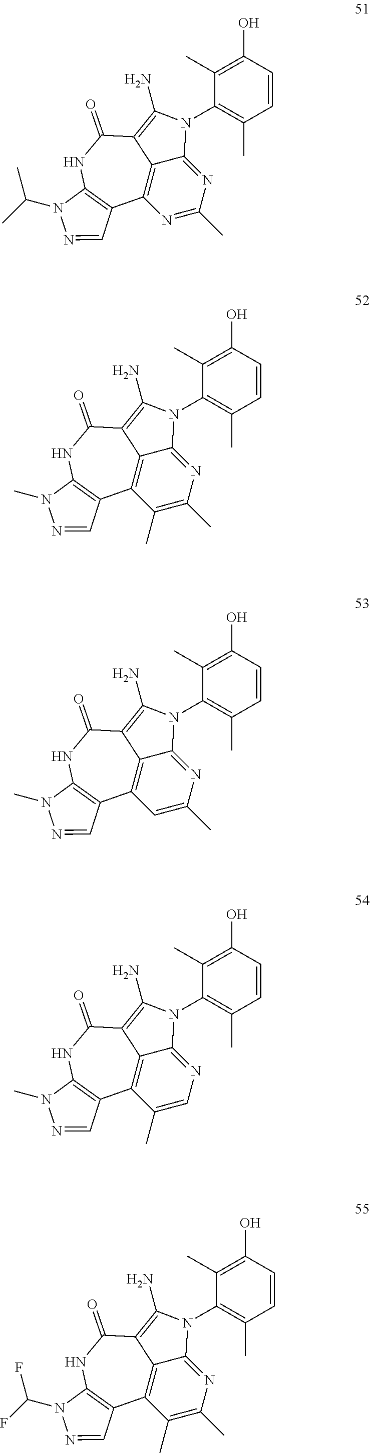 [SMILES: Cc1nc2c3c(c(N)n(-c4c(C)ccc(O)c4C)c3n1)C(=O)Nc1c-2cnn1C(C)C ; Cc1ccc(O)c(C)c1-n1c(N)c2c3c(c(C)c(C)nc31)-c1cnn(C)c1NC2=O ; Cc1cc2c3c(c(N)n(-c4c(C)ccc(O)c4C)c3n1)C(=O)Nc1c-2cnn1C ; Cc1ccc(O)c(C)c1-n1c(N)c2c3c(c(C)cnc31)-c1cnn(C)c1NC2=O ; Cc1ccc(O)c(C)c1-n1c(N)c2c3c(c(C)c(C)nc31)-c1cnn(C(F)F)c1NC2=O]

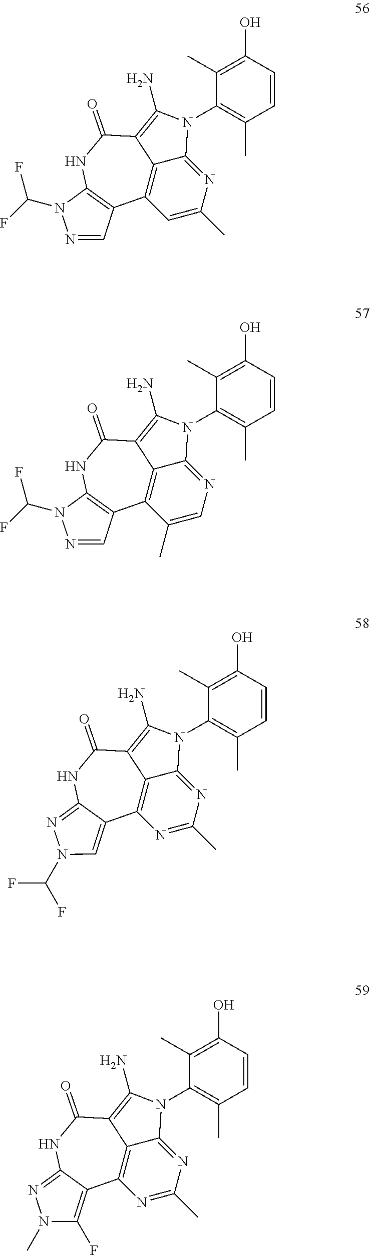 [SMILES: Cc1cc2c3c(c(N)n(-c4c(C)ccc(O)c4C)c3n1)C(=O)Nc1c-2cnn1C(F)F ; Cc1ccc(O)c(C)c1-n1c(N)c2c3c(c(C)cnc31)-c1cnn(C(F)F)c1NC2=O ; Cc1nc2c3c(c(N)n(-c4c(C)ccc(O)c4C)c3n1)C(=O)Nc1nn(C(F)F)cc1-2 ; Cc1nc2c3c(c(N)n(-c4c(C)ccc(O)c4C)c3n1)C(=O)Nc1nn(C)c(F)c1-2]

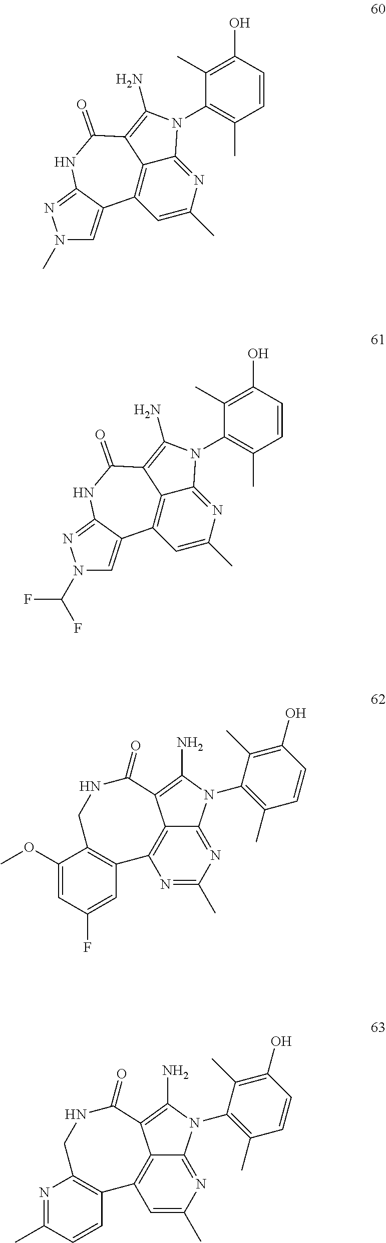 [SMILES: Cc1cc2c3c(c(N)n(-c4c(C)ccc(O)c4C)c3n1)C(=O)Nc1nn(C)cc1-2 ; Cc1cc2c3c(c(N)n(-c4c(C)ccc(O)c4C)c3n1)C(=O)Nc1nn(C(F)F)cc1-2 ; COc1cc(F)cc2c1CNC(=O)c1c(N)n(-c3c(C)ccc(O)c3C)c3nc(C)nc-2c13 ; Cc1ccc2c(n1)CNC(=O)c1c(N)n(-c3c(C)ccc(O)c3C)c3nc(C)cc-2c13]

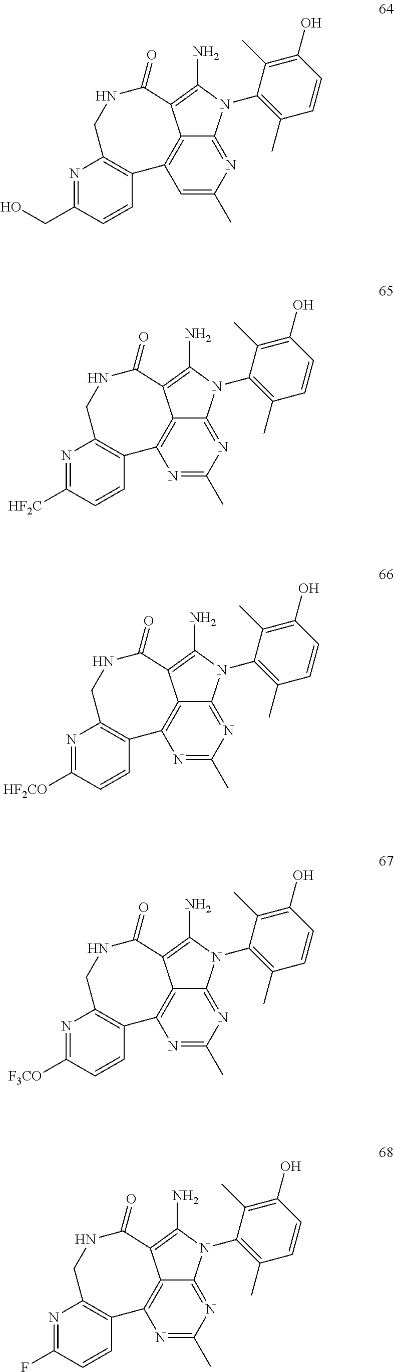

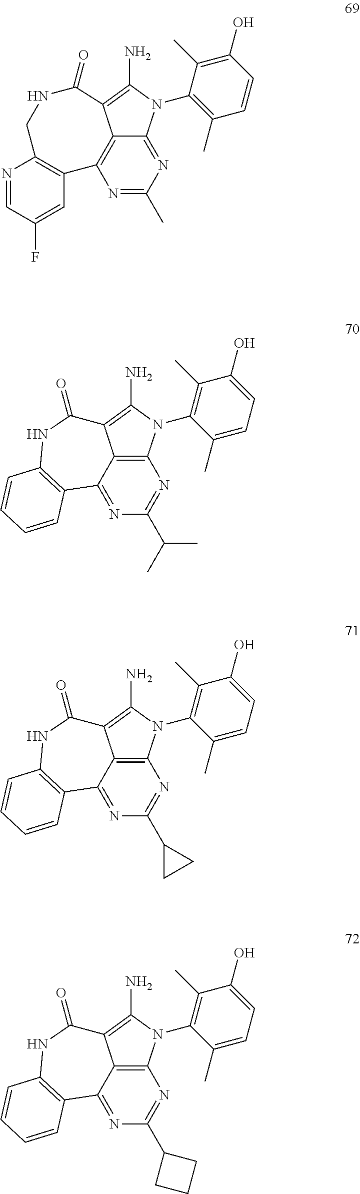 [SMILES: Cc1nc2c3c(c(N)n(-c4c(C)ccc(O)c4C)c3n1)C(=O)NCc1ncc(F)cc1-2 ; Cc1ccc(O)c(C)c1-n1c(N)c2c3c(nc(C(C)C)nc31)-c1ccccc1NC2=O ; Cc1ccc(O)c(C)c1-n1c(N)c2c3c(nc(C4CC4)nc31)-c1ccccc1NC2=O ; Cc1ccc(O)c(C)c1-n1c(N)c2c3c(nc(C4CCC4)nc31)-c1ccccc1NC2=O]

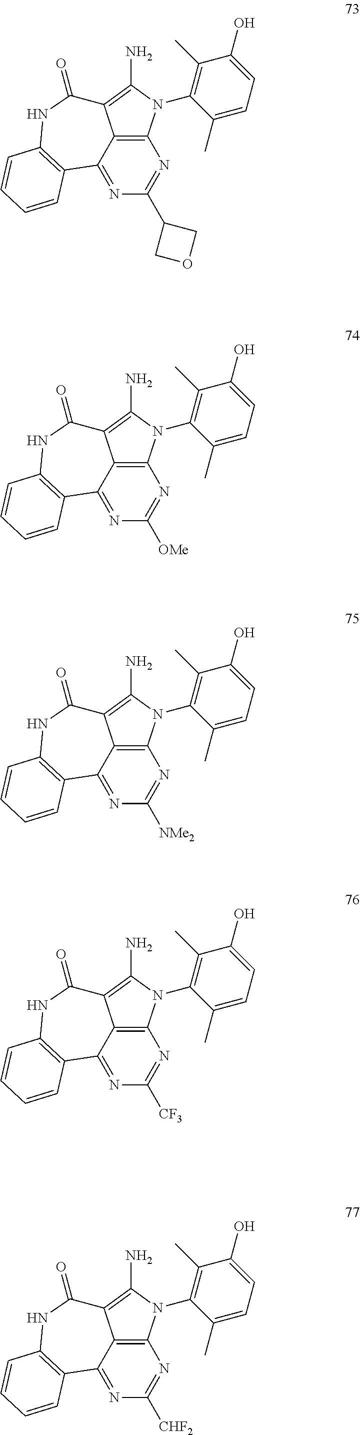 [SMILES: Cc1ccc(O)c(C)c1-n1c(N)c2c3c(nc(C4COC4)nc31)-c1ccccc1NC2=O ; COc1nc2c3c(c(N)n(-c4c(C)ccc(O)c4C)c3n1)C(=O)Nc1ccccc1-2 ; Cc1ccc(O)c(C)c1-n1c(N)c2c3c(nc(N(C)C)nc31)-c1ccccc1NC2=O ; Cc1ccc(O)c(C)c1-n1c(N)c2c3c(nc(C(F)(F)F)nc31)-c1ccccc1NC2=O ; Cc1ccc(O)c(C)c1-n1c(N)c2c3c(nc(C(F)F)nc31)-c1ccccc1NC2=O]

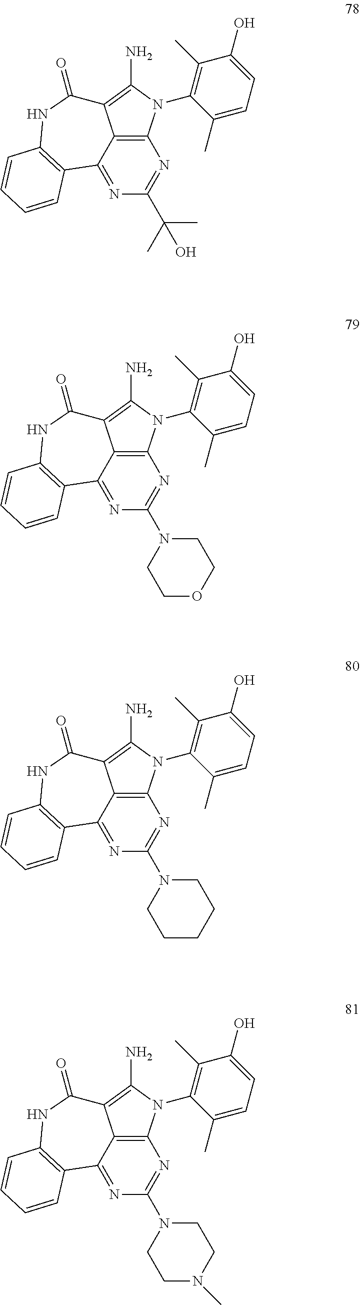 [SMILES: Cc1ccc(O)c(C)c1-n1c(N)c2c3c(nc(C(C)(C)O)nc31)-c1ccccc1NC2=O ; Cc1ccc(O)c(C)c1-n1c(N)c2c3c(nc(N4CCOCC4)nc31)-c1ccccc1NC2=O ; Cc1ccc(O)c(C)c1-n1c(N)c2c3c(nc(N4CCCCC4)nc31)-c1ccccc1NC2=O ; Cc1ccc(O)c(C)c1-n1c(N)c2c3c(nc(N4CCN(C)CC4)nc31)-c1ccccc1NC2=O]

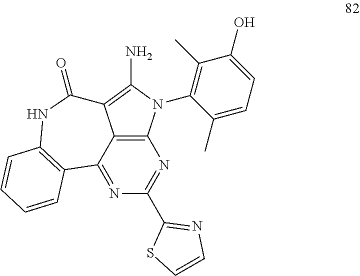 [SMILES: Cc1ccc(O)c(C)c1-n1c(N)c2c3c(nc(-c4nccs4)nc31)-c1ccccc1NC2=O]

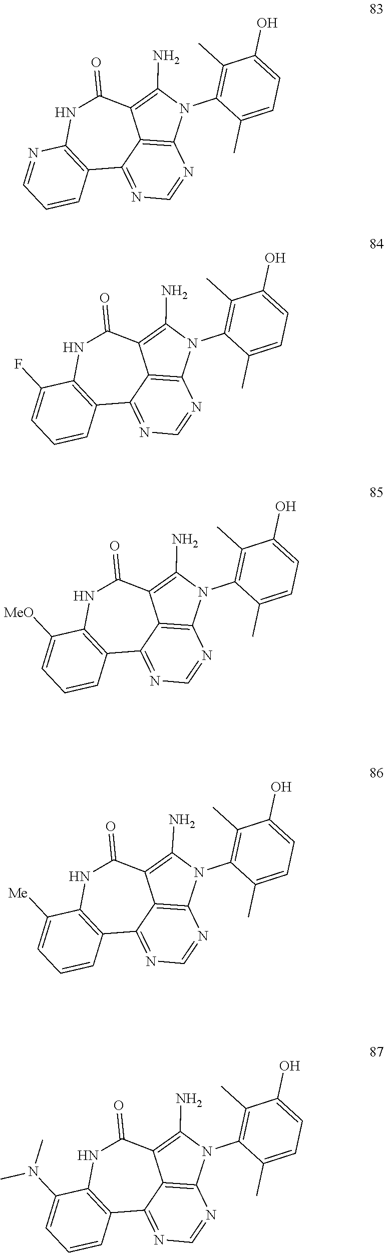 [SMILES: Cc1ccc(O)c(C)c1-n1c(N)c2c3c(ncnc31)-c1cccnc1NC2=O ; Cc1ccc(O)c(C)c1-n1c(N)c2c3c(ncnc31)-c1cccc(F)c1NC2=O ; COc1cccc2c1NC(=O)c1c(N)n(-c3c(C)ccc(O)c3C)c3ncnc-2c13 ; Cc1cccc2c1NC(=O)c1c(N)n(-c3c(C)ccc(O)c3C)c3ncnc-2c13 ; Cc1ccc(O)c(C)c1-n1c(N)c2c3c(ncnc31)-c1cccc(N(C)C)c1NC2=O]

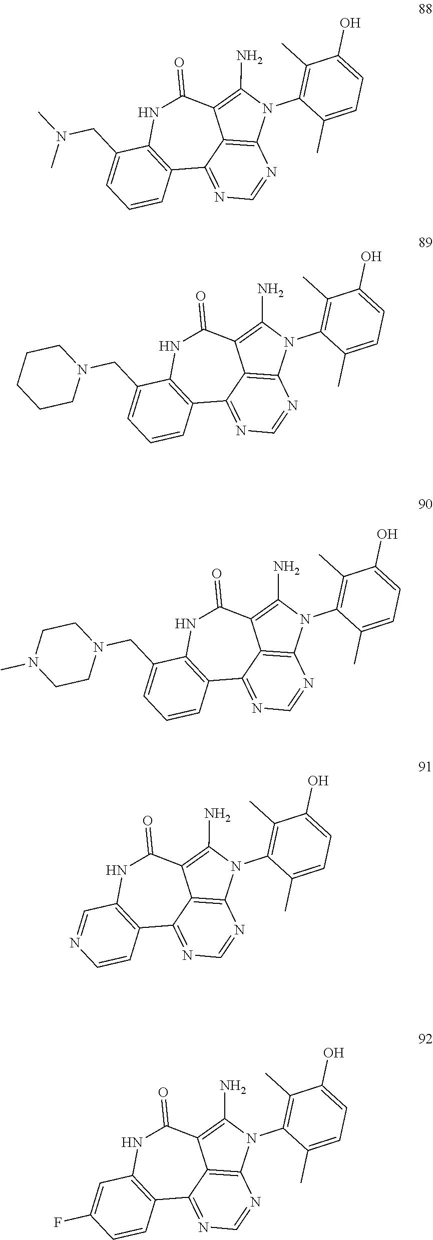 [SMILES: Cc1ccc(O)c(C)c1-n1c(N)c2c3c(ncnc31)-c1cccc(CN(C)C)c1NC2=O ; Cc1ccc(O)c(C)c1-n1c(N)c2c3c(ncnc31)-c1cccc(CN3CCCCC3)c1NC2=O ; Cc1ccc(O)c(C)c1-n1c(N)c2c3c(ncnc31)-c1cccc(CN3CCN(C)CC3)c1NC2=O ; Cc1ccc(O)c(C)c1-n1c(N)c2c3c(ncnc31)-c1ccncc1NC2=O ; Cc1ccc(O)c(C)c1-n1c(N)c2c3c(ncnc31)-c1ccc(F)cc1NC2=O]

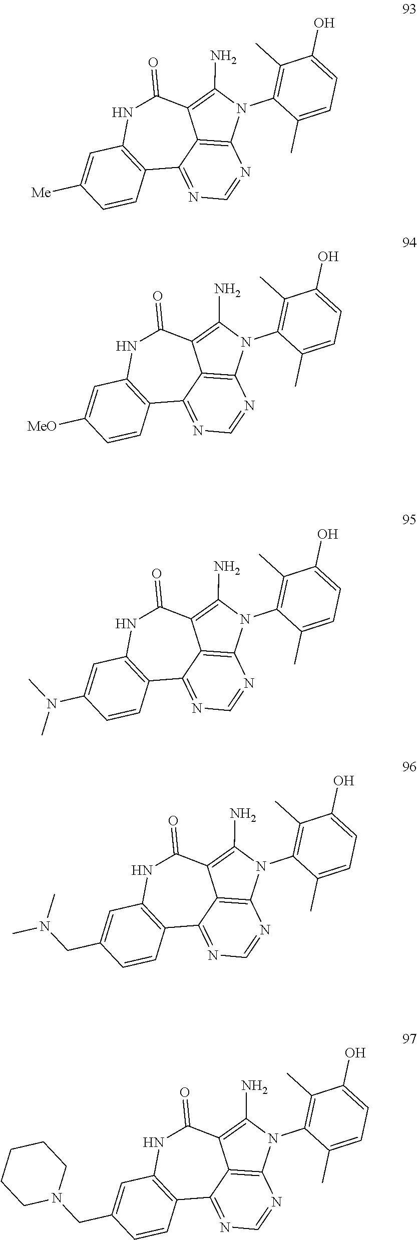 [SMILES: Cc1ccc2c(c1)NC(=O)c1c(N)n(-c3c(C)ccc(O)c3C)c3ncnc-2c13 ; COc1ccc2c(c1)NC(=O)c1c(N)n(-c3c(C)ccc(O)c3C)c3ncnc-2c13 ; Cc1ccc(O)c(C)c1-n1c(N)c2c3c(ncnc31)-c1ccc(N(C)C)cc1NC2=O ; Cc1ccc(O)c(C)c1-n1c(N)c2c3c(ncnc31)-c1ccc(CN(C)C)cc1NC2=O ; Cc1ccc(O)c(C)c1-n1c(N)c2c3c(ncnc31)-c1ccc(CN3CCCCC3)cc1NC2=O]

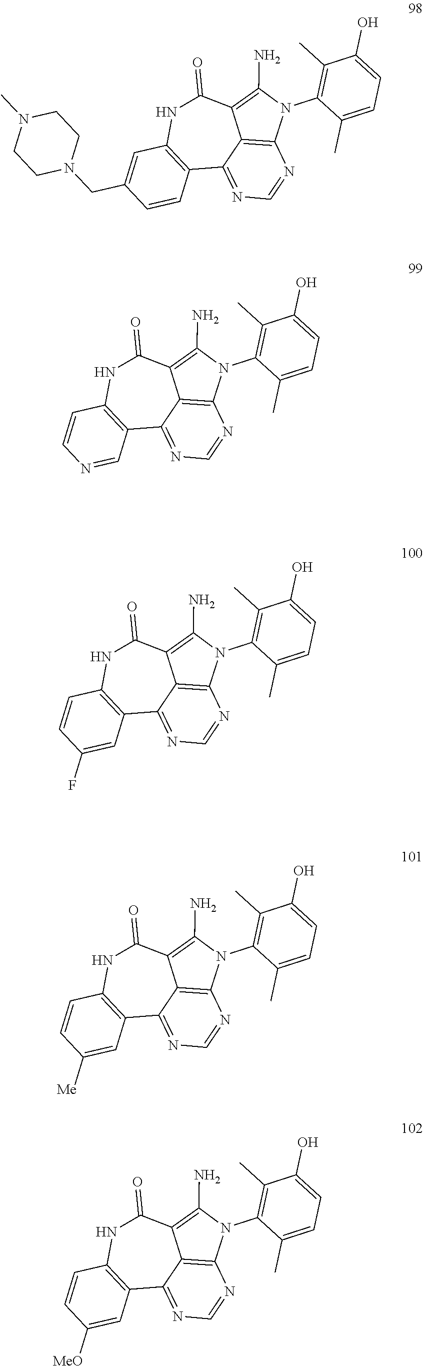 [SMILES: Cc1ccc(O)c(C)c1-n1c(N)c2c3c(ncnc31)-c1ccc(CN3CCN(C)CC3)cc1NC2=O ; Cc1ccc(O)c(C)c1-n1c(N)c2c3c(ncnc31)-c1cnccc1NC2=O ; Cc1ccc(O)c(C)c1-n1c(N)c2c3c(ncnc31)-c1cc(F)ccc1NC2=O ; Cc1ccc2c(c1)-c1ncnc3c1c(c(N)n3-c1c(C)ccc(O)c1C)C(=O)N2 ; COc1ccc2c(c1)-c1ncnc3c1c(c(N)n3-c1c(C)ccc(O)c1C)C(=O)N2]

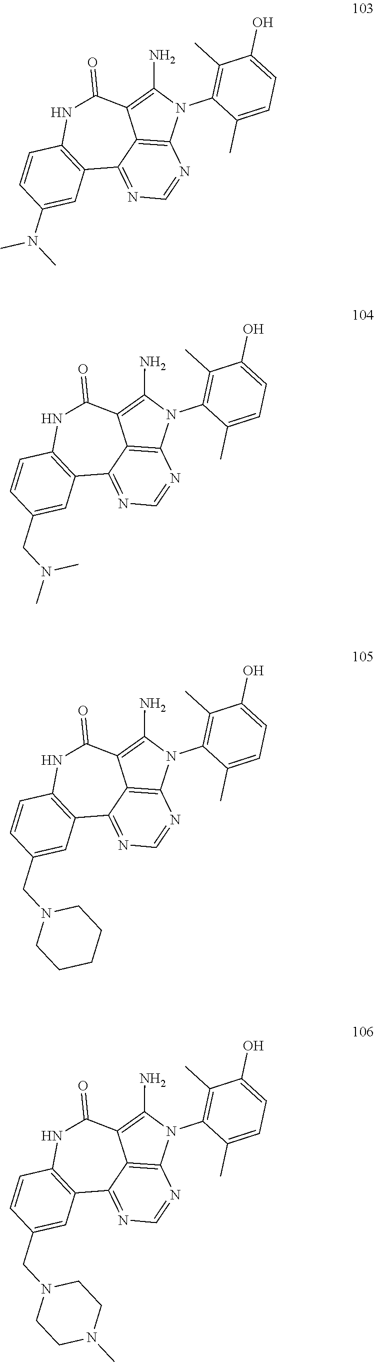 [SMILES: Cc1ccc(O)c(C)c1-n1c(N)c2c3c(ncnc31)-c1cc(N(C)C)ccc1NC2=O ; Cc1ccc(O)c(C)c1-n1c(N)c2c3c(ncnc31)-c1cc(CN(C)C)ccc1NC2=O ; Cc1ccc(O)c(C)c1-n1c(N)c2c3c(ncnc31)-c1cc(CN3CCCCC3)ccc1NC2=O ; Cc1ccc(O)c(C)c1-n1c(N)c2c3c(ncnc31)-c1cc(CN3CCN(C)CC3)ccc1NC2=O]

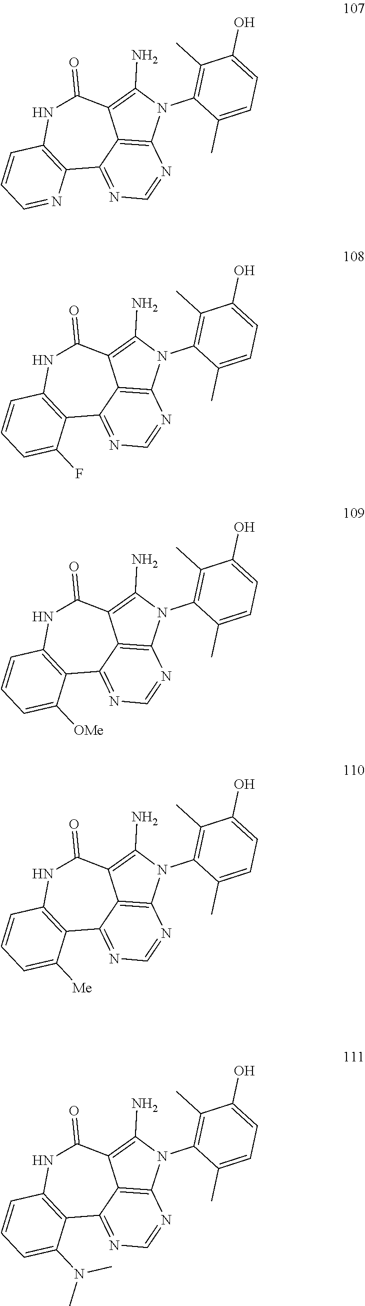 [SMILES: Cc1ccc(O)c(C)c1-n1c(N)c2c3c(ncnc31)-c1ncccc1NC2=O ; Cc1ccc(O)c(C)c1-n1c(N)c2c3c(ncnc31)-c1c(F)cccc1NC2=O ; COc1cccc2c1-c1ncnc3c1c(c(N)n3-c1c(C)ccc(O)c1C)C(=O)N2 ; Cc1cccc2c1-c1ncnc3c1c(c(N)n3-c1c(C)ccc(O)c1C)C(=O)N2 ; Cc1ccc(O)c(C)c1-n1c(N)c2c3c(ncnc31)-c1c(cccc1N(C)C)NC2=O]

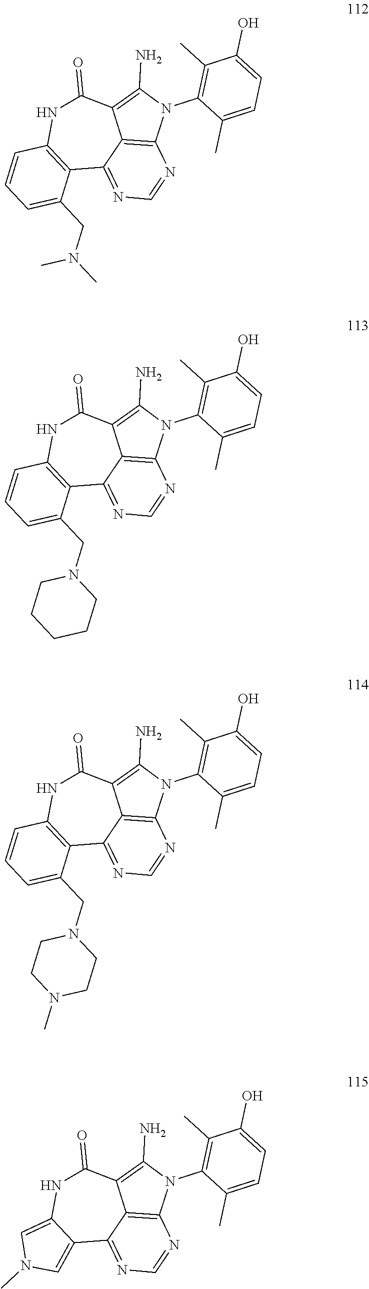 [SMILES: Cc1ccc(O)c(C)c1-n1c(N)c2c3c(ncnc31)-c1c(CN(C)C)cccc1NC2=O ; Cc1ccc(O)c(C)c1-n1c(N)c2c3c(ncnc31)-c1c(CN3CCCCC3)cccc1NC2=O ; Cc1ccc(O)c(C)c1-n1c(N)c2c3c(ncnc31)-c1c(CN3CCN(C)CC3)cccc1NC2=O ; Cc1ccc(O)c(C)c1-n1c(N)c2c3c(ncnc31)-c1cn(C)cc1NC2=O]

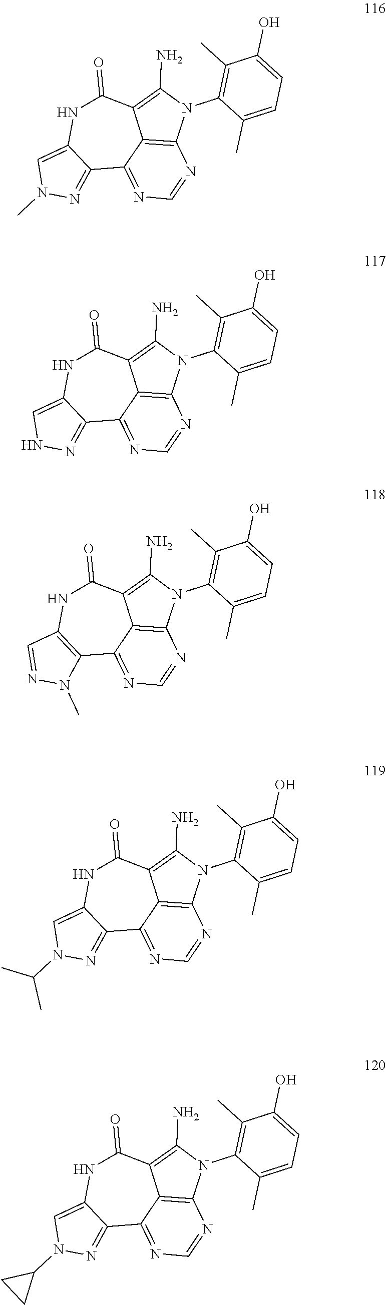 [SMILES: Cc1ccc(O)c(C)c1-n1c(N)c2c3c(ncnc31)-c1nn(C)cc1NC2=O ; Cc1ccc(O)c(C)c1-n1c(N)c2c3c(ncnc31)-c1n[nH]cc1NC2=O ; Cc1ccc(O)c(C)c1-n1c(N)c2c3c(ncnc31)-c1c(cnn1C)NC2=O ; Cc1ccc(O)c(C)c1-n1c(N)c2c3c(ncnc31)-c1nn(C(C)C)cc1NC2=O ; Cc1ccc(O)c(C)c1-n1c(N)c2c3c(ncnc31)-c1nn(C3CC3)cc1NC2=O]

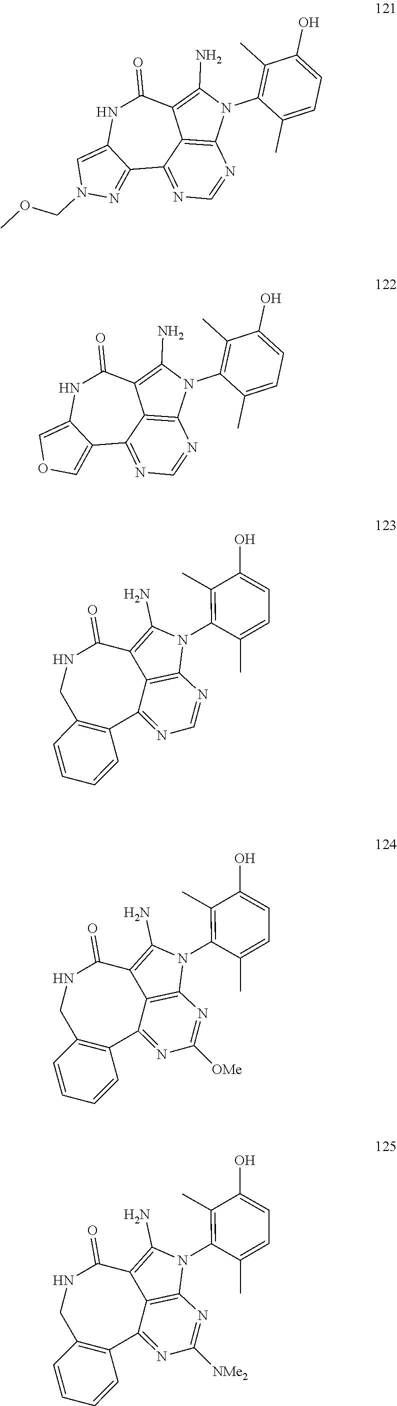 [SMILES: COCn1cc2c(n1)-c1ncnc3c1c(c(N)n3-c1c(C)ccc(O)c1C)C(=O)N2 ; Cc1ccc(O)c(C)c1-n1c(N)c2c3c(ncnc31)-c1cocc1NC2=O ; Cc1ccc(O)c(C)c1-n1c(N)c2c3c(ncnc31)-c1ccccc1CNC2=O ; COc1nc2c3c(c(N)n(-c4c(C)ccc(O)c4C)c3n1)C(=O)NCc1ccccc1-2 ; Cc1ccc(O)c(C)c1-n1c(N)c2c3c(nc(N(C)C)nc31)-c1ccccc1CNC2=O]

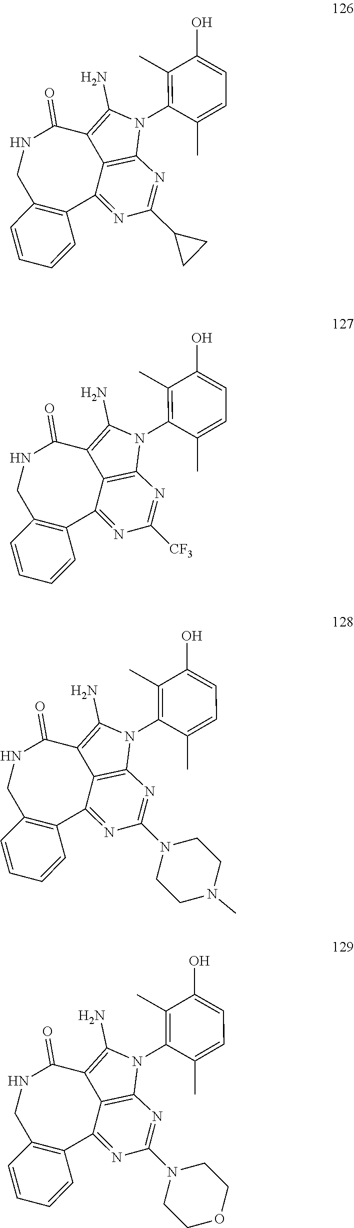 [SMILES: Cc1ccc(O)c(C)c1-n1c(N)c2c3c(nc(C4CC4)nc31)-c1ccccc1CNC2=O ; Cc1ccc(O)c(C)c1-n1c(N)c2c3c(nc(C(F)(F)F)nc31)-c1ccccc1CNC2=O ; Cc1ccc(O)c(C)c1-n1c(N)c2c3c(nc(N4CCN(C)CC4)nc31)-c1ccccc1CNC2=O ; Cc1ccc(O)c(C)c1-n1c(N)c2c3c(nc(N4CCOCC4)nc31)-c1ccccc1CNC2=O]

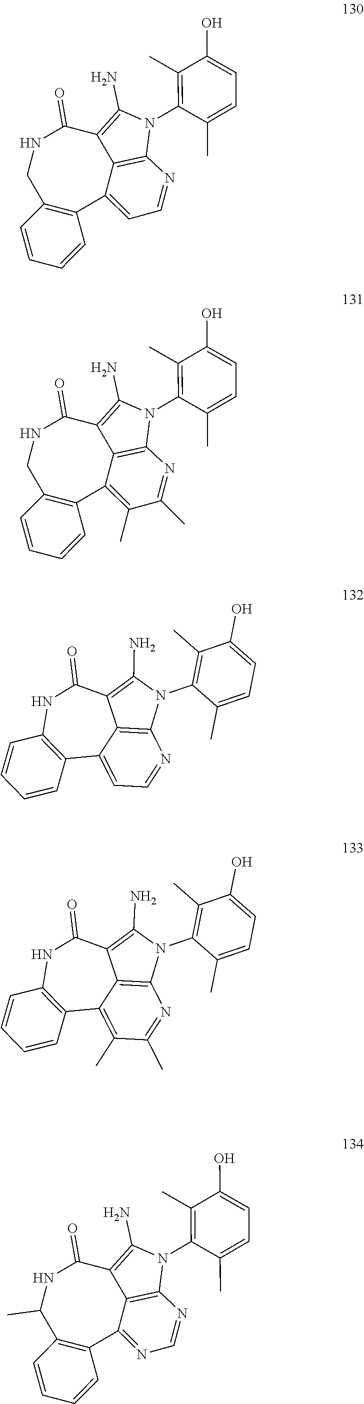 [SMILES: Cc1ccc(O)c(C)c1-n1c(N)c2c3c(ccnc31)-c1ccccc1CNC2=O ; Cc1ccc(O)c(C)c1-n1c(N)c2c3c(c(C)c(C)nc31)-c1ccccc1CNC2=O ; Cc1ccc(O)c(C)c1-n1c(N)c2c3c(ccnc31)-c1ccccc1NC2=O ; Cc1ccc(O)c(C)c1-n1c(N)c2c3c(c(C)c(C)nc31)-c1ccccc1NC2=O ; Cc1ccc(O)c(C)c1-n1c(N)c2c3c(ncnc31)-c1ccccc1C(C)NC2=O]

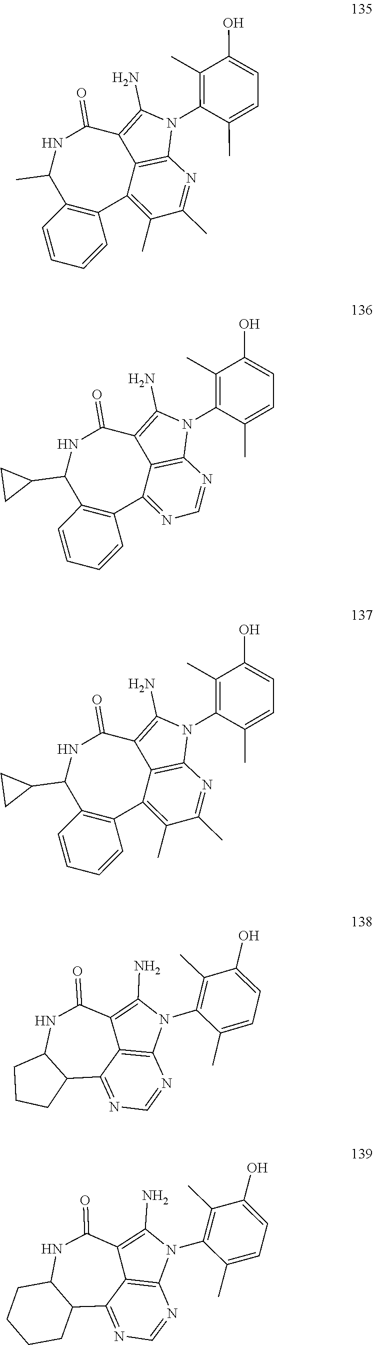 [SMILES: Cc1ccc(O)c(C)c1-n1c(N)c2c3c(c(C)c(C)nc31)-c1ccccc1C(C)NC2=O ; Cc1ccc(O)c(C)c1-n1c(N)c2c3c(ncnc31)-c1ccccc1C(C1CC1)NC2=O ; Cc1ccc(O)c(C)c1-n1c(N)c2c3c(c(C)c(C)nc31)-c1ccccc1C(C1CC1)NC2=O ; Cc1ccc(O)c(C)c1-n1c(N)c2c3c(ncnc31)C1CCCC1NC2=O ; Cc1ccc(O)c(C)c1-n1c(N)c2c3c(ncnc31)C1CCCCC1NC2=O]

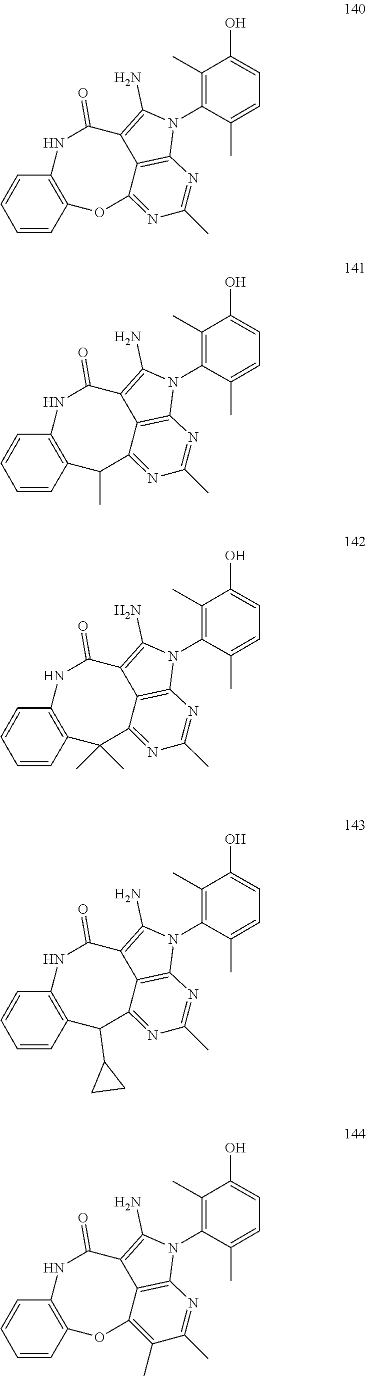 [SMILES: Cc1nc2c3c(c(N)n(-c4c(C)ccc(O)c4C)c3n1)C(=O)Nc1ccccc1O2 ; Cc1nc2c3c(c(N)n(-c4c(C)ccc(O)c4C)c3n1)C(=O)Nc1ccccc1C2C ; Cc1nc2c3c(c(N)n(-c4c(C)ccc(O)c4C)c3n1)C(=O)Nc1ccccc1C2(C)C ; Cc1nc2c3c(c(N)n(-c4c(C)ccc(O)c4C)c3n1)C(=O)Nc1ccccc1C2C1CC1 ; Cc1ccc(O)c(C)c1-n1c(N)c2c3c(c(C)c(C)nc31)Oc1ccccc1NC2=O]

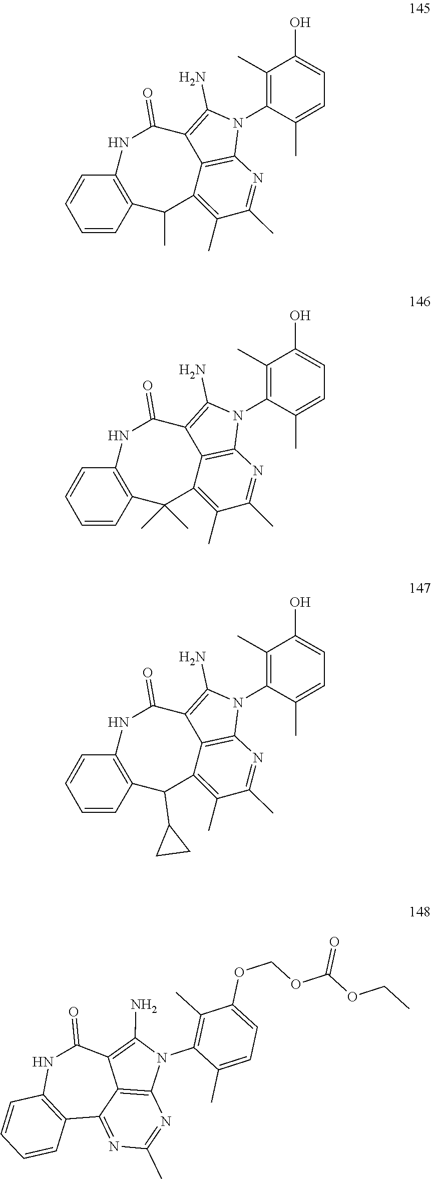 [SMILES: Cc1ccc(O)c(C)c1-n1c(N)c2c3c(c(C)c(C)nc31)C(C)c1ccccc1NC2=O ; Cc1ccc(O)c(C)c1-n1c(N)c2c3c(c(C)c(C)nc31)C(C)(C)c1ccccc1NC2=O ; Cc1ccc(O)c(C)c1-n1c(N)c2c3c(c(C)c(C)nc31)C(C1CC1)c1ccccc1NC2=O ; CCOC(=O)OCOc1ccc(C)c(-n2c(N)c3c4c(nc(C)nc42)-c2ccccc2NC3=O)c1C]

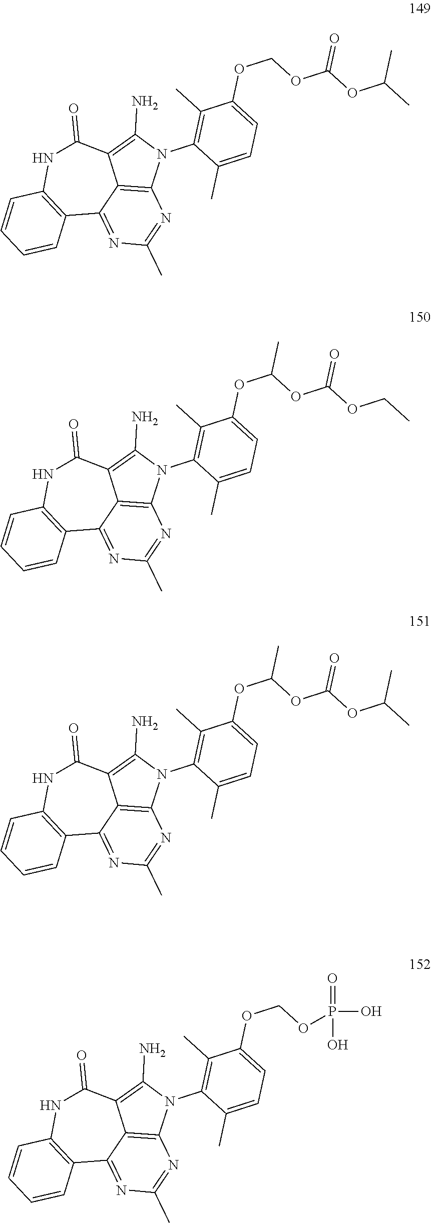 [SMILES: Cc1nc2c3c(c(N)n(-c4c(C)ccc(OCOC(=O)OC(C)C)c4C)c3n1)C(=O)Nc1ccccc1-2 ; CCOC(=O)OC(C)Oc1ccc(C)c(-n2c(N)c3c4c(nc(C)nc42)-c2ccccc2NC3=O)c1C ; Cc1nc2c3c(c(N)n(-c4c(C)ccc(OC(C)OC(=O)OC(C)C)c4C)c3n1)C(=O)Nc1ccccc1-2 ; Cc1nc2c3c(c(N)n(-c4c(C)ccc(OCOP(=O)(O)O)c4C)c3n1)C(=O)Nc1ccccc1-2]

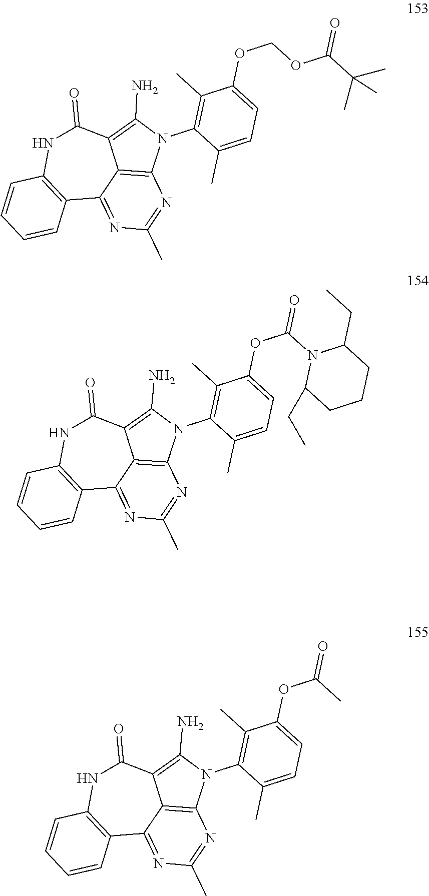 [SMILES: Cc1nc2c3c(c(N)n(-c4c(C)ccc(OCOC(=O)C(C)(C)C)c4C)c3n1)C(=O)Nc1ccccc1-2 ; CCC1CCCC(CC)N1C(=O)Oc1ccc(C)c(-n2c(N)c3c4c(nc(C)nc42)-c2ccccc2NC3=O)c1C ; CC(=O)Oc1ccc(C)c(-n2c(N)c3c4c(nc(C)nc42)-c2ccccc2NC3=O)c1C]

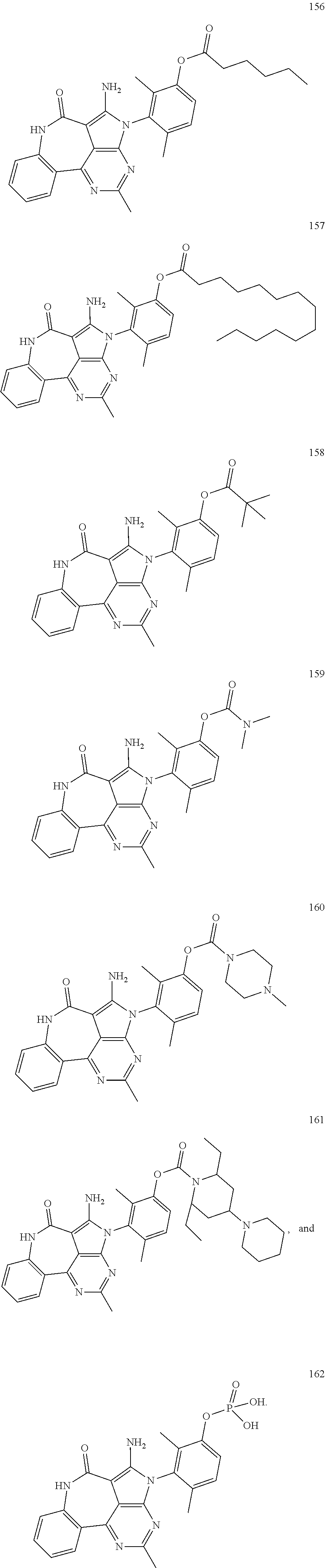

18. A pharmaceutical composition, comprising the compound of formula (I) or the pharmaceutically acceptable salt thereof according to claim 1 and a pharmaceutically acceptable excipient.

19. A method for treating diseases mediated by PKMYT1 in a mammal, comprising administering to a mammal, preferably a human, in need of such treatment a therapeutically effective amount of the compound of formula (I) or the pharmaceutically acceptable salt thereof according to claim 1; preferably, said diseases mediated by PKMYT1 are tumors, or said diseases mediated by PKMYT1 are selected from the group consisting of liver cancer and breast cancer.

20. A method for treating diseases mediated by PKMYT1 in a mammal, comprising administering to a mammal, preferably a human, in need of such treatment a therapeutically effective amount of the pharmaceutical composition according to claim 18; preferably, said diseases mediated by PKMYT1 are tumors, or said diseases mediated by PKMYT1 are selected from the group consisting of liver cancer and breast cancer.
